SEQUENCE LISTING (50 sequences)

SEQ ID NO: 1 - protein, 95 aa, synthetic construct
  REGION 1..95: note = Synthetic
QAGGSLRLSCAASGYIFSDTYMGWYRQAPGKEREFVAGINGGGTTNYADSVKGRFTISRDNAKNTVYLQMNSLKPEDTAVYYCAVYYGSFSWSLL

SEQ ID NO: 2 - protein, 114 aa, synthetic construct
  REGION 1..114: note = Synthetic
AGGSLRLSCAASGNISYSYGMGWYRQAPGKEREFVAGITFGGSTYYADSVKGRFTISRDNAKNTVYLQMNSLKPEDTAVYYCAVYTRHVDTTFARHWYWGQGTQVTVSSLEHHH

SEQ ID NO: 3 - protein, 109 aa, synthetic construct
  REGION 1..109: note = Synthetic
AGGSLRLSCAASGNIFYGQPMGWYRQAPGKEREFVAGIGRGGSTYYADSVKGRFTISRDNAKNTVYLQMNSLKPEDTAVYYCAVLSQYPYRHTYWGQGTQVTVSSLEHH

SEQ ID NO: 4 - protein, 113 aa, synthetic construct
  REGION 1..113: note = Synthetic
QAGGSLRLSCAASGTISTYGMGWYRQAPGKEREFVAGIATGGTTYYADSVKGRFTISRDNAKNTVYLQMNSLKPEDTAVYYCAALDKYARHYVYWGQGTQVTVSSLEHHHHHH

SEQ ID NO: 5 - protein, 112 aa, synthetic construct
  REGION 1..112: note = Synthetic
QAGGSLRLSCAASGTIFYRYSMGWYRQAPGKEREFVAGITEGSNTYYADSVKGRFTISRDNAKNTVYLQMNSLKPEDTAVYYCAVVQRVDLTYWGQGTQVTVSSLEHHHHHH

SEQ ID NO: 6 - protein, 118 aa, synthetic construct
  REGION 1..118: note = Synthetic
QAGGSLRLSCAASGNIFRVIGMGWYRQAPGKEREFVAGIGSGSSTYYADSVKGRFTISRDNAKNTVYLQMNSLKPEDTAVYYCAAFTYPYYDQTKLLPYWGQGTQVTVSSLEHHHHHH

SEQ ID NO: 7 - protein, 118 aa, synthetic construct
  REGION 1..118: note = Synthetic
QAGGSLRLSCAASGTIFPRANMGWYRQAPGKEREFVAGITLGGTTYYADSVKGRFTISRDNAKNTVYLQMNSLKPEDTAVYYCAVVYKTYRYQEILYYYWGQGTQVTVSSLEHHHHHH

SEQ ID NO: 8 - protein, 7 aa, synthetic construct
  REGION 1..7: note = Synthetic
NISYSYG

SEQ ID NO: 9 - protein, 7 aa, synthetic construct
  REGION 1..7: note = Synthetic
GITFGGS

SEQ ID NO: 10 - protein, 14 aa, synthetic construct
  REGION 1..14: note = Synthetic
VYTRHVDTTFARHW

SEQ ID NO: 11 - protein, 7 aa, synthetic construct
  REGION 1..7: note = Synthetic
NIFYGQP

SEQ ID NO: 12 - protein, 7 aa, synthetic construct
  REGION 1..7: note = Synthetic
GIGRGGS

SEQ ID NO: 13 - protein, 10 aa, synthetic construct
  REGION 1..10: note = Synthetic
VLSQYPYRHT

SEQ ID NO: 14 - protein, 6 aa, synthetic construct
  REGION 1..6: note = Synthetic
TISTYG

SEQ ID NO: 15 - protein, 7 aa, synthetic construct
  REGION 1..7: note = Synthetic
GIATGGT

SEQ ID NO: 16 - protein, 10 aa, synthetic construct
  REGION 1..10: note = Synthetic
ALDKYARHYV

SEQ ID NO: 17 - protein, 7 aa, synthetic construct
  REGION 1..7: note = Synthetic
TIFYRYS

SEQ ID NO: 18 - protein, 7 aa, synthetic construct
  REGION 1..7: note = Synthetic
GITEGSN

SEQ ID NO: 19 - protein, 8 aa, synthetic construct
  REGION 1..8: note = Synthetic
VVQRVDLT

SEQ ID NO: 20 - protein, 7 aa, synthetic construct
  REGION 1..7: note = Synthetic
NIFRVIG

SEQ ID NO: 21 - protein, 7 aa, synthetic construct
  REGION 1..7: note = Synthetic
GIGSGSS

SEQ ID NO: 22 - protein, 14 aa, synthetic construct
  REGION 1..14: note = Synthetic
AFTYPYYDQTKLLP

SEQ ID NO: 23 - protein, 7 aa, synthetic construct
  REGION 1..7: note = Synthetic
TIFPRAN

SEQ ID NO: 24 - protein, 7 aa, synthetic construct
  REGION 1..7: note = Synthetic
GITLGGT

SEQ ID NO: 25 - protein, 14 aa, synthetic construct
  REGION 1..14: note = Synthetic
VVYKTYRYQEILYY

SEQ ID NO: 26 - DNA, 351 bp, synthetic construct
  misc_feature 1..351: note = Synthetic
  source 1..351: mol_type = other DNA
gcgggcggcagcctgcgcctgagctgcgcggcgagcggcaatatttcttactcttacggtatgggctggtatcgccaggcgccgggcaaagaacgcgaatttgttgccggtattactttcggtggtagtacctattatgcggatagcgtgaaaggccgctttaccattagccgcgataacgcgaaaaacaccgtgtatctgcagatgaacagcctgaaaccggaagataccgcggtgtattattgcgcggtttacactcgtcacgttgacactactttcgctcgtcattggtattggggccagggcacccaggtgaccgtgagcagcctcgagcaccaccaccaccaccac

SEQ ID NO: 27 - DNA, 339 bp, synthetic construct
  misc_feature 1..339: note = Synthetic
  source 1..339: mol_type = other DNA
gcgggcggcagcctgcgcctgagctgcgcggcgagcggcaatattttttacggtcagccgatgggctggtatcgccaggcgccgggcaaagaacgcgaatttgttgccggtattggtcgtggtggtagtacctattatgcggatagcgtgaaaggccgctttaccattagccgcgataacgcgaaaaacaccgtgtatctgcagatgaacagcctgaaaccggaagataccgcggtgtattattgcgcggttctgtctcagtacccgtaccgtcatacttattggggccagggcacccaggtgaccgtgagcagcctcgagcaccaccaccaccaccac

SEQ ID NO: 28 - DNA, 330 bp, synthetic construct
  misc_feature 1..330: note = Synthetic
  source 1..330: mol_type = other DNA
ggcagcctgcgcctgagctgcgcggcgagcggcactatttctacttacggtatgggctggtatcgccaggcgccgggcaaagaacgcgaatttgttgccggtattgctactggtggtactacctattatgcggatagcgtgaaaggccgctttaccattagccgcgataacgcgaaaaacaccgtgtatctgcagatgaacagcctgaaaccggaagataccgcggtgtattattgcgcggctctggacaaatacgctcgtcattatgtttattggggccagggcacccaggtgaccgtgagcagcctcgagcaccaccaccaccaccac

SEQ ID NO: 29 - DNA, 333 bp, synthetic construct
  misc_feature 1..333: note = Synthetic
  source 1..333: mol_type = other DNA
gcgggcggcagcctgcgcctgagctgcgcggcgagcggcactattttttaccgttactctatgggctggtatcgccaggcgccgggcaaagaacgcgaatttgttgccggtattactgaaggtagtaatacctattatgcggatagcgtgaaaggccgctttaccattagccgcgataacgcgaaaaacaccgtgtatctgcagatgaacagcctgaaaccggaagataccgcggtgtattattgcgcggttgttcagcgtgttgaccttacttattggggccagggcacccaggtgaccgtgagcagcctcgagcaccaccaccaccaccac

SEQ ID NO: 30 - DNA, 351 bp, synthetic construct
  misc_feature 1..351: note = Synthetic
  source 1..351: mol_type = other DNA
gcgggcggcagcctgcgcctgagctgcgcggcgagcggcaatatttttcgtgttatcggtatgggctggtatcgccaggcgccgggcaaagaacgcgaatttgttgccggtattggttctggtagtagtacctattatgcggatagcgtgaaaggccgctttaccattagccgcgataacgcgaaaaacaccgtgtatctgcagatgaacagcctgaaaccggaagataccgcggtgtattattgcgcggctttcacttacccgtactacgaccagactaaactgcttccgtattggggccagggcacccaggtgaccgtgagcagcctcgagcaccaccaccaccaccac

SEQ ID NO: 31 - DNA, 351 bp, synthetic construct
  misc_feature 1..351: note = Synthetic
  source 1..351: mol_type = other DNA
gcgggcggcagcctgcgcctgagctgcgcggcgagcggcactatttttccgcgtgctaacatgggctggtatcgccaggcgccgggcaaagaacgcgaatttgttgccggtattactctgggtggtactacctattatgcggatagcgtgaaaggccgctttaccattagccgcgataacgcgaaaaacaccgtgtatctgcagatgaacagcctgaaaccggaagataccgcggtgtattattgcgcggttgtttacaaaacttaccgttaccaggaaatcctgtattactattggggccagggcacccaggtgaccgtgagcagcctcgagcaccaccaccaccaccac

SEQ ID NO: 32 - protein, 20 aa, synthetic construct
  REGION 1..20: note = Synthetic
  REPEAT 1..20: note = This sequence may encompass 1-4 "GGGGS" repeating units
GGGGSGGGGSGGGGSGGGGS

SEQ ID NO: 33 - protein, 8 aa, synthetic construct
  REGION 1..8: note = Synthetic
GGGGGGGG

SEQ ID NO: 34 - protein, 6 aa, synthetic construct
  REGION 1..6: note = Synthetic
GGGGGG

SEQ ID NO: 35 - protein, 15 aa, synthetic construct
  REGION 1..15: note = Synthetic
EAAAKEAAAKEAAAK

SEQ ID NO: 36 - protein, 15 aa, synthetic construct
  REGION 1..15: note = Synthetic
  REPEAT 1..15: note = This sequence may encompass 1-3 "EAAAK" repeating units
EAAAKEAAAKEAAAK

SEQ ID NO: 37 - protein, 46 aa, synthetic construct
  REGION 1..46: note = Synthetic
AEAAAKEAAAKEAAAKEAAAKALEAEAAAKEAAAKEAAAKEAAAKA

SEQ ID NO: 38 - protein, 5 aa, synthetic construct
  REGION 1..5: note = Synthetic
PAPAP

SEQ ID NO: 39 - protein, 12 aa, synthetic construct
  REGION 1..12: note = Synthetic
AEAAAKEAAAKA

SEQ ID NO: 40 - protein, 68 aa, synthetic construct
  REGION 1..68: note = Synthetic
  REPEAT 1..68: note = This sequence may encompass 10-34 "AP" repeating units
APAPAPAPAPAPAPAPAPAPAPAPAPAPAPAPAPAPAPAPAPAPAPAPAPAPAPAPAPAPAPAPAPAP

SEQ ID NO: 41 - protein, 10 aa, synthetic construct
  REGION 1..10: note = Synthetic
GGGGSGGGGS

SEQ ID NO: 42 - protein, 5 aa, synthetic construct
  REGION 1..5: note = Synthetic
GGGGS

SEQ ID NO: 43 - protein, 50 aa, synthetic construct
  REGION 1..50: note = Synthetic
  REPEAT 1..50: note = This sequence may encompass 1-10 "GGGGS" repeating units
GGGGSGGGGSGGGGSGGGGSGGGGSGGGGSGGGGSGGGGSGGGGSGGGGS

SEQ ID NO: 44 - protein, 50 aa, synthetic construct
  REGION 1..50: note = Synthetic
  REPEAT 1..50: note = This sequence may encompass 1-10 "SGGGG" repeating units
SGGGGSGGGGSGGGGSGGGGSGGGGSGGGGSGGGGSGGGGSGGGGSGGGG

SEQ ID NO: 45 - protein, 54 aa, synthetic construct
  REGION 1..54: note = Synthetic
  REPEAT 5..54: note = This sequence may encompass 1-10 "SGGGG" repeating units
GGGGSGGGGSGGGGSGGGGSGGGGSGGGGSGGGGSGGGGSGGGGSGGGGSGGGG

SEQ ID NO: 46 - protein, 32 aa, synthetic construct
  REGION 1..32: note = Synthetic
  REPEAT 3..32: note = This sequence may encompass 1-10 "SGG" repeating units
GGSGGSGGSGGSGGSGGSGGSGGSGGSGGSGG

SEQ ID NO: 47 - protein, 45 aa, Homo sapiens
  REGION 1..45: note = misc_feature - Human PMEPA-sp (Peptide 5)
MGVNSTAAAAAGQPNVSCTCNCKRSLFQSMEITELEFVQAAAAAK

SEQ ID NO: 48 - protein, 45 aa, Mus musculus
  REGION 1..45: note = misc_feature - Mouse PMEPA-sp (Peptide 6)
MGVNGTAAAAAGQPNVSCACNCQRSLFPSMEITELEFVQAAAAAK

SEQ ID NO: 49 - protein, 45 aa, Homo sapiens
  REGION 1..45: note = misc_feature - Human control (Peptide 5S)
AVMSQTSNKACVSCINVETNGPQCGFALRQSELTAAEFMAAAAAK

SEQ ID NO: 50 - protein, 45 aa, Mus musculus
  REGION 1..45: note = misc_feature - Mouse control (Peptide 6S)
NSGVRSASPCAACFQLNCVENAGLQAVQAGTFPMTMIEEAAAAAK

(19) United States
(12) Patent Application Publication
(10) Pub. No.: US 2026/0083866 A1
(43) Pub. Date: Mar. 26, 2026

(54) COMPOSITIONS TARGETING ENDOTHELIAL CELLS AND USES THEREOF

(71) Applicant: President and Fellows of Harvard College, Cambridge, MA (US)

(72) Inventors: Ulrich H. von Andrian, Cambridge, MA (US); Irina Borisovna Mazo, Cambridge, MA (US); Harold Roderick Neely, Cambridge, MA (US); Mark Cerasoli, Cambridge, MA (US)

(73) Assignee: President and Fellows of Harvard College, Cambridge, MA (US)

(21) Appl. No.: 19/398,555

(22) Filed: Nov. 24, 2025

Related U.S. Application Data
(63) Continuation of application No. PCT/US2024/031073, filed on May 24, 2024.
(60) Provisional application No. 63/504,170, filed on May 24, 2023.
(60) Provisional application No. 63/504,171, filed on May 24, 2023.
(60) Provisional application No. 63/504,173, filed on May 24, 2023.
(60) Provisional application No. 63/504,175, filed on May 24, 2023.

(51) Int. Cl. A61K 51/10; A61K 40/11; A61K 40/31; A61K 40/42; A61K 47/68; A61P 35/00; C07K 16/30

(52) U.S. Cl. CPC A61K 51/1045; A61K 40/11; A61K 40/31; A61K 40/4202; A61K 47/6803; A61K 47/68031; A61K 47/68037; A61K 47/6813; A61K 47/6851; A61P 35/00; C07K 16/30; C07K 16/3007; A61K 2121/00; A61K 2123/00; C07K 2317/31; C07K 2317/569

(57) ABSTRACT

Provided herein are compositions, including pharmaceutical compositions, comprising a binder, linker, and a radioactive isotope, and optionally a masking moiety. The compositions can also comprise a first binder connected to a second binder via a first linker, or a binder connected to a payload via a first linker. In some embodiments, the binder or first binder may specifically bind to a protein (e.g., PMEPA-1) expressed on an endothelial cell. Also provided herein are cells comprising a binder that binds to a protein expressed on an endothelial cell. Also provided herein are engineered immune cells comprising a polynucleotide that encodes a chimeric antigen receptor (CAR) that binds to a protein expressed on an endothelial cell. Also provided herein are methods for using these compositions for selective delivery to cells, treating diseases and disorders, and imaging. In some embodiments, the method comprises administering to the subject an effective amount of the composition disclosed herein.

### RELATED APPLICATIONS

This application is a continuation of International Application No. PCT/US2024/031073, filed on May 24, 2024, which claims the benefit of priority to U.S. Provisional Application No. 63/504,170, filed on May 24, 2023; U.S. Provisional Application No. 63/504,171, filed on May 24, 2023; U.S. Provisional Application No. 63/504,173, filed on May 24, 2023; and U.S. Provisional Application No. 63/504,175, filed on May 24, 2023. The entire contents of each of the foregoing applications are expressly incorporated by reference herein.

### STATEMENT AS TO FEDERALLY SPONSORED RESEARCH

This invention was made with government support under AI155865 awarded by National Institutes of Health (NIH). The government has certain rights in this invention.

### STATEMENT REGARDING SEQUENCE LISTING

The instant application contains a Sequence Listing XML file which has been submitted electronically in .XML format and is hereby incorporated by reference in its entirety. Said .XML copy, created on May 23, 2024, is named 117823-36020.xml and is 68,806 bytes in size. The sequence listing contained in this .XML file is part of the specification and is hereby incorporated by reference herein in its entirety.

### BACKGROUND OF THE INVENTION

Advances in immuno-oncology indicate that a cancer patient's immune system can be therapeutically harnessed to eliminate malignant tumors, providing long lasting responses in some cancers. However, despite this progress, current immunotherapy regimens have only shown efficacy in a subset of malignancies and/or a minority of patients. The high failure rate of cancer immunotherapy is inversely correlated with the presence of tumor-infiltrating T cells. The reason(s) for the paucity of T cells in so-called non-inflammatory tumors (which have a poor prognosis) are not well understood, but likely involve the inability of circulating tumor antigen-specific T cells to adhere to and emigrate from tumor microvessels into the surrounding tissue. As such, there is a need to develop therapies that selectively target tumor endothelial cells compared to non-tumor endothelial cells for detection and treatment of ailments such as cancer.

### SUMMARY OF THE INVENTION

Provided herein is a composition comprising a binder connected to a radioactive isotope via a first linker, wherein the binder specifically binds to a protein expressed on an endothelial cell.

In another aspect, this disclosure provides a composition comprising a first binder connected to a second binder via a first linker, wherein the first binder specifically binds to a protein expressed on an endothelial cell.

In another aspect, this disclosure provides a composition comprising a binder connected to a payload via a first linker, wherein the binder specifically binds to a protein expressed on an endothelial cell.

In some embodiments, the protein is expressed on a venule endothelial cell (VEC) or a non-venule endothelial cell (NVEC). In some embodiments, the protein is expressed on a microvascular endothelial cell. In some embodiments, the protein expression is upregulated in VEC compared to NVEC.

In some embodiments, the protein is overexpressed in VEC by at least about 50% compared to NVEC. In some embodiments, the protein expression is upregulated in NVEC compared to VEC. In some embodiments, the protein is overexpressed in NVEC by at least about 50% compared to VEC.

In some embodiments, the protein is an organ-restricted endothelial cell protein or tissue-specific endothelial cell protein. In some embodiments, the organ-restricted endothelial cell protein or tissue-specific endothelial cell protein is in the liver, kidney, brain, retina, lymph node, bone marrow, small intestine, colon, adipose tissue, skin, lung, heart, any other organ, or a combination thereof.

In some embodiments, the protein has low expression levels on non-tumor endothelial cells. In some embodiments, the protein is not expressed in circulating blood cells. In some embodiments, the protein has greater expression levels on tumor endothelial cells than on non-tumor endothelial cells.

In some embodiments, the protein is encoded by a gene selected from the group consisting of genes set forth in Tables 1-3. In some embodiments, the protein is encoded by a gene selected from VMP1, LAPTM5, EVL, PCDH17, ARRDC3, PMEPA-1, MYOF, MMP14, or PLEKHO1. In some embodiments, the protein is PMEPA-1.

In some embodiments, the binder, first binder, and/or the second binder are an antibody or an antigen-binding fragment thereof. In some embodiments, the binder, first binder, and/or the second binder
(a) is a monoclonal antibody, a polyclonal antibody, a bispecific antibody, a trispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a nanobody, or an antigen-binding fragment thereof; or (b) an aptamer, a dendrimer, a peptide, RNAi, siRNA, shRNA, miRNA.

In some embodiments, the binder, first binder, and/or the second binder are a nanobody, a single chain variable fragment (scFv), a single-chain antibody, a single-domain antibody, a diabody, a Fab fragment, or a combination thereof. In some embodiments, the binder, first binder, and/or the second binder are a nanobody. In some embodiments, the binder, first binder, and/or the second binder are is an endothelial cell specific antibody or an antigen-binding fragment thereof.

In some embodiments, the binder, first binder, and/or the second binder are is an anti-PMEPA-1 antibody or an antigen-binding fragment thereof. In some embodiments, the anti-PMEPA-1 antibody or the antigen-binding fragment thereof comprises (a) a CDR1 having at least 80% sequence identity to the amino acid sequence set out in any one of SEQ ID NOs: 8, 11, 14, 17, 20, or 23, (b) a CDR2 having at least 80% sequence identity to the amino acid sequence set out in any one of SEQ ID NOs: 9, 12, 15, 18, 21, or 24, and (c) a CDR3 having at least 80% sequence identity to the amino acid sequence set out in any one of SEQ ID NOs: 10, 13, 16, 19, 22, or 25. In some embodiments, the anti-PMEPA-1 antibody or the antigen-binding fragment thereof comprises (a) a CDR1 having the amino acid sequence set out in any one of SEQ ID NOs: 8, 11, 14, 17, 20, or 23, (b) a CDR2 having the amino acid sequence set out in any one of SEQ ID NOs: 9, 12, 15, 18, 21, or 24, and (c) a CDR3 having the amino acid sequence set out in any one of SEQ ID NOs: 10, 13, 16, 19, 22, or 25. In some embodiments, the anti-PMEPA-1 antibody or the antigen-binding fragment thereof comprises having at least 80% sequence identity to an amino acid sequence set out in any one of SEQ ID NOs: 1-7. In some embodiments, the anti-PMEPA-1 antibody or the antigen-binding fragment thereof comprises an amino acid sequence set out in any one of SEQ ID NOs: 1-7.

In some embodiments, the binder, first binder, and/or the second binder specifically bind to the protein expressed on the endothelial cell with a Kd value of 1 mM or less, as measured by surface plasmon resonance (SPR). In some embodiments, the binder, first binder, and/or the second binder specifically bind to the protein expressed on the endothelial cell with a Ka value of 1 mM or less, as measured by surface plasmon resonance (SPR).

In some embodiments, the second binder specifically binds to an antigen on the surface of a T cell. In some embodiments, the antigen on the surface of a T cell is selected from CD8b, CD4, CD2, CD28, CD45RA, CD45RO, and CD58. In some embodiments, the second binder specifically binds to an antigen on the surface of a CAR cell, an NK cell, a granulocyte, a macrophage or a monocyte. In some embodiments, the antigen on the surface of a CAR cell, an NK cell, a granulocyte, a macrophage or a monocyte comprises CD15, CD11b, NKG2D, CD16, NKp30, NKp44, NKp46, DNAM, PSGL-1, CD44, CD11a, or CD49a.

In some embodiments, the second binder binds to a viral epitope. In some embodiments, the viral epitope is expressed on an AAV. In some embodiments, the AAV is selected form AAV1, AAV2, AAV3, AAV4, AAV5, AAV6, AAV7, AAV8, AAV9, AAV10, AAV11.

In some embodiments, the second binder binds to a nanoparticle. In some embodiments, the nanoparticle is a lipid nanoparticle. In some embodiments, the nanoparticle is a polymer nanoparticle.

In some embodiments, (a) the binder further comprises a masking moiety; and/or (b) the first binder further comprises a first masking moiety; and/or (c) the second binder further comprises a second masking moiety.

In some embodiments, (a) the masking moiety is covalently attached to a binding domain of the binder via a second linker; or (b) the first masking moiety is covalently attached to a binding domain of the first binder via a second linker; or (c) the second masking moiety is covalently attached to a binding domain of the second binder via a third linker.

In some embodiments, (a) the masking moiety is covalently attached to a heavy chain variable domain and/or a light chain variable chain domain of the binder via a second linker; or (b) the first masking moiety is covalently attached to a heavy chain variable domain and/or a light chain variable chain domain of the first binder via a second linker; or (c) the second masking moiety is covalently attached to a heavy chain variable domain and/or a light chain variable chain domain of the second binder via a third linker.

In some embodiments, the masking moiety, the first masking moiety, and/or the second masking moiety are an anti-idiotypic antibody or fragment thereof. In some embodiments, the first masking moiety, and/or the second masking moiety are is an anti-idiotypic scFv or fragment thereof.

In some embodiments, the first linker and/or the second linker is a non-cleavable linker or a cleavable linker. In some embodiments, the first linker and/or the second linker is a non-cleavable linker.

In some embodiments, the first linker and/or the second linker is a polypeptide linker. In some embodiments, the polypeptide linker comprises about 2-20 amino acids. In some embodiments, the polypeptide linker comprises (G4S)n, (SG4)n, G4(SG4)n or G2(SG2)n, wherein n is selected from 1 to 10. In some embodiments, the polypeptide linker comprises GGGGSGGGGS (SEQ ID NO: 41) or GGGGS (SEQ ID NO: 42).

In some embodiments, the first linker and/or the second linker comprises at least one group selected from the group consisting of alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene is optionally substituted. In some embodiments, the first linker and/or the second linker comprises substituted or unsubstituted C1-C6 alkylene or substituted or unsubstituted C1-C6 heteroalkylene.

In some embodiments, the first linker and/or the second linker comprises one or more groups selected from the group consisting of —O—, —S—, —NH—, —NH—(CH2)y-NH, —NH—(CH2)y-O, —O—(CH2)y-O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH2CH2)w-(C═O)—, —(C═O)— (CH═CH)w-(C═O), —(C═O)—(OCH2CH2O)w-(C═O)—, —(CH2CH2O)w-, —(C═O)— (CH2CH2O)w-, and —(CH(CH3)C(═O)O)w-, wherein w is 1-20 and y is 1-20.

In some embodiments, the first linker and/or the second linker is a cleavable linker. In some embodiments, the cleavable linker is a protease-cleavable linker, a self-immolative linker, or a pH-sensitive linker. In some embodiments, the protease-cleavable linker comprises at least one protease recognition site. In some embodiments, the protease is selected from metalloproteinase (MMP) 1-28; A Disintegrin And Metalloproteinase (ADAM) 2, 7-12, 15, 17-23, 28-30 and 33; serine protease; urokinase-type plasminogen activator; Matriptase; cysteine protease; aspartic protease; and cathepsin protease. In some embodiments, the protease is MMP2 or MMP9.

In some embodiments, the self-immolative linker is selected from para-amino benzoic acid (PAB), para-aminobenzyl alcohol (PABA), 3,3-dimethyl-4-hydroxybutyric acid, ethylenediamine, γ-aminobutyric acid (GABA), 2-hydroxycinnamic acid, “Trimethyl Lock”, or ethanolamine. In some embodiments, the self-immolative linker is para-amino benzoic acid (PAB).

In some embodiments, the pH-sensitive linker is cleaved upon exposure to a target pH. In some embodiments, the target pH is less than about 7. In some embodiments, the pH-sensitive linker is selected from an optionally substituted tetrahydropyranyl ether, an optionally substituted tetrahydropyranyl ester, an optionally substituted azide, an optionally substituted histidine, an optionally substituted hydrazone, or an optionally substituted β-amino ester. In some embodiments, the pH-sensitive linker is selected from-(tetrahydropyran ether)-(azide), -(hydrazone)-, -(hydrazone)-(azide)-, -(β-amino ester)-, -(β-amino ester)-(azide)-, or -(tetrahydropyran ester)-.

In some embodiments, the radioactive isotope is an alpha emitter, beta emitter, or gamma emitter. In some embodiments, the radioactive isotope emits a particle or ray in the range of 10-7,000 keV. In some embodiments, the radioactive isotope emits a particle or ray in the range of 50-1,500 keV. In some embodiments, the radioactive isotope emits a particle or ray in the range of 80-250 keV. In some embodiments, the radioactive isotope is selected from Actinium-225, Astatine-211, Iodine-123, Iodine-125, Iodine-126, Iodine-131, Iodine-133, Bismuth-212, Bromine-77, Indium-111, Indium-113m, Gallium-67, Gallium-68, Lead-212, Ruthenium-95, Ruthenium-97, Ruthenium-103, Ruthenium-105, Mercury-107, Mercury-203, Rhenium-186, Rhenium-188, Tellurium-121m, Tellurium-122m, Tellurium-125m, Thulium-165, Thulium-167, Thulium-168, Technetium-99m, Fluorine-18, Silver-111, Platinum-197, Palladium-109, Copper-67, Phosphorus-32, Phosphorus-33, Yttrium-90, Scandium-47, Samarium-153, Lutetium-177, Rhodium-105, Praseodymium-142, Praseodymium-143, Terbium-161, Holmium-166, Gold-199, Cobalt-57, Cobalt-58, Chromium-51, Iron-59, Selenium-75, Thallium-201, Zirconium-89, and Ytterbium-169. In some embodiments, the radioactive isotope is selected from Iodine-123, Iodine-131, Indium-111, Gallium-67, Lead-212, Ruthenium-97, Technetium-99m, Cobalt-57, Cobalt-58, Chromium-51, Iron-59, Selenium-75, Thallium-201, and Ytterbium-169. In some embodiments, the radioactive isotope is Actinium-225, Gallium-67, Lead-21, or Lutetium-177. In some embodiments, the radioactive isotope is Technetium-99m. In some embodiments, the radioactive isotope is Lead-212.

In some embodiments, the payload is a toxin. In some embodiments, the payload is a cytokine. In some embodiments, the payload is a cytotoxic payload. In some embodiments, the payload is cytotoxic to a tumor cell upon internalization into the tumor cell.

In some embodiments, the payload comprises a antitumor antibiotic, microtubule inhibitor, cytotoxic or cytostatic, topoisomerase inhibitor, a pyrrolobenzodiazepine, a DNA-alkylating drug, a DNA-binding drug, a DNA-cleaving drug, or an RNA polymerase inhibitor.

In some embodiments, the payload comprises pyrrolobenzodiazepine, duocarmycin, auristatin, maytansinoid, uncialamycin, dynemicin, thailanstatin, camptothecin, exatecan, tubulysin compound, lurbinectedin, trabectedin, safracin, lenalidomide, eribulin, vincristine, vinblastine, vindesine, vinorelbine, an epothilone, a taxane (e.g., paclitaxel, docetaxel, cabazitaxel, etc.), a cryptophycin, a hemiasterlin, an anthracyclin, a bisnaphthylamide (e.g., elinafide), or a cytotoxic molecular glue/PROTAC compound.

In some embodiments, the payload is camptothecin, exatecan, lurbinectedin, trabectedin, belotecan, atiratecan, namitecan, 7-nbutyl-10-amino-camptothecin, or 7-n-butyl-9-amino-10,11-methylenedixoy-camptothecin.

In some embodiments, the payload comprises dolastatin 10, auristatin E, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), or 5-benzoylvaleric acid-AE ester (AEVB).

In some embodiments, the DAR (drug to antibody ratio) of the composition is about 1 to about 40, about 1 to about 35, about 1 to about 30, about 1 to about 25, about 1 to about 20, about 1 to about 15, about 1 to about 12, about 1 to about 10, about 1 to about 9, about 1 to about 8, about 1 to about 7, about 1 to about 6, about 1 to about 5, about 1 to about 4, about 1 to about 3, about 1 to about 2, about 2 to about 40, about 2 to about 35, about 2 to about 30, about 2 to about 25, about 2 to about 20, about 2 to about 15, about 2 to about 12, about 2 to about 10, about 2 to about 9, about 2 to about 8, about 2 to about 7, about 2 to about 6, about 2 to about 5, about 2 to about 4, about 2 to about 3, about 3 to about 40, about 3 to about 35, about 3 to about 30, about 3 to about 25, about 3 to about 20, about 3 to about 15, about 3 to about 12, about 3 to about 10, about 3 to about 9, about 3 to about 8, about 3 to about 7, about 3 to about 6, about 3 to about 5, about 3 to about 4, about 4 to about 40, about 4 to about 35, about 4 to about 30, about 4 to about 25, about 4 to about 20, about 4 to about 15, about 4 to about 12, about 4 to about 10, about 4 to about 9, about 4 to about 8, about 4 to about 7, about 4 to about 6, about 4 to about 5, [0037]5 to about 40, about 5 to about 35, about 5 to about 30, about 5 to about 25, about 5 to about 20, about 5 to about 15, about 5 to about 12, about 5 to about 10, about 5 to about 9, about 5 to about 8, about 5 to about 7, about 5 to about 6, about 6 to about 40, about 6 to about 35, about 6 to about 30, about 6 to about 25, about 6 to about 20, about 6 to about 15, about 6 to about 12, about 6 to about 10, about 6 to about 9, about 6 to about 8, about 6 to about 7, about 7 to about 40, about 7 to about 35, about 7 to about 30, about 7 to about 25, about 7 to about 20, about 7 to about 15, about 7 to about 12, about 7 to about 10, about 7 to about 9, about 7 to about 8,

8 to about 40, about 8 to about 35, about 8 to about 30, about 8 to about 25, about 8 to about 20, about 8 to about 15, about 8 to about 12, about 8 to about 10, about 8 to about 9, about 9 to about 40, about 9 to about 35, about 9 to about 30, about 9 to about 25, about 9 to about 20, about 9 to about 15, about 9 to about 12, about 9 to about 10, about 10 to about 40, about 10 to about 35, about 10 to about 30, about 10 to about 25, about 10 to about 20, about 10 to about 15, about 10 to about 12, about 12 to about 40, about 12 to about 35, about 12 to about 30, about 12 to about 25, about 12 to about 20, about 12 to about 15, about 15 to about 40, about 15 to about 35, about 15 to about 30, about 15 to about 25, about 15 to about 20, about 20 to about 40, about 20 to about 35, about 20 to about 30, about 20 to about 25, about 25 to about 40, about 25 to about 35, about 25 to about 30, about 30 to about 40, about 30 to about 35, or about 35 to about 40.

In some embodiments, DAR (drug to antibody ratio) of the composition is about 20.

In some embodiments, DAR (drug to antibody ratio) of the composition is about 10. In some embodiments, DAR (drug to antibody ratio) of the composition is about 5. In some embodiments, DAR (drug to antibody ratio) of the composition is about 4. In some embodiments, DAR (drug to antibody ratio) of the composition is about 3. In some embodiments, DAR (drug to antibody ratio) of the composition is about 2.

In another aspect, this disclosure provides a composition having a structure of Formula (XII): AB-(L)m-(P)n (XII) wherein: AB is an antibody or antigen-binding fragment; L is a linker; P is a payload; m is 0 to 10; and n is 1 to 20.

In some embodiments, the antibody or antigen-binding fragment is (a) a monoclonal antibody, a polyclonal antibody, a bispecific antibody, a trispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a nanobody, or an antigen-binding fragment; or (b) an aptamer, a dendrimer, a peptide, RNAi, siRNA, shRNA, miRNA.

In some embodiments, the antibody or antigen-binding fragment is a nanobody, a single chain variable fragment (scFv), a single-chain antibody, a single-domain antibody, a diabody, a Fab fragment, or a combination thereof. In some embodiments, the antibody or antigen-binding fragment is a nanobody. In some embodiments, the antibody or antigen-binding fragment is an endothelial cell specific antibody.

In some embodiments, the antibody or antigen-binding fragment is an anti-PMEPA-1 antibody or antigen-binding fragment. In some embodiments, the anti-PMEPA-1 antibody or antigen-binding fragment comprises (a) a CDR1 having at least about 80% sequence identity to the amino acid sequence set out in any one of SEQ ID NOs: 8, 11, 14, 17, 20, or 23, (b) a CDR2 having at least about 80% sequence identity to the amino acid sequence set out in any one of SEQ ID NOs: 9, 12, 15, 18, 21, or 24, and (c) a CDR3 having at least about 80% sequence identity to the amino acid sequence set out in any one of SEQ ID NOs: 10, 13, 16, 19, 22, or 25.

In some embodiments, the anti-PMEPA-1 antibody or antigen-binding fragment comprises (a) a CDR1 having the amino acid sequence set out in any one of SEQ ID NOs: 8, 11, 14, 17, 20, or 23, (b) a CDR2 having the amino acid sequence set out in any one of SEQ ID NOs: 9, 12, 15, 18, 21, or 24, and (c) a CDR3 having the amino acid sequence set out in any one of SEQ ID NOs: 10, 13, 16, 19, 22, or 25. In some embodiments, the anti-PMEPA-1 antibody or antigen-binding fragment comprises having at least about 80% sequence identity to an amino acid sequence set out in any one of SEQ ID NOs: 1-7. In some embodiments, the anti-PMEPA-1 antibody or antigen-binding fragment comprises an amino acid sequence set out in any one of SEQ ID NOs: 1-7.

In some embodiments, the antibody or antigen-binding fragment specifically binds to the protein expressed on the endothelial cell with a Kd value of about 1 mM or less, as measured by surface plasmon resonance (SPR). In some embodiments, the antibody or antigen-binding fragment specifically binds to the protein expressed on the endothelial cell with a Ka value of about 1 mM or less, as measured by surface plasmon resonance (SPR).

In some embodiments, the linker is a non-cleavable linker or a cleavable linker. In some embodiments, the linker is a non-cleavable linker.

In some embodiments, the linker is polypeptide linker. In some embodiments, the polypeptide linker comprises about 2-20 amino acids. In some embodiments, the polypeptide linker comprises (G4S)n, (SG4)n, G4(SG4)n or G2(SG2)n, wherein n is selected from 1 to 10. In some embodiments, the polypeptide linker comprises GGGGSGGGGS (SEQ ID NO: 41) or GGGGS (SEQ ID NO: 42).

In some embodiments, the linker comprises at least one group selected from the group consisting of alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene is optionally substituted. In some embodiments, the linker comprises substituted or unsubstituted C₁-C₆ alkylene or substituted or unsubstituted C₁-C₆ heteroalkylene. In some embodiments, the linker comprises one or more groups selected from the group consisting of —O—, —S—, —NH—, —NH—(CH2)p-NH, —NH—(CH2)p-O, —O—(CH2)p-O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH2CH2)q-(C═O)—, —(C═O)— (CH═CH)q-(C═O), —(C═O)—(OCH2CH2O)q-(C═O)—, —(CH2CH2O)q-, —(C═O)— (CH2CH2O)q-, and —(CH(CH3)C(═O)O)q-, wherein q is 1-20 and p is 1-20.

In some embodiments, the linker is a cleavable linker. In some embodiments, the cleavable linker is a protease-cleavable linker, a self-immolative linker, or a pH-sensitive linker. In some embodiments, the protease-cleavable linker comprises at least one protease recognition site. In some embodiments, the protease is selected from metalloproteinase (MMP) 1-28; A Disintegrin And Metalloproteinase (ADAM) 2, 7-12, 15, 17-23, 28-30 and 33; serine protease; urokinase-type plasminogen activator; Matriptase; cysteine protease; aspartic protease; and cathepsin protease. In some embodiments, the protease is MMP2 or MMP9.

In some embodiments, the self-immolative linker is selected from para-amino benzoic acid (PAB), para-aminobenzyl alcohol (PABA), 3,3-dimethyl-4-hydroxybutyric acid, ethylenediamine, γ-aminobutyric acid (GABA), 2-hydroxycinnamic acid, “Trimethyl Lock”, or ethanolamine. In some embodiments, the self-immolative linker is para-amino benzoic acid (PAB).

In some embodiments, the pH-sensitive linker is cleaved upon exposure to a target pH. In some embodiments, the target pH is less than about 7. In some embodiments, the pH-sensitive linker is selected from an optionally substituted tetrahydropyranyl ether, an optionally substituted tetrahydropyranyl ester, an optionally substituted azide, an optionally substituted histidine, an optionally substituted hydrazone, or an optionally substituted β-amino ester. In some embodiments, the pH-sensitive linker is selected from-(tetrahydropyran ether)-(azide), -(hydrazone)-, -(hydrazone)-(azide)-, -(β-amino ester)-, -(β-amino ester)-(azide)-, or -(tetrahydropyran ester)-.

In some embodiments, the payload is a toxin. In some embodiments, the payload is a cytokine. In some embodiments, the payload is a cytotoxic payload. In some embodiments, the payload is cytotoxic to a tumor cell upon internalization into the tumor cell. In some embodiments, the payload comprises a antitumor antibiotic, microtubule inhibitor, cytotoxic or cytostatic, topoisomerase inhibitor, a pyrrolobenzodiazepine, a DNA-alkylating drug, a DNA-binding drug, a DNA-cleaving drug, or an RNA polymerase inhibitor. In some embodiments, the payload comprises pyrrolobenzodiazepine, duocarmycin, auristatin, maytansinoid, uncialamycin, dynemicin, thailanstatin, camptothecin, exatecan, tubulysin compound, lurbinectedin, trabectedin, safracin, lenalidomide, eribulin, vincristine, vinblastine, vindesine, vinorelbine, an epothilone, a taxane (e.g., paclitaxel, docetaxel, cabazitaxel, etc.), a cryptophycin, a hemiasterlin, an anthracyclin, a bisnaphthylamide (e.g., elinafide), or a cytotoxic molecular glue/PROTAC compound. In some embodiments, the payload is camptothecin, exatecan, lurbinectedin, trabectedin, belotecan, atiratecan, namitecan, 7-nbutyl-10-amino-camptothecin, or 7-n-butyl-9-amino-10,11-methylenedixoy-camptothecin. In some embodiments, the payload comprises dolastatin 10, auristatin E, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), or 5-benzoylvaleric acid-AE ester (AEVB).

In some embodiments, the DAR (drug to antibody ratio) of the composition is about 1 to about 40, about 1 to about 35, about 1 to about 30, about 1 to about 25, about 1 to about 20, about 1 to about 15, about 1 to about 12, about 1 to about 10, about 1 to about 9, about 1 to about 8, about 1 to about 7, about 1 to about 6, about 1 to about 5, about 1 to about 4, about 1 to about 3, about 1 to about 2, about 2 to about 40, about 2 to about 35, about 2 to about 30, about 2 to about 25, about 2 to about 20, about 2 to about 15, about 2 to about 12, about 2 to about 10, about 2 to about 9, about 2 to about 8, about 2 to about 7, about 2 to about 6, about 2 to about 5, about 2 to about 4, about 2 to about 3, about 3 to about 40, about 3 to about 35, about 3 to about 30, about 3 to about 25, about 3 to about 20, about 3 to about 15, about 3 to about 12, about 3 to about 10, about 3 to about 9, about 3 to about 8, about 3 to about 7, about 3 to about 6, about 3 to about 5, about 3 to about 4, about 4 to about 40, about 4 to about 35, about 4 to about 30, about 4 to about 25, about 4 to about 20, about 4 to about 15, about 4 to about 12, about 4 to about 10, about 4 to about 9, about 4 to about 8, about 4 to about 7, about 4 to about 6, about 4 to about 5,

5 to about 40, about 5 to about 35, about 5 to about 30, about 5 to about 25, about 5 to about 20, about 5 to about 15, about 5 to about 12, about 5 to about 10, about 5 to about 9, about 5 to about 8, about 5 to about 7, about 5 to about 6, about 6 to about 40, about 6 to about 35, about 6 to about 30, about 6 to about 25, about 6 to about 20, about 6 to about 15, about 6 to about 12, about 6 to about 10, about 6 to about 9, about 6 to about 8, about 6 to about 7, about 7 to about 40, about 7 to about 35, about 7 to about 30, about 7 to about 25, about 7 to about 20, about 7 to about 15, about 7 to about 12, about 7 to about 10, about 7 to about 9, about 7 to about 8,

8 to about 40, about 8 to about 35, about 8 to about 30, about 8 to about 25, about 8 to about 20, about 8 to about 15, about 8 to about 12, about 8 to about 10, about 8 to about 9, about 9 to about 40, about 9 to about 35, about 9 to about 30, about 9 to about 25, about 9 to about 20, about 9 to about 15, about 9 to about 12, about 9 to about 10, about 10 to about 40, about 10 to about 35, about 10 to about 30, about 10 to about 25, about 10 to about 20, about 10 to about 15, about 10 to about 12, about 12 to about 40, about 12 to about 35, about 12 to about 30, about 12 to about 25, about 12 to about 20, about 12 to about 15, about 15 to about 40, about 15 to about 35, about 15 to about 30, about 15 to about 25, about 15 to about 20, about 20 to about 40, about 20 to about 35, about 20 to about 30, about 20 to about 25, about 25 to about 40, about 25 to about 35, about 25 to about 30, about 30 to about 40, about 30 to about 35, or about 35 to about 40.

In some embodiments, DAR (drug to antibody ratio) of the composition is about 20.

In some embodiments, DAR (drug to antibody ratio) of the composition is about 10. In some embodiments, DAR (drug to antibody ratio) of the composition is about 5. In some embodiments, DAR (drug to antibody ratio) of the composition is about 4. In some embodiments, DAR (drug to antibody ratio) of the composition is about 3. In some embodiments, DAR (drug to antibody ratio) of the composition is about 2.

In another aspect, this disclosure provides a cell comprising a first binder that specifically binds to a protein expressed on an endothelial cell.

In some embodiments, the cell is an immune cell or an engineered immune cell. In some embodiments, the immune cell is a T cell, macrophage, monocyte, granulocyte, or natural killer (NK) cell, or natural killer T (NKT) cell. In some embodiments, the T cell is a tumor-infiltrating lymphocyte (TIL) or a cytotoxic T lymphocyte (CTL). In some embodiments, the cell expresses a tumor-specific T-cell receptor. In some embodiments, the immune cell is CD4+ or CD8+ T cell.

In some embodiments, the engineered immune call is a CAR-immune cell. In some embodiments, the CAR-immune cell is a CAR-T cell, CAR-macrophages, CAR-monocyte, CAR-granulocyte, CAR-NK cell, or a CAR-NKT cell.

In some embodiments, the first binder is not a chimeric antigen receptor. In some embodiments, the cell further comprises a second binder that specifically binds to a tumor-associated antigen.

In some embodiments, the tumor-associated antigen is selected from CD19, CD20, CD22, CD30, CD37, CD38, CEA, EpCAM, or BCMA.

In some embodiments, the second binder is linked to the cell via a first linker.

In some embodiments, the protein is expressed on a venule endothelial cell (VEC) or a non-venule endothelial cell (NVEC). In some embodiments, the protein is expressed on a microvascular endothelial cell. In some embodiments, the protein expression is upregulated in VEC compared to NVEC. In some embodiments, the protein is overexpressed in VEC by at least 50% compared to NVEC. In some embodiments, the protein expression is upregulated in NVEC compared to VEC. In some embodiments, the protein is overexpressed in NVEC by at least 50% compared to VEC. In some embodiments, the protein is an organ-restricted or tissue-specific endothelial cell protein.

In some embodiments, the organ-restricted or tissue-specific endothelial cell protein is in the brain, liver, kidney, retina, lymph node, bone marrow, small intestine, colon, adipose tissue, skin, lung, heart, or any other organ. In some embodiments, the protein has low expression levels on non-tumor endothelial cells. In some embodiments, the protein is not expressed in circulating blood cells. In some embodiments, the protein has greater expression levels on tumor endothelial cells than on non-tumor endothelial cells.

In some embodiments, the protein is encoded by a gene selected from the group consisting of molecules set forth in Tables 1-3. In some embodiments, the protein is encoded by a gene selected from VMP1, LAPTM5, EVL, PCDH17, ARRDC3, PMEPA1, MYOF, MMP14, or PLEKHO1. In some embodiments, the protein is PMEPA-1.

In some embodiments, the first binder and/or the second binder is an antibody or antigen-binding fragment. In some embodiments, the first binder and/or the second binder is (a) a monoclonal antibody, a polyclonal antibody, a bispecific antibody, a trispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a nanobody, or an antigen-binding fragment thereof, or (b) aptamer, dendrimer, a peptide, RNAi, siRNA, shRNA, or miRNA or ab antigen-binding fragments thereof.

In some embodiments, the first binder and/or the second binder is a nanobody, a single chain variable fragment (scFv), a single-chain antibody, a single-domain antibody, a diabody, a Fab fragment, or a combination thereof. In some embodiments, the first binder and/or the second binder is a nanobody. In some embodiments, the first binder is an endothelial cell specific antibody.

In some embodiments, the first binder is an anti-PMEPA-1 antibody or antigen-binding fragment. In some embodiments, the anti-PMEPA-1 antibody or antigen-binding fragment thereof comprises (a) a CDR1 having at least 80% sequence identity to the amino acid sequence set out in any one of SEQ ID NOs: 8, 11, 14, 17, 20, or 23, (b) a CDR2 having at least 80% sequence identity to the amino acid sequence set out in any one of SEQ ID NOs: 9, 12, 15, 18, 21, or 24, and (c) a CDR3 having at least 80% sequence identity to the amino acid sequence set out in any one of SEQ ID NOs: 9, 12, 15, 18, 21, or 24. In some embodiments, the anti-PMEPA-1 antibody or antigen-binding fragment thereof comprises (a) a CDR1 having the amino acid sequence set out in any one of SEQ ID NOs: 8, 11, 14, 17, 20, or 23, (b) a CDR2 having the amino acid sequence set out in any one of SEQ ID NOs: 9, 12, 15, 18, 21, or 24, and (c) a CDR3 having the amino acid sequence set out in any one of SEQ ID NOs: 10, 13, 16, 19, 22, or 25. In some embodiments, the anti-PMEPA-1 antibody or antigen-binding fragment thereof comprises having at least 80% sequence identity to an amino acid sequence set out in any one of SEQ ID NOs: 1-7. In some embodiments, the anti-PMEPA-1 antibody or antigen-binding fragment thereof comprises an amino acid sequence set out in any one of SEQ ID NOs: 1-7.

In some embodiments, the first binder specifically binds to the protein expressed on the endothelial cell with a Kd value of 1 mM or less, as measured by surface plasmon resonance (SPR). In some embodiments, the first binder specifically binds to the protein expressed on the endothelial cell with a Ka value of 1 mM or less, as measured by surface plasmon resonance (SPR).

In some embodiments, the first binder and/or the second binder further comprises a masking moiety. In some embodiments, the masking moiety is covalently attached to a binding domain of the first binder and/or the second binder via a second linker. In some embodiments, the masking moiety is covalently attached to a heavy chain variable domain and/or a light chain variable chain domain of the first binder and/or the second binder via a second linker. In some embodiments, the masking moiety is an anti-idiotypic antibody or fragment thereof. In some embodiments, the masking moiety is an anti-idiotypic scFv or fragment thereof. In some embodiments, the first linker and/or the second linker is a non-cleavable linker or a cleavable linker.

In some embodiments, the first linker and/or the second linker is a non-cleavable linker.

In some embodiments, the first linker and/or the second linker is polypeptide linker. In some embodiments, the polypeptide linker comprises about 2-20 amino acids. In some embodiments, the polypeptide linker comprises (G4S)n, (SG4)n, G4(SG4)n or G2(SG2)n, wherein n is selected from 1 to 10. In some embodiments, the polypeptide linker comprises GGGGSGGGGS (SEQ ID NO: 41) or GGGGS (SEQ ID NO: 42).

In some embodiments, the first linker and/or the second linker comprises at least one group selected from the group consisting of alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene is optionally substituted. In some embodiments, the first linker and/or the second linker comprises substituted or unsubstituted C1-C6 alkylene or substituted or unsubstituted C1-C6 heteroalkylene. In some embodiments, the first linker and/or the second linker comprises one or more groups selected from the group consisting of —O—, —S—, —NH—, —NH—(CH2)p-NH, —NH—(CH2)p-O, —O—(CH2)p-O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH2CH2)q-(C═O)—, —(C═O)— (CH═CH)q-(C═O), —(C═O)—(OCH2CH2O)q-(C═O)—, —(CH2CH2O)q-, —(C═O)— (CH2CH2O)q-, and —(CH(CH3)C(═O)O)q-, wherein q is 1-20 and p is 1-20.

In some embodiments, the first linker and/or the second linker is a cleavable linker. In some embodiments, the cleavable linker is a protease-cleavable linker, a self-immolative linker, or a pH-sensitive linker. In some embodiments, the protease-cleavable linker comprises at least one protease recognition site. In some embodiments, the protease is selected from metalloproteinase (MMP) 1-28; A Disintegrin And Metalloproteinase (ADAM) 2, 7-12, 15, 17-23, 28-30 and 33; serine protease; urokinase-type plasminogen activator; Matriptase; cysteine protease; aspartic protease; and cathepsin protease. In some embodiments, the protease is MMP2 or MMP9.

In some embodiments, the self-immolative linker is selected from para-amino benzoic acid (PAB), para-aminobenzyl alcohol (PABA), 3,3-dimethyl-4-hydroxybutyric acid, ethylenediamine, γ-aminobutyric acid (GABA), 2-hydroxycinnamic acid, “Trimethyl Lock”, or ethanolamine. In some embodiments, the self-immolative linker is para-amino benzoic acid (PAB).

In some embodiments, the pH-sensitive linker is cleaved upon exposure to a target pH. In some embodiments, the target pH is less than about 7. In some embodiments, the pH-sensitive linker is selected from an optionally substituted tetrahydropyranyl ether, an optionally substituted tetrahydropyranyl ester, an optionally substituted azide, an optionally substituted histidine, an optionally substituted hydrazone, or an optionally substituted β-amino ester. In some embodiments, the pH-sensitive linker is selected from -(tetrahydropyran ether)-(azide), -(hydrazone)-, -(hydrazone)-(azide)-, -(β-amino ester)-, -(β-amino ester)-(azide)-, or -(tetrahydropyran ester)-.

In another aspect, this disclosure provides a engineered immune cell comprising: (a) a first polynucleotide that encodes a first binder that specifically binds to a protein expressed on an endothelial cell; and (b) a second polynucleotide that encodes a chimeric antigen receptor (CAR).

In some embodiments, the immune cell is a T cell, macrophage, monocyte, granulocyte, natural killer (NK) cell, or natural killer T (NKT) cell. In some embodiments, the immune cell is CD4+ or CD8+ T cell. In some embodiments, the first binder is not a CAR.

In some embodiments, the first binder comprises a membrane anchoring domain. In some embodiments, the membrane anchoring domain is a transmembrane domain or a Gpi linker. In some embodiments, the transmembrane domain comprises a sequence from L-selectin (CD62L), PSGL-1, or alpha4-integrin transmembrane domain.

In some embodiments, the first binder further comprises an intracellular segment. In some embodiments, the intracellular segment comprise a sequence from an intracellular region of L-selectin (CD62L), PSGL-1, or alpha4-integrin.

In some embodiments, the CAR comprises a target domain, a hinge domain, a transmembrane domain, and an intracellular signaling domain. In some embodiments, the target domain comprises a second binder that specifically binds to a tumor-associated antigen.

In some embodiments, the tumor-associated antigen is selected from CD19, CD20, CD22, CD30, CD37, CD38, CEA, EpCAM, or BCMA. In some embodiments, the hinge domain is selected from IgG1, IgG2, IgG3, IgG4, IgA, IgD, IgE, IgM, CD28, or CD8. In some embodiments, the hinge domain is CD28 or CD8. In some embodiments, the transmembrane domain is selected from alpha, beta or zeta chain of the T-cell receptor, CD28, CD3 epsilon, CD45, CD4, CD5, CD8, CD9, CD16, CD22, CD33, CD37, CD64, CD80, CD86, CD123, CD134, CD137, or CD154. In some embodiments, the intracellular signaling domain is selected from CD3ζ, CD3γ, CD3δ, CD3ε, CD5, CD22, CD79a, CD79b, CD66d, CD2, CD4, CD5, CD28, CD134, CD137, ICOS, CD154, 41-BB, or OX40.

In some embodiments, the CAR further comprises a co-stimulatory domain. In some embodiments, the co-stimulatory domain is selected from CD3ζ, CD3γ, CD3δ, CD3ε, CD5, CD22, CD79a, CD79b, CD66d, CD2, CD4, CD5, CD28, CD134, CD137, ICOS, CD154, 41-BB, or OX40. In some embodiments, the co-stimulatory domain is 41-BB or OX40.

In some embodiments, the first binder is not connected to the intracellular signaling domain.

In another aspect, this disclosure provides a pharmaceutical composition comprising the composition of any one of claims 1-122, the cell of any one of claims 123-185, or the engineered immune cell of any one of claims 186-205, and a pharmaceutically acceptable carrier and/or excipient. In some embodiments, the pharmaceutical composition further comprises at least one additional therapeutic agent.

In another aspect, this disclosure provides a method of selectively delivering a radiopharmaceutical composition to a tumor endothelial cell compared to a non-tumor endothelial cell in a subject in need thereof, the method comprising administering to the subject an effective amount of any one of the compositions or pharmaceutical compositions provided herein to the subject.

In another aspect, this disclosure provides a method of selectively delivering a therapeutic composition to a tumor endothelial cell compared to a non-tumor endothelial cell in a subject in need thereof, the method comprising administering to the subject an effective amount of any one of the compositions, cells, engineered immune cells, or pharmaceutical compositions provided herein to the subject. In some embodiments, the composition has a greater binding affinity for a tumor endothelial cell compared to a binding affinity for a non-tumor endothelial cell.

In another aspect, this disclosure provides a method of treating a disease or disorder in a subject in a need thereof, the method comprising administering to the subject an effective amount of the composition of any one of the compositions, cells, engineered immune cells, or pharmaceutical compositions provided herein to the subject.

In some embodiments, the disease or disorder is endotoxemia, sepsis, cancer, obesity-related insulin resistance, diabetes, polycystic ovary syndrome, metabolic syndrome, hypertension, cerebrovascular accident, myocardial infarction, congestive heart failure, cholecystitis, gout, osteoarthritis, Pickwickian syndrome, sleep apnea, atherosclerosis, inflammatory bowel disease, rheumatoid arthritis, vasculitis, transplant rejection, asthma, ischemic heart disease, appendicitis, peptic, gastric and duodenal ulcers, peritonitis, pancreatitis, ulcerative, pseudomembranous, acute and ischemic colitis, diverticulitis, epiglottitis, achalasia, cholangitis, hepatitis, Crohn's disease, enteritis, Whipple's disease, allergy, anaphylactic shock, immune complex disease, organ ischemia, reperfusion injury, organ necrosis, hay fever, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, alveolitis, bronchiolitis, pharyngitis, pleurisy, sinusitis, a parasitic infection, a bacterial infection, a viral infection, an autoimmune disease, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasculitis, angiitis, endocarditis, arteritis, thrombophlebitis, pericarditis, myocarditis, myocardial ischemia, periarteritis nodosa, rheumatic fever, celiac disease, adult respiratory distress syndrome, meningitis, encephalitis, cerebral infarction, cerebral embolism, Guillain-Barre syndrome, neuritis, neuralgia, spinal cord injury, paralysis, uveitis, arthritides, arthralgias, osteomyelitis, fasciitis, Paget's disease, periodontal disease, synovitis, myasthenia gravis, thyroiditis, systemic lupus erythematosus, Goodpasture's syndrome, Behcets's syndrome, allograft rejection, graft-versus-host disease, ankylosing spondylitis, Berger's disease, Reiter's syndrome, Hodgkin's disease, endometriosis, hemangioma, diseases associated with tissue fibrosis, Raynaud syndrome, Sjogren's syndrome, scleroderma, or fibrosis of liver, lung, heart, kidney, skin, pancreas, or intestine.

In some embodiments, the disease or disorder is non-venular disease. In some embodiments, the non-venular disease is selected from vessel coronary disease, thrombotic microangiopathy, microangiopathic hemolytic anemia, microvascular occlusion, cutaneous diabetic microangiopathy, Susac's syndrome, cerebral microangiopathy, early diabetic microangiopathy, diabetic microangiopathy, glomerular microangiopathy, non-neoplastic nevus, pulmonary microangiopathy, pulmonary capillaritis, coronary microvascular disease, chronic microvascular diseases, small vessel ischemia, thrombotic thrombocytopenic purpura, endometriosis, arteriolosclerosis, hemangioma, diseases associated with tissue fibrosis, Raynaud syndrome, Sjogren's syndrome, scleroderma, or fibrosis of liver, lung, heart, kidney, skin, pancreas, or intestine. In some embodiments, the non-venular disease is hemangioma, endometriosis, diseases associated with tissue fibrosis, Raynaud syndrome, Sjogren's syndrome, scleroderma, or fibrosis of liver, lung, heart, kidney, skin, pancreas, or intestine.

In some embodiments, whereby upon administration of the composition, cell, engineered immune cell, or pharmaceutical composition in the subject, more than 50% of the composition, cell, engineered immune cell, or pharmaceutical composition that is retained in the subject within 2 hours following administration is localized in a tumor microenvironment within the subject.

In some embodiments, the tumor microenvironment comprises tumor-associated endothelial cells. In some embodiments, the composition, cell, engineered immune cell, or pharmaceutical composition has a greater binding affinity for the tumor-associated endothelial cells than a binding affinity for normal endothelial cells.

In some embodiments, whereby upon administration of the composition, cell, engineered immune cell, or pharmaceutical composition in the subject, more than 5% of the composition, cell, engineered immune cell, or pharmaceutical composition that is retained in the subject within 12 hours following administration is localized in a tumor microenvironment within the subject.

In some embodiments, the whereby upon administration of the composition, cell, engineered immune cell, or pharmaceutical composition in the subject, more than 5%, more than 10%, more than 15%, more than 20%, more than 25%, more than 30%, more than 35%, more than 40%, more than 45%, more than 50%, or more than 55% of the composition, cell, engineered immune cell, or pharmaceutical composition that is retained in the subject within 2 hours, 3 hours, 4 hours, 5 hours, 6 hours, 7 hours, 8 hours, 9 hours, 10 hours, 11 hours, 12 hours, 13 hours, 14 hours, or 15 hours following administration is localized in a tumor microenvironment within the subject. In some embodiments, the whereby upon administration of the composition, cell, engineered immune cell, or pharmaceutical composition in the subject, at least 5%, at least 10%, at least 15%, at least 20%, at least 25%, at least 30%, at least 35%, at least 40%, at least 45%, at least 50%, or at least 55% of the composition, cell, engineered immune cell, or pharmaceutical composition that is retained in the subject within 2 hours, 3 hours, 4 hours, 5 hours, 6 hours, 7 hours, 8 hours, 9 hours, 10 hours, 11 hours, 12 hours, 13 hours, 14 hours, or 15 hours following administration is localized in a tumor microenvironment within the subject. In some embodiments, the whereby upon administration of the composition, cell, engineered immune cell, or pharmaceutical composition in the subject, between 5% and 55%, between 5% and 50%, between 5% and 45%, between 5% and 40%, between 5% and 35%, between 5% and 30%, between 5% and 25%, between 5% and 20%, between 5% and 15%, between 5% and 10%,
between 10% and 55%, between 10% and 50%, between 10% and 45%, between 10% and 40%, between 10% and 35%, between 10% and 30%, between 10% and 25%, between 10% and 20%, between 10% and 15%, between 15% and 55%, between 15% and 50%, between 15% and 45%, between 15% and 40%, between 15% and 35%, between 15% and 30%, between 15% and 25%, between 15% and 20%, between 20% and 55%, between 20% and 50%, between 20% and 45%, between 20% and 40%, between 20% and 35%, between 20% and 30%, between 20% and 25%, between 25% and 55%, between 25% and 50%, between 25% and 45%, between 25% and 40%, between 25% and 35%, between 25% and 30%, between 30% and 55%, between 30% and 50%, between 30% and 45%, between 30% and 40%, between 30% and 35%, between 35% and 55%, between 35% and 50%, between 35% and 45%, between 35% and 40%, between 40% and 55%, between 40% and 50%, between 40% and 45%, between 45% and 55%, between 45% and 50%, or between 50% and 55% of the composition, cell, engineered immune cell, or pharmaceutical composition that is retained in the subject within 2 hours, 3 hours, 4 hours, 5 hours, 6 hours, 7 hours, 8 hours, 9 hours, 10 hours, 11 hours, 12 hours, 13 hours, 14 hours, or 15 hours following administration is localized in a tumor microenvironment within the subject.

In some embodiments, whereby upon administration of the composition, cell, engineered immune cell, or pharmaceutical composition in a subject, more than 50% of the composition, cell, engineered immune cell, or pharmaceutical composition is excreted from the subject within 12 hours following administration. In some embodiments, whereby upon administration of the composition, cell, engineered immune cell, or pharmaceutical composition in a subject, the renal toxicity metrics in the subject within 24 hours following administration remains within 20% of the levels of the renal toxicity metrics prior to administration.

In some embodiments, the renal toxicity metrics are one or more of serum creatinine, glomerular filtration rate, and blood urea nitrogen.

In another aspect, this disclosure provides a kit comprising the any one of the compositions, cells, engineered immune cells, or pharmaceutical compositions provided herein, and instructions for use.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1D. FIG. 1A shows imaging shows the proximity between VEC and T cells in MC38. Scale bar: 50 m. FIG. 1B shows gating strategy for EC and T cell. FIG. 1C shows selection of CD31+ and/or DARC+ cells from tumors, peritumoral and healthy tissues from mice with MC38 tumors. FIG. 1D shows selection of CD31+ and/or DARC+ cells from tumors, peritumoral and healthy tissues from mice with B16F10 tumors.

FIGS. 2A-2G. FIG. 2A shows number of VEC per gram of tissue in MC38 and B16 subcutaneous (SubQ) tumors, peritumoral tissues and healthy skin. FIG. 2B shows number of CD8+ T cells per gram of tissue in MC38 and B16 subcutaneous (SubQ) tumors, peritumoral tissues and healthy skin. FIG. 2C shows correlation between the number of VEC and the number of CD8+ T cells in MC38 tumor models in mice. FIG. 2D shows correlation between the number of VEC and the number of CD8+ T cells in B16 tumor models in mice. FIG. 2E shows percentage of CD31+DARC+blood endothelial cells in MC38 and B16 subcutaneous (SubQ) tumors, peritumoral tissues and healthy skin. FIG. 2F shows percentage of CD3+CD8+ T cells among all CD45+ cells in MC38 and B16 subcutaneous (SubQ) tumors, peritumoral tissues and healthy skin. FIG. 2G shows correlation between the number of VEC and the number of CD45+CD3+CD8+ T cells per gram of tissue in MC38 and B16 subcutaneous (SubQ) tumors.

FIGS. 3A-3G. FIG. 3A shows percentage of CD31+DARC+blood endothelial cells (BEC) in pancreatic Panc02 tumors, pancreatic M8 organoid tumors and healthy pancreas in mice. FIG. 3B shows number of CD31+DARC+ venular cells per gram of tissue in pancreatic Panc02 tumors, pancreatic M8 organoid tumors and healthy pancreas in mice. FIG. 3C shows percentage of CD45+CD3+CD8+ T cells among all CD45+ cells in pancreatic Panc02 tumors, pancreatic M8 organoid tumors and healthy pancreas in mice. FIG. 3D shows number of CD45+CD3+CD8+ T cells per gram of tissue in pancreatic Panc02 tumors, pancreatic M8 organoid tumors and healthy pancreas in mice. FIG. 3E shows correlation between the number of VEC and the number of CD45+CD3+CD8+ T cells per gram of tissue in pancreatic Panc02 tumors in mice. FIG. 3F shows correlation between the number of VEC and the number of CD45+CD3+CD8+ T cells per gram of tissue pancreatic M8 organoid tumors in mice. FIG. 3G shows correlation between the number of VEC and the number of CD45+CD3+CD8+ T cells per gram of tissue in pancreatic Panc02 tumors and pancreatic M8 organoid tumors in mice.

FIGS. 4A-4F. FIG. 4A shows percentage of CD31+DARC+ VEC cells among BEC from human melanoma, peritumoral tissue and healthy skin. FIG. 4B shows number of DARC+ VEC per gram of tissue from human melanoma, peritumoral tissue and healthy skin. FIG. 4C shows percent of CD45+CD3+CD8+ T cells among CD45+ cells from human melanoma, peritumoral tissue and healthy skin. FIG. 4D shows number of CD8+ T cells per gram of tissue from human melanoma, peritumoral tissue and healthy skin. FIG. 4E shows correlation between percent of VEC among BEC and percent of CD45+CD3+CD8+ T cells among CD45+ cells from human melanoma. FIG. 4F shows correlation between number of VEC and number of CD8+ T cells per gram of tissue from human melanoma.

FIGS. 5A-5B. FIG. 5A shows a circle graph of the percentages of CD8+, CD4+, CD11c+CD103+, CD11c+CD103-, and CD11c−cells in human skin sample and melanoma sample. FIG. 5B shows the percentage of CD45-CD31+DARC+ VEC among BEC in human skin tissue and melanoma tumor.

FIGS. 6A-6I. FIG. 6A shows correlation between the number of CD8+ T cells and DARC+ VEC per gram of tissue in patient melanoma samples. R: patients who responded positively to immunotherapy using anti PD1 or a combination of anti PD1 and anti CTL4 therapy. PD: patients with progressive disease, unresponsive to immunotherapy. FIG. 6B shows the percentage of CD31+DARC+ VEC of BEC in patient pancreatic tumor samples and peritumoral tissue. FIG. 6C shows number of CD31+CARD+ VEC per gram in patient pancreatic tumor samples and peritumoral tissue. FIG. 6D shows percent of CD45+CD3+CD8+ T cells of CD45+ cells in patient pancreatic tumor samples and peritumoral tissue. FIG. 6E shows number of CD45+CD3+CD8+ T cells per gram in patient pancreatic tumor samples and peritumoral tissue. FIG. 6F shows correlation between the percentage of CD8+ T cells per amount of CD45+ cells and DARC+ VEC per amount of BEC in patient pancreatic tumor samples. FIG. 6G shows correlation between number of VEC and number of CD8+ T cells per gram in pancreatic tumor samples. FIG. 6H shows comparison between pancreatic tumor, NM pancreas and duodenum. For each sample the percentage of CD8+ T cells per amount of CD45+ cells (open circle) and DARC+ VEC per amount of blood EC (full circle) were calculated and plotted using a line to connect them. FIG. 6I shows homing experiment in RAG KO mouse. After MC38 and B16 tumors were allowed to grow in RAG KO mice, the mice were injected with fluorescent activated T cells. 24 hours after injection the number of CD8+ T cells per gram of tissue (open circle) and DARC+ VEC per gram of tissue (full circle) were calculated and plotted. Lines connect the data points form the same sample.

FIGS. 7A-7E. FIG. 7A shows the correlation between the number of CD45+CD3+CD8+ T cells per gram of tissue and the number of CD31+DARC+ VEC per gram of tissue in MC38 and B16F10 tumor models in mice. FIG. 7B shows the correlation between the number of CD45+CD3+CD8+ T cells per gram of tissue and the number of CD31+DARC+ VEC per gram of tissue in Panc02 and M8 tumors. FIG. 7C shows the correlation of the number of CD8+ T cells per gram of tissue and the number of DARC+ VEC per gram of tissue in human melanoma and pancreatic tumor tissues. FIG. 7D shows the number of CD8+ T cells per gram of tissue in pancreatic and melanoma tissue samples. FIG. 7E shows the number of DARC+ VEC per gram of tissue in pancreatic and melanoma tissue samples.

FIGS. 8A and 8B. FIG. 8A shows gating strategy for isolating CD8 T cells after differentiation of b-actin GFP splenocyte from a b-actin GFP mouse. Cells were cultured in anti-CD3 for 48 hours. Cells were washed in resuspended in media containing IL-2 (20 ng/mL) and used after 8-10 days. FIG. 8B shows presence of transferred differentiated b-actin GFP CD8+ T cells in tumors: upper panel—overnight after transfer in WT mice; lower panel—4 hours after transfer in WT and RAG KO mice.

FIGS. 9A-9F. FIG. 9A shows processing pipeline. After sample collection (mouse or human), cells are dissociated and, for most samples, enriched for CD31+ cells using magnetic beads. Cells are then loaded on an array pre-loaded with sequencing beads. SeqWell protocol libraries were prepared and the data was analyzed. FIG. 9B shows EC isolation: despite enriching for EC, many other cell types were identified. Therefore, using a combination of differential expression markers, EC scoring and cell type specific genes, EC were identified and isolated before proceeding with the next steps of the analysis. FIG. 9C shows UMAP of various EC subsets in mouse healthy skin EC. Further, specialized EC subsets were identified based on previously validated gene markers for those subsets (FIG. 9D). FIG. 9E shows UMAP of various EC subsets in human healthy skin EC and FIG. 9F shows specialized EC subsets identified based on previously validated gene markers for those subsets.

FIGS. 10A-10C. FIG. 10A shows a schematic of in silico gating of ECs. FIG. 10B shows a sample of t-SNE of cells in healthy mouse skin. FIG. 10C shows a sample of t-SNE and doublets overlay.

FIGS. 11A-11G. FIG. 11A shows an iterative process to identify and select ECs in mouse healthy skin. In each iteration cell clusters containing ECs were identified based on cell markers, EC score and differentially expressed genes. FIG. 11B shows healthy mouse skin ECs. Lymphatic ECs (LECs); non-venular ECs (NVECs); venular ECs (VECs). FIG. 11C shows expression of EC genes, Darc, Pecam1, Cdh5, and Lyve1 overlaid on the UMAP (Uniform Manifold Approximation and Projection). FIG. 11D shows expression of EC genes, Selp, Sele, Sdpr, and Vwf overlaid on the UMAP (Uniform Manifold Approximation and Projection). FIG. 11E shows violin graph of VEC score. FIG. 11F shows violin graph of Pecam1 expression. FIG. 1G shows violin graph of Darc expression.

FIGS. 12A-12E. FIG. 12A shows unbiased clustering (UMAP) and heatmap results in VEC and NVEC clusters from healthy skin. FIG. 12B shows unbiased clustering (UMAP) and heatmap results in VEC and NVEC clusters from MC38 tumor samples. FIG. 12C shows unbiased clustering (UMAP) and heatmap results in VEC and NVEC clusters from B16F10 tumor samples. VECs could not be identified by clustering alone. A VEC scoring with a cutoff of 0.2 to identify VECs. In the heatmap VECs were separated and put on the right to check their gene expression against the other clusters. FIG. 12D shows in silico gating (unbiased clustering (UMAP)) of EC from B16F10 melanoma samples. FIG. 12E shows a violin graph of each cluster based on VEC score from B16F10 samples in FIG. 12D. VEC scoring with a cutoff of 0.2 was used to identify VECs.

FIGS. 13A-13I. FIG. 13A shows unsupervised clustering of all mouse EC colored by cluster. FIG. 13B shows unsupervised clustering of all mouse EC colored by cell and sample type. FIG. 13C shows division of cell type by cluster. Heatmaps of top and bottom 50 differentially expressed genes in (FIG. 13D) Healthy skin VECs, (FIG. 13E) MC38 VECs, (FIG. 13F) B16 VECs. FIG. 13G shows cell reassignment per cluster using the Silhouette algorithm. GSVA analysis of (FIG. 13H) MC38 VECs and (FIG. 13I) B16 VECs using a curated list of EC pathways. Bolded: pathways unique to MC38 or B16. Numbers in brackets: number of similar pathways.

FIGS. 14A-14F. shows similarity scoring in mouse. FIG. 14A shows healthy VEC, FIG. 14B shows MC38 VEC, and FIG. 14C shows B16 VEC using the VEC signature (top 50 upregulated genes in VECs) all cells were scored and compared. FIG. 14D shows healthy VEC, FIG. 14E shows MC38 VEC, and FIG. 14F shows B16 VEC plotted on an axis. Here, for each sample type, a “VEC score” was calculated using the top 50 up-regulated genes and a “NVEC score” using the top 50 down-regulated genes. Those 2 scores were combined as follow: combined_score=(average_VEC_score_for_that_cluster)−(average_NVEC_score_for_that_cluster). Cells are then plotted on axis which ends up going from NVEC to VEC thus assessing the relative similarities of the subsets.

FIGS. 15A-15D. FIG. 15A shows a UMAP of healthy human skin. FIG. 15B shows in silico gating of EC from healthy human skin. FIG. 15C shows expression of EC genes, Darc, CLDN5, PROX1, CDH5, and LYVE1 overlaid on the UMAP. FIG. 15D shows a heatmap of healthy human skin.

FIGS. 16A-16E. FIG. 16A shows heatmap of the gene signature in human healthy skin samples. FIG. 16B shows heatmap of the gene signature in non-malignant pancreas VEC samples. FIG. 16C shows heatmap of the gene signature in melanoma VEC samples. FIG. 16D shows heatmap of the gene signature in pancreatic tumor VEC samples. FIG. 16E shows cell reassignment per cluster using the Silhouette algorithm in human samples.

FIGS. 17A-17C. FIG. 17A shows UMAPs and DotPlots of typical cell type markers for human Melanoma samples. FIG. 17B shows UMAPs and DotPlots of typical cell type markers for human Non-malignant Pancreas samples. FIG. 17C shows UMAPs and DotPlots of typical cell type markers for human Pancreatic tumor samples.

FIGS. 18A-18F. FIG. 18A shows unsupervised clustering of human healthy skin and melanoma ECs colored by cluster and sample type. FIG. 18B shows unsupervised clustering of human healthy skin and melanoma ECs colored by cell and sample type. FIG. 18C shows division of cell type by cluster.

FIG. 18D shows unsupervised clustering of human (NM) pancreas and pancreatic tumor EC colored by cluster and sample type. FIG. 18E shows unsupervised clustering of human (NM) pancreas and pancreatic tumor EC colored by cell and sample type. FIG. 18F shows division of cell type by cluster.

FIGS. 19A-19D. FIG. 19A shows the correlation between number of CD45+CD3+CD8+ T cells per gram of tissue and number CD31+DARC+ VEC per gram of tissue in Panc02 and M8 tumors. FIG. 19B shows the correlation between number of CD8+ T cells and number of DARC+ VEC per gram of tissue in human melanoma and pancreatic tumor samples. FIG. 19C shows the number of CD8+ T cells per gram of tissue from pancreatic and melanoma tumor samples. FIG. 19D shows the number of DARC+ VEC per gram of tissue from pancreatic and melanoma tumor samples.

FIG. 20 shows a heatmap of non-malignant NVEC, pancreatic tumor VEC, pancreatic tumor NVEC, human melanoma VEC, human melanoma NVEC, non-malignant pancreatic VEC, human skin VEC, and human skin NVEC tissue samples.

FIGS. 21A-21F. FIG. 21A shows Venn diagrams of genes upregulated in immunogenic tumors. FIG. 21B shows the top 10 enriched pathways in the shared gene list in immunogenic tumors using EnrichR. FIG. 21C shows enriched transcription factors in immunogenic tumors. FIG. 21D shows Venn diagrams of genes upregulated in non-immunogenic tumors. FIG. 21E shows the top 10 enriched pathways in the shared gene list in non-immunogenic tumors using EnrichR. FIG. 21F shows enriched transcription factors in non-immunogenic tumors. The combined score is a combination of the p-value and z-score calculated by multiplying the two scores as follows: c=ln(p)*z. Where c is the combined score, p is the p-value computed using Fisher's exact test, and z is the z-score computed to assess the deviation from the expected rank. The combined score provides a compromise between both methods and has been shown to reports the best rankings when compared with other scoring schemes.

FIGS. 22A-22D. FIG. 22A shows DotPlots of transcription factors, TR1, TR2, and TR3, in various human tumors and healthy skin, such as healthy skin (LEC, VEC, and NVEC), melanoma (NVEC, VEC, and LEC), healthy pancreas (LEC, NVEC, and VEC), and tumor pancreas (VEC and NVEC). FIG. 22B shows DotPlots of transcription factors, TR1, TR2, and TR3, in mouse tumors and healthy skin, such as MC38 tumor (VEC and NVEC), B16 tumor (VEC and NVE), healthy skin (VEC, NVEC, and LEC). FIG. 22C shows expression levels of mouse TR3 from all ECs in healthy skin, MC38 tumor, and B16 tumor cells. FIG. 22D shows expression levels of human TR3 from all ECs in healthy skin, melanoma, non-malignant pancreas, and pancreatic tumor cells.

FIGS. 23A-23D. FIG. 23A shows expression levels of mouse HIF1a in healthy skin endothelial cells (VEC, NVEC, and LEC), MC38 tumor endothelial cells (VEC, NVEC, and LEC), and B16F10 tumor endothelial cells (VEC, NVEC, and LEC). FIG. 23B shows expression levels of mouse HIF1a from all ECs in healthy skin, MC38 tumor, and B16 tumor cells. FIG. 23C shows expression levels of human HIF1a in healthy skin endothelial cells (VEC, NVEC, and LEC), melanoma tumor endothelial cells (VEC, NVEC, and LEC), non-malignant pancreas endothelial cells (VEC, NVEC, and LEC), and pancreatic tumor endothelial cells (VEC, NVEC, and LEC). FIG. 23D shows expression levels of human HIF1a from all ECs in healthy skin, melanoma tumor, non-malignant pancreas, and pancreatic tumor cells.

FIGS. 24A-24B. FIG. 24A shows tumor size (volume mm³) from MC38 tumors implanted in wild type and TR3 knockout mice. FIG. 24B isolation of CD31+DARC+ cells from MC38 tumors implanted in wild type and TR3 knockout mice.

FIGS. 25A-25D. FIG. 25A shows percentage of CD31+DARC+ BECs per gram of tissue in MC38 tumors and peritumoral tissues from wild type and TR3 knockout mice. FIG. 25B shows number of CD31+DARC+ BECs per gram of tissue in MC38 tumors and peritumoral tissues from wild type and TR3 knockout mice. FIG. 25C shows percentage of CD31+DARC− NVEC BECs per gram of tissue in MC38 tumors and peritumoral tissues from wild type and TR3 knockout mice. FIG. 25D shows number of CD31+DARC− BECs per gram of tissue in MC38 tumors and peritumoral tissues from wild type and TR3 knockout mice.

FIGS. 26A-26C shows Venn diagrams of up-regulated genes in murine and human tumor microvasculature compared to healthy tissues. Single cell suspensions of murine MC38 colorectal adenocarcinoma (MC38, blue), murine B16F10 melanoma (B16, red), human pancreatic cancer (hPanT, yellow), human melanoma (hMel, green) and peri-tumoral tissue were isolated by Seq Well and processed for scRNA-seq. VECs and NVECs were identified based on characteristic gene expression patterns and each EC subset in tumors and matched peri-tumoral tissue was compared to identify tumor-specific overexpressed genes. Venn diagrams of the number of upregulated genes as compared to healthy in FIG. 26A) all blood ECs, FIG. 26B) NVECs and FIG. 26C) VECs.

FIGS. 27A-27E. FIGS. 27A and 27B shows validation assays of a candidate tumor EC target, PMEPA-1. EC mRNA levels of PMEPA-1 were compared in VEC, NVEC and lymphatic EC (LEC) in (FIG. 27A) healthy mouse skin and subcutaneous MC38 and B16F10 tumors and (FIG. 27B) human non-malignant pancreas and pancreatic cancer. FIG. 27C FACS analysis of PMEPA-1 on ECs in MC38 tumors and healthy skin. FIG. 27D FACS analysis of PMEPA-1 on ECs in MC38 tumors and healthy skin using Iso control antibody. FIG. 27E Percentage of PMEPA-1+blood EC (BEC).

FIGS. 28A-28D shows the generation of nanobody against PMEPA-1. FIG. 28A L1.2 cells were transfected with either a linearized or a circular plasmid. FIG. 28B The cells were expanded in the presence of G418 and GFP expression was assessed by FACS. FIG. 28C Cells with the highest MFI were single sorted and expanded. FIG. 28D Clones demonstrating the highest level of PMEPA-1-GFP expression (which is enhanced by treatment with sodium butyrate) is ready for use in selection of sdAb from the yeast display library.

FIG. 29 shows enrichment of receptor positive cells. Receptor-negative and receptor-positive cells were labeled with different fluorescent dyes and mixed in 1:1 ratio. Yeast expressing sdAb library was added to the cultures in proportion Yeast:Targets=25:1. The cultures were incubated on a gentle shaker at 4° C. for 1 h, the cells were then collected and stained for FACS to determine the percentage of HA+ cells in each population.

FIG. 30 shows a schematic strategy to generate sdAb against PMEPA-1 to target CAR-T cells to solid tumors.

FIGS. 31A-31C. FIG. 31A depicts an overview schematic of a synthetic homing receptor (SHORE)-expressing T cells targeting tumor EC surface antigen PMEPA1 to infiltrate the tumor parenchyma to engage in CAR-driven anti-tumor immunity. (FIG. 31B) Experimental setup for a competitive homing assay to analyze differential recruitment into tumors driven by SHOREs. (FIG. 31C) Ratio of accumulated targeted SHORE T cells against PMEPA1 to untargeted control SHORE T cells in the spleen and tumor of MC38-bearing mice 20 hours after adoptive transfer.

FIGS. 32A-32E. FIG. 32A shows amino acid sequence of synthetized biotinylated human PMEPA-1 specific (Peptide 5), mouse PMEPA-1 specific (Peptide 6), human control (Peptide 5S) and mouse control (Peptide 6S) peptides. Peptides 5 and 6 represent the sequence of full length ectodomain of human and mouse PMEPA-1a with 5× alanine linker at C terminus followed by biotinylated lysine. FIG. 32B shows the process of sdAB selection from yeast display library. For negative selection, sdAB-expressing yeast is incubated with anti-biotin beads and applied to magnetic column. Beads can bind non-specifically to some yeast with sdAB expression. This population sticks in the column and is discarded. Flow Through fraction from the column immediately incubated with PMEPA-1 specific peptide and, subsequently, with anti-biotin beads. Yeast cells expressing sdAB and bound to PMEPA-1 specific peptide plus anti-biotin magnetic beads stick in the column. Un-bound yeast in Flow Through fraction is discarded, and sdAB-expressing yeast bound in the column flushed from the column and kept for the next cycle of negative=>positive selection after recovery and expansion. FIG. 32C shows FACS-based analysis of binding of selected yeast to PMEPA-1 specific peptide. HA expression demonstrates expression of the sdAB on the surface of yeast cells. SA binding demonstrated the ability of yeast cells bind to PMEPA-1 specific peptide. With each round of selection, the percentage of SA+HA+ yeast cells increases, confirming increased presence of clones with specificity for PMEPA-1 peptide and decreased library diversity. Shown selection 1, 2, and 3. FIG. 32D shows schematic drawing of single cell sort to generate individual PMEPA-1 specific subclones. FIG. 32E shows best selected subclones. “Positive” subclones demonstrate finding to both mouse and human PMEPA-1 specific peptides (although selection was performed on mouse PMEPA-1 specific peptide only, high (90%) identity between mouse and human PMEPA-1 specific peptides allows binding of selected subclones to both peptides. At the same time, “positive” subclones to not bind to control mouse and human peptides (6S and 5S), demonstrating the absence of non-specific binding. “Negative” subclones demonstrate no binding to any of the peptides.

FIGS. 33A-33F. FIG. 33A demonstrates “negative” (IG11, IG12 and IIG12) and “positive” (ID5, ID8, IIB2, IIC10) sdABs binding to PMEPA-1 expressing mouse (MEF) and human (293T) cell lines as well as to PMEPA-1 negative cell line BW a-b-. FIG. 33B shows gating strategy for EC in MC38 tumor and healthy skin. BEC are determined as live (not shown) CD45−CD31+gp38− cells, which were further subdivided to VEC (DARC+) and NVEC (DARC−). FIG. 33C shows binding of “negative” and “positive” sdABs to various subsets of BEC (Total fraction, DARC+ VEC and DARC− NVEC) in MC38 tumor and healthy skin. FIG. 33D represents a micrograph of IHC staining of MC38 tumor with sdAB IIB2. Tumor EC were identified by anti-CD31 staining. Note colocalization of the staining. FIG. 33E shows IHC staining of MC38 tumor with Fc-sdAB fusions. Human Fc fused to PMEPA-1 specific sdAb and mouse Fc fused to control sdAB were co-injected in MC38 tumor bearing mouse. The mouse was sacrificed 20 min after injection and perfused to wash out the unbound AB. Tumor was dissected, frozen and sectioned for further in vitro staining with anti-CD31, anti-human IgG1 and anti-mouse IgG1. Note binding of hFc-PMEPA-1 specific AB to most microvessels (identified by CD31 expression) and absence of binding of the mFc-Control AB. FIG. 33F represents a combined table of PMEPA-1 expression in all analyzed mouse and human tumors as well as healthy tissues. Analysis was performed by IHC staining and, in some cases, by FACS.

### DETAILED DESCRIPTION

The present disclosure provides compositions and methods for imaging and treating solid tumors by targeting clinically relevant molecules that are upregulated in tumor cells. Provided herein are compositions and methods for treating and imaging tumors by targeting clinically relevant molecules that are upregulated in tumor vascular endothelial cells. The endothelial cells may comprise non-venular or venular endothelial cells in and surrounding a tumor, forming the tumor microvasculature, but they are not upregulated in vasculature from healthy tissues, e.g., non-tumor vascular endothelial cells. Characterization of tumor microvasculature, human and murine immune cell infiltrates of immunogenic tumors (T-cell rich and onco-immunotherapy responders), and nonimmunogenic tumors (T-cell poor and onco-immunotherapy non-responders) demonstrated the importance of the vasculature in recruiting intra-tumoral T cells and allowed for the identification of genes that are over-represented in tumor microvasculature of solid tumors, globally in all tumor vascular endothelial cells within the tumors or selectively in venules or non-venules (capillaries and arterioles).

The compositions provided herein comprise radioactive isotope that may provide for imaging capabilities via alpha, beta, or gamma emission. In some instances, the radioactive decay (e.g., alpha decay) may provide for treatment of diseases, such as cancer. The combination of the binders provided herein, which may allow for selective delivery of the compositions provided herein may allow for targeted imaging and treatment of diseases, such as cancer. Targeted imaging and treatment of cancer may lead to enhanced clinical outcomes.

In another aspect, the present disclosure also provides cells comprising a binder that specifically binds to a protein expressed on an endothelial cells. In some instances, the cell is an immune cell or an engineered immune cell. In some instances, the cell is a T cell, macrophage, monocyte, granulocyte, natural killer (NK) cell, or natural killer T (NKT) cell. In some instances, the cell is CD4+ or CD8+ T cell. In some instances, the engineered immune cells is a CAR-T cell, CAR-macrophages, CAR-monocyte, CAR-granulocyte, CAR-NK cell, or a CAR-NKT cell. The compositions comprising the cells or engineered cells described herein may selectively bind to endothelial cells in a tumor environment, which can lead to enhanced clinical outcomes.

In another aspect, the present disclosure provides compositions and methods for treating solid tumors by targeting clinically relevant molecules that are upregulated in tumor cells. In some embodiments, the clinically relevant molecules may be upregulated in tumor vascular endothelial cells. The endothelial cells may comprise non-venular or venular endothelial cells in and surrounding a tumor, forming the tumor microvasculature, but they are not upregulated in vasculature from healthy tissues, e.g., non-tumor vascular endothelial cells. The present disclosure provides a composition comprising a first binder connected to a second binder via a first linker. In some instances, the first binder may specifically bind to a protein expressed on an endothelial cell. In some instances, the first binder and the second binder are different. In some instances, the first binder and the second binder are the same.

In another aspect, the present disclosure provides compositions and methods for treating solid tumors by targeting clinically relevant molecules that are upregulated in tumor cells. In some embodiments, the clinically relevant molecules may be upregulated in tumor vascular endothelial cells. The endothelial cells may comprise non-venular or venular endothelial cells in and surrounding a tumor, forming the tumor microvasculature, but they are not upregulated in vasculature from healthy tissues, e.g., non-tumor vascular endothelial cells. In one aspect, the present disclosure provides a composition and methods to deliver a therapeutic payload to a target either globally or in distinct organs. In another aspect, this disclosure provides a composition comprising a binder connected to a payload via a first linker. In some instances, the binder may specifically bind to a protein expressed on an endothelial cell.

### Definitions

In order that the present invention may be more readily understood, certain terms are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” means within 5%, or more preferably within 1%, of a given value or range.

As used herein, the term “encode” or “encoding” refers to a property of sequences of nucleic acids, such as a vector, a plasmid, a gene, cDNA, mRNA, to serve as templates for synthesis of other molecules such as proteins.

The terms “increased,” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

It should be noted that whenever a value or range of values of a parameter are recited, it is intended that values and ranges intermediate to the recited values are also intended to be part of this invention.

As used herein, the term “tumor vascular endothelial cell” and “tumor endothelial cell” can be used interchangeably and refer to the endothelial cells that are associated with a tumor. Vascular endothelial cells form the lining of the inner surface of all blood vessels, and constitute a non-thrombogenic interface between blood and tissue. In addition, vascular endothelial cells are an important component for the development of new capillaries and blood vessels. Tumor vascular endothelial cells proliferate during the angiogenesis, or neovascularization, associated with tumor growth and metastasis.

Tumor vascular endothelial cells are associated with new capillaries and blood vessels associated with tumors. (See Dudley A C. Tumor endothelial cells. Cold Spring Harb Perspect Med. 2012; 2(3):a006536. doi: 10.1101/cshperspect.a006536). The tumor vascular endothelial cells include both venular and non-venular endothelial cells found in or surrounding tumor.

As used herein, the term “non-tumor vascular endothelial cell” and “non-tumor endothelial cell” are used interchangeably and refer to endothelial cells that are not associated with a tumor and, for example, are found in normal “healthy” tissues.

The vascular endothelium is a dynamic cellular “organ” that controls passage of nutrients into tissues, maintains the flow of blood, and regulates the trafficking of leukocytes (e.g., T cell). In normal tissues, the endothelial cells form a continuous and uniform monolayer, while tumor endothelial cells are irregular in shape and size and have cytoplasmic projection extending into the vessel lumen. Tumor vascular endothelial cells can block T cells from entry into the tumor through the deregulation of adhesion molecules in the vessels. (See Lanitis E, Irving M, Coukos G. Targeting the tumor vasculature to enhance T cell activity. Curr Opin Immunol. 2015; 33:55-63. doi:10.1016/j.coi.2015.01.011).

As used herein, the term “tumor stroma” refers to a heterogeneous component of a tumor microenvironment. The “tumor stroma” is made up of noncellular and cellular components such as the extracellular matrix, the tumor-vasculature and tumor stromal cells.

As used herein, the term “tumor stromal cell” refers to a non-cancerous cell and non-immune cell within a tumor, and the tumor stromal cell is within the “tumor stroma.” Tumor stromal cells include connective tissue cells such as fibroblasts, e.g., cancer-associated fibroblasts, mesenchymal stromal cells, and pericytes. In solid tumors, the stromal cells interact with neoplastic cells to influence the behavior of a tumor.

As used herein, the term “non-tumor stromal cells” refers to stromal cells that are not associated with a tumor and, for example, are found in normal “healthy” tissues.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The expressions “at least about A, B, and C” and “at least about A, B, or C” may be construed to mean at least about A, at least about B, or at least about C. The expressions “at most about A, B, and C” and “at most about A, B, or C” may be construed to mean at most about A, at most about B, or at most about C.

The expression “about A to B and C to D” may be construed to mean between about A and about B and between about C and about D. The expression “about A to B or C to D” may be construed to mean between about A and about B or between about C and about D.

The term “exemplary” as used herein means “serving as an example, instance, or illustration.” Any embodiment described herein as “exemplary” is not to be construed as preferred or advantageous over other embodiments.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject.

An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “diagnosis” as used herein refers to methods by which the skilled artisan can estimate and/or determine whether or not a patient is suffering from a given disease or condition. The skilled artisan often makes a diagnosis on the basis of one or more diagnostic indicators, e.g., a biomarker, the presence, absence, amount, or change in amount of which is indicative of the presence, severity, or absence of the condition.

As used herein the term “prognosis” shall be taken to mean an indicator of the predicted progression of the disease (including but not limited to aggressiveness and metastatic potential) and/or predicted patient survival time.

As used herein, the term “identifying” or grammatical variations thereof refer to determining the presence of a diagnostic indicators, e.g., tumor vascular endothelial cells expressing one or more transmembrane molecules from Tables 1-3, wherein the one or more transmembrane molecules are upregulated when compared to a non-diseased control.

The term “control” or “control sample,” as used herein, refers to any clinically relevant control sample, including, for example, a sample from a healthy subject not afflicted with the disease or condition being assayed (e.g., cancer), a sample from a subject having a less severe or slower progressing disease or condition (e.g., cancer) than the subject to be assessed, a sample from a subject having some other type of cancer or disease, and the like. A control sample may include a sample derived from one or more subjects. A control sample may also be a sample made at an earlier timepoint from the subject to be assessed. For example, the control sample could be a sample taken from the subject to be assessed before the onset of the disease or condition being assayed (e.g., cancer), at an earlier stage of disease, or before the administration of treatment or of a portion of treatment. The control sample may also be a sample from an animal model, or from a tissue or cell lines derived from the animal model, of the disease or condition being assayed (e.g., cancer). For example, the expression level of a molecule, such as the proteins listed in Tables 1-3, in a control sample that consists of a group of measurements may be determined based on any appropriate statistical measure, such as, for example, measures of central tendency including average, median, or modal values.

The term “control level” refers to an accepted or pre-determined expression level of a molecule, such as the proteins listed in Tables 1-3 which is used to compare with the expression level of a molecule, such as the proteins listed in Tables 1-3 in a sample derived from a subject. In one embodiment, the control level of a molecule, such as the proteins listed in Tables 1-3 is based on the expression level of the molecule in sample(s) from a subject(s) having slow disease progression. In another embodiment, the control level of the molecule, such as proteins listed in Tables 1-3, is based on the expression level in a sample from a subject(s) having rapid disease progression. In another embodiment, the control level of the molecule, such as proteins listed in Tables 1-3, in based on sample(s) from an unaffected, i.e., non-diseased, subject(s), i.e., a subject who does not have a disease or disorder (e.g., cancer). In yet another embodiment, the control level of the molecule, such as proteins listed in Tables 1-3, is based on the expression level of the molecule in a sample from a subject(s) prior to the administration of a therapy for the disease or disorder (e.g., cancer). In yet another embodiment, the control level of the molecule, such as proteins listed in Tables 1-3, is based on the expression level of the molecule in a sample from a subject(s) after the administration of a therapy for the disease or disorder (e.g., cancer). In one embodiment, the control level of the molecule, such as proteins listed in Tables 1-3, is based on the level in a sample(s) from an animal model of a disease or disorder, (e.g., cancer), a cell, or a cell line derived from the animal model of a disease or disorder, (e.g., cancer).

It should be noted that whenever a value or range of values of a parameter are recited, it is intended that values and ranges intermediate to the recited values are also intended to be part of this invention.

As used herein, a “targeting molecule” refers to any molecule that binds to a component associated with an organ, tissue, cell, extracellular matrix, and/or subcellular locale. In some embodiments, such a component is referred to as a “target” or a “marker”. In some embodiments, the target or marker is PMEPA-1. In some embodiments, a targeting molecule, e.g., antibody or antigen binding fragment thereof, in accordance with the present invention may be a polypeptide. In certain embodiments, polypeptides range from about 75 to about 300, from about 100 to about 250, from about 100 to about 200, from about 75 to about 250, or from about 75 to about 200 amino acids in size.

The terms “polypeptide” and “peptide” are used interchangeably herein, with “peptide” typically referring to a polypeptide having a length of less than about 100 amino acids. Polypeptides may contain L-amino acids, D-amino acids; or both and may contain any of a variety of amino acid modifications or analogs known in the art. Useful modifications include, e.g., terminal acetylation, amidation, lipidation, phosphorylation, glycosylation, acylation, farnesylation, sulfation, etc.

As used herein, the terms “fragment,” “derivative,” and “analog” refer to a polypeptide that substantially retains the same biological function or activity of a protein, e.g., nanobody, of the invention. Polypeptide fragments, derivatives or analogs of the invention may be (i) polypeptides having one or more conservative or non-conservative amino acid residues (preferably non-conservative amino acid residues) substituted. Such substituted amino acid residues may or may not be encoded by the genetic code; or (ii) a polypeptide having a substituent group in one or more amino acid residues; or (iii) a polypeptide formed by fusing a mature polypeptide and another compound (such as a compound that increases the half-life of the polypeptide, for example, polyethylene glycol); or (iv) a polypeptide formed by fusing an additional amino acid sequence to the polypeptide sequence (e.g., a leader or secretory sequence or a sequence used to purify this polypeptide or a proprotein sequence, or a fusion protein formed with a His tag). According to the teachings herein, these fragments, derivatives, and analogs are within the scope of one of ordinary skill in the art.

As used herein, an “antibody fragment” or “antigen binding fragment” (i.e. characteristic portion of an antibody) refers to any derivative of an antibody which is less than full-length. In some embodiments, an antibody fragment retains at least a significant portion of the full-length antibody's specific binding ability. Examples of such antibody fragments include, but are not limited to, Fab, Fab′, F(ab′)2, scFv, Fv, dsFv diabody, VHH, and Fd fragments. Antibody fragments also include, but are not limited, to Fc fragments.

As used herein, the terms “single domain antibody (VHH)” and “nanobodies” have the same meaning referring to a variable region of a heavy chain of an antibody, and construct a single domain antibody (VHH) consisting of only one heavy chain variable region. It is the smallest antigen-binding fragment with complete function. Generally, the antibodies with a natural deficiency of the light chain and the heavy chain constant region 1 (CH1) are first obtained, the variable regions of the heavy chain of the antibody are therefore cloned to construct a single domain antibody (VHH) consisting of only one heavy chain variable region.

As used herein, the term “variable” refers that certain portions of the variable region in the nanobodies vary in sequences, which forms the binding and specificity of various specific antibodies to their particular antigen. However, variability is not uniformly distributed throughout the nanobody variable region. It is concentrated in three segments called complementarity-determining regions (CDRs) or hypervariable regions in the variable regions of the light and heavy chain. The more conserved part of the variable region is called the framework region (FR). The variable regions of the natural heavy and light chains each contain four FR regions, which are substantially in a β-folded configuration, joined by three CDRs which form a linking loop, and in some cases can form a partially β-folded structure. The CDRs in each chain are closely adjacent to the others by the FR regions and form an antigen-binding site of the nanobody with the CDRs of the other chain (see Kabat et al., NIH Publ. No. 91-3242, Volume I, pages 647-669. (1991)). The constant regions are not directly involved in the binding of the nanobody to the antigen, but they exhibit different effects or functions, for example, involve in antibody-dependent cytotoxicity of the antibodies.

As used herein, the term “heavy chain variable region” and “VH” can be used interchangeably. As used herein, the term “light chain variable region” and “VL” can be used interchangeably. As used herein, the terms “variable region” and “complementary determining region (CDR)” can be used interchangeably.

Exemplary proteins that may be used as targeting molecules in accordance with the present invention include, but are not limited to, antibodies, or antigen-binding fragments thereof, receptors, cytokines, peptide hormones, glycoproteins, glycopeptides, proteoglycans, proteins derived from combinatorial libraries (e.g., Avimers™, Affibodies®, etc.), and characteristic portions thereof synthetic binding proteins such as Nanobodies™, AdNectins™, etc., can be used. In some embodiments, protein targeting molecules can be a nanobody.

As used herein, the term “excipient” refers to pharmacologically inactive ingredients that are included in a formulation with the API, e.g., ceDNA and/or lipid nanoparticles to bulk up and/or stabilize the formulation when producing a dosage form. General categories of excipients include, for example, bulking agents, fillers, diluents, antiadherents, binders, coatings, disintegrants, flavours, colors, lubricants, glidants, sorbents, preservatives, sweeteners, and products used for facilitating drug absorption or solubility or for other pharmacokinetic considerations.

“Amino” refers to the —NH₂ radical.

“Cyano” refers to the —CN radical.

“Nitro” refers to the —NO₂ radical.

“Oxo” refers to the ═O radical.

“Hydroxyl” refers to the —OH radical.

“Alkyl” generally refers to an acyclic (e.g., straight or branched) or cyclic hydrocarbon (e.g., chain) radical consisting solely of carbon and hydrogen atoms, such as having from one to fifteen carbon atoms (e.g., C₁-C₁₅ alkyl). Unless otherwise state, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In certain embodiments, an alkyl comprises one to thirteen carbon atoms (e.g., C₁-C₁₃ alkyl). In certain embodiments, an alkyl comprises one to eight carbon atoms (e.g., C1-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. Each recitation of “alkyl” provided herein, unless otherwise stated, includes a specific and explicit recitation of an unsaturated “alkyl” group. Similarly, unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is optionally substituted as described for “alkyl” groups.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like. Unless stated otherwise specifically in the specification, an alkylene chain is optionally substituted as described for alkyl groups herein.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R—OR^{a}, —R—OC(O)—R^{a}, —R—OC(O)—OR^{a}, —R—OC(O)—N(R^{a})₂, —R—N(R^{a})₂, —R—C(O)R^{a}, —R—C(O)OR^{a}, —R—C(O)N(R^{a})₂, —R—OR—C(O)N(R^{a})₂, —R—N(R^{a})C(O)OR^{a}, —R—N(R^{a})C(O)R^{a}, —R—N(R^{a})S(O)ₜR^{a} (where t is 1 or 2), —R—S(O)ₜR^{a} (where t is 1 or 2), —R—S(O)ₜOR^{a} (where t is 1 or 2) and —R—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkenyl” refers to a radical of the formula —R^{c}-carbocyclyl where R^{c} is an alkenylene chain as defined above. The alkenylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

“Halo” or “halogen” refers to fluoro, bromo, chloro, or iodo substituents.

“Haloalkyl” refers to an alkyl radical, as defined above, that is substituted by one or more halogen radicals, as defined above, for example, trihalomethyl, dihalomethyl, halomethyl, and the like. In some embodiments, the haloalkyl is a fluoroalkyl, such as, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal carbon atoms of the alkyl are substituted with a heteroatom (with the appropriate number of substituents or valencies—for example, —CH₂— may be replaced with —NH— or —O—). For example, each substituted carbon atom is independently substituted with a heteroatom, such as wherein the carbon is substituted with a nitrogen, oxygen, sulfur, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁₈ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. In some embodiments, heteroalkyl includes alkylamino, alkylaminoalkyl, aminoalkyl, heterocycloalkyl, heterocycloalkyl, heterocyclyl, and heterocycloalkylalkyl, as defined herein. Unless stated otherwise specifically in the specification, heteroalkyl does not include alkoxy as defined herein. Unless stated otherwise specifically in the specification, a heteroalkyl group is optionally substituted as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl radical is saturated (i.e., containing single C—C bonds only) or unsaturated (e.g., containing one or more double bonds or triple bonds in the ring system). In some instances, the heterocyclyl radical is saturated. In some instances, the heterocyclyl radical is saturated and substituted. In some instances, the heterocyclyl radical is unsaturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{b}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—OR^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

In general, optionally substituted groups are each independently substituted or unsubstituted. Each recitation of a optionally substituted group provided herein, unless otherwise stated, includes an independent and explicit recitation of both an unsubstituted group and a substituted group (e.g., substituted in certain embodiments, and unsubstituted in certain other embodiments). Unless otherwise stated, a substituted group provided herein (e.g., substituted alkyl) is substituted by one or more substituent, each substituent being independently selected from the group consisting of halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2), where each R^{a} is independently hydrogen, alkyl (e.g., optionally substituted with halogen, hydroxy, methoxy, or trifluoromethyl), fluoroalkyl, carbocyclyl (e.g., optionally substituted with halogen, hydroxy, methoxy, or trifluoromethyl), carbocyclylalkyl (e.g., optionally substituted with halogen, hydroxy, methoxy, or trifluoromethyl), aryl (e.g., optionally substituted with halogen, hydroxy, methoxy, or trifluoromethyl), aralkyl (e.g., optionally substituted with halogen, hydroxy, methoxy, or trifluoromethyl), heterocyclyl (e.g., optionally substituted with halogen, hydroxy, methoxy, or trifluoromethyl), heterocyclylalkyl (e.g., optionally substituted with halogen, hydroxy, methoxy, or trifluoromethyl), heteroaryl (e.g., optionally substituted with halogen, hydroxy, methoxy, or trifluoromethyl), or heteroarylalkyl (e.g., optionally substituted with halogen, hydroxy, methoxy, or trifluoromethyl).

The section headings used herein are for organizational purposes only and are not to be construed as limiting the subject matter described.

### Radiopharmaceutical Composition

Provided herein, are compositions comprising a binder, a radioactive isotope, and a linker. In some embodiments, the compositions provided herein comprise a binder connected to a radioactive isotope via a first linker. In some embodiments, the binder is connected to a masking moiety via a second linker and the binder is connected to a radioactive isotope via a first linker. In some embodiments, the compositions provided herein comprise more than one binder (e.g., such as 2 binders, 3 binders, 4 binders, or 5 binders). In some embodiments, the compositions provided herein comprise more than one radioactive isotope (e.g., such as 2 isotopes, 3 isotopes, 4 isotopes, or 5 isotopes). In some embodiments, the radioactive isotope comprises a chelate comprising the radioactive isotope (e.g., such that the chelate stabilizes the radioactive isotope). In some embodiments, the chelate is connected to the linker. In some embodiments, the compositions provided herein comprise more than one linker, such as when the composition comprises more than one radioactive isotope or more than one binder (e.g., 2 linkers, 3 linkers, 4 linkers, or 5 linkers). In some embodiments, the binder binds to a protein, such as a protein provided elsewhere herein, expressed on a cell. In some embodiments, the binder specifically binds to a protein, such as a protein provided elsewhere herein, expressed on an endothelial cell.

In some embodiments, provided herein is a compound of Formula (I):

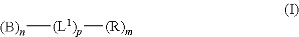

In some embodiments, B is a binder, such as a binder described elsewhere herein. In some embodiments, a binder is an antibody or antigen-binding fragment (e.g., such as anti-PMEPA-1), which can (e.g., substantially) specifically bind to an endothelial cell. In some embodiments, n is 1, 2, 3, 4, or 5. In some embodiments, n is 1. In some embodiments, n is 2. In some embodiments, L¹ is a first linker, such as a first linker described elsewhere herein. In some embodiments, p is 1, 2, 3, 4, or 5. In some embodiments, p is 1. In some embodiments p is 2. When two or more linkers are present (e.g., when p>1), the multiple linkers may be branched or organized in a chain. In some embodiments, the first linker is a cleavable or non-cleavable linker. In some embodiments, the first linker is a protease-cleavable linker, a self-immolative linker, or a pH-sensitive linker. In some embodiments, R is a radioactive isotope, such as a radioactive isotope described elsewhere herein. In some embodiments, the radioactive isotope is an alpha emitter, beta emitter, or gamma emitter. In some embodiments, the radioactive isotope may be used for imaging, such as targeting imaging (e.g., of endothelial cells of tumors). In some embodiments, m is 1, 2, 3, 4, or 5. In some embodiments, m is 1. In some embodiments, m is 2. In some instances, for example, such as when n is 1, m is 1, and p is 1, the compound of Formula (I) is B-L¹-R.

In some embodiments, provided herein is a compound of Formula (II):

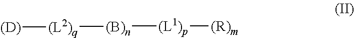

In some embodiments, D is a masking moiety, such as a masking moiety provided elsewhere herein. In some embodiments, the masking moiety is an anti-idiotypic antibody or fragment thereof. In some embodiments, the masking moiety is an anti-idiotypic scFv or fragment thereof. In some instances, the masking moiety allows for binding of the binder (e.g., B) in the presence of acidic conditions of a tumor microenvironment. In some embodiments, B is a binder, such as a binder described elsewhere herein. In some embodiments, a binder is an antibody or antigen-binding fragment (e.g., such as anti-PMEPA-1), which can (e.g., substantially) specifically bind to an endothelial cell. In some embodiments, n is 1, 2, 3, 4, or 5. In some embodiments, n is 1. In some embodiments, n is 2. In some embodiments, L¹ is a first linker, such as a first linker described elsewhere herein. In some embodiments, p is 1, 2, 3, 4, or 5. In some embodiments, p is 1. In some embodiments p is 2. When two or more linkers are present (e.g., when p>1), the multiple linkers may be branched or organized in a chain. In some embodiments, the first linker is a cleavable or non-cleavable linker. In some embodiments, the first linker is a protease-cleavable linker, a self-immolative linker, or a pH-sensitive linker. In some embodiments, L² is a second linker, such as a second linker described elsewhere herein. In some embodiments, q is 0, 1, 2, or 3. In some embodiments, p is 0. In some embodiments p is 1. In some embodiments, the second linker is a cleavable or non-cleavable linker. In some embodiments, the second linker is a protease-cleavable linker, a self-immolative linker, or a pH-sensitive linker. In some embodiments, R is a radioactive isotope, such as a radioactive isotope described elsewhere herein. In some embodiments, m is 1, 2, 3, 4, or 5. In some embodiments, the radioactive isotope is an alpha emitter, beta emitter, or gamma emitter. In some embodiments, the radioactive isotope may be used for imaging, such as targeting imaging (e.g., of endothelial cells of tumors). In some embodiments, m is 1. In some embodiments, m is 2. In some instances, for example, such as when n is 1, m is 1, q is 1, and p is 1, the compound of Formula (II) is D-L²-B-L¹-R.

In some embodiments, provided herein is a compound of Formula (III):

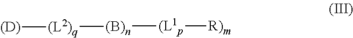

In some embodiments, D is a masking moiety, such as a masking moiety provided elsewhere herein. In some embodiments, the masking moiety is an anti-idiotypic antibody or fragment thereof. In some embodiments, the masking moiety is an anti-idiotypic scFv or fragment thereof. In some instances, the masking moiety allows for binding of the binder (e.g., B) in the presence of acidic conditions of a tumor microenvironment. In some embodiments, B is a binder, such as a binder described elsewhere herein. In some embodiments, a binder is an antibody or antigen-binding fragment (e.g., such as anti-PMEPA-1), which can (e.g., substantially) specifically bind to an endothelial cell. In some embodiments, n is 1, 2, 3, 4, or 5. In some embodiments, n is 1. In some embodiments, n is 2. In some embodiments, L¹ is a first linker, such as a first linker described elsewhere herein. In some embodiments, p is 1, 2, 3, 4, or 5. In some embodiments, p is 1. In some embodiments p is 2. When two or more linkers are present (e.g., when p>1), the multiple linkers may be branched or organized in a chain. In some embodiments, the first linker is a cleavable or non-cleavable linker. In some embodiments, the first linker is a protease-cleavable linker, a self-immolative linker, or a pH-sensitive linker. In some embodiments, L² is a second linker, such as a second linker described elsewhere herein. In some embodiments, q is 0, 1, 2, or 3. In some embodiments, p is 0. In some embodiments p is 1. In some embodiments, the second linker is a cleavable or non-cleavable linker. In some embodiments, the second linker is a protease-cleavable linker, a self-immolative linker, or a pH-sensitive linker. In some embodiments, R is a radioactive isotope, such as a radioactive isotope described elsewhere herein. In some embodiments, m is 1, 2, 3, 4, or 5. In some embodiments, the radioactive isotope is an alpha emitter, beta emitter, or gamma emitter. In some embodiments, the radioactive isotope may be used for imaging, such as targeting imaging (e.g., of endothelial cells of tumors). In some embodiments, m is 1. In some embodiments, m is 2. In some instances, for example, such as when n is 1, m is 2, q is 1, and p is 1, the compound of Formula (III) is D-L²-B-(L¹R)₂.

### Immune Cells

Provided herein are cells comprising a first binder that specifically binds to a protein expressed on an endothelial cells. In some embodiments, the cell is an immune cell or an engineered immune cell. Examples of the immune cell include, but not limited to, a T cell, macrophage, monocyte, granulocyte, natural killer (NK) cell, or natural killer T (NKT) cell. In some embodiments, the cells are isolated T cells. In some embodiments, the cells are isolated from a patient's tumor. In some embodiment, the cells are isolated from a patient's tumor, cultured in vitro, and infused back to the patient. In some embodiments, the T cell is a tumor-infiltrating lymphocyte (TIL) or a cytotoxic T lymphocyte (CTL). In some embodiments, the cell expresses a tumor-specific T-cell receptor. In some embodiments, the immune cell is CD4+ or CD8+ T cell. Examples of the engineered immune cell include, but not limited to, a CAR-T cell, CAR-macrophages, CAR-monocyte, CAR-granulocyte, CAR-NK cell, or a CAR-NKT cell. In some embodiments, the engineered immune cell is a CAR-T cell, CAR-NK cell, or a CAR-NKT cell. In some embodiments, the engineered immune cell is a CAR-T cell.

As used herein, a “chimeric antigen receptor” (CAR) is an artificially constructed hybrid protein or polypeptide comprising a specificity or recognition (i.e. binding) domain linked to an immune receptor responsible for signal transduction in lymphocytes. The binding domain is typically derived from a Fab antibody fragment that has been fashioned into a single chain scFv via the introduction of a flexible linker between the antibody chains within the specificity domain. Other possible specificity domains can include the signaling portions of hormone or cytokine molecules, the extracellular domains of receptors, and peptide ligands or peptides isolated by library (e.g. phage) screening (see Ramos and Dotti, (2011) Expert Opin Bio Ther 11(7): 855). Flexibility between the signaling and the binding portions of the CAR may be a desirable characteristic to allow for more optimum interaction between the target and the binding domain, so often a hinge region is included. One example of a structure that can be used is the CH2-CH3 region from an immunoglobulin such as an IgG molecule. The signaling domain of the typical CAR comprises intracellular domains of the TCR-CD3 complex such as the zeta chain. Alternatively, the γ chain of an Fe receptor may be used. The transmembrane portion of the typical CAR can comprise transmembrane portions of proteins such as CD4, CD8 or CD28 (Ramos and Dotti, ibid). Characteristics of some CARs include their ability to redirect T-cell specificity and reactivity toward a selected target in a non-MHC-restricted manner. The non-MHC-restricted target recognition gives T-cells expressing CARs the ability to recognize a target independent of antigen processing, thus bypassing a major mechanism of tumor escape.

In some embodiments, the surface of the CAR T cells is decorated with one or more binders. In some embodiments, the one or more binders are embedded in the lipid membrane of the CAR T cell. In some embodiments, the one or more binders are associated with the lipid membrane of the CAR T cell (e.g., binding to molecule on the exterior of the CAR T cell, covalently linked to a molecule on the exterior of the CAR T cell). In some embodiments, there are two or more different types of binders on the exterior of the CAR T cells.

In some embodiments, provided herein is a compound of Formula (IV):

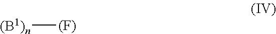

In some embodiments, B is a binder, such as a binder described elsewhere herein. In some embodiments, a binder is an antibody or antigen-binding fragment (e.g., such as anti-PMEPA-1), which can (e.g., substantially) specifically bind to an endothelial cell. In some embodiments, n is 1, 2, 3, 4, or 5. In some embodiments, n is 1. In some embodiments, n is 2. In some embodiments, F is a cell, such as an an immune cell or an engineered immune cell. In some embodiments, the immune cell is a T cell, macrophage, monocyte, granulocyte, or natural killer (NK) cell, or natural killer T (NKT) cell. In some embodiments, the T cell is a tumor-infiltrating lymphocyte (TIL) or a cytotoxic T lymphocyte (CTL). In some embodiments, the immune cell CD4+ or CD8+ T cell. In some embodiments, the engineered immune call is a CAR-immune cell. In some embodiments, the CAR-immune cell is a CAR-T cell, CAR-macrophages, CAR-monocyte, CAR-granulocyte, CAR-NK cell, or a CAR-NKT cell. In some instances, for example, such as when n is 1, the compound of Formula (IV) is B-F.

In some embodiments, provided herein is a compound of Formula (V):

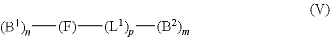

In some embodiments, B is a binder, such as a binder described elsewhere herein. In some embodiments, a binder is an antibody or antigen-binding fragment (e.g., such as anti-PMEPA-1), which can (e.g., substantially) specifically bind to an endothelial cell. In some embodiments, B¹ is a first binder and B² is a second binder. In some embodiments, n is 1, 2, 3, 4, or 5. In some embodiments, n is 1. In some embodiments, n is 2. In some embodiments, m is 1, 2, 3, 4, or 5. In some embodiments, m is 1. In some embodiments, m is 2. In some embodiments, F is a cell, such as an immune cell or an engineered immune cell. In some embodiments, the immune cell is a T cell, macrophage, monocyte, granulocyte, or natural killer (NK) cell, or natural killer T (NKT) cell. In some embodiments, the T cell is a tumor-infiltrating lymphocyte (TIL) or a cytotoxic T lymphocyte (CTL). In some embodiments, the immune cell CD4+ or CD8+ T cell. In some embodiments, the engineered immune call is a CAR-immune cell. In some embodiments, the CAR-immune cell is a CAR-T cell, CAR-macrophages, CAR-monocyte, CAR-granulocyte, CAR-NK cell, or a CAR-NKT cell. In some embodiments, L¹ is a first linker, such as a first linker described elsewhere herein. In some embodiments, p is 1, 2, 3, 4, or 5. In some embodiments, p is 1. In some embodiments p is 2. When two or more linkers are present (e.g., when p>1), the multiple linkers may be branched or organized in a chain. In some embodiments, the first linker is a cleavable or non-cleavable linker. In some embodiments, the first linker is a protease-cleavable linker, a self-immolative linker, or a pH-sensitive linker.

In some embodiments, n is 1. In some embodiments, n is 2. In some embodiments, m is 1. In some embodiments, m is 2. In some embodiments, p is 1. In some embodiments, p is 2. In some instances, for example, such as when n is 1, m is 1, and p is 1 the compound of Formula (V) is B¹-F-L¹-B².

In some embodiments, provided herein is a compound of Formula (VI) and Formula (VII):

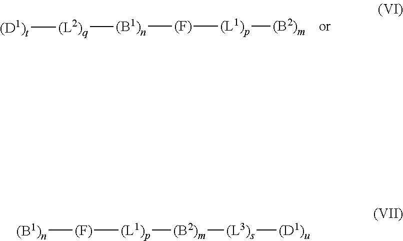

In some embodiments, D is a masking moiety, such as a masking moiety provided elsewhere herein. In some instances, D¹ is a first masking moiety. In some embodiments, the masking moiety is an anti-idiotypic antibody or fragment thereof. In some embodiments, the masking moiety is an anti-idiotypic scFv or fragment thereof. In some instances, the masking moiety allows for binding of the binder (e.g., B¹ and/or B²) in the presence of acidic conditions of a tumor microenvironment. In some embodiments, t is 1, 2, 3, 4, or 5. In some embodiments, t is 1. In some embodiments, t is 2.

In some embodiments, B is a binder, such as a binder described elsewhere herein. In some embodiments, a binder is an antibody or antigen-binding fragment (e.g., such as anti-PMEPA-1), which can (e.g., substantially) specifically bind to an endothelial cell. In some embodiments, B¹ is a first binder and B² is a second binder. In some embodiments, n is 1, 2, 3, 4, or 5. In some embodiments, n is 1. In some embodiments, n is 2. In some embodiments, m is 1, 2, 3, 4, or 5. In some embodiments, m is 1. In some embodiments, m is 2.

In some embodiments, F is a cell, such as an immune cell or an engineered immune cell. In some embodiments, the immune cell is a T cell, macrophage, monocyte, granulocyte, or natural killer (NK) cell, or natural killer T (NKT) cell. In some embodiments, the T cell is a tumor-infiltrating lymphocyte (TIL) or a cytotoxic T lymphocyte (CTL). In some embodiments, the immune cell CD4+ or CD8+ T cell. In some embodiments, the engineered immune call is a CAR-immune cell. In some embodiments, the CAR-immune cell is a CAR-T cell, CAR-macrophages, CAR-monocyte, CAR-granulocyte, CAR-NK cell, or a CAR-NKT cell.

In some embodiments, L¹ is a first linker, such as a first linker described elsewhere herein. In some embodiments, L² is a second linker, such as a second linker described elsewhere herein. In some embodiments, L³ is a third linker, such as a third linker described elsewhere herein. In some embodiments, p is 1, 2, 3, 4, or 5. In some embodiments, p is 1. In some embodiments p is 2. In some embodiments, q is 1, 2, 3, 4, or 5. In some embodiments, q is 1. In some embodiments q is 2. In some embodiments, s is 1, 2, 3, 4, or 5. In some embodiments, s is 1. In some embodiments s is 2. When two or more linkers are present (e.g., when p>1), the multiple linkers may be branched or organized in a chain. In some embodiments, the first linker is a cleavable or non-cleavable linker. In some embodiments, the first linker is a protease-cleavable linker, a self-immolative linker, or a pH-sensitive linker.

In some embodiments, n is 1. In some embodiments, n is 2. In some embodiments, m is 1. In some embodiments, m is 2. In some embodiments, p is 1. In some embodiments, p is 2. In some embodiments, q is 1. In some embodiments, q is 2. In some embodiments, t is 1. In some embodiments, t is 2. In some embodiments, s is 1. In some embodiments, s is 2. In some embodiments, u is 1. In some embodiments, u is 2. In some instances, for example, such as when n is 1, m is 1, p is 1, q is 1, and t is 1, the compound of Formula (VI) is D¹-L²-B¹-F-L¹-B². In some instances, for example, such as when n is 1, m is 1, p is 1, s is 1, and u is 1, the compound of Formula (VI) is B¹-F-L¹-B²-L³-D¹.

In some embodiments, provided herein is a compound of Formula (VIII):

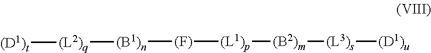

In some embodiments, D is a masking moiety, such as a masking moiety provided elsewhere herein. In some instances, D¹ is a first masking moiety. In some instances, D² is a second masking moiety.

In some embodiments, the masking moiety is an anti-idiotypic antibody or fragment thereof. In some embodiments, the masking moiety is an anti-idiotypic scFv or fragment thereof. In some instances, the masking moiety allows for binding of the binder (e.g., B¹ and/or B²) in the presence of acidic conditions of a tumor microenvironment. In some embodiments, t is 1, 2, 3, 4, or 5. In some embodiments, t is 1. In some embodiments, t is 2. In some embodiments, u is 1, 2, 3, 4, or 5. In some embodiments, u is 1. In some embodiments, u is 2.

In some embodiments, B is a binder, such as a binder described elsewhere herein. In some embodiments, a binder is an antibody or antigen-binding fragment (e.g., such as anti-PMEPA-1), which can (e.g., substantially) specifically bind to an endothelial cell. In some embodiments, B¹ is a first binder and B² is a second binder. In some embodiments, n is 1, 2, 3, 4, or 5. In some embodiments, n is 1. In some embodiments, n is 2. In some embodiments, m is 1, 2, 3, 4, or 5. In some embodiments, m is 1. In some embodiments, m is 2.

In some embodiments, F is a cell, such as an immune cell or an engineered immune cell. In some embodiments, the immune cell is a T cell, macrophage, monocyte, granulocyte, or natural killer (NK) cell, or natural killer T (NKT) cell. In some embodiments, the T cell is a tumor-infiltrating lymphocyte (TIL) or a cytotoxic T lymphocyte (CTL). In some embodiments, the immune cell CD4+ or CD8+ T cell. In some embodiments, the engineered immune call is a CAR-immune cell. In some embodiments, the CAR-immune cell is a CAR-T cell, CAR-macrophages, CAR-monocyte, CAR-granulocyte, CAR-NK cell, or a CAR-NKT cell.

In some embodiments, L¹ is a first linker, such as a first linker described elsewhere herein. In some embodiments, L² is a second linker, such as a second linker described elsewhere herein. In some embodiments, L³ is a third linker, such as a third linker described elsewhere herein. In some embodiments, p is 1, 2, 3, 4, or 5. In some embodiments, p is 1. In some embodiments p is 2. In some embodiments, q is 1, 2, 3, 4, or 5. In some embodiments, q is 1. In some embodiments q is 2. In some embodiments, s is 1, 2, 3, 4, or 5. In some embodiments, s is 1. In some embodiments s is 2. When two or more linkers are present (e.g., when p>1), the multiple linkers may be branched or organized in a chain. In some embodiments, the first linker is a cleavable or non-cleavable linker. In some embodiments, the first linker is a protease-cleavable linker, a self-immolative linker, or a pH-sensitive linker.

In some embodiments, n is 1. In some embodiments, n is 2. In some embodiments, m is 1. In some embodiments, m is 2. In some embodiments, p is 1. In some embodiments, p is 2. In some embodiments, q is 1. In some embodiments, q is 2. In some embodiments, s is 1. In some embodiments, s is 2. In some embodiments, t is 1. In some embodiments, t is 2. In some embodiments, u is 1. In some embodiments, u is 2. In some instances, for example, such as when n is 1, m is 1, p is 1, q is 1, t is 1, s, is 1 and u is 1, the compound of Formula (VIII) is D¹-L²-B¹-F-L-B²-L³-D².

### Bispecific Antibody

In one aspect, this disclosure provides a composition comprising a first binder connected to a second binder via a first linker. In some embodiments, the first binder may specifically bind to a protein expressed on an endothelial cell. In some embodiments, the first binder and the second binder are different. In some embodiments, the first binder and the second binder are the same.

In some embodiments, provided herein is a compound of Formula (IX):

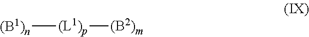

In some embodiments, B is a binder, such as a binder described elsewhere herein. In some embodiments, a binder is an antibody or antigen-binding fragment (e.g., such as anti-PMEPA-1), which can (e.g., substantially) specifically bind to an endothelial cell. In some embodiments, B¹ is a first binder and B² is a second binder. In some embodiments, n is 1, 2, 3, 4, or 5. In some embodiments, n is 1. In some embodiments, n is 2. In some embodiments, m is 1, 2, 3, 4, or 5. In some embodiments, m is 1. In some embodiments, m is 2.

In some embodiments, L¹ is a first linker, such as a first linker described elsewhere herein. In some embodiments, p is 1, 2, 3, 4, or 5. In some embodiments, p is 1. In some embodiments p is 2. When two or more linkers are present (e.g., when p>1), the multiple linkers may be branched or organized in a chain. In some embodiments, the first linker is a cleavable or non-cleavable linker. In some embodiments, the first linker is a protease-cleavable linker, a self-immolative linker, or a pH-sensitive linker.

In some embodiments, n is 1. In some embodiments, n is 2. In some embodiments, m is 1. In some embodiments, m is 2. In some embodiments, p is 1. In some embodiments, p is 2. In some instances, for example, such as when n is 1, m is 1, and p is 1, the compound of Formula (VIII) is B¹-L¹-B².

In some embodiments, provided herein is a compound of Formula (IX) or Formula (X):

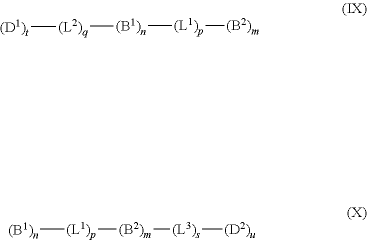

In some embodiments, D is a masking moiety, such as a masking moiety provided elsewhere herein. In some instances, D¹ is a first masking moiety. In some instances, D² is a second masking moiety. In some embodiments, the masking moiety is an anti-idiotypic antibody or fragment thereof. In some embodiments, the masking moiety is an anti-idiotypic scFv or fragment thereof. In some instances, the masking moiety allows for binding of the binder (e.g., B¹ and/or B²) in the presence of acidic conditions of a tumor microenvironment. In some embodiments, t is 1, 2, 3, 4, or 5. In some embodiments, t is 1. In some embodiments, t is 2. In some embodiments, u is 1, 2, 3, 4, or 5. In some embodiments, u is 1. In some embodiments, u is 2.

In some embodiments, B is a binder, such as a binder described elsewhere herein. In some embodiments, a binder is an antibody or antigen-binding fragment (e.g., such as anti-PMEPA-1), which can (e.g., substantially) specifically bind to an endothelial cell. In some embodiments, B¹ is a first binder and B² is a second binder. In some embodiments, n is 1, 2, 3, 4, or 5. In some embodiments, n is 1. In some embodiments, n is 2. In some embodiments, m is 1, 2, 3, 4, or 5. In some embodiments, m is 1. In some embodiments, m is 2.

In some embodiments, L¹ is a first linker, such as a first linker described elsewhere herein. In some embodiments, L² is a second linker, such as a second linker described elsewhere herein. In some embodiments, L³ is a third linker, such as a third linker described elsewhere herein. In some embodiments, p is 1, 2, 3, 4, or 5. In some embodiments, p is 1. In some embodiments p is 2. In some embodiments, q is 1, 2, 3, 4, or 5. In some embodiments, q is 1. In some embodiments q is 2. In some embodiments, s is 1, 2, 3, 4, or 5. In some embodiments, s is 1. In some embodiments s is 2. When two or more linkers are present (e.g., when p>1), the multiple linkers may be branched or organized in a chain. In some embodiments, the first linker is a cleavable or non-cleavable linker. In some embodiments, the first linker is a protease-cleavable linker, a self-immolative linker, or a pH-sensitive linker.

In some embodiments, n is 1. In some embodiments, n is 2. In some embodiments, m is 1. In some embodiments, m is 2. In some embodiments, p is 1. In some embodiments, p is 2. In some embodiments, q is 1. In some embodiments, q is 2. In some embodiments, s is 1. In some embodiments, s is 2. In some embodiments, t is 1. In some embodiments, t is 2. In some embodiments, u is 1. In some embodiments, u is 2. In some instances, for example, such as when n is 1, m is 1, p is 1, q is 1, and t is 1, the compound of Formula (IX) is D¹-L²-B¹-L¹-B². In some instances, for example, such as when n is 1, m is 1, p is 1, s is 1, and u is 1, the compound of Formula (X) is B¹-L¹-B²-L³-D².

In some embodiments, provided herein is a compound of Formula (XI):

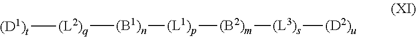

In some embodiments, D is a masking moiety, such as a masking moiety provided elsewhere herein. In some instances, D¹ is a first masking moiety. In some instances, D² is a second masking moiety.

In some embodiments, the masking moiety is an anti-idiotypic antibody or fragment thereof. In some embodiments, the masking moiety is an anti-idiotypic scFv or fragment thereof. In some instances, the masking moiety allows for binding of the binder (e.g., B¹ and/or B²) in the presence of acidic conditions of a tumor microenvironment. In some embodiments, t is 1, 2, 3, 4, or 5. In some embodiments, t is 1. In some embodiments, t is 2. In some embodiments, u is 1, 2, 3, 4, or 5. In some embodiments, u is 1. In some embodiments, u is 2.

In some embodiments, B is a binder, such as a binder described elsewhere herein. In some embodiments, a binder is an antibody or antigen-binding fragment (e.g., such as anti-PMEPA-1), which can (e.g., substantially) specifically bind to an endothelial cell. In some embodiments, B¹ is a first binder and B² is a second binder. In some embodiments, n is 1, 2, 3, 4, or 5. In some embodiments, n is 1. In some embodiments, n is 2. In some embodiments, m is 1, 2, 3, 4, or 5. In some embodiments, m is 1. In some embodiments, m is 2.

In some embodiments, L¹ is a first linker, such as a first linker described elsewhere herein. In some embodiments, L² is a second linker, such as a second linker described elsewhere herein. In some embodiments, L³ is a third linker, such as a third linker described elsewhere herein. In some embodiments, p is 1, 2, 3, 4, or 5. In some embodiments, p is 1. In some embodiments p is 2. In some embodiments, q is 1, 2, 3, 4, or 5. In some embodiments, q is 1. In some embodiments q is 2. In some embodiments, s is 1, 2, 3, 4, or 5. In some embodiments, s is 1. In some embodiments s is 2. When two or more linkers are present (e.g., when p>1), the multiple linkers may be branched or organized in a chain. In some embodiments, the first linker is a cleavable or non-cleavable linker. In some embodiments, the first linker is a protease-cleavable linker, a self-immolative linker, or a pH-sensitive linker.

In some embodiments, n is 1. In some embodiments, n is 2. In some embodiments, is 1. In some embodiments, n is 2. In some embodiments, p is 1. In some embodiments, p is 2. In some embodiments, q is 1. In some embodiments, q is 2. In some embodiments, s is 1. In some embodiments, s is 2. In some embodiments, t is 1. In some embodiments, t is 2. In some embodiments, is 1. In some embodiments, t instances, for example, such as when n is 1, m is 1, p is 1, q is 1, t is 1, and u is 1 the compound of Formula (XI) is D¹-L²-B¹-L¹-B²-L³-D².

### Antibody Drug Conjugate

In a certain aspect, the present disclosure provides a composition and methods to deliver a therapeutic payload to a target either globally or in distinct organs.

In one aspect, the present disclosure also provides a composition having a structure of Formula (XII):

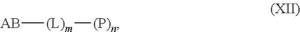

wherein:
   AB is an antibody or an antigen-binding fragment;
   L is a linker;
   P is a payload;
   m is 0 to 10; and
   n is 1 to 20.

In one aspect, the present disclosure also provides a composition having a structure of Formula (XII):

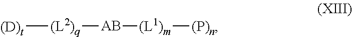

wherein:
   AB is an antibody or an antigen-binding fragment;
   L is a linker;
   P is a payload;
   D is a masking moiety;
   m is 0 to 10;
   n is 1 to 20;
   q is 1 to 5; and
   t is 1 to 5.

In some embodiments, AB may be an antibody. In some embodiments, AB may be an antigen-binding fragment. An antibody may be monoclonal or polyclonal. An antibody may be a member of any immunoglobulin class, including any of the human classes: IgG, IgM, IgA, IgD, and IgE. In some embodiments, AB may be an antibody fragment or antigen binding fragment. Examples of such antibody fragments include, but are not limited to, Fab, Fab′, F(ab′)2, scFv, Fv, dsFv diabody, VHH, and Fd fragments. Antibody fragments also include, but are not limited, to Fc fragments. In some embodiments, antibodies may comprise chimeric or single chain (recombinant) antibodies. In some embodiments, antibodies may have reduced effector functions and/or bispecific molecules. In some embodiments, antibodies may comprise fragments produced by a Fab expression library.

In some embodiments, the antibody or antigen binding fragment may be a nanobody. Nanobodies are recombinant antibody fragments consisting of one variable heavy chain. In some embodiments, the variable heavy chain of a nanobody comprises a CDR1, CDR2, and CDR3. The CDR1 and CDR2 segments can be short in comparison to the CDR3 segment, which is longer than the typical CDR3 in a conventional antibody or scFv molecule.

In some embodiments, the nanobodies may comprise multiple (two or more) VH segments, such as a dimer. Peptide linker can be between VH segments. Each VH segment in a multimer nanobody can be the same VH sequence binding to the same antigen, or different VH sequence binding to different antigens, or different VH sequences binding the same antigen at non-overlapping epitopes. In some embodiments, the nanobodies can comprise multiple segments of VH segments as described above and scFv molecules.

In some embodiments, AB may comprise antibody fragments, characteristic portions of antibodies, or single chain antibodies. In some embodiments, AB may be synthetic binding proteins comprising Affibodies®, Nanobodies™, AdNectins™, or Avimers™.

In some embodiments, an association between AB and P may be mediated by L. Any suitable linker can be used.

In some embodiments, L may be a polypeptide linker. In some embodiments, the polypeptide linker may comprise about 2-20 amino acids. In some embodiments, L¹ is a first linker, such as a first linker described elsewhere herein. In some embodiments, L² is a second linker, such as a second linker described elsewhere herein. In some embodiments, L³ is a third linker, such as a third linker described elsewhere herein.

In some embodiments, m is 1, 2, 3, 4, or 5. In some embodiments, m is 1. In some embodiments m is 2. In some embodiments, q is 1, 2, 3, 4, or 5. In some embodiments, q is 1. In some embodiments q is 2. When two or more linkers are present (e.g., when q or m>1), the multiple linkers may be branched or organized in a chain.

In some embodiments, the polypeptide linker may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, the polypeptide linker may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, the polypeptide linker comprises (G₄S)ₙ, (SG₄)ₙ, G₄(SG₄)ₙ or G₂(SG₂)ₙ, wherein n is selected from 1 to 10. In some embodiments, the peptide linkers may comprise the dipeptide Val Cit (VC), the tripeptide AAN, or a longer peptide such as (GGGGS)(n=1, 2, 3, or 4) (SEQ ID NO: 32), (Gly)₈ (SEQ ID NO: 33), (Gly)₆ (SEQ ID NO: 34), (EAAAK)₃(SEQ ID NO: 35), (EAAAK)(n=1-3) (SEQ ID NO: 36), A(EAAAK)₄ALEA(EAAAK)₄A (SEQ ID NO: 37), PAPAP (SEQ ID NO: 38), AEAAAKEAAAKA (SEQ ID NO: 39), (Ala-Pro)n (10-34 aa) (SEQ ID NO: 40).

In some embodiments, L may comprise GPI-anchors or cross-linked polymers. In some embodiments, L may be a flexible.

In some embodiments, L may comprise at least one group selected from the group consisting of alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene is optionally substituted.

In some embodiments, L may comprise substituted C₁-C₆ alkylene. In some embodiments, L may comprise unsubstituted C₁-C₆ alkylene. In some embodiments, L may comprise substituted C₁-C₆ heteroalkylene. In some embodiments, L may comprise unsubstituted C₁-C₆ heteroalkylene.

In some embodiments, L may comprise one or more groups selected from the group consisting of —O—, —S—, —NH—, —NH—(CH2)y-NH, —NH—(CH2)_{y}—O, —O—(CH2)_{y}—O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH₂CH₂)_{w}—(C═O)—, —(C═O)—(CH═CH), —(C═O), —(C═O)—(OCH₂CH₂O)_{w}—(C═O)—, —(CH₂CH₂O)_{w}—, —(C═O)—(CH2CH2O)_{w}—, and —(CH(CH3)C(═O)O)_{w}—, wherein w is 1-20 and y is 1-20.

In some embodiments, L may be a cleavable linker. In some embodiments, a cleavable linker may comprise an acid-labile linker, a protease cleavable linker, an enzyme cleavable linker, or a reducible disulfide linkage. In some embodiments, the cleavable linkers may comprise those comprising an ester bond such as a glutaryl linker, those comprising an amide bond, and those comprising a carbamate bond.

In some embodiments, an acid-labile linker may be a hydrazone linker. In some embodiments, the cleavable linker may be a protease-cleavable linker, a self-immolative linker, or a pH-sensitive linker.

In some embodiments, the protease-cleavable linker may comprise at least one protease recognition site. In some embodiments, the protease may be selected from metalloproteinase (MMP) 1-28; A Disintegrin and Metalloproteinase (ADAM) 2, 7-12, 15, 17-23, 28-30 and 33; serine protease; urokinase-type plasminogen activator; Matriptase; cysteine protease; aspartic protease; and cathepsin protease. In some embodiments, the protease may be MMP2 or MMP9.

In some embodiments, the self-immolative linker may be selected from para-amino benzoic acid (PAB), para-aminobenzyl alcohol (PABA), 3,3-dimethyl-4-hydroxybutyric acid, ethylenediamine, γ-aminobutyric acid (GABA), 2-hydroxycinnamic acid, “Trimethyl Lock”, or ethanolamine. In some embodiments, the self-immolative linker may be para-amino benzoic acid (PAB).

In some embodiments, the pH-sensitive linker may be cleaved upon exposure to a target pH. In some embodiments, the target pH may be less than about 7. In some embodiments, the target pH may be less than at least about 2, 3, 4, 5, 6, 7, 8, or 9. In some embodiments, the target pH may be less than at most about 2, 3, 4, 5, 6, 7, 8, or 9. In some embodiments, the target pH may be about 2 to 9, 3 to 8, 4 to 7, or 5 to 6. In some embodiments, the pH-sensitive linker may be selected from an optionally substituted tetrahydropyranyl ether, an optionally substituted tetrahydropyranyl ester, an optionally substituted azide, an optionally substituted histidine, an optionally substituted hydrazone, or an optionally substituted β-amino ester. In some embodiments, the pH-sensitive linker may be selected from -(tetrahydropyran ether)-(azide), -(hydrazone)-, -(hydrazone)-(azide)-, -(β-amino ester)-, -(β-amino ester)-(azide)-, or -(tetrahydropyran ester)-.

In some embodiments, L may be a non-cleavable linker. In some embodiments, the uncleavable linkers may comprise an amide bond, a succinimidyl thioester linker, a triazole linker, or an oxime linker.

In some embodiments, P may be a toxin. In some embodiments, P may be a cytokine. In some embodiments, P may be cytotoxic. In some embodiments, P may be cytotoxic to a tumor cell upon internalization into the tumor cell.

In some embodiments, P may comprise an antitumor antibiotic, microtubule inhibitor, a cytotoxic drug, a cytostatic drug, topoisomerase inhibitor, a DNA-alkylating drug, a DNA-binding drug, a DNA-cleaving drug, or an RNA polymerase inhibitor. In some embodiments, the payload may comprise pyrrolobenzodiazepine, duocarmycin, auristatin, maytansinoid, uncialamycin, dynemicin, thailanstatin, camptothecin, exatecan, tubulysin compound, lurbinectedin, trabectedin, safracin, lenalidomide, eribulin, vincristine, vinblastine, vindesine, vinorelbine, an epothilone, a taxane (e.g., paclitaxel, docetaxel, cabazitaxel, etc.), a cryptophycin, a hemiasterlin, an anthracyclin, a bisnaphthylamide (e.g., elinafide), or a cytotoxic molecular glue/PROTAC compound.

In some embodiments, P comprises dolastatin 10, auristatin E, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), or 5-benzoylvaleric acid-AE ester (AEVB).

In some embodiments, a ratio of P to AB may be about 1-30. In some embodiments, a ratio of P to AB may be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35, 36, 37, 38, 39, or 40. In some embodiments, a ratio of P to AB may be at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35, 36, 37, 38, 39, or 40.

In some embodiments, D is a masking moiety, such as a masking moiety provided elsewhere herein. In some embodiments, the masking moiety is an anti-idiotypic antibody or fragment thereof. In some embodiments, the masking moiety is an anti-idiotypic scFv or fragment thereof. In some embodiments, t is 1, 2, 3, 4, or 5. In some embodiments, t is 1. In some embodiments, t is 2.

In some embodiments, m may be 0. In some embodiments, m may be 1. In some embodiments, m may be 2. In some embodiments, m may be 3. In some embodiments, m may be 4. In some embodiments, m may be 5. In some embodiments, m may be 6. In some embodiments, m may be 7. In some embodiments, m may be 8. In some embodiments, m may be 9. In some embodiments, m may be 10. In some embodiments, q may be 0. In some embodiments, q may be 1. In some embodiments, q may be 2. In some embodiments, q may be 3. In some embodiments, q may be 4. In some embodiments, q may be 5. In some embodiments, q may be 6. In some embodiments, q may be 7. In some embodiments, q may be 8. In some embodiments, q may be 9. In some embodiments, q may be 10.

In some embodiments, n may be 1. In some embodiments, n may be 2. In some embodiments, n may be 3. In some embodiments, n may be 4. In some embodiments, n may be 5. In some embodiments, n may be 6. In some embodiments, n may be 7. In some embodiments, n may be 8. In some embodiments, n may be 9. In some embodiments, n may be 10. In some embodiments, n may be 11. In some embodiments, n may be 12. In some embodiments, n may be 13. In some embodiments, n may be 14. In some embodiments, n may be 15. In some embodiments, n may be 16. In some embodiments, n may be 17. In some embodiments, n may be 18. In some embodiments, n may be 19. In some embodiments, n may be 20.

In some embodiments, t may be 0. In some embodiments, t may be 1. In some embodiments, t may be 2. In some embodiments, t may be 3. In some embodiments, t may be 4. In some embodiments, t may be 5. In some embodiments, t may be 6. In some embodiments, t may be 7. In some embodiments, t may be 8. In some embodiments, t may be 9. In some embodiments, t may be 10.

### Target Protein

In one aspect, this disclosure provides a composition comprising a binder connected to a radioactive isotope or a cell via a first linker. In some embodiments, the binder may specifically bind to a protein expressed on an endothelial cell.

In some embodiments, the protein may be expressed on a venule endothelial cell (VEC) or a non-venule endothelial cell (NVEC). In some embodiments, the protein may be expressed on a venule endothelial cell (VEC). In some embodiments, the protein may be expressed on a non-venule endothelial cell (NVEC). In some embodiments, the protein may be expressed on a microvascular endothelial cell.

In some embodiments, the protein expression may be upregulated in a VEC compared to NVEC. In some embodiments, the protein may be overexpressed in a VEC by at least about 50% compared to NVEC. In some embodiments, the protein may be overexpressed in a VEC by at least about 10%, 20%, 30%, 40%, 50%, 60%, 70%, 80%, 90%, or 100% compared to a NVEC. In some embodiments, the protein may be overexpressed in a VEC by at most about 10%, 20%, 30%, 40%, 50%, 60%, 70%, 80%, 90%, or 100% compared to a NVEC. In some embodiments, the protein may be overexpressed in a VEC by about 10% to 100%, 20% to 90%, 30% to 80%, 40% to 70%, or 50% to 60% compared to a NVEC.

In some embodiments, the protein expression may be upregulated in a NVEC compared to VEC. In some embodiments, the protein may be overexpressed in a NVEC by at least about 50% compared to VEC. In some embodiments, the protein may be overexpressed in a NVEC by at least about 10%, 20%, 30%, 40%, 50%, 60%, 70%, 80%, 90%, or 100% compared to a VEC. In some embodiments, the protein may be overexpressed in a NVEC by at most about 10%, 20%, 30%, 40%, 50%, 60%, 70%, 80%, 90%, or 100% compared to a VEC. In some embodiments, the protein may be overexpressed in a NVEC by about 10% to 100%, 20% to 90%, 30% to 80%, 40% to 70%, or 50% to 60% compared to a VEC.

In some embodiments, the protein may be an organ-restricted or tissue-specific endothelial cell protein. In some embodiments, the protein may be an organ-restricted endothelial cell protein. In some embodiments, the protein may be a tissue-specific endothelial cell protein. In some embodiments, the organ-restricted endothelial cell protein may be in the liver, kidney, brain, retina, lymph node, bone marrow, small intestine, colon, adipose tissue, skin, lung, heart, any other organ, or a combination thereof. In some embodiments, the tissue specific endothelial cell protein may be in the brain, retina, lymph node, bone marrow, small intestine, colon, adipose tissue, skin, lung, or heart. In some embodiments, the tissue specific endothelial cell protein may be in the liver. In some embodiments, the tissue specific endothelial cell protein may be in the kidney. In some embodiments, the organ-restricted or tissue-specific endothelial cell protein is in the brain. In some embodiments, the organ-restricted or tissue-specific endothelial cell protein is in the retina. In some embodiments, the organ-restricted or tissue-specific endothelial cell protein is in the lymph node. In some embodiments, the organ-restricted or tissue-specific endothelial cell protein is in bone marrow. In some embodiments, the organ-restricted or tissue-specific endothelial cell protein is in the small intestine. In some embodiments, the organ-restricted or tissue-specific endothelial cell protein is in the colon. In some embodiments, the organ-restricted or tissue-specific endothelial cell protein is in adipose tissue. In some embodiments, the organ-restricted or tissue-specific endothelial cell protein is in skin. In some embodiments, the organ-restricted or tissue-specific endothelial cell protein is in the lung. In some embodiments, the organ-restricted or tissue-specific endothelial cell protein is in the heart.

In some embodiments, the protein may have low expression levels on non-tumor endothelial cells. In some embodiments, the protein has greater expression levels on tumor endothelial cells than on non-tumor endothelial cells. In some embodiments, the expression of the protein on a tumor endothelial cell is at least 10× higher (e.g., at least 5× higher, at least 15× higher, at least 20× higher, at least 30× higher, at least 50× higher, at least 100× higher) than on a non-tumor endothelial cell. In some embodiments, the expression of the protein on a tumor endothelial cell is at most 10,000× higher (e.g., at most 5,000× higher, at most 2,500× higher, at most 1,000× higher) than on a non-tumor endothelial cell. In some embodiments, the expression of the protein on a tumor endothelial cell is about 5× higher to about 10,000× higher than on a non-tumor endothelial cell. In some instances, expression on a tumor endothelial cell or non-tumor endothelial cell may be assessed by detection of mRNA (e.g., by RNA sequencing or reverse transcription PCR) or by staining of the protein using antibodies or other reagents.

In some embodiments, the protein may be expressed on the endothelial cell at a cell surface density of greater than about 100 receptors per squared micrometer (μm²). In some embodiments, the protein may be expressed on the endothelial cell at a cell surface density of greater than about 10, 30, 50, 60, 70, 80, 90, 100, 120, 150, 180, 200, 250, or 300 receptors per squared micrometer (μm²). In some embodiments, the protein may be expressed on the endothelial cell at a cell surface density of at least about 10, 30, 50, 60, 70, 80, 90, 100, 120, 150, 180, 200, 250, or 300 receptors per squared micrometer (μm²). In some embodiments, the protein may be expressed on the endothelial cell at a cell surface density of about 10 to 300, 30 to 250, 50 to 200, 60 to 180, 70 to 150, 80 to 120, or 90 to 300 receptors per squared micrometer (μm²).

In some embodiments, the protein may include clinically relevant plasma membrane molecules that are overrepresented in all endothelial cells (Table 1) and specific segment of the vasculature such as venular endothelial cells (Table 2) and non-venular endothelial cells (Table 3) from murine and human tumors compared to their respective non-malignant tissues. In some embodiments, the protein may include clinically relevant plasma membrane molecules that are overrepresented in all endothelial cells (Table 1) from murine and human tumors compared to their respective non-malignant tissues. In some embodiments, the protein may include clinically relevant specific segment of the vasculature such as venular endothelial cells (Table 2) from murine and human tumors compared to their respective non-malignant tissues. In some embodiments, the protein may include clinically relevant non-venular endothelial cells (Table 3) from murine and human tumors compared to their respective non-malignant tissues.

In some embodiments, the target may be a transmembrane molecule on tumor vascular endothelial cells. In some embodiments, the transmembrane molecule is upregulated on the tumor vascular endothelial cells as compared expression on a non-tumor vascular endothelial cell. In some embodiments, the tumor vascular endothelial cell may be a venular cell.

In some embodiments, the transmembrane molecule may be typically expressed at levels at least about 1.5-fold, at least about 2-fold, at least about 2.5-fold, at least about 3-fold, at least about 3.5-fold, at least about 4-fold, at least about 4.5-fold, or at least about 5-fold greater in tumor vascular endothelial cells than in a reference population of cells (e.g., non-tumor vascular endothelial cell) which may consist, for example, of a mixture containing an approximately equal amount of cells (e.g., approximately equal numbers of cells, approximately equal volume of cells, approximately equal mass of cells, etc.). In some embodiments, the transmembrane molecule is present at levels at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 50 fold, at least about 100 fold, at least about 500 fold, at least about 1000 fold, at least about 5000 fold, or at least about 10,000 fold greater than its average expression in a reference population. Detection or measurement of the transmembrane molecule may make it possible to distinguish the cell type or types of interest from cells of many, most, or all other types. In some embodiments, the protein may be encoded by a gene selected from the group consisting of molecules set forth in Tables 1-3. In some embodiments, the protein is encoded by a gene selected from VMP1, LAPTM5, EVL, PCDH17, ARRDC3, PMEPA-1, MYOF, MMP14, or PLEKHO1.

TABLE 1
Plasma membrane molecules over-represented in endothelial cells

A. Plasma membrane molecules over-represented in EC from tumor vs healthy shared by human
melanoma and pancreatic tumor (6 genes):
ENTPD1, MARCKS, SELP, APLNR, ROBO1, PLEKHO1
B. Plasma membrane molecules over-represented in EC from tumor vs healthy unique to human
melanoma (57 genes):
BTN3A2, SLCO2A1, SLC35G2, TNFSF10, PLIN2, ENG, PLVAP, PODXL, PPAP2A, RAMP3,
KDR, HLA-C, SLC6A6, INSR, TGFBR2, MLEC, HLA-DRA, VASP, C1QTNF5, EHD4, ITGA2,
HLA-DRB1, IFITM3, EFNA1, CALCRL, F2R, RELL1, VAMP5, CD40, SLC30A1, NRP1, HLA-
DOA, ESAM, THY1, BMPR2, ACVRL1, TM2B, MOB1A, SFRP1, SLC38A2, HEG1, CD99,
PPAP2B, SPRY4, ATP8B1, FZD6, ANXA5, CNIH1, DLL4, CSF2RB, CD164, TMEM165, PLXND1,
NT5E, RAB13, CD200, TMED10
C. Plasma membrane molecules over-represented in EC from tumor vs healthy unique to human
pancreatic tumor (23 genes):
CNKSR3, ASPH, STAB1, KCTD12, LEPR, PTP4A3, SVIL, ENPP2, TGFBR3, ITPR2, DSP, FAP,
BACE2, NRP2, CADM3, ACKR1, THSD7A, DST, CD93, SULF2, MCTP1, ADGRG6, TIE1
D. Plasma membrane molecules over-represented in EC from tumor vs healthy shared by human
pancreatic tumor, murine melanoma and/or murine colorectal cancer (9 genes):
VMP1, LAPTM5, EVL, PCDH17, ARRDC3, PMEPA-1, MYOF, MMP14, PLEKHO1
E. Plasma membrane molecules over-represented in EC from tumor vs healthy shared by human
melanoma, murine melanoma and/or murine colorectal cancer (8 genes):
ACTR3, CD74, CLIC1, LAPTM4B, HLA-DQA1, TAPBP, MCAM, PLEKHO1
F. Plasma membrane molecules over-represented in EC from tumor vs healthy shared by human
melanoma, human pancreatic tumor, murine melanoma and murine colorectal cancer (1 gene):
PLEKHO1

TABLE 2
Plasma membrane molecules over-represented in VEC

A. Plasma membrane molecules over-represented in VEC from tumor vs healthy shared by
human melanoma and pancreatic tumor (17 genes):
ENG, KDR, INSR, NRP1, ACVRL1, ROBO1, PKP4, CD200, ITGA2, CSF2RB, ENTPD1, RELL1,
TNFRSF1B, LAPTM4B, MARCKS, DYSF, PLXND1
B. Plasma membrane molecules over-represented in VEC from tumor vs healthy unique to
human melanoma (50 genes):
SIGIRR, PTAFR, RAP1A, HLA-DMA, SYPL1, FAT4, HLA-DRB1, CERK, SYT15, CPD, PTPRN2,
HLADOA, THY1, FZD6, CNIH1, HLA-DQB2, IL3RA, BTN3A2, SLCO2A1, TNFSF10, PLVAP,
GPR68, CLSTN3, RAMP3, KL, HLA-C, HLA-DPB1, GBP5, DIAPH1, HLA-DRA, EHD4, TMEM59,
CX3CL1, ATP8A1, TSPAN7, LRRC8A, CD40, TACR1, IL10RB, SLC30A1, SFRP1, BTN3A3,
IL17RA, YIPF3, DLL1, ABI3, SEMA4C, SLC29A1, RAB13, TMED10
C. Plasma membrane molecules over-represented in VEC from tumor vs healthy unique to
human pancreatic tumor (63 genes):
TLR4, LEPR, ITGA5, ESYT1, RAC1, PAM, GNA14, ORAI2, ADGRL4, ASAP1, CADPS2,
TGFBR3, LRRC32, DSP, MET, LRP6, PPFIA1, OSBPL8, KRIT1, ANO2, PGRMC1, CLDN5, EPS8,
ADCY4, TMEM127, GRK5, IL13RA1, PLXDC2, NECTIN2, CADM3, PON2, ACKR1, F2RL3,
ITGA6, MFAP3, TIE1, CNKSR3, TJP1, FZD4, ENPP2, C1QTNF5, ITPR2, CALCRL, EFNB2,
CLECIA, PNN, BACE2, ATP1B3, NRP2, FLT4, ITGA1, PPP4R3A, GPR146, CTTN, CLTC,
ATP2B4, ERLIN1, RIT1, USP9X, MCTP1, ADGRG6, ADGRF5, NCKAP1
D. Plasma membrane molecules over-represented in VEC from tumor vs healthy shared by
human pancreatic tumor, murine melanoma and/or murine colorectal cancer (22 genes):
PCDH1, THSD7A, STAB1, PTPRG, PHACTR4, MLEC, GPR107, SEMA3F, CD93, EVL, PCDH17,
VMP1, BST2, MMP14, TM9SF2, ENTPD1, RELL1, TNFRSF1B, LAPTM4B, MARCKS, DYSF,
PLXND1
E. Plasma membrane molecules over-represented in VEC from tumor vs healthy shared by
human melanoma, murine melanoma and/or murine colorectal cancer (23 genes):
TGFBR2, ESAM, IFNAR1, CD74, VAMP5, APLNR, HLA-DQA1, TMEM204, PCDH12, MPZL1,
F2R, GLG1, CLIC1, ACTR3, AMOTL1, PLEKHO1, MARCKS, DYSF, PLXND1, ENTPD1, RELL1,
TNFRSF1B, LAPTM4B, ACTR3, AMOTL1, PLEKHO1

TABLE 3
Plasma membrane molecules over-represented in NVEC

A. Plasma membrane molecules over-represented in NVEC from tumor vs healthy shared by
human melanoma and pancreatic tumor (7 genes):
ENTPD1, MARCKS, APLNR, ROBO1, CD93, PCDH17, PLEKHO1
B. Plasma membrane molecules over-represented in NVEC from tumor vs healthy unique to
human melanoma (71 genes):
APCDD1, JAG2, STX3, SLC35G2, HECW2, PLIN2, ENG , PLVAP, PODXL, RAMP3, MPZL2,
KDR, HLA-C, SLC6A6, INSR, TGFBR2, PLPP3, MLEC, HLA-DRA, VASP, JCAD, C1QTNF5,
ITGA2, MAGED2, HLA-DRB1, IFITM3, EFNA1, B4GALT1, CALCRL, F2R, VAMP5, TSPAN12,
LGALS9, PLPP1, TMEM30A, SLC30A1, GNB2, SELP, NRP1, FLT4, ESAM, ABHD12, BMPR2,
ACVRL1, ADGRL2, ITM2B, MOB1A, PKD2, SFRP1, SLC38A2, CDH5, HEG1, CD99, CLIC4,
GRK5, SPRY4, CLTC, ATP8B1, TAPBP, CNIH1, APP, DLL4, FLRT2, DYSF, CD164, TMEM165,
PLXND1, RAB13, GPR4, CD200, BSG
C. Plasma membrane molecules over-represented in NVEC from tumor vs healthy unique to
human pancreatic tumor (30 genes):
STAB1, DLG1, KCTD12, LEPR, CACNA1C, PTP4A3, VMP1, TM4SF1, ATP11C, SVIL, SLC4A7,
ENPP2, DSP, PTPRE, NRP2, JAG1, EVL, PCSK5, SLC26A2, ACKR1, ATP2B1, THSD7A, DST,
ABCB1, FBLIM1, MCTP1, SULF2, ARRDC3, TIE1, KCNN3
D. Plasma membrane molecules over-represented in NVEC from tumor vs healthy shared by
human pancreatic tumor, murine melanoma and/or murine colorectal cancer (6 genes):
FCGR2A, LAPTM5, PCDH17, PLEKHO1, PMEPA-1, MMP14
E. Plasma membrane molecules over-represented in NVEC from tumor vs healthy shared by
human melanoma, murine melanoma and/or murine colorectal cancer (13 genes):
SLCO2A1, VCAM1, GJA4, CD74, LAPTM4B, NT5E, TNFAIP1, EDNRB, ANXA5, PCDH17,
PLEKHO1, MCAM, CLIC1
F. Plasma membrane molecules over-represented in NVEC from tumor vs healthy shared by
human melanoma, human pancreatic tumor, murine melanoma and murine colorectal cancer (1
gene):
PLEKHO1

In some embodiments, the protein is encoded by a gene selected from VMP1, LAPTM5, EVL, PCDH17, ARRDC3, PMEPA-1, MYOF, MMP14, or PLEKHO1. In some embodiments, the protein may be Prostate Transmembrane Protein, Androgen Induced 1 (PMEPA-1).

### Binder (Anchorbody)

Provided herein, in some embodiments, are binders (e.g., anchorbodies) connected to a radioactive isotope via a first linker (e.g., such as in Formula (I)). In some embodiments, the binder is connected to a masking moiety via a second linker and a radioactive isotope via a first linker (e.g., such as in Formula (II)). In some embodiments, this disclosure provides a composition comprising a first binder connected to a second binder via a first linker. In some embodiments, the first binder may specifically bind to a protein expressed on an endothelial cell. In some embodiments, the first binder and the second binder are different. In some embodiments, the first binder and the second binder are the same.

As used herein, a “binder (e.g., anchorbody)” refers to any molecule that binds to a component associated with an organ, tissue, cell, extracellular matrix, and/or subcellular locale. In some embodiments, such a component is referred to as a “target” or a “marker”.

In some embodiments, a binder may be a nucleic acid, polypeptide, glycoprotein, carbohydrate, lipid, small molecule, etc. In some embodiments, a binder may be an antibody, which term is intended to include antibody fragments, characteristic portions of antibodies, single chain antibodies, etc. In some embodiments, a binder may be synthetic binding proteins comprising Affibodies®, Nanobodies™, AdNectins™, or Avimers™. In some embodiments, a binder may be a nanobody.

In some embodiments, a binder provided herein recognizes one or more proteins (e.g., target or markers) associated with a particular organ, tissue, cell, and/or subcellular locale. In some embodiments, a target may be a marker that is exclusively or primarily associated with one or a few cell types, with one or a few diseases, and/or with one or a few developmental stages.

In some embodiments, the proteins may include, but are not limited to, antibodies, receptors, cytokines, peptide hormones, glycoproteins, glycopeptides, proteoglycans, proteins derived from combinatorial libraries (e.g., Avimers™, Affibodies®, etc.), and characteristic portions thereof. In some embodiments, the proteins may comprise synthetic binding proteins comprising Nanobodies™, or AdNectins™, etc. In some embodiments, a binder may be a nanobody. One of ordinary skill in the art will appreciate that any protein and/or peptide that specifically binds to a desired target as described herein, can be used in accordance with the present invention.

In some embodiments, a binder may be an antibody, an antibody fragment, or an antigen-binding fragment. In some embodiments, a binder may be an antibody and/or characteristic portion thereof. The term “antibody” refers to any immunoglobulin, whether natural or wholly or partially synthetically produced and to derivatives thereof and characteristic portions thereof. An antibody may be monoclonal or polyclonal. An antibody may be a member of any immunoglobulin class, including any of the human classes: IgG, IgM, IgA, IgD, and IgE. Antibodies or immunoglobulins are heterotetrameric glycosaminoglycan proteins of about 150,000 Dalton with the same structural features, consisting of two identical light (L) chains and two identical heavy (H) chains. Each light chain is linked to the heavy chain through a covalent disulfide bond, and the number of disulfide bonds between the heavy chains of different immunoglobulin isoforms is different. Each heavy and light chain also has intra-chain disulfide bonds which are regular spaced. Each heavy chain has a variable region (VH) at one end followed by a plurality of constant regions. Each light chain has a variable region (VL) at one end and a constant region at the other end; the constant region of the light chain is opposite to the first constant region of the heavy chain, and the variable region of the light chain is opposite to the variable region of the heavy chain. Special amino acid residues form an interface between the variable regions of the light and heavy chains.

In some embodiments, a binder provided herein may be an antibody fragment. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may comprise multiple chains which are linked together, for example, by disulfide linkages. An antibody fragment may optionally comprise a multimolecular complex. A functional antibody fragment may comprise at least about 50 amino acids or may comprise at least about 200 amino acids. In some embodiments, the antibody fragment may comprise at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 400, or 500 amino acids. In some embodiments, the antibody fragment may comprise at most about 20, 30, 40, 50, 60, 70, 80, 90, 100, 110, 120, 130, 140, 150, 160, 170, 180, 190, 200, 210, 220, 230, 240, 250, 260, 270, 280, 290, 300, 400, or 500 amino acids.

An antibody fragment may be produced by any means. In some embodiments, an antibody fragment may be enzymatically or chemically produced by fragmentation of an intact antibody. In some embodiments, an antibody fragment may be recombinantly produced from a gene encoding the partial antibody sequence. In some embodiments, an antibody fragment may comprise multiple chains which are linked together. In some embodiments, an antibody fragment may comprise multiple chains which are linked together by disulfide linkages. An antibody fragment may optionally comprise a multimolecular complex.

In some embodiments, antibodies may include chimeric (e.g. “humanized”) and single chain (recombinant) antibodies. In some embodiments, antibodies may include chimeric antibodies. In some embodiments, antibodies may include single chain (recombinant) antibodies. In some embodiments, antibodies may have reduced effector functions and/or bispecific molecules. In some embodiments, antibodies may have reduced effector functions. In some embodiments, antibodies may have bispecific molecules. In some embodiments, antibodies may include fragments produced by a Fab expression library.

In a particular embodiment, the antibody or antigen binding fragment may be a nanobody. Nanobodies are recombinant antibody fragments consisting of one variable heavy chain. In some embodiments, the variable heavy chain of a nanobody comprises a CDR1, CDR2, and CDR3. The CDR1 and CDR2 segments can be short in comparison to the CDR3 segment, which is longer than the typical CDR3 in a conventional antibody or scFv molecule.

The present disclosure also provides other polypeptides, such as a fusion protein comprising nanobodies or fragments thereof. In some embodiments, the fragment may comprise at least about 50 contiguous amino acids of the nanobody as disclosed herein. In some embodiments, the fragment may comprise at least about 50 contiguous amino acids. In some embodiments, the fragment may comprise at least about 80 contiguous amino acids. In some embodiments, the fragment may comprise at least about 100 contiguous amino acids. In some embodiments, the fusion protein may comprise at least about 20, 30, 40, 50, 60, 70, 80, 90, 100, 110, 120, 130, 140, 150, 160, 170, 180, 190, or 200 contiguous amino acids of the nanobody as disclosed herein. In some embodiments, the fusion protein may comprise at most about 20, 30, 40, 50, 60, 70, 80, 90, 100, 110, 120, 130, 140, 150, 160, 170, 180, 190, or 200 contiguous amino acids of the nanobody as disclosed herein. In some embodiments, the fusion protein may comprise at least about 20, 30, 40, 50, 60, 70, 80, 90, 100, 110, 120, 130, 140, 150, 160, 170, 180, 190, or 200 contiguous amino acids. In some embodiments, the fusion protein may comprise at most about 20, 30, 40, 50, 60, 70, 80, 90, 100, 110, 120, 130, 140, 150, 160, 170, 180, 190, or 200 contiguous amino acids.

In some embodiments, binders provided herein may be single-chain Fvs. Single-chain Fvs (scFvs) are recombinant antibody fragments consisting of only the variable light chain (VL) and variable heavy chain (VH) covalently connected to one another by a polypeptide linker. Either VL or VH may comprise the NH₂-terminal domain. The polypeptide linker may be of variable length and composition so long as the two variable domains are bridged without significant steric interference. Typically, linkers primarily comprise stretches of glycine and serine residues with some glutamic acid or lysine residues interspersed for solubility.

In some embodiments, binders provided herein may be diabodies. Diabodies are dimeric scFvs. Diabodies typically have shorter peptide linkers than most scFvs, and they often show a preference for associating as dimers.

In some embodiments, binders provided herein may be Fv fragments. An Fv fragment is an antibody fragment which consists of one VH and one VL domain held together by noncovalent interactions. The term “dsFv” as used herein refers to an Fv with an engineered intermolecular disulfide bond to stabilize the VH-VL pair.

In some embodiments, binders provided herein may be F(ab′)2 fragments. An F(ab′)2 fragment is an antibody fragment essentially equivalent to that obtained from immunoglobulins by digestion with an enzyme pepsin at pH 4.0-4.5. The fragment may be recombinantly produced.

In some embodiments, a binder provided herein may be a Fab′ fragment. A Fab′ fragment is an antibody fragment essentially equivalent to that obtained by reduction of the disulfide bridge or bridges joining the two heavy chain pieces in the F(ab′)2 fragment. The Fab′ fragment may be recombinantly produced.

In some embodiments, a binder provided herein may be a Fab fragment. A Fab fragment is an antibody fragment essentially equivalent to that obtained by digestion of immunoglobulins with an enzyme (e.g., papain). The Fab fragment may be recombinantly produced. The heavy chain segment of the Fab fragment is the Fd piece.

In some embodiments, the antibodies or antigen-binding fragments thereof comprise a variant of the disclosed antibodies or antigen-binding fragments thereof herein. In some embodiments, antibodies or antigen-binding fragments thereof in which there are up to about 10, preferably up to about 8, more preferably up to about 5, and most preferably up to about 3 amino acids substituted by amino acids having analogical or similar properties, compared to the amino acid sequences set forth herein. In some embodiments, antibodies or antigen-binding fragments thereof in which there are up to about 15, 14, 13, 12, 11, 10, 9, 8, 7, 6, 5, 4, 3, 2, or 1 amino acids substituted by amino acids having analogical or similar properties, compared to the amino acid sequences set forth herein. These conservative variant antibodies or antigen-binding fragments thereof are preferably produced according to the amino acid substitutions in Table 4.

TABLE 4
Amino Acid Substitutions

| Original Residue | Possible substitution |
|---|---|
| Ala (A) | Val; Leu; Ile |
| Arg (R) | Lys; Gln; Asn |
| Asn (N) | Gln; His; Lys; Arg |
| Asp (D) | Glu |
| Cys (C) | Ser |
| Gln (Q) | Asn |
| Glu (E) | Asp |
| Gly (G) | Pro; Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Leu; Val; Ile; Ala; Tyr |
| Pro (P) | Ala |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala |

In accordance with the present invention, the antibody, or antigen binding fragment thereof comprising a nanobody, recognizes PMEPA-1. PMEPA-1, which can be recognized by the antibody, or antigen binding fragment thereof, e.g., nanobody, is a cell type specific marker that may be expressed at levels at least about 1.5-fold, at least about 2-fold, at least about 2.5-fold, at least about 3-fold, at least about 3.5-fold, at least about 4-fold, at least about 4.5-fold, or at least about 5-fold greater in tumor vascular endothelial cells than in a reference population of cells (e.g., non-tumor vascular endothelial cells) which may consist, for example, of a mixture containing an approximately equal amount of cells (e.g., approximately equal numbers of cells, approximately equal volume of cells, approximately equal mass of cells, etc.). In some embodiments, the cell type specific marker PMEPA-1 may be present at levels at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 50 fold, at least about 100 fold, at least about 500 fold, at least about 1000 fold, at least about 5000 fold, or at least about 10,000 fold greater than its average expression in a reference population. Detection or measurement of the cell type specific marker PMEPA-1 may make it possible to distinguish the cell type or types of interest from cells of many, most, or all other types.

In some embodiments, the nanobodies can be covalently linked to a drug (e.g., chemotherapeutic drug), imaging probe, or displayed on the surface of nanoparticles, viruses, or CAR T cells.

In some embodiment, the binder (e.g., antibody or antigen-binding fragment thereof) is (e.g., covalently) linked to one or more detectable markers (e.g., imaging probe or detectable labels) or other signal-generating groups or moieties, depending on the intended use of the labeled nanobody. Suitable markers and techniques for attaching, using and detecting them will be clear to the skilled person and, for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person, for example, include moieties that can be detected using NMR or ESR spectroscopy. In some embodiments, the binder (e.g., antibody or antigen-binding fragment thereof) is (e.g., covalently) linked to one or more imaging probes (e.g., radioactive isotopes and fluorescent labels). In some embodiments, the binder (e.g., antibody or antigen-binding fragment thereof (e.g., covalently) linked to one or more different types of imaging probes (e.g., radioactive isotope and fluorescent labels) to provide multi-modal imaging capability.

In some embodiments, the binder may be a monoclonal antibody, a polyclonal antibody, a bispecific antibody, a trispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a nanobody, an aptamer, a dendrimer, a peptide, RNAi, siRNA, shRNA, miRNA, or an antigen-binding fragments thereof. In some embodiments, the binder is an aptamer. In some embodiments, the binder is a dendrimer. In some embodiments, the binder is a peptide. In some embodiments, the binder is RNAi. In some embodiments, the binder is siRNA. In some embodiments, the binder is shRNA. In some embodiments, the binder is miRNA. In some embodiments, the binder is a monoclonal antibody. In some embodiments, the binder is a polyclonal antibody. In some embodiments, the binder is a humanized antibody. In some embodiments, the binder is a synthetic antibody. In some embodiments, the binder is a chimeric antibody. In some embodiments, the binder is a camelized antibody. In some embodiments, the binder is a single-chain Fvs (scFV). In some embodiments, the binder is a single chain antibody. In some embodiments, the binder is a Fab fragment. In some embodiments, the binder is a F(ab′)2 fragment. In some embodiments, the binder is a Fd fragment. In some embodiments, the binder is a Fv fragment. In some embodiments, the binder is a single-domain antibody. In some embodiments, the binder is a diabody. In some embodiments, the binder is a fragment comprised of only a single monomeric variable domain. In some embodiments, the binder is a disulfide-linked Fvs (sdFV). In some embodiments, the binder is an intrabody. In some embodiments, the binder is an anti-idiotypic (anti-Id) antibody. In some embodiments, the binder is a nanobody. In some embodiments, the binder is an ab antigen-binding fragment.

In some embodiments, the binder may be a nanobody, a single chain variable fragment (scFv), a single-chain antibody, a single-domain antibody, a diabody, a Fab fragment, or a combination thereof.

In some embodiments, the binder may be a nanobody. In some embodiments, the nanobodies can comprise multiple (two or more) VH segments, such as a dimer. Peptide linker can be between VH segments. Each VH segment in a multimer nanobody can be the same VH sequence binding to the same antigen, or different VH sequence binding to different antigens, or different VH sequences binding the same antigen at non-overlapping epitopes. In some embodiments, the nanobodies can comprise multiple segments of VH segments as described above and scFv molecules.

In some embodiments, the nanobodies provided herein include one or more variations. These variations include, but are not limited to, deletion, insertions and/or substitutions of 1, 2, 3, 4, 5, 6, 7, 8, 9 or 10 (usually 1-50, preferably 1-30, more preferably 1-20, optimally 1-10) amino acids, and addition of 1, 2, 3, 4, 5, 6, 7, 8, 9, or 10 (generally less than 20, preferably less than 10, and more preferably less than 5) amino acids at C-terminus and/or N-terminus. For example, in the art, the substitution of amino acids with analogical or similar properties usually does not alter the function of the protein. By way of further example, addition of one or several amino acids at the C-terminus and/or N-terminus usually does not change the function of the protein. The term also includes active fragments and active derivatives of the disclosed nanobodies.

In some embodiments, the nanobodies provided herein can be covalently linked to a therapeutic agent provided elsewhere herein, such as a drug (e.g., chemotherapeutic drug), imaging probe, or displayed on the surface of nanoparticles, viruses, or CAR T cells.

In some embodiments, the antibody, or antigen binding fragment thereof, e.g., nanobody, may be covalently linked to one or more detectable markers (e.g., imaging probe or detectable labels) or other signal-generating groups or moieties, depending on the intended use of the labeled antibody, or antigen binding fragment thereof, e.g., nanobody. Suitable markers and techniques for attaching, using and detecting them will be clear to the skilled person and, for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person and, for example, include moieties that can be detected using NMR or ESR spectroscopy.

In some embodiments, the binder may specifically bind to the protein expressed on the endothelial cell with a dissociation constant (K_{d}) value of about 1 mM or less, such as measured by surface plasmon resonance. In some embodiments, the binder may specifically bind to the protein expressed on the endothelial cell with a K_{d} value of at least about 0.001 nM (e.g., about 0.01 nM, about 0.1 nM, about 0.5 nM, about 1 nM, about 5 nM, about 10 nM, about 50 nM, about 100 nM, about 500 nM, about 1,000 nM, or about 5,000 nM), such as measured by surface plasmon resonance. In some embodiments, the binder may specifically bind to the protein expressed on the endothelial cell with a Ka value of at most about 0.1 nM (e.g., about 0.5 nM, about 1 nM, about 5 nM, about 10 nM, about 50 nM, about 100 nM, about 500 nM, about 1,000 nM, about 5,000 nM, or about 10,000 nM), such as measured by surface plasmon resonance. In some embodiments, the binder may specifically bind to the protein expressed on the endothelial cell with a Ka value of about 0.001 nM to about 10,000 nM, such as measured by surface plasmon resonance. In some embodiments, the binder may specifically bind to the protein expressed on the endothelial cell with a Ka value of about 0.01 nM to about 10,000 nM, such as measured by surface plasmon resonance. In some embodiments, the binder may specifically bind to the protein expressed on the endothelial cell with a Ka value of about 0.1 nM to about 1,000 nM, such as measured by surface plasmon resonance. In some embodiments, the binder may specifically bind to the protein expressed on the endothelial cell with a Ka value of about 0.1 nM to about 100 nM, such as measured by surface plasmon resonance. In some embodiments, the binder may specifically bind to the protein expressed on the endothelial cell with a Ka value of about 0.1 nM to about 1 nM, such as measured by surface plasmon resonance.

In some embodiments, the binder may specifically bind to the protein expressed on the endothelial cell with an association constant (Kₐ) value of about 1 mM or less, such as measured by surface plasmon resonance. In some embodiments, the binder may specifically bind to the protein expressed on the endothelial cell with an Kₐ value of at least about 0.001 nM (e.g., about 0.01 nM, about 0.1 nM, about 0.5 nM, about 1 nM, about 5 nM, about 10 nM, about 50 nM, about 100 nM, about 500 nM, about 1,000 nM, or about 5,000 nM), such as measured by surface plasmon resonance. In some embodiments, the binder may specifically bind to the protein expressed on the endothelial cell with a Ka value of at most about 0.1 nM (e.g., about 0.5 nM, about 1 nM, about 5 nM, about 10 nM, about 50 nM, about 100 nM, about 500 nM, about 1,000 nM, about 5,000 nM, or about 10,000 nM), such as measured by surface plasmon resonance. In some embodiments, the binder may specifically bind to the protein expressed on the endothelial cell with a Ka value of about 0.001 nM to about 10,000 nM, such as measured by surface plasmon resonance. In some embodiments, the binder may specifically bind to the protein expressed on the endothelial cell with a Ka value of about 0.01 nM to about 10,000 nM, such as measured by surface plasmon resonance. In some embodiments, the binder may specifically bind to the protein expressed on the endothelial cell with a Ka value of about 0.1 nM to about 1,000 nM, such as measured by surface plasmon resonance. In some embodiments, the binder may specifically bind to the protein expressed on the endothelial cell with a Ka value of about 0.1 nM to about 100 nM, such as measured by surface plasmon resonance. In some embodiments, the binder may specifically bind to the protein expressed on the endothelial cell with a Ka value of about 0.1 nM to about 1 nM, such as measured by surface plasmon resonance.

### First Binder

In some embodiments, the binder may be an endothelial cell specific antibody.

In some embodiments, the binder may be an anti-PMEPA-1 antibody or antigen-binding fragment. In some embodiments, the anti-PMEPA-1 antibody or antigen-binding fragment may comprise an amino acid sequence identity set out in any one of SEQ ID NOs: 1-7.

In some embodiments, the anti-PMEPA-1 antibody or antigen-binding fragment may comprise having at least 80% (e.g., at least 85%, at least 90%, at least 95%) sequence identity to an amino acid sequence set out in any one of SEQ ID NOs: 1-7.

In some embodiments, the anti-PMEPA-1 antibody or antigen-binding fragment comprises an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 1-7. In some embodiments the anti-PMEPA-1 antibody or antigen-binding fragment may comprises an amino acid sequence at least 60%, 70%, 80%, 90%, 95%, 100% identity to at least about 5, 10, 15, 20, 25, 30, 35, 40, 45, 50, 60, 70, 90, 100, consecutive amino acid of CDR, variable region, constant region or a frame work region disclosed herein.

In some embodiments, the anti-PMEPA-1 antibody or antigen-binding fragment may comprise amino acid set out in any one of SEQ ID NOs: 1-7 is in Table 5.

In some embodiments, the anti-PMEPA-1 antibody or antigen-binding fragment may comprise (a) a CDR1 having at least 80% (e.g., at least 85%, at least 90%, or at least 95%) sequence identity to the amino acid sequence set out in any one of SEQ ID NOs: 8, 11, 14, 17, 20, or 23, (b) a CDR2 having at least 80% (e.g., at least 85%, at least 90%, or at least 95%) sequence identity to the amino acid sequence set out in any one of SEQ ID NOs: 9, 12, 15, 18, 21, or 24, and (c) a CDR3 having at least 80% (e.g., at least 85%, at least 90%, or at least 95%) sequence identity to the amino acid sequence set out in any one of SEQ ID NOs: 10, 13, 16, 19, 22, or 25.

In some embodiments, the anti-PMEPA-1 antibody or antigen-binding fragment comprises an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 8, 11, 14, 17, 20, or 23. In some embodiments, the anti-PMEPA-1 antibody or antigen-binding fragment comprises an amino acid sequence at least 60%, 70%, 80%, 90%, 95%, 100% identity to at least about 5, 10, 15, 20, 25, 30, 35, 40, 45, 50, 60, 70, 90, 100, consecutive amino acid of CDR, variable region, constant region or a frame work region disclosed herein.

In some embodiments, the anti-PMEPA-1 antibody or antigen-binding fragment comprises an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 9, 12, 15, 16, 21, or 24. In some embodiments, the anti-PMEPA-1 antibody or antigen-binding fragment may comprises an amino acid sequence at least 60%, 70%, 80%, 90%, 95%, 100% identity to at least about 5, 10, 15, 20, 25, 30, 35, 40, 45, 50, 60, 70, 90, 100, consecutive amino acid of CDR, variable region, constant region or a frame work region disclosed herein.

In some embodiments, the anti-PMEPA-1 antibody or antigen-binding fragment comprises an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NOs: 10, 13, 16, 19, 22, or 25. In some embodiments, the anti-PMEPA-1 antibody or antigen-binding fragment may comprises an amino acid sequence at least 60%, 70%, 80%, 90%, 95%, 100% identity to at least about 5, 10, 15, 20, 25, 30, 35, 40, 45, 50, 60, 70, 90, 100, consecutive amino acid of CDR, variable region, constant region or a frame work region disclosed herein.

In some embodiments, the amino acid sequence set out in any one of SEQ ID NOs: 8-25 corresponding to CDR1, CDR2, or CDR3 of the anti-PMEPA-1 antibody or antigen-binding fragment is in Table 6.

The present invention also provides a polynucleotide molecule encoding the antibody or antigen binding fragment thereof, e.g., nanobody, to PMEPA-1. In some embodiments, the polynucleotide encodes an antibody or antigen-binding fragment thereof, to PMEPA-1. In a particular embodiment, the polynucleotide encodes a nanobody to PMEPA-1. In some embodiments, the polynucleotide may be in the form of DNA or RNA. DNA forms include cDNA, genomic DNA, or synthetic DNA. DNA can be single-stranded or double-stranded. DNA can be a coding strand or a non-coding strand.

In various embodiments, the present invention provides for a polynucleotide molecule encoding a nanobody comprising
(i) a CDR1 sequence comprising the amino acid sequence of SEQ ID NO: 8, a CDR2 sequence comprising the amino acid sequence of SEQ ID NO: 9, and a CDR3 sequence comprising the amino acid sequence of SEQ ID NO: 10;
(ii) a CDR1 sequence comprising the amino acid sequence of SEQ ID NO: 11, a CDR2 sequence comprising the amino acid sequence of SEQ ID NO: 12, and a CDR3 sequence comprising the amino acid sequence of SEQ ID NO: 13;
(iii) a CDR1 sequence comprising the amino acid sequence of SEQ ID NO: 14, a CDR2 sequence comprising the amino acid sequence of SEQ ID NO: 15, and a CDR3 sequence comprising the amino acid sequence of SEQ ID NO: 16;
(iv) a CDR1 sequence comprising the amino acid sequence of SEQ ID NO: 17, a CDR2 sequence comprising the amino acid sequence of SEQ ID NO: 18, and a CDR3 sequence comprising the amino acid sequence of SEQ ID NO: 19;
(v) a CDR1 sequence comprising the amino acid sequence of SEQ ID NO: 20, a CDR2 sequence comprising the amino acid sequence of SEQ ID NO: 21, and a CDR3 sequence comprising the amino acid sequence of SEQ ID NO: 22; or
(vi) a CDR1 sequence comprising the amino acid sequence of SEQ ID NO: 23, a CDR2 sequence comprising the amino acid sequence of SEQ ID NO: 24, and a CDR3 sequence comprising the amino acid sequence of SEQ ID NO: 25.

TABLE 5

| | |
|---|---|
| SEQ ID NO: 1 | QAGGSLRLSCAASGYIFSDTYMGWYRQAPGKEREFVAGINGGGTTN |
| | YADSVKGRFTISRDNAKNTVYLQMNSLKPEDTAVYYCAVYYGSFS |
| | WSLL |
| SEQ ID NO: 2 | AGGSLRLSCAASGNISYSYGMGWYRQAPGKEREFVAGITFGGSTYY |
| | ADSVKGRFTISRDNAKNTVYLQMNSLKPEDTAVYYCAVYTRHVDT |
| | TFARHWYWGQGTQVTVSSLEHHH |
| SEQ ID NO: 3 | AGGSLRLSCAASGNIFYGQPMGWYRQAPGKEREFVAGIGRGGSTYY |
| | ADSVKGRFTISRDNAKNTVYLQMNSLKPEDTAVYYCAVLSQYPYRH |
| | TYWGQGTQVTVSSLEHH |
| SEQ ID NO: 4 | QAGGSLRLSCAASGTISTYGMGWYRQAPGKEREFVAGIATGGTTYY |
| | ADSVKGRFTISRDNAKNTVYLQMNSLKPEDTAVYYCAALDKYARH |
| | YVYWGQGTQVTVSSLEHHHHHH |
| SEQ ID NO: 5 | QAGGSLRLSCAASGTIFYRYSMGWYRQAPGKEREFVAGITEGSNTY |
| | YADSVKGRFTISRDNAKNTVYLQMNSLKPEDTAVYYCAVVQRVDL |
| | TYWGQGTQVTVSSLEHHHHHH |
| SEQ ID NO: 6 | QAGGSLRLSCAASGNIFRVIGMGWYRQAPGKEREFVAGIGSGSSTYY |
| | ADSVKGRFTISRDNAKNTVYLQMNSLKPEDTAVYYCAAFTYPYYDQ |
| | TKLLPYWGQGTQVTVSSLEHHHHHH |
| SEQ ID NO: 7 | QAGGSLRLSCAASGTIFPRANMGWYRQAPGKEREFVAGITLGGTTY |
| | YADSVKGRFTISRDNAKNTVYLQMNSLKPEDTAVYYCAVVYKTYR |
| | YQEILYYYWGQGTQVTVSSLEHHHHHH |

TABLE 6
Amino Acid Sequence

| CDR 1 | CDR2 | CDR3 |
|---|---|---|
| NISYSYG | GITFGGS | VYTRHVDTTFARHW |
| SEQ ID NO: 8 | SEQ ID NO: 9 | SEQ ID NO: 10 |
| NIFYGQP | GIGRGGS | VLSQYPYRHT |
| SEQ ID NO: 11 | SEQ ID NO: 12 | SEQ ID NO: 13 |
| TISTYG | GIATGGT | ALDKYARHYV |
| SEQ ID NO: 14 | SEQ ID NO: 15 | SEQ ID NO: 16 |
| TIFYRYS | GITEGSN | VVQRVDLT |
| SEQ ID NO: 17 | SEQ ID NO: 18 | SEQ ID NO: 19 |
| NIFRVIG | GIGSGSS | AFTYPYYDQTKLLP |
| SEQ ID NO: 20 | SEQ ID NO: 21 | SEQ ID NO: 22 |
| TIFPRAN | GITLGGT | VVYKTYRYQEILYY |
| SEQ ID NO: 23 | SEQ ID NO: 24 | SEQ ID NO: 25 |

In some embodiments, the polynucleotide molecule comprises the nucleotide sequence of SEQ ID NO: 26, SEQ ID NO: 27, SEQ ID NO: 28, SEQ ID NO: 29, SEQ ID NO: 30, or SEQ ID NO: 31.

In some embodiments, the polynucleotide molecule comprises an amino acid sequence that is at least 60% identical (e.g., at least 62%, at least 64%, at least 66%, at least 68%, at least 70%, at least 72%, at least 74%, at least 76%, at least 78%, at least 80%, at least 82%, at least 84%, at least 85%, at least 86%, at least 88%, at least 90%, at least 91%, at least 92%, at least 93%, at least 94%, at least 95%, at least 96%, at least 97%, at least 98%, at least 99%, or 100% identical) to any one of SEQ ID NO: 26, SEQ ID NO: 27, SEQ ID NO: 28, SEQ ID NO: 29, SEQ ID NO: 30, or SEQ ID NO: 31.

In some embodiments, the polynucleotide molecule comprises the nucleotide sequence provided in Table 7.

TABLE 7

| SEQ ID NO | Nucleotide Sequence |
|---|---|
| SEQ ID NO: 26 | GCGGGCGGCAGCCTGCGCCTGAGCTGCGCGGCGAGCGGCAATATT |
| | TCTTACTCTTACGGTATGGGCTGGTATCGCCAGGCGCCGGGCAAAG |
| | AACGCGAATTTGTTGCCGGTATTACTTTCGGTGGTAGTACCTATTA |
| | TGCGGATAGCGTGAAAGGCCGCTTTACCATTAGCCGCGATAACGC |
| | GAAAAACACCGTGTATCTGCAGATGAACAGCCTGAAACCGGAAGA |
| | TACCGCGGTGTATTATTGCGCGGTTTACACTCGTCACGTTGACACT |
| | ACTTTCGCTCGTCATTGGTATTGGGGCCAGGGCACCCAGGTGACCG |
| | TGAGCAGCCTCGAGCACCACCACCACCACCAC |
| SEQ ID NO: 27 | GCGGGCGGCAGCCTGCGCCTGAGCTGCGCGGCGAGCGGCAATATT |
| | TTTTACGGTCAGCCGATGGGCTGGTATCGCCAGGCGCCGGGCAAA |
| | GAACGCGAATTTGTTGCCGGTATTGGTCGTGGTGGTAGTACCTATT |
| | ATGCGGATAGCGTGAAAGGCCGCTTTACCATTAGCCGCGATAACG |
| | CGAAAAACACCGTGTATCTGCAGATGAACAGCCTGAAACCGGAAG |
| | ATACCGCGGTGTATTATTGCGCGGTTCTGTCTCAGTACCCGTACCG |
| | TCATACTTATTGGGGCCAGGGCACCCAGGTGACCGTGAGCAGCCT |
| | CGAGCACCACCACCACCACCAC |
| SEQ ID NO: 28 | GGCAGCCTGCGCCTGAGCTGCGCGGCGAGCGGCACTATTTCTACTT |
| | ACGGTATGGGCTGGTATCGCCAGGCGCCGGGCAAAGAACGCGAAT |
| | TTGTTGCCGGTATTGCTACTGGTGGTACTACCTATTATGCGGATAG |
| | CGTGAAAGGCCGCTTTACCATTAGCCGCGATAACGCGAAAAACAC |
| | CGTGTATCTGCAGATGAACAGCCTGAAACCGGAAGATACCGCGGT |
| | GTATTATTGCGCGGCTCTGGACAAATACGCTCGTCATTATGTTTAT |
| | TGGGGCCAGGGCACCCAGGTGACCGTGAGCAGCCTCGAGCACCAC |
| | CACCACCACCAC |
| SEQ ID NO: 29 | GCGGGCGGCAGCCTGCGCCTGAGCTGCGCGGCGAGCGGCACTATT |
| | TTTTACCGTTACTCTATGGGCTGGTATCGCCAGGCGCCGGGCAAAG |
| | AACGCGAATTTGTTGCCGGTATTACTGAAGGTAGTAATACCTATTA |
| | TGCGGATAGCGTGAAAGGCCGCTTTACCATTAGCCGCGATAACGC |
| | GAAAAACACCGTGTATCTGCAGATGAACAGCCTGAAACCGGAAGA |
| | TACCGCGGTGTATTATTGCGCGGTTGTTCAGCGTGTTGACCTTACT |
| | TATTGGGGCCAGGGCACCCAGGTGACCGTGAGCAGCCTCGAGCAC |
| | CACCACCACCACCAC |
| SEQ ID NO: 30 | GCGGGCGGCAGCCTGCGCCTGAGCTGCGCGGCGAGCGGCAATATT |
| | TTTCGTGTTATCGGTATGGGCTGGTATCGCCAGGCGCCGGGCAAAG |
| | AACGCGAATTTGTTGCCGGTATTGGTTCTGGTAGTAGTACCTATTA |
| | TGCGGATAGCGTGAAAGGCCGCTTTACCATTAGCCGCGATAACGC |
| | GAAAAACACCGTGTATCTGCAGATGAACAGCCTGAAACCGGAAGA |
| | TACCGCGGTGTATTATTGCGCGGCTTTCACTTACCCGTACTACGAC |
| | CAGACTAAACTGCTTCCGTATTGGGGCCAGGGCACCCAGGTGACC |
| | GTGAGCAGCCTCGAGCACCACCACCACCACCAC |
| SEQ ID NO: 31 | GCGGGCGGCAGCCTGCGCCTGAGCTGCGCGGCGAGCGGCACTATT |
| | TTTCCGCGTGCTAACATGGGCTGGTATCGCCAGGCGCCGGGCAAA |
| | GAACGCGAATTTGTTGCCGGTATTACTCTGGGTGGTACTACCTATT |
| | ATGCGGATAGCGTGAAAGGCCGCTTTACCATTAGCCGCGATAACG |
| | CGAAAAACACCGTGTATCTGCAGATGAACAGCCTGAAACCGGAAG |
| | ATACCGCGGTGTATTATTGCGCGGTTGTTTACAAAACTTACCGTTA |
| | CCAGGAAATCCTGTATTACTATTGGGGCCAGGGCACCCAGGTGAC |
| | CGTGAGCAGCCTCGAGCACCACCACCACCACCAC |

In some embodiments, the polynucleotide molecule is contained in a vector. In some embodiments, the vectors comprise suitable promoters and/or control sequences. These vectors can be used to transform an appropriate host cell so that it can express the protein (e.g., antibody or antigen-binding fragment, thereof, e.g., a nanobody to PMEPA-1).

In some embodiments, the host cell can be a prokaryotic cell (e.g., a bacterial cell); a eukaryotic cell (e.g., a yeast cell, insect cell, or mammalian cell). In some embodiments, the prokaryotic cell or eukaryotic cell comprises the polynucleotide molecule encoding the antibody or antigen-binding fragment thereof, e.g., a nanobody, to PMEPA-1. In some embodiments, the prokaryotic cell or eukaryotic cell expresses the antibody or antigen-binding fragment thereof, e.g., a nanobody, to PMEPA-1 for purification of the antibody or antigen-binding fragment thereof. In some embodiments, the prokaryotic cell or eukaryotic cell expresses the antibody or antigen-binding fragment thereof, e.g., a nanobody, to PMEPA-1 for displaying the antibody or antigen-binding fragment thereof on the surface of the cell.

In some embodiments, the binder may be an anti-PMEPA-1 antibody or antigen-binding fragment. In some embodiments, the antibody or antigen binding fragment thereof, e.g., nanobody, to PMEPA-1 are coupled (e.g., covalently associated) with an agent that is capable of inducing cell death to a tumor vascular endothelial cell in which the expression of PMEPA-1 (and, optionally, an additional transmembrane molecule from Tables 2-4) is upregulated as compared to a non-tumor vascular endothelial cell. In some embodiments, covalent association is mediated by a linker. In some embodiments, the antibody or an antigen binding fragment thereof, e.g., nanobody, to PMEPA-1 is not covalently associated with an agent that is capable of inducing cell death to a tumor vascular endothelial cell in which the expression of PMEPA-1 (and, optionally, an additional transmembrane molecule from Tables 2-4) is upregulated as compared to a non-tumor vascular endothelial control cell.

In some embodiments, the first binder is not a chimeric antigen receptor.

In some embodiments, the first binder comprises a membrane anchoring domain. In some embodiments, the membrane anchoring domain is a transmembrane domain or a Gpi linker. In some embodiments, the transmembrane domain comprises a sequence from L-selectin (CD62L), PSGL-1, or alpha4-integrin transmembrane domain. In some embodiments, the first binder further comprises an intracellular segment. In some embodiments, the intracellular segment comprise a sequence from an intracellular region of L-selectin (CD62L), PSGL-1, or alpha4-integrin.

### Second Binder

In some embodiments, the composition disclosed herein comprises a second binder connected to the first binder via a first linker. In some embodiments, the first binder and the second binder may be different. In some embodiments, the first binder and the second binder may be the same.

In some embodiments, the second binder may be a monoclonal antibody, a polyclonal antibody, a bispecific antibody, a trispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a nanobody, or an antigen-binding fragment thereof.

In some embodiments, the second binder may be a nanobody, a single chain variable fragment (scFv), a single-chain antibody, a single-domain antibody, a diabody, a Fab fragment, or a combination thereof. In some embodiments, the second binder may be a nanobody.

In some embodiments, the second binder may specifically bind to an antigen on the surface of a T cell. In some embodiments, the antigen on the surface of a T cell may be selected from CD8b, CD4, CD2, CD28, CD45RA, CD45RO, and CD58.

In some embodiments, the second binder may specifically bind to an antigen on the surface of a CAR cell, an NK cell, a granulocyte, a macrophage or a monocyte. In some embodiments, the antigen on the surface of a CAR cell, an NK cell, a granulocyte, a macrophage or a monocyte comprises CD15, CD11b, NKG2D, CD16, NKp30, NKp44, NKp46, DNAM, PSGL-1, CD44, CD11a, or CD49a.

In some embodiments, the second binder may bind to a viral epitope. In some embodiments, the viral epitope may be expressed on an Adeno-associated viruses (AAV). In some embodiments, the AAV may be selected form AAV1, AAV2, AAV3, AAV4, AAV5, AAV6, AAV7, AAV8, AAV9, AAV10, and AAV11.

In some embodiments, an AAV viral vector may be used to express the second binder. As used herein, the term “AAV vector” in the context of the present invention includes without limitation AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, and ovine AAV and any other AAV now known or later discovered. See, e.g., BERNARD N. FIELDS et al., VIROLOGY, volume 2, chapter 69 (4th ed., Lippincott-Raven Publishers). A number of additional AAV serotypes and clades have been identified (see, e.g., Gao et al., (2004) J. Virol. 78:6381-6388), which are also encompassed by the term “AAV.” Adenovirus is particularly suitable for use as a gene transfer vector because of its mid-sized genome, ease of manipulation, high titer, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

Adeno-Associated Virus (AAV) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of about 20 to about 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

AAVs stand out for use within the current disclosure because of their superb safety profile and because their capsids and genomes can be tailored to allow expression in selected cell populations. scAAV refers to a self-complementary AAV. pAAV refers to a plasmid adeno-associated virus. rAAV refers to a recombinant adeno-associated virus.

Other viral vectors may also be employed. For example, vectors derived from viruses such as vaccinia virus, polioviruses and herpes viruses may be employed. They offer several attractive features for various mammalian cells.

In some embodiments, vectors (e.g., AAV) include AAV9 (Gombash et al., Front Mol Neurosci. 2014; 7:81), AAVrh.10 (Yang, et al., Mol Ther. 2014; 22(7): 1299-1309), AAV1 R6, AAV1 R7 (Albright et al., Mol Ther. 2018; 26(2): 510), rAAVrh.8 (Yang, et al., supra), AAV-BR1 (Marchio et al., EMBO Mol Med. 2016; 8(6): 592), AAV-PHP.S (Chan et al., Nat Neurosci. 2017; 20(8): 1 172), AAV-PHP.B (Deverman et al., Nat Biotechnol. 2016; 34(2): 204), and AAV-PPS (Chen et al., Nat Med. 2009; 15: 1215). The PHP.eB capsid differs from AAV9 such that, using AAV9 as a reference, the sequence DGTLAVPFK (SEQ ID NO: 41) is inserted between amino acids residues 586 and 587 of AAV9.

In some embodiments, AAV comprises AAV type 1 (AAV1), AAV type 2 (AAV2), AAV type 3 (including types AAV3A and AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), and AAV type 11 (AAV 11) and any other AAV now known or later discovered.

In some embodiments, the second binder may bind to a nanoparticle. In some embodiments, the nanoparticle may be a lipid nanoparticle. In some embodiments, the nanoparticle may be a polymer nanoparticle. In some embodiments, the second binder may be associated with the surface of, encapsulated within, surrounded by, and/or distributed throughout the lipid formulation or polymeric matrix of an lipid nanoparticle, nanosphere, nanocarrier, microsphere, or microparticle. For example, in some embodiments, the second binder may, can be encapsulated within, surrounded by, and/or dispersed throughout the liposomal membrane and/or polymeric matrix of a lipid nanoparticle, nanosphere, nanocarrier, microsphere, or microparticle. Alternatively or additionally, in some embodiments, the second binder may can be associated with a lipid nanoparticle, nanosphere, nanocarrier, microsphere, or microparticle by charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof.

In some embodiments, the nanoparticles, nanospheres, nanocarriers, microparticles or microspheres may comprise one or more of polysaccharides, proteins, lipids, chitosan, alginate, pectin, xanthan gum, and cellulose. The nanoparticles, nanospheres or nanocarriers may be liposomes, polymeric micelles, dendrimers. In some embodiments, the dendrimers comprise those comprising poly-L-lysine, olyamidoamine (PAMAM), polypropylene imine (PPI), liquid crystalline, core-shell, chiral, peptide, glycodendrimers and PAMAMOS dendrimers. In some embodiments, the nanoparticles, nanospheres, nanocarrier, microparticles or microspheres may comprise an inorganic compound such as silver, gold, iron oxide, silica, zinc oxide, titanium oxide, platinum, selenium, gadolinium, palladium, or cerium dioxide.

In some embodiments, the second binder may be covalently linked to a lipid nanoparticle, nanosphere, nanocarrier, microsphere or microparticle. In some embodiments, the second binder may be linked to a nanoparticle, nanosphere, nanocarrier, microsphere or microparticle by a peptide linker. In some embodiments, the peptide linkers may comprise the dipeptide Val Cit (VC), the tripeptide AAN, or a longer peptide such as (GGGGS)ₙ (n=1, 2, 3, or 4) (SEQ ID NO: 32), (Gly)₈ (SEQ ID NO: 33), (Gly)₆ (SEQ ID NO: 34), (EAAAK)₃(SEQ ID NO: 35), (EAAAK)ₙ (n=1-3) (SEQ ID NO: 36), A(EAAAK)₄ALEA(EAAAK)₄A (SEQ ID NO: 37), PAPAP (SEQ ID NO: 38), AEAAAKEAAAKA (SEQ ID NO: 39), (Ala-Pro)ₙ (10-34 aa) (SEQ ID NO: 40). In some embodiments, the linkers may comprise GPI-anchors or cross-linked polymers.

In some embodiments, the second binder may be linked to a nanoparticle, nanosphere, nanocarrier, microsphere or microparticle by a cleavable linker comprising an acid-labile linker, a protease cleavable linker, an enzyme cleavable linker, or a reducible disulfide linkage. In some embodiments, the cleavable linkers may comprise those comprising an ester bond such as a glutaryl linker, those comprising an amide bond, and those comprising a carbamate bond. In some embodiments, the acid-labile linker may be hydrazone linkers.

In some embodiments, the second binder may be linked to a nanoparticle, nanosphere, nanocarrier, microsphere or microparticle by an uncleavable comprising an amide bond, a succinimidyl thioester linker, a triazole linker, or an oxime linker.

### Masking Moiety

The binder (e.g., anchorbody) provided herein may further comprise (e.g., at least one) masking moiety. In some embodiments, a masking moiety is connected to the binder via a second linker and the binder is connected to a radioactive isotope via a first linker (e.g., as provided in Formula (II)). The masking moieties provided herein may (e.g., selectively) prevent the binder (e.g., anchorbody) from binding to a (e.g., target) protein or “marker”, such as a (e.g., target) protein or “marker” provided elsewhere herein. In some embodiments, the masking moieties provided herein reversibly (e.g., selectively) prevent the binder (e.g., anchorbody) from binding to a (e.g., target) protein provided elsewhere herein. In some embodiments, the masking moiety selectively allows for binding of the binder to a (e.g., target) protein or “marker”, such as a protein or “marker” provided elsewhere herein, such as in the presence of an external stimuli. In some instances, the external stimuli comprise pH, enzymatic activity, temperature, ions, effector molecules, or antigen combinations.

In some embodiments, the first binder further comprises a first masking moiety. In some embodiments, the first masking moiety is covalently attached to a binding domain of the first binder via a second linker. In some embodiments, the first masking moiety is covalently attached to a heavy chain variable domain of the first binder via a second linker. In some embodiments, the first masking moiety is an anti-idiotypic antibody or fragment thereof. In some embodiments, the first masking moiety is an anti-idiotypic scFv or fragment thereof.

In some embodiments, the second binder further comprises a second masking moiety. In some embodiments, the second masking moiety is covalently attached to a binding domain of the second binder via a third linker. In some embodiments, the second masking moiety is covalently attached to a heavy chain variable domain of the second binder via a third linker. In some embodiments, the second masking moiety is an anti-idiotypic antibody or fragment thereof. In some embodiments, the second masking moiety is an anti-idiotypic scFv or fragment thereof.

In some embodiments, the second linker and the third linker are different. In some embodiments, the second linker and the third linker are the same. In some embodiments, the first masking moiety and the second masking moiety are different. In some embodiments, the first masking moiety and the second masking moiety are the same.

Others have tried to mask binding of an antibody by capping the binding moiety with a fragment of the antigen recognized by the binding moiety, however this approach has several limitations. For instance, using the antigen may allow for less flexibility in reducing the affinity of the binding moiety. This may be because the affinity must be high enough to be reliably masked by the antigen mask. In some instances, dissociated antigen could potentially bind to and interact with its cognate receptor(s) in vivo and cause undesirable signals to the cell expressing such receptor. In contrast, in some embodiments, described herein is a mask comprising an anti-idiotype antibody or fragment thereof. Two considerations for designing an effective masking moiety are (1) effectiveness of the masking and (2) reversibility of the masking. In some instances, if the affinity is too low, masking is inefficient. However, if affinity is too high, the masking process may not be readily reversible.

The masking moiety provided herein may be (e.g., covalently) attached to a binding domain of the binder. The masking moiety provided herein may be (e.g., covalently) attached to a binding domain of the binder via a second linker. In some embodiments, the masking moiety provided herein may be (e.g., covalently) attached to the binding domain of the binder via a second linker wherein the binder (e.g., specifically) binds to a protein expressed on an epithelial cell. In some embodiments, the compositions provided herein comprise a masking moiety connected to a binder via a second linker, such as a linker provided elsewhere herein, the binder being connected to a radioactive isotope via first linker, such as a linker provided elsewhere herein, wherein the binder specifically binds to a protein expressed on an endothelial cell.

The masking moiety provided herein may be (e.g., covalently) attached, such as via a second linker, to a binder, such as a nucleic acid, polypeptide, glycoprotein, carbohydrate, lipid, or small molecule. The masking moiety provided herein may be (e.g., covalently) attached, such as via a second linker, to a binder, such an antibody, antibody fragment, characteristic portions of antibodies, or single chain antibodies. In some embodiments, the masking moiety provided herein may be (e.g., covalently) attached, such as via a second linker, to a nanobody.

In some embodiments, the masking moiety provided herein may be covalently attached via a second linker to a nanobody. In some embodiments, the masking moiety provided herein may be covalently attached via a second linker to an antibody. In some embodiments, the masking moiety provided herein may be covalently attached via a second linker to a receptor. In some embodiments, the masking moiety provided herein may be covalently attached via a second linker to a cytokine. In some embodiments, the masking moiety provided herein may be covalently attached via a second linker to a peptide hormone. In some embodiments, the masking moiety provided herein may be covalently attached via a second linker to a glycoprotein. In some embodiments, the masking moiety provided herein may be covalently attached via a second linker to a glycopeptide. In some embodiments, the masking moiety provided herein may be covalently attached via a second linker to a proteoglycan. In some embodiments, the masking moiety provided herein may be covalently attached via a second linker to a protein derived from combinatorial libraries (e.g., Avimers™, Affibodies®, etc.). In some embodiments, the masking moiety provided herein may be covalently attached via a second linker to an antibody. In some embodiments, the masking moiety provided herein may be covalently attached via a second linker to an antibody fragment. In some embodiments, the masking moiety provided herein may be covalently attached via a second linker to an antibody-binding fragment.

The masking moieties provided herein may be (e.g., covalently) attached to a heavy chain variable domain of the binder. In some embodiments, the masking moieties provided herein may be (e.g., covalently) attached to a heavy chain variable domain (e.g., heavy chain variable region) of the binder via a second linker. In some embodiments, the heavy chain is a heavy chain (e.g. region) of a binder provided elsewhere herein. In some embodiments, the heavy chain is a heavy chain (e.g., region) of an antibody provided elsewhere herein. In some embodiments, the heavy chain is a heavy chain (e.g., region) of the anti-PMEPA-1 antibody.

The masking moieties provided herein may be (e.g., covalently) attached to a light chain variable domain of the binder. In some embodiments, the masking moieties provided herein may be (e.g., covalently) attached to a light chain variable domain (e.g., light chain variable region) of the binder via a second linker. In some embodiments, the light chain is a light chain (e.g. region) of a binder provided elsewhere herein. In some embodiments, the light chain is a light chain (e.g., region) of an antibody provided elsewhere herein. In some embodiments, the light chain is a light chain (e.g., region) of the anti-PMEPA-1 antibody.

The masking moiety provided herein may be an anti-idiotypic antibody or a fragment thereof. The masking moiety provided herein may be an anti-idiotypic antibody. The masking moiety provided herein may be a fragment of an anti-idiotypic antibody. Anti-idiotypic antibodies are antibodies that bind to (e.g., an idiotope of) another antibody. The masking moiety provided herein may be an anti-idiotypic antibody or a fragment thereof that may be covalently attached to a binder, such as a binder provided elsewhere herein, via a second linker. The masking moiety provided herein may be an anti-idiotypic antibody or a fragment thereof that may be non-covalently attached to a binder, such as a binder provided elsewhere herein.

The masking moiety provided herein may be an anti-idiotypic single chain variable fragment or a fragment thereof. The masking moiety provided herein may be an anti-idiotypic single chain variable fragment. The masking moiety provided herein may be a fragment of an anti-idiotypic single chain variable fragment. The masking moiety provided herein may be an anti-idiotypic single chain variable fragment or a fragment thereof that may be covalently attached to a binder, such as a binder provided elsewhere herein, via a second linker. The masking moiety provided herein may be an anti-idiotypic single chain variable fragment or a fragment thereof that may be non-covalently attached to a binder, such as a binder provided elsewhere herein.

In some instances, the masking moieties provided herein provide activatable binding of the binder (e.g., anchorbody) to a (e.g., target) protein or “marker”, such as a (e.g., target) protein or “marker” provided elsewhere herein. In some embodiments, the activation (e.g., stimulus) comprises acidic conditions (e.g., protonation of the masking moiety). In some embodiments, protonation of the masking moiety in acidic conditions is reversible. Acidic conditions herein may include any appropriate pH to provide reversible masking of the binder. In some embodiments, acidic conditions include a pH of at least 1.0. In some embodiments, non-limiting examples of acidic conditions include a pH of no more than 6.9. In some embodiments, acidic conditions include a pH of about 1.0 to about 6.9.

In some embodiments, activation (e.g., stimulus) comprises acidic conditions of tumor microenvironments. In some embodiments, acidic conditions include a pH of about 5.6 to about 6.8. In some embodiments, the binder (e.g., anchorbody) binds to a (e.g., target) protein or “marker” such as a (e.g., target) protein or “marker” provided elsewhere herein when exposed to a tumor microenvironment. In some embodiments, the binder (e.g., anchorbody) does not bind to a (e.g., protein) or “marker” such as a (e.g., target) protein or “marker” provided elsewhere herein in the presence of a pH above about 6.9. In some embodiments, the binder (e.g., anchorbody) does not bind to a (e.g., target protein) or “marker”, such as a (e.g., target) protein or “marker” provided elsewhere herein in the presence of a pH below about 5.6.

In some instances, the masking moieties provided herein provide activatable binding of the binder (e.g., anchorbody) to a (e.g., target) protein or “marker”, such as a (e.g., target) protein or “marker” provided elsewhere herein. In some embodiments, the activation (e.g., stimulus) comprises enzymatic activity, temperature, ions, effector molecules, or antigen combinations.

In some embodiments, the masking moieties provided herein have a dissociation constant (K_{D}) of at least 0.1 nM. In some embodiments, the masking moieties provided herein have a dissociation constant of at least 1 nM. In some embodiments, the masking moieties provided herein have a dissociation constant of at most 15 nM. In some embodiments, the masking moieties provided herein have a dissociation constant of at most 10 nM. In some embodiments, the masking moieties provided herein have a dissociation constant of about 0.1 nM to about 15 nM. In some embodiments, the masking moieties provided herein have a dissociation constant of about 1 nM to about 10 nM.

### Linker

In some embodiments, the compositions provided herein may comprise a (e.g., first) linker connecting the binder and the radioactive isotope or cell. In some embodiments, the compositions provided herein comprise a (e.g., second) linker connecting a masking moiety and the binder. In some embodiments, the compositions provided herein comprise a binder connected to a radioactive isotope via a first linker (e.g., L¹), such as provided in Formula (I). In some embodiments, the compositions provided herein comprise a masking moiety connected to a binder via a second linker (e.g., L²) and the binder is connected to a radioactive isotope via a first linker (e.g., L¹), as provided in Formula (II). In some embodiments, an association between a binder and a radioactive isotope or a cell may be mediated by a first linker. In some embodiments, an association between a binder and a masking moiety may be mediated by a second linker. In some embodiments, the first linker may be a flexible linker. In some embodiments, the second linker may be a flexible linker.

In some embodiments, the first linker (L¹) and second linker (L²) are the same. In some embodiments, the first linker (L¹) and second linker (L²) are different.

In some embodiments, any suitable linker can be used.

In some embodiments, the linker (e.g., first linker (L¹) or (L²)) may be a polypeptide linker. In some embodiments, the polypeptide linker may comprise about 2-20 amino acids. In some embodiments, the polypeptide linker may comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, the polypeptide linker may comprise at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, the polypeptide linker comprises (G₄S)ₙ, (SG₄)ₙ, G₄(SG₄)ₙ or G₂(SG₂)ₙ, wherein n is selected from 1 to 10. In some embodiments, peptide linkers include the dipeptide Val Cit (VC), the tripeptide AAN, or longer peptide such as (GGGGS)n(n=1, 2, 3, or 4) (SEQ ID NO: 32), (Gly)₈ (SEQ ID NO: 33), (Gly)₆ (SEQ ID NO: 34), (EAAAK)3(SEQ ID NO: 35), (EAAAK)n(n=1-3) (SEQ ID NO: 36), A(EAAAK)₄ALEA(EAAAK)₄A (SEQ ID NO: 37), PAPAP (SEQ ID NO: 38), AEAAAKEAAAKA (SEQ ID NO: 39), (Ala-Pro)n (10-34 aa) (SEQ ID NO: 40). In some embodiments, the polypeptide linker may comprise GGGGSGGGGS (SEQ ID NO: 41) or GGGGS (SEQ ID NO: 42).

Other types of linkers (e.g., first linker (L¹) or second linker (L²)) include GPI-anchors and cross-linked polymers. In some embodiments, the linkers (e.g., first linker (L¹) or second linker (L²)) may comprise GPI-anchors. In some embodiments, the linkers (e.g., first linker (L¹) or second linker (L²)) may comprise cross-linked polymers.

In some embodiments, the linker (e.g., first linker (L¹) or second linker (L²)) may comprise at least one group selected from the group consisting of alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene is optionally substituted.

In some embodiments, the linker (e.g., first linker (L¹) or second linker (L²)) may comprise substituted C₁-C₆ alkylene. In some embodiments, the linker (e.g., first linker (L¹) or second linker (L²)) may comprise unsubstituted C₁-C₆ alkylene. In some embodiments, the linker (e.g., first linker (L¹) or second linker (L²)) may comprise substituted C₁-C₆ heteroalkylene. In some embodiments, the first linker may comprise unsubstituted C₁-C₆ heteroalkylene. In some embodiments, the second linker may comprise substituted C₁-C₆ alkylene.

In some embodiments, the linker (e.g., first linker (L¹) or second linker (L²)) may comprise one or more groups selected from the group consisting of —O—, —S—, —NH—, —NH—(CH2)γ-NH, —NH—(CH2)_{y}—O, —O—(CH2)_{y}—O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH₂CH₂)_{w}—(C═O)—, —(C═O)—(CH═CH), —(C═O), —(C═O)—(OCH₂CH₂O)_{w}—(C═O)—, —(CH₂CH₂O)_{w}—, —(C═O)—(CH2CH2O)_{w}—, and —(CH(CH3)C(═O)O)_{w}—, wherein w is 1-20 and y is 1-20.

In some embodiments, the linker (e.g., first linker (L′) or second linker (L²)) may be a cleavable linker.

In some embodiments, a cleavable linker (e.g., first linker (L′) or second linker (L²)) provided herein may comprise an acid-labile linker, a protease cleavable linker, an enzyme cleavable linker, or a reducible disulfide linkage. In some embodiments, a cleavable linker comprises an acid-labile linker. In some embodiments, a cleavable linker is a protease cleavable linker. In some embodiments, a cleavable linker is an enzyme cleavable linker. In some embodiments, a cleavable linker is a reducible disulfide linkage. In some embodiments, the cleavable linkers may comprise an ester bond such as a glutaryl linker, an amide bond, and/or a carbamate bond. In some embodiments, an acid-labile linker may be a hydrazone linker.

In some embodiments, the cleavable linker (e.g., first linker (L¹) or second linker (L²)) provided herein may be a protease-cleavable linker, a self-immolative linker, or a pH-sensitive linker. In some embodiments, the cleavable linker is a protease-cleavable linker. In some embodiments, the cleavable linker is a self-immolative linker. In some embodiments, the cleavable linker is a pH-sensitive linker.

In some embodiments, the protease-cleavable linker (e.g., first linker (L¹) or second linker (L²)) comprises at least one protease recognition site. In some embodiments, the protease is selected from metalloproteinase (MMP) 1-28; A Disintegrin and Metalloproteinase (ADAM) 2, 7-12, 15, 17-23, 28-30 and 33; serine protease; urokinase-type plasminogen activator; Matriptase; cysteine protease; aspartic protease; and cathepsin protease. In some embodiments, the protease is MMP2 or MMP9.

In some embodiments, the self-immolative linker (e.g., first linker (L) or second linker (L²)) is selected from para-amino benzoic acid (PAB), para-aminobenzyl alcohol (PABA), 3,3-dimethyl-4-hydroxybutyric acid, ethylenediamine, γ-aminobutyric acid (GABA), 2-hydroxycinnamic acid, “Trimethyl Lock”, or ethanolamine. In some embodiments, the self-immolative linker may be para-amino benzoic acid (PAB).

In some embodiments, the pH-sensitive linker (e.g., first linker (L¹) or second linker (L²)) may be cleaved upon exposure to a target pH. In some embodiments, the target pH may be less than about 7. In some embodiments, the target pH may be less than at least about 2, 3, 4, 5, 6, 7, 8, or 9. In some embodiments, the target pH may be less than at most about 2, 3, 4, 5, 6, 7, 8, or 9. In some embodiments, the target pH may be about 2 to 9, 3 to 8, 4 to 7, or 5 to 6. In some embodiments, the target pH is the pH of a tumor microenvironment, such as a pH of about 5.6 to about 6.8. In some embodiments, the pH-sensitive linker may be selected from an optionally substituted tetrahydropyranyl ether, an optionally substituted tetrahydropyranyl ester, an optionally substituted azide, an optionally substituted histidine, an optionally substituted hydrazone, or an optionally substituted β-amino ester. In some embodiments, the pH-sensitive linker may be selected from -(tetrahydropyran ether)-(azide), -(hydrazone)-, -(hydrazone)-(azide)-, -(β-amino ester)-, -(β-amino ester)-(azide)-, or -(tetrahydropyran ester)-.

In some embodiments, the linker (e.g., first linker (L¹) or (L²)) may be a non-cleavable linker. In some embodiments, the non-cleavable linkers provided herein may comprise an amide bond, a succinimidyl thioester linker, a triazole linker, an oxime linker, or a triazole linker. In some embodiments, a non-cleavable linkers comprises an amide bond. In some embodiments, a non-cleavable linkers comprises a succinimidyl thioester linker (e.g., and an amide bond). In some embodiments, a non-cleavable linkers comprises a triazole linker (e.g., and an amide bond). In some embodiments, a non-cleavable linkers comprises an oxime linker. In some embodiments, a non-cleavable linkers comprises a triazole linker.

In some embodiments, the linker (e.g., first linker (L¹) or second linker (L²)) may be a flexible linker.

### Payload

In a certain aspect, the present disclosure provides a composition and methods to deliver a therapeutic payload to a target either globally or in distinct organs.

In some embodiments, the payload may be a toxin. In some embodiments, the payload may be a cytokine. In some embodiments, the payload may be cytotoxic. In some embodiments, the payload may be cytotoxic to a tumor cell upon internalization into the tumor cell.

In some embodiments, the payload may be an enzyme or enzyme inhibitor. In some embodiments, the payload may be a metal or metal particle that is responsive to certain types of radiation or detectable by NMR imaging. In some embodiments, the payload may be a detectable marker is selected from the group consisting of fluorescent labels, phosphorescent labels, chemiluminescent labels or bioluminescent labels, radio-isotopes, metals, metals chelates or metallic cations, chromophores and enzymes. In some embodiments, suitable markers and techniques for attaching, using and detecting them will be clear to the skilled person and, for example, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person and, for example, include moieties that can be detected using NMR or ESR spectroscopy.

In some embodiments, the payload may comprise an antitumor antibiotic, a microtubule inhibitor, a cytotoxic drug, a cytostatic drug, a topoisomerase inhibitor, a DNA-alkylating drug, a DNA-binding drug, a DNA-cleaving drug, or an RNA polymerase inhibitor. In some embodiments, the payload may comprise pyrrolobenzodiazepine, duocarmycin, auristatin, maytansinoid, uncialamycin, dynemicin, thailanstatin, camptothecin, exatecan, tubulysin compound, lurbinectedin, trabectedin, safracin, lenalidomide, eribulin, vincristine, vinblastine, vindesine, vinorelbine, an epothilone, a taxane (e.g., paclitaxel, docetaxel, cabazitaxel, etc.), a cryptophycin, a hemiasterlin, an anthracyclin, a bisnaphthylamide (e.g., elinafide), or a cytotoxic molecular glue/PROTAC compound.

In some embodiments, the payload may comprise dolastatin 10, auristatin E, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), or 5-benzoylvaleric acid-AE ester (AEVB).

In some embodiments, a drug to antibody ratio (DAR) of the composition may be about 1-30, wherein the drug is a payload and antibody is a binder. In some embodiments, a DAR of the composition may be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35, 36, 37, 38, 39, or 40. In some embodiments, a DAR of the composition may be at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 35, 36, 37, 38, 39, or 40.

### Radioactive Isotopes

Provided herein are radioactive isotopes (radioisotopes) connected to a binder, such as a binder provided elsewhere herein, via a first linker, wherein the binder specifically binds to a protein expressed on an endothelial cell. In some embodiments, the radioactive isotopes are connected to a binder via a first linker (e.g., such as provided in Formula (I)). In some embodiments, the radioactive isotopes are connected to a binder via a first linker and the binder is connected to a masking moiety vi a second linker (e.g., as provided in Formula (II)). The radioactive isotopes provided herein may be suitable for imaging and disease diagnosis. In some instances, positron emission tomography (PET) scans may be used to image the compositions provided herein, such as the radioactive isotopes provided herein. In some instances, the compositions provided herein target endothelial cells, such as the endothelial cells of tumors, and may allow for targeted imaging of tumors. In some instances, targeted imaging of tumors provides earlier stage detection of cancers as opposed to alternative general imaging techniques such as magnetic resonance imaging, computed tomography, or ultrasound. In some instances, earlier stage detection and diagnosis may provide better clinical outcomes. In some instances, imaging of the compositions provided herein, such as the radioactive isotopes provided herein, such as using PET scans, may be coupled with computed tomography.

In some embodiments, the radioactive isotopes provided herein kill cells, such as with alpha-particle emission. In some instances, alpha radiation causes direct, irreparable double-strand DNA breaks compared with gamma and beta radiation, which can cause single-stranded breaks via indirect DNA damage. In some instances, the alpha radiation produced by certain radioactive isotopes provides for treatment of certain ailments. In some instances, the targeting provided by the binder, such as the binder provided herein, and the radioactive isotope provided herein (e.g., such as a radioactive isotope that undergoes alpha emission), may be useful in targeted treatment, such as treatment of cancer.

After contacting a cell with the compositions provided herein, the compositions may be internalized by the cell, mediated by cell receptors, cell membrane endocytosis, or the like. In some embodiments, rapid internalization rate into cancer cells accompanied by a slow externalization rate can offer therapeutic benefit.

In some instances, the radioactive isotopes provided herein are useful for imaging (e.g., such as imaging using PET or SPECT) as well as treatment (e.g., such as via alpha particle emission).

The compositions provided herein may comprise one or more independent radioactive isotopes. In some embodiments, a composition provided herein comprises one radioactive isotope (e.g., through a linker of the composition). In some embodiments, a composition provided herein comprises two radioactive isotopes (e.g., through a linker of the composition).

In some embodiments, the radioactive isotope (radioisotope) provided herein is an alpha emitter, beta emitter, or gamma emitter.

In some embodiments, the radioactive isotope (radioisotope) is an alpha emitter. An alpha emitter is a radioactive isotope that undergoes alpha decay. Alpha decay is radioactive decay in which a nucleus emits an alpha particle (e.g., a helium nucleus). Alpha particles travel about one inch in air and can be shielded by a single sheet of paper and cannot penetrate the outer dead layer of skin.

In some embodiments, the radioactive isotope (radioisotope) is a beta emitter. Beta emitters undergo beta decay. In some embodiments, beta emitters emit energetic electrons from their nuclei. Beta particles can travel up to several meters and up to one-half an inch through skin and into the body.

In some embodiments, the radioactive isotope (radioisotope) is a gamma emitter. Gamma emitters emit gamma radiation (e.g., a penetrating form of electromagnetic radiation arising from radioactive decay of atomic nuclei). In some embodiments, gamma rays have wavelengths shorter than that of x-rays. In some embodiments, gamma rays have frequencies of above 3×10¹⁹ Hz. Gamma rays can travel tens of meters in air and can easily penetrate the human body.

In some instances, the radioactive isotope is stabilized in a chelate (e.g., wherein the chelate is attached to the linker). In some embodiments, the chelate increases the stability of the radioactive isotope. In some embodiments, the chelator is bidentate. In some embodiments, the chelator is tridentate. In some embodiments, the chelator is tetradentate. In some embodiments, the chelator is pentadentate. In some embodiments, the chelator is hexadentate. In some embodiments, the chelator is heptadentate. In some embodiments, the chelator is octadentate. In some embodiments, chelator prevents leaching of the radioactive isotope. In some embodiments, the radioactive isotope is stable against leaching in the absence of a chelator (e.g., such as a due to attachment to the linker). In some embodiments, the radioactive isotope has enhanced stability connected to the chelator or linker as compared to freely in solution. In some instances, the radioactive isotope connected to the chelator or linker is (e.g., substantially) stable against transmetallation.

In some embodiments, the chelator (e.g., comprising the radioactive isotope) is DOTA, DOTP, DOTMA, DOTAM, DOTAGA, DTPA, NTA, EDTA, D03A, D02A, NOC, NOTA, TETA, DiAmSar, CB-Cyclam, CB-TE2A, DOTA-4AMP, H₄pypa, H₄octox, H₄octapa, p-N0₂-Bn-neunpa, or NOTP. In some embodiments, the chelator is connected to the linker.

In some embodiments, the radioactive isotopes provided herein emit particles or rays. In some embodiments, the radioactive isotopes emit particles. In some embodiments, the radioactive isotopes emit rays.

In some embodiments, the radioactive isotopes emit a particle with an energy of at least 1 keV. In some embodiments, the radioactive isotopes emit a particle with an energy of at least 10 keV. In some embodiments, the radioactive isotopes emit a particle with an energy of at least 50 keV. In some embodiments, the radioactive isotopes emit a particle with an energy of at least 80 keV. In some embodiments, the radioactive isotopes emit a particle with an energy of at most 10,000 keV. In some embodiments, the radioactive isotopes emit a particle with an energy of at most 7,000 keV. In some embodiments, the radioactive isotopes emit a particle with an energy of at most 1,500 keV. In some embodiments, the radioactive isotopes emit a particle with an energy of at most 250 keV. In some embodiments, the radioactive isotopes emit a particle with an energy of about 1 keV to about 10,000 keV. In some embodiments, the radioactive isotopes emit a particle with an energy of about 10 keV to about 7,000 keV. In some embodiments, the radioactive isotopes emit a particle with an energy of about 50 keV to about 1,500 keV. In some embodiments, the radioactive isotopes emit a particle with an energy of about 80 keV to about 250 keV.

In some embodiments, the radioactive isotopes emit a ray with an energy of at least 1 keV. In some embodiments, the radioactive isotopes emit a ray with an energy of at least 10 keV. In some embodiments, the radioactive isotopes emit a ray with an energy of at least 50 keV. In some embodiments, the radioactive isotopes emit a ray with an energy of at least 80 keV. In some embodiments, the radioactive isotopes emit a ray with an energy of at most 10,000 keV. In some embodiments, the radioactive isotopes emit a ray with an energy of at most 7,000 keV. In some embodiments, the radioactive isotopes emit a ray with an energy of at most 1,500 keV. In some embodiments, the radioactive isotopes emit a ray with an energy of at most 250 keV. In some embodiments, the radioactive isotopes emit a ray with an energy of about 1 keV to about 10,000 keV. In some embodiments, the radioactive isotopes emit a ray with an energy of about 10 keV to about 7,000 keV. In some embodiments, the radioactive isotopes emit a ray with an energy of about 50 keV to about 1,500 keV. In some embodiments, the radioactive isotopes emit a ray with an energy of about 80 keV to about 250 keV.

The radioactive isotope (radioisotope) provided herein may be any radioactive isotope (radioisotope) which can undergo alpha emission, beta emission, or gamma emission. In some embodiments, the radioactive isotope is selected from Actinium-225, Astatine-211, Iodine-123, Iodine-125, Iodine-126, Iodine-131, Iodine-133, Bismuth-212, Bromine-77, Indium-111, Indium-113m, Gallium-67, Gallium-68, Ruthenium-95, Ruthenium-97, Ruthenium-103, Ruthenium-105, Mercury-107, Mercury-203, Rhenium-186, Rhenium-188, Tellurium-121m, Tellurium-122m, Tellurium-125m, Thulium-165, Thulium-167, Thulium-168, Technetium-99m, Fluorine-18, Silver-111, Platinum-197, Palladium-109, Copper-67, Phosphorus-32, Phosphorus-33, Yttrium-90, Scandium-47, Samarium-153, Lutetium-177, Rhodium-105, Praseodymium-142, Praseodymium-143, Terbium-161, Holmium-166, Gold-199, Cobalt-57, Cobalt-58, Chromium-51, Iron-59, Selenium-75, Thallium-201, Zirconium-89, and Ytterbium-169.

In some embodiments, the radioactive isotope is selected from Iodine-123, Iodine-131, Indium-111, Gallium-67, Ruthenium-97, Technetium-99m, Cobalt-57, Cobalt-58, Chromium-51, Iron-59, Selenium-75, Thallium-201, and Ytterbium-169. In some embodiments, the radioactive isotope is Iodine-123. In some embodiments, the radioactive isotope is Iodine-131. In some embodiments, the radioactive isotope is Indium-111. In some embodiments, the radioactive isotope is Gallium-67. In some embodiments, the radioactive isotope is Ruthenium-97. In some embodiments, the radioactive isotope is Technetium-99m. In some embodiments, the radioactive isotope is Cobalt-57. In some embodiments, the radioactive isotope is Cobalt-58. In some embodiments, the radioactive isotope is Chromium-51. In some embodiments, the radioactive isotope is Iron-59. In some embodiments, the radioactive isotope is Selenium-75. In some embodiments, the radioactive isotope is Thallium-201. In some embodiments, the radioactive isotope is Ytterbium-169. In some embodiments, the radioactive isotope is Zirconium-89.

In some embodiments, the radioactive isotope is Actinium-225, Gallium-67, or Lutetium-177. In some embodiments, the radioactive isotope is Actinium-225. In some embodiments, the radioactive isotope is Gallium-67. In some embodiments, the radioactive isotope is Lutetium-177. In some embodiments, the radioactive isotope is Zirconium-89.

In some embodiments, the radioactive isotope is Technetium-99m.

In some embodiments, the radioactive isotope is Lutetium-177, Indium-111, Gallium-68, Copper-64, Zirconium-89, or Cerium-134.

In some embodiments, the radioactive isotopes provide imaging capabilities by undergoing radioactive decay (e.g., alpha, beta, or gamma emission). In some instances, the decay of radioactive isotopes used in PET scans produce small particles called positrons. These react with electrons in the body and when these two particles combine, they annihilate each other. This annihilation produces a small amount of energy in the form of two photons that shoot off in opposite directions. The detectors in the PET scanner measure these photons and use this information to create images of internal organs. In other embodiments, imaging is completed using single photon emission computed tomography (SPECT). In some instances, SPECT imagers have gamma emission detectors that can detect the gamma ray emissions from the radioactive isotopes that have been injected into the subject. SPECT allows for 3D imaging of the subject.

### Chimeric Receptor (Car)

Also provided herein are engineered immune cells comprising a polynucleotide that encodes a chimeric receptor (CAR). In some embodiments, the engineered immune cell comprises (a) a first polynucleotide that encodes a first binder that specifically binds to a protein expressed on an endothelial cell; and (b) a second polynucleotide that encodes a chimeric antigen receptor (CAR). In some embodiments, the immune cells is a T cell, macrophage, monocyte, granulocyte, natural killer (NK) cell, or natural killer T (NKT) cell. In some embodiments, the immune cell is CD4+ or CD8+ T cell. In some embodiments, the engineered cell is a CAR-T cell, CAR-macrophages, CAR-monocyte, CAR-granulocyte, CAR-NK cell, or a CAR-NKT cell.

In some embodiments, the engineered immune cell is a CAR-T cell, CAR-NK cell, or a CAR-NKT cell. In some embodiments, the engineered immune cell is a CAR-T cell. In some embodiments, the engineered immune cell is an engineered CAR-T cell. In some embodiments, the engineered CAR-T cells comprise a nucleic acid molecule comprising a nucleotide sequence having at least 90%, 91%, 92%, 93%, 94%, 95%, 96%, 97%, 98%, 99% sequence identity to SEQ ID NOs: 42-47.

In some embodiments, the CAR comprises a target domain, a hinge domain, a transmembrane domain, a co-stimulatory domain, or a signaling domain. In some embodiments, the CAR comprises any one or more of a target domain, a hinge domain, a transmembrane domain, a co-stimulatory domain, or a signaling domain. In some embodiments, the CAR comprises a target domain. In some embodiments, the CAR comprises a hinge domain. In some embodiments, the CAR comprises a transmembrane domain. In some embodiments, the CAR comprises a co-stimulatory domain. In some embodiments, the CAR comprises a signaling domain.

### Target Domain

In some embodiments, the CAR comprises a target domain. In some embodiments, the target domain comprises a second binder that specifically binds to a tumor-associated antigen. In some embodiments, the tumor-associated antigen is selected from CD19, CD20, CD22, CD30, CD37, CD38, CEA, EpCAM, or BCMA. In some embodiments, the tumor-associated antigen is CD19 or CD20. In some embodiments, the tumor-associated antigen is CD22 or CD30. In some embodiments, the tumor-associated antigen is CD37 or CD38. some embodiments, the tumor-associated antigen is CEA. In some embodiments, the tumor-associated antigen is EpCAM. In some embodiments, the tumor-associated antigen is BCMA.

### Hinge Domain

In some embodiments, the CAR comprise a hinge domain. In some embodiments, the hinge domain is selected from IgG1, IgG2, IgG3, IgG4, IgA, IgD, IgE, IgM, CD28, or CD8. In some embodiments, the hinge domain is IgG1 or IgG2. In some embodiments, the hinge domain is IgG3 or IgG4. In some embodiments, the hinge domain is IgA or IgD. In some embodiments, the hinge domain is IgE or IgM. In some embodiments, the hinge domain is CD28 or CD8. In some embodiments.

### Transmembrane Domain

In some embodiments, the CAR comprises a transmembrane domain. In some embodiments, the transmembrane domain is selected from alpha, beta or zeta chain of the T-cell receptor, CD28, CD3 epsilon, CD45, CD4, CD5, CD8, CD9, CD16, CD22, CD33, CD37, CD64, CD80, CD86, CD123, CD134, CD137, CD154, L-selectin (CD62L), PSGL-1, or alpha4-integrin. In some embodiments, the transmembrane domain is alpha, beta or zeta chain of the T-cell receptor. In some embodiments, the transmembrane domain is selected from CD28, CD3 epsilon, CD45, CD4, CD5, CD8, CD9, CD16, CD22, CD33, CD37, CD64, CD80, CD86, CD123, CD134, CD137, or CD154. In some embodiments, the transmembrane domain is selected from L-selectin (CD62L), PSGL-1, or alpha4-integrin. In some embodiments, the transmembrane domain is selectin (CD62L). In some embodiments, the transmembrane domain is PSGL-1. In some embodiments, the transmembrane domain is alpha4-integrin.

### Co-Stimulatory Domain

In some embodiments, the CAR comprise a co-stimulatory domain. In some embodiments, the co-stimulatory domain is selected from CD3ζ, CD3γ, CD3δ, CD3ε, CD5, CD22, CD79a, CD79b, CD66d, CD2, CD4, CD5, CD28, CD134, CD137, ICOS, CD154, 41-BB, or OX40. In some embodiments, the co-stimulatory domain is CD3ζ or CD3γ. In some embodiments, the co-stimulatory domain is CD3δ or CD3ε. In some embodiments, the co-stimulatory domain is CD5 or CD22. In some embodiments, the co-stimulatory domain is CD79a or CD79b. In some embodiments, the co-stimulatory domain is CD66d or CD2. In some embodiments, the co-stimulatory domain is CD4 or CD5. In some embodiments, the co-stimulatory domain is CD28 or CD134. In some embodiments, the co-stimulatory domain is CD137 or ICOS. In some embodiments, the co-stimulatory domain is CD154 or 41-BB. In some embodiments, the co-stimulatory domain is 41BB or OX40.

### Signaling Domain

In some embodiments, the CAR comprise a signaling domain. In some embodiments, the signaling domain is selected from CD3ζ, CD3γ, CD3δ, CD3ε, CD5, CD22, CD79a, CD79b, CD66d, CD2, CD4, CD5, CD28, CD134, CD137, ICOS, CD154, 41-BB, or OX40. In some embodiments, the signaling domain is CD3ζ or CD3γ. In some embodiments, the signaling domain is CD3δ or CD3ε. In some embodiments, the signaling domain is CD5 or CD22. In some embodiments, the signaling domain is CD79a or CD79b. In some embodiments, the signaling domain is CD66d or CD2. In some embodiments, the signaling domain is CD4 or CD5. In some embodiments, the signaling domain is CD28 or CD134. In some embodiments, the signaling domain is CD137 or ICOS. In some embodiments, the signaling domain is CD154 or 41-BB. In some embodiments, the signaling domain is 41BB or OX40. In some embodiments, the first binder is connected to the signaling domain. In some embodiments, the first binder is not connected to the signaling domain. In some embodiments, the second binder is connected to the signaling domain. In some embodiments, the second binder is not connected to the signaling domain.

### Pharmaceutical Compositions

The present disclosure provides a pharmaceutical composition comprising the composition disclosed herein and a pharmaceutically acceptable carrier and/or excipient.

In some embodiments, the pharmaceutical compositions comprising any one of the compositions provided herein (e.g., such as a binder connected to a radioactive isotope via a first linker). Provided herein are pharmaceutical compositions comprising any one of the compositions provided in Formula (I) or Formula (II). In some embodiments, the pharmaceutical composition comprises any one of the compositions provided herein (e.g., such as a binder connected to a radioactive isotope via a first linker) and a pharmaceutically acceptable carrier and/or excipient.

The phrase “pharmaceutically-acceptable carriers” refer to carriers that do not produce an allergic or similar untoward reaction when administered to a human, and in some embodiments, when administered intravenously.

Provided herein are pharmaceutical compositions comprising any one of the compositions provided herein (e.g., such as a binder connected to a radioactive isotope via a first linker or a binder connected to a masking moiety connected via second linker and the binder is connected to the radioactive linker via a first linker) further comprising at least one additional therapeutic agent.

In some embodiments, the at least one additional therapeutic agent may act synergistically with the any one of the compositions (e.g., such as a binder connected to a radioactive isotope via a first linker or a binder connected to a masking moiety connected via second linker and the binder is connected to the radioactive linker via a first linker) described herein, or they may independently exert their intended effects.

The therapeutic agent may be any agent which a skilled artisan would use in connection with a method, composition, or kit described herein.

The therapeutic agent may be any agent that when administered to a subject has a therapeutic, prophylactic, diagnostic, biological, and/or pharmacological effect.

In some embodiments, the therapeutic agent may be selected from the group consisting of a small molecule, saccharide, oligosaccharide, polysaccharide, peptide, protein, peptide analog and derivatives, peptidomimetic, siRNAs, shRNAs, antisense RNAs, ribozymes, dendrimers, aptamers, and any combination thereof.

In some embodiments, the therapeutic agent may be a chemotherapeutic, an antibiotic, a steroid, a non-steroid anti-inflammatory drug, or an analgesic. In some embodiments, the therapeutic agent is a steroid. In some embodiments, the steroid is dexamethasone. In some embodiments, the steroid is prednisone. In some embodiments, the steroid is hydrocortisone. In some embodiments, the steroid is cortisone. In some embodiments, the therapeutic agent is a mToR inhibitor. In some embodiments, the mToR inhibitor is rapamycin. In some embodiments, the therapeutic agent is a complement inhibitor. In some embodiments, the therapeutic agent is a JAK-STAT inhibitor.

In some embodiments, the therapeutic agent is a toxin. In some embodiments, the therapeutic agent is a cytokine. In some embodiments, the therapeutic agent may be cytotoxic to a tumor cell upon internalization into a tumor cell.

In some embodiments, the therapeutic agent comprises an antitumor antibiotic, microtubule inhibitor, cytotoxic or cytostatic, topoisomerase inhibitor, a pyrrolobenzodiazepine, a DNA-alkylating drug, a DNA-binding drug, a DNA-cleaving drug, or an RNA polymerase inhibitor. In some embodiments, the therapeutic agent may comprise a pyrrolobenzodiazepine, duocarmycin, auristatin, maytansinoid, uncialamycin, dynemicin, thailanstatin, camptothecin, exatecan, tubulysin compound, lurbinectedin, trabectedin, safracin, lenalidomide, eribulin, vincristine, vinblastine, vindesine, vinorelbine, an epothilone, a taxane (e.g., paclitaxel, docetaxel, cabazitaxel, etc.), a cryptophycin, a hemiasterlin, an anthracyclin, a bisnaphthylamide (e.g., elinafide), or a cytotoxic molecular glue/PROTAC compound.

In some embodiments, the therapeutic agent comprises dolastatin 10, auristatin E, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), or 5-benzoylvaleric acid-AE ester (AEVB).

In some embodiments, the therapeutic agent may be cell death stimulating agents. The term “cell death stimulating agents” refers to agents that induce cell (e.g., cancer cell) death. In some embodiments, the cell death stimulating agents may be immunogenic or non-immunogenic in nature. Cell death can be classified according to the morphological appearance of the lethal process (that may be apoptotic, necrotic, autophagic or associated with mitosis), enzymological criteria (with and without the involvement of nucleases or distinct classes of proteases, like caspases), functional aspects (programmed or accidental, physiological or pathological) or immunological characteristics (immunogenic or non-immunogenic) (Kroemer et al., 2009).

As used herein, the term “immunogenic cell death” or “immunogenic apoptosis” refers to dying cells that alert the immune system, which then mounts a therapeutic anti-cancer immune response and contributes to the eradication of residual tumor cells. Conversely, when cancer cells succumb to a non-immunogenic death modality, i.e., non-immunogenic cell death, they fail to elicit such a protective immune response.

The term “anti-cancer immune response” refers to when an immune response is directed against tumor cells, in particular cancerous cells. The anti-cancer immune response is allowed by a reaction from the immune system of the subject to the presence of cells, preferably of tumor cells, dying from an immunogenic cell death (as defined previously).

The terms “agent that induces an immunogenic cell death” or “immunogenic cell death stimulating agent” refer to an agent that induces cell death which then in turn induces an anti-cancer immune response. In some embodiments, the composition described herein may target and/or transport one or more immunogenic cell death stimulating agents (e.g., an agent that induces an immunogenic cell death, e.g., chemotherapeutic agent or CAR T cells) which can help stimulate immune responses. In some embodiments, immunogenic cell death stimulating agents boost immune responses by activating APCs to enhance their immunostimulatory capacity. In some embodiments, immunogenic cell death stimulating agents boost immune responses by amplifying lymphocyte responses to specific antigens. In some embodiments, immunogenic cell death stimulating agents boost immune responses by inducing the local release of mediators, such as cytokines from a variety of cell types. In some embodiments, the immunogenic cell death stimulating agents suppress or redirect an immune response. In some embodiments, the immunogenic cell death stimulating agents induce regulatory T cells. In some embodiments, the immunogenic cell death stimulating agents increase the levels or activity of intra-tumoral T cells.

As used herein, the term “agent that induces a non-immunogenic cell death” refers to an agent that induces cell death, but fails to elicit a corresponding protective immune response in doing so.

In some embodiments, the term “agent that induces an inflammatory response” refers to an agent that induces an inflammatory response which in turn induces a pro-inflammatory cytokine cascade.

Cytokines activate immune cells such as T cells and macrophages, stimulating them to produce more cytokines resulting in so-called cytokine storms or cascades. In some embodiments, the agent that induces an inflammatory response is a TLR4 agonist or a GP-130 agonist.

In some embodiments, the composition described herein and cell death stimulating agents or inflammatory response stimulating agents may be coupled (e.g., covalently associated or within the same structure such as within a nanoparticle, or the composition, is decorating the cell membrane of a CAR T cell). In some embodiments, a nanoparticle comprises a lipid membrane (e.g., lipid bilayer, lipid monolayer, etc.), wherein at least one type of cell death stimulating agent is associated with the lipid membrane of the nanoparticle and composition described herein is associated with the lipid membrane of the nanoparticle. In some embodiments, at least one type of cell death stimulating agent is embedded within the lipid membrane of the nanoparticle and composition described herein is embedded within the lipid membrane of the nanoparticle. In some embodiments, the at least type of cell death stimulating agent is encapsulated by the lipid membrane of the nanoparticle and composition described herein is associated and/or embedded in the lipid membrane or the nanoparticle.

In certain embodiments, cell death stimulating agents or inflammatory response stimulating agents may be interleukins, interferon, cytokines, etc. In specific embodiments, cell death stimulating agent may be a natural or synthetic agonist for a Toll-like receptor (TLR). In specific embodiments, nanoparticles incorporate a ligand for toll-like receptor (TLR)-7, such, as CpGs, which induce type I interferon production. In specific embodiments, cell death stimulating agent may be an agonist for the DC surface molecule CD40. In certain embodiments, to stimulate immunity rather than tolerance, a nanoparticle incorporates a cell death stimulating agent that promotes DC maturation (needed for priming of naive T cells) and the production of cytokines, such as type I interferons, which promote antibody responses and anti-viral immunity. In some embodiments, cell death stimulating agent may be a TLR-4 agonist, such as bacterial lipopolysaccharide (LPS), VSV-G, and/or HMGB-1. In some embodiments, cell death stimulating agent are cytokines, which are small proteins or biological factors (in the range of 5 kD-20 kD) that are released by cells and have specific effects on cell-cell interaction, communication and behavior of other cells. In some embodiments, cell death stimulating agent may be proinflammatory stimuli released from necrotic cells (e.g., urate crystals). In some embodiments, cell death stimulating agents or inflammatory response stimulating agents may be activated components of the complement cascade (e.g., CD21, CD35, etc.). In some embodiments, cell death stimulating agents or inflammatory response stimulating agents may be activated components of immune complexes. The cell death stimulating agents include TLR-1, TLR-2, TLR-3, TLR-4, TLR-5, TLR-6, TLR-7, TLR-8, TLR-9, and TLR-10 agonists. The inflammatory response stimulating agents include, but are not limited to, TLR-4 agonist and GP-130 agonist. The cell death stimulating agents also include complement receptor agonists, such as a molecule that binds to CD21 or CD35. In some embodiments, the complement receptor agonist induces endogenous complement opsonization of the nanocarrier. In some embodiments, the cell death stimulating agents also include cytokine receptor agonists, such as a cytokine. In some embodiments, the cytokine receptor agonist is a small molecule, antibody, fusion protein, or aptamer.

In some embodiments, there are more than one type of cell death stimulating agent, e.g., immunogenic and/or non-immunogenic cancer cell death stimulating agent. In some embodiments, the different cell death stimulating agents or different inflammatory response stimulating agents each act on a different pathway. The cell death stimulating agents, therefore, can be different Toll-like receptors, a Toll-like receptor and CD40, a Toll-like receptor and a component of the inflammasome, etc.

In some embodiments, the cell death stimulating agents or inflammatory response stimulating agents may be an adjuvant. Thus, in some embodiments, the present invention provides pharmaceutical compositions comprising nanoparticles formulated with one or more adjuvants. The term “adjuvant”, as used herein, refers to an agent that does not constitute a specific antigen, but boosts the immune response to the administered antigen.

In some embodiments, the cell death stimulating agent may be a chimeric antigen receptor T cell (CAR T cell). As used herein, a “chimeric antigen receptor” (CAR) is an artificially constructed hybrid protein or polypeptide comprising a specificity or recognition (i.e. binding) domain linked to an immune receptor responsible for signal transduction in lymphocytes. The binding domain is typically derived from a Fab antibody fragment that has been fashioned into a single chain scFv via the introduction of a flexible linker between the antibody chains within the specificity domain. Other possible specificity domains can include the signaling portions of hormone or cytokine molecules, the extracellular domains of receptors, and peptide ligands or peptides isolated by library (e.g. phage) screening (see Ramos and Dotti, (2011) Expert Opin Bio Ther 11(7): 855). Flexibility between the signaling and the binding portions of the CAR may be a desirable characteristic to allow for more optimum interaction between the target and the binding domain, so often a hinge region is included. One example of a structure that can be used is the CH₂—CH₃ region from an immunoglobulin such as an IgG molecule. The signaling domain of the typical CAR comprises intracellular domains of the TCR-CD3 complex such as the zeta chain. Alternatively, the γ chain of an Fe receptor may be used. The transmembrane portion of the typical CAR can comprise transmembrane portions of proteins such as CD4, CD8 or CD28 (Ramos and Dotti, ibid). Characteristics of some CARs include their ability to redirect T-cell specificity and reactivity toward a selected target in a non-MHC-restricted manner. The non-MHC-restricted target recognition gives T-cells expressing CARs the ability to recognize a target independent of antigen processing, thus bypassing a major mechanism of tumor escape.

In some embodiments, the compositions can be formulated for intravenous, intraocular, intravitreal, parenteral, subcutaneous, intracerebro-ventricular, intramuscular, intracerebroventricular, intravenous injection into the cisterna magna (ICM), intrathecal, intraspinal, oral, intraperitoneal, oral or nasal inhalation, or by direct injection in or application to one or more cells, tissues, or organs.

As used herein, the term “excipient” refers to pharmacologically inactive ingredients that are included in a formulation with the API, e.g., ceDNA and/or lipid nanoparticles to bulk up and/or stabilize the formulation when producing a dosage form. General categories of excipients include, for example, bulking agents, fillers, diluents, antiadherents, binders, coatings, disintegrants, flavours, colors, lubricants, glidants, sorbents, preservatives, sweeteners, and products used for facilitating drug absorption or solubility or for other pharmacokinetic considerations.

In some embodiments, the pharmaceutical composition may comprise a pharmaceutically acceptable nanocapsule formulation. Nanocapsules can generally entrap compounds in a stable and reproducible way (Quintanar-Guerrero et al., Drug Dev Ind Pharm 24(12): 11 13-1 128, 1998; Quintanar-Guerrero et al, Pharm Res. 15(7): 1056-1062, 1998; Quintanar-Guerrero et al., J. Microencapsul. 15(1): 107-1 19, 1998; Douglas et al, Crit Rev Ther Drug Carrier Syst 3(3):233-261, 1987). To avoid side effects due to intracellular polymeric overloading, such ultrafine particles can be designed using polymers able to be degraded in vivo. Biodegradable polyalkyl-cyanoacrylate nanoparticles that meet these requirements are contemplated for use in the present disclosure. Such particles can be easily made, as described in Couvreur et al., J Pharm Sci 69(2): 199-202, 1980; Couvreur et al., Crit Rev Ther Drug Carrier Syst. 5(1)1-20, 1988; zur Muhlen et al., EurJ Pharm Biopharm, 45(2): 149-155, 1998; Zambau x et al., J Control Release 50(1-3):31-40, 1998; and U.S. Pat. No. 5,145,684.

In some embodiments, the pharmaceutical composition may be injectable. In some embodiments, injectable pharmaceutical composition may comprise sterile aqueous solutions or dispersions and sterile powders for the extemporaneous preparation of sterile injectable solutions or dispersions (U.S. Pat. No. 5,466,468). For delivery via injection, the form is sterile and fluid to the extent that it can be delivered by syringe. In some embodiments, it may be stable under the conditions of manufacture and storage, and optionally contains one or more preservative compounds against the contaminating action of microorganisms, such as bacteria and fungi. In some embodiments, the carrier may be a solvent or dispersion medium comprising water, ethanol, polyol (e.g., glycerol, propylene glycol, and liquid polyethylene glycol, and the like), suitable mixtures thereof, or vegetable oils. In some embodiments, proper fluidity may be maintained by the use of a coating comprising lecithin. In some embodiments, proper fluidity may be maintained by the maintenance of the required particle size in the case of dispersion. In some embodiments, proper fluidity may be maintained by the use of surfactants. In some embodiments, the action of microorganisms may be prevented by various antibacterial and/or antifungal agents. In some embodiments, the antibacterial and/or antifungal agents comprise parabens, chlorobutanol, phenol, sorbic acid, thimerosal, and the like. In various embodiments, the pharmaceutical composition may comprise an isotonic agent comprising sugar(s) or sodium chloride. In some embodiments, prolonged absorption of the injectable compositions may be accomplished by an agent that delay absorption comprising aluminum monostearate or gelatin. In some embodiments, the injectable compositions may be suitably buffered, if necessary, and the liquid diluent first rendered isotonic with sufficient saline or glucose.

In some embodiments, the pharmaceutical composition may be formulated as a dispersion. In some embodiments, dispersions may be prepared in glycerol, liquid polyethylene glycols, and mixtures thereof and in oils. In some embodiments, the pharmaceutical composition may comprise a preservative to prevent the growth of microorganisms.

In some embodiments, the pharmaceutical composition may be sterile. Sterile pharmaceutical composition may be prepared by incorporating the physiologically active component in an appropriate amount of a solvent with other optional ingredients (e.g., as enumerated above), followed by filtered sterilization. In some embodiments, dispersions may be prepared by incorporating the various sterilized physiologically active components into a sterile vehicle comprising the basic dispersion medium and the required other ingredients (e.g., from those enumerated above). In the case of sterile powders for the preparation of sterile injectable solutions, vacuum-drying or freeze-drying techniques may be used to yield a powder of the physiologically active components and any additional desired ingredient from a previously sterile-filtered solution thereof.

In some embodiments, the pharmaceutical composition may be an oral formulation. In some embodiments, the oral formulations may be in liquid form comprising solutions, syrups, or suspensions. In some embodiments, the oral formulations may be presented as a drug product for reconstitution with water or other suitable vehicle before use. In some embodiments, liquid may be prepared by conventional means with pharmaceutically acceptable additives comprising suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); or preservatives (e.g., methyl or propyl-p-hydroxybenzoates or sorbic acid). In some embodiments, the pharmaceutical composition may take the form of tablets or capsules prepared by conventional means with pharmaceutically acceptable excipients comprising binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well-known in the art.

In some embodiments, the pharmaceutical composition may be inhalable. In some embodiments, the inhalable pharmaceutical composition may be delivered in the form of an aerosol spray presentation from pressurized packs or a nebulizer, with the use of a suitable propellant comprising dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

In some embodiments, the pharmaceutical composition comprises a lipid formulation. In some embodiments, the lipid formulation comprises a lipid nanoparticle.

As used herein, the term “lipid nanoparticle” refers to a vesicle formed by one or more lipid components. Lipid nanoparticles are typically used as carriers for nucleic acid delivery in the context of pharmaceutical development. They work by fusing with a cellular membrane and repositioning its lipid structure to deliver a drug or active pharmaceutical ingredient (API). Generally, lipid nanoparticle compositions for such delivery are composed of synthetic ionizable or cationic lipids, phospholipids (especially compounds having a phosphatidylcholine group), cholesterol, and a polyethylene glycol (PEG) lipid; however, these compositions may also include other lipids. The sum composition of lipids typically dictates the surface characteristics and thus the protein (opsonization) content in biological systems thus driving biodistribution and cell uptake properties.

The pharmaceutical composition described herein may be associated with the surface of, encapsulated within, surrounded by, and/or distributed throughout the lipid formulation or polymeric matrix of an lipid nanoparticle, nanosphere, nanocarrier, microsphere, or microparticle. For example, in some embodiments, the pharmaceutical composition described herein can be encapsulated within, surrounded by, and/or dispersed throughout the liposomal membrane and/or polymeric matrix of a lipid nanoparticle, nanosphere, nanocarrier, microsphere, or microparticle. Alternatively or additionally, the pharmaceutical composition described herein can be associated with a lipid nanoparticle, nanosphere, nanocarrier, microsphere, or microparticle by charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof.

The nanoparticles, nanospheres, nanocarriers, microparticles or microspheres may comprise one or more of polysaccharides, proteins, lipids, chitosan, alginate, pectin, xanthan gum, and cellulose. The nanoparticles, nanospheres or nanocarriers may be liposomes, polymeric micelles, dendrimers. Exemplary dendrimers include those comprising poly-L-lysine, olyamidoamine (PAMAM), polypropylene imine (PPI), liquid crystalline, core-shell, chiral, peptide, glycodendrimers and PAMAMOS dendrimers.

Alternatively, the nanoparticles, nanospheres, nanocarrier, microparticles or microspheres may comprise an inorganic compound such as silver, gold, iron oxide, silica, zinc oxide, titanium oxide, platinum, selenium, gadolinium, palladium, or cerium dioxide.

In some embodiments, the pharmaceutical composition described herein is covalently linked to a lipid nanoparticle, nanosphere, nanocarrier, microsphere or microparticle. For example, the composition described herein is linked to a nanoparticle, nanosphere, nanocarrier, microsphere or microparticle by a peptide linker. In some embodiments, the peptide linkers may comprise the dipeptide Val Cit (VC), the tripeptide AAN, or a longer peptide such as (GGGGS)n (n=1, 2, 3, or 4) (SEQ ID NO: 32), (Gly)8 (SEQ ID NO: 33), (Gly)6 (SEQ ID NO: 34), (EAAAK)3(SEQ ID NO: 35), (EAAAK)(n=1-3) (SEQ ID NO: 36), A(EAAAK)4ALEA(EAAAK)4A (SEQ ID NO: 37), PAPAP (SEQ ID NO: 38), AEAAAKEAAAKA (SEQ ID NO: 39), (Ala-Pro)n (10-34 aa) (SEQ ID NO: 40). Other types of linkers include GPI-anchors and cross-linked polymers.

As used herein, the term “conjugated lipid” refers to a lipid molecule conjugated with a non-lipid molecule, such as a PEG, polyoxazoline, polyamide, or polymer (e g., cationic polymer).

In some embodiments, the pharmaceutical composition may comprise supplementary active ingredients.

In some embodiments, the pharmaceutical composition may comprise at least about 0.1% of the components disclosed herein or more. In some embodiments, the pharmaceutical composition may comprise about 1 or about 2% of the components disclosed herein. In some embodiments, the pharmaceutical composition may comprise about 70% or about 80% or more of the components disclosed herein. In some embodiments, the pharmaceutical composition may comprise about 0.5 to about 99% of the components disclosed herein out of the weight or volume of the total composition. In some embodiments, the pharmaceutical composition may comprise at least about 0.1, 0.5, 1, 2, 5, 8, 10, 15, 20, 25, 30, 35, 40, 45, 50, 55, 60, 65, 70, 75, 80, 85, 90, 95, 98, or 99% of the components disclosed herein out of the weight or volume of the total composition.

In some embodiments, for administration to humans, the pharmaceutical composition meet sterility, pyrogenicity, and the general safety and purity standards as required by United States Food and Drug Administration (FDA) or other applicable regulatory agencies in other countries.

In some embodiments, the pharmaceutical composition may further comprise at least one additional therapeutic agent. As used herein, the term “therapeutic agent” refers to any agent that, when administered to a subject, has a therapeutic, prophylactic, and/or diagnostic effect and/elicits a desired biological and/or pharmacological effect. In some embodiments, the at least one additional therapeutic agent may act synergistically with the composition described herein, or they may independently exert their intended effects. The disclosure contemplates any therapeutic agent which a skilled artisan would use in connection with a method, composition, or kit described herein.

### Methods of Use

Provided herein are methods of using the compositions, pharmaceutical formulations, or formulations provided herein.

In one aspect, the present disclosure also provides a method of selectively delivering a radiopharmaceutical composition, such as a composition provided elsewhere herein, to a tumor endothelial cell compared to a non-tumor endothelial cell in a subject in need thereof, the method comprising administering to the subject an effective amount of the composition.

In some embodiments, the method comprises selectively delivering a (e.g., radiopharmaceutical) composition, such as a (e.g., radiopharmaceutical) composition provided elsewhere herein comprising a binder connected to a radioactive isotope via a first linker.

In some embodiments, the method comprises selectively delivering a (e.g., radiopharmaceutical) composition, such as a composition provided elsewhere herein, to a tumor endothelial cell compared to a non-tumor endothelial cell in a subject in need thereof comprising an effective amount of the composition or pharmaceutical composition.

In some embodiments, selectively delivering the compositions or pharmaceutical compositions provides (e.g., targeted) imaging capabilities of the radioactive isotopes. In some embodiments, the targeted imaging capabilities of the radioactive isotopes is provided by the compositions selective binding (e.g., increased binding affinity) to endothelial cells, and especially tumor endothelial cells as compared to non-tumor endothelial cells. In some embodiments, the methods provided herein can be used in combination with any imaging method known to one skilled in the art. In some embodiments, the methods provided herein can be used in combination with one or more or of SPECT imaging and PET imaging. In some embodiments, PET and/or SPECT imaging can be used in combination with one or more of MRI or CT.

In some embodiments, provided herein is a method of selectively delivering a (e.g., radiopharmaceutical) composition, such as a (e.g, radiopharmaceutical) composition provided elsewhere herein, to an subject in need thereof, and imaging the subject (e.g., or composition), such as by PET or SPECT.

In one aspect, the present disclosure also provides a method of selectively delivering a composition to a tumor endothelial cell compared to a non-tumor endothelial cell in a subject in need thereof. In some embodiments, the method comprises administering to the subject an effective amount of the composition disclosed herein, or the pharmaceutical compositions disclosed herein to the subject. In some embodiments, the composition may have a greater binding affinity for a tumor endothelial cell compared to a binding affinity for a non-tumor endothelial cell.

In some embodiments, composition, such as a composition provided elsewhere herein, has a greater binding affinity for a tumor endothelial cell compared to a binding affinity for a non-tumor endothelial cell. In some embodiments, the selectively delivering is provided by the greater binding affinity for a tumor endothelial cell by a composition provided herein compared to a binding affinity for a non-tumor endothelial cell. The greater binding affinity for a tumor endothelial cell compared to a binding affinity for a non-tumor endothelial cell may be a result of the binder's interaction with the (e.g., transmembrane) protein of the endothelial cell, as described elsewhere herein.

In some embodiments, the composition provided herein has at least 5% (e.g., at least 10%, at least 20%, at least 40%, at least 60%, at least 100%, at least 200%) higher binding affinity for a tumor endothelial cell compared to a binding affinity for a non-tumor endothelial cell.

Provided herein, in some embodiments, is a method of treating a disease or disorder in a subject in need thereof comprising administering to the subject an effective amount of a composition provided elsewhere herein (e.g., a composition comprising a binder connected to a radioactive isotope via a first linker) or a pharmaceutical composition provided elsewhere herein.

In some embodiments, the radioactive isotopes provided herein kill cells (e.g., and as such provide treatment of a disease or disorder provided herein), such as via alpha-particle emission. In some instances, alpha particle emission causes direct, irreparable double-strand DNA breaks compared with gamma and beta radiation, which can cause single-stranded breaks via indirect DNA damage. In some instances, the alpha radiation produced by certain radioactive isotopes provides for treatment of certain diseases and disorders, such as cancer. In some instances, the targeting provided by the binder, such as the binder provided herein, and the radioactive isotope provided herein (e.g., such as a radioactive isotope that undergoes alpha emission), may be useful in targeted treatment, such as treatment of cancer. In compositions without a binder provided herein, where targeting of epithelial cells is not present, delivery of radioactive nuclei have increased potential of killing healthy cells. In some instances, as a result of targeting provided by the binders provided herein (e.g., selectively delivering), healthy cell death may be minimized and target cell (e.g., cancerous cell) death may be increased.

After contacting a cell with the compositions provided herein, the compositions may be internalized by the cell, mediated by cell receptors, cell membrane endocytosis, or the like. In some embodiments, rapid internalization rate into cancer cells accompanied by a slow externalization rate can offer therapeutic benefit.

In a certain aspect, the present disclosure provides a method of treating a disease or disorder in a subject in a need thereof. In some embodiments, the method comprises administering to the subject an effective amount of the composition disclosed herein, or the pharmaceutical composition disclosed herein to the subject.

In some embodiments, the disease or disorder may include, but is not limited to, endotoxemia, sepsis, cancer, obesity-related insulin resistance, diabetes, polycystic ovary syndrome, metabolic syndrome, hypertension, cerebrovascular accident, myocardial infarction, congestive heart failure, cholecystitis, gout, osteoarthritis, Pickwickian syndrome, sleep apnea, atherosclerosis, inflammatory bowel disease, rheumatoid arthritis, vasculitis, transplant rejection, asthma, ischemic heart disease, appendicitis, peptic, gastric and duodenal ulcers, peritonitis, pancreatitis, ulcerative, pseudomembranous, acute and ischemic colitis, diverticulitis, epiglottitis, achalasia, cholangitis, hepatitis, Crohn's disease, enteritis, Whipple's disease, allergy, anaphylactic shock, immune complex disease, organ ischemia, reperfusion injury, organ necrosis, hay fever, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, alveolitis, bronchiolitis, pharyngitis, pleurisy, sinusitis, a parasitic infection, a bacterial infection, a viral infection, an autoimmune disease, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasculitis, angiitis, endocarditis, arteritis, thrombophlebitis, pericarditis, myocarditis, myocardial ischemia, periarteritis nodosa, rheumatic fever, celiac disease, adult respiratory distress syndrome, meningitis, encephalitis, cerebral infarction, cerebral embolism, Guillain-Barre syndrome, neuritis, neuralgia, spinal cord injury, paralysis, uveitis, arthritides, arthralgias, osteomyelitis, fasciitis, Paget's disease, periodontal disease, synovitis, myasthenia gravis, thyroiditis, systemic lupus erythematosus, Goodpasture's syndrome, Behcets's syndrome, allograft rejection, graft-versus-host disease, ankylosing spondylitis, Berger's disease, Reiter's syndrome, Hodgkin's disease, endometriosis, hemangioma, diseases associated with tissue fibrosis, Raynaud syndrome, Sjogren's syndrome, scleroderma, or fibrosis of liver, lung, heart, kidney, skin, pancreas, or intestine. In some embodiments, the disease or disorder is endometriosis, hemangioma, diseases associated with tissue fibrosis, Raynaud syndrome, Sjogren's syndrome, scleroderma, or fibrosis of liver, lung, heart, kidney, skin, pancreas, or intestine.

In some embodiments, the disease or disorder is cancer. In some embodiments, the cancer is breast cancer, brain cancer, intestinal cancer, colorectal cancer, prostate cancer, leukemia, lung cancer, melanoma, kidney cancer, non-Hodgkin lymphoma, bladder cancer, thyroid cancer, endometrial cancer, head/neck cancer, multiple myeloma, or carcinoma. In some embodiments, the disease or disorder is a solid tumor. In some embodiments, the disease or disorder is a benign tumor. In some embodiments, the cancer may be a non-immunogenic cancer. In some embodiments, the cancer may be a hematological cancer. In some embodiments, the cancer may be a solid tumor. In some embodiments, the cancer may be melanoma, pancreatic cancer, and colorectal cancer.

In some embodiments, the cancer may be breast cancer, prostate cancer, renal cell carcinoma, bone metastasis, lung cancer or metastasis, osteosarcoma, multiple myeloma, astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma, teratoma, gangliogliomas, gangliocytoma, central gangliocytoma, primitive neuroectodermal tumors (PNET, e.g. medulloblastoma, medulloepithelioma, neuroblastoma, retinoblastoma, ependymoblastoma), tumors of the pineal parenchyma (e.g. pineocytoma, pineoblastoma), ependymal cell tumors, choroid plexus tumors, neuroepithelial tumors of uncertain origin (e.g. gliomatosis cerebri, astroblastoma), esophageal cancer, colorectal cancer, CNS, ovarian, melanoma pancreatic cancer, squamous cell carcinoma, hematologic cancer (e.g., leukemia, lymphoma, and multiple myeloma), colon cancer, rectum cancer, stomach cancer, kidney cancer, pancreas cancer, skin cancer, or a combination thereof.

In some embodiments, the cancer may include, but is not limited to, a cancer of an organ selected from the group consisting of pancreas, colon, cecum, stomach, brain, head, neck, ovary, kidney, larynx, sarcoma, lung, bladder, melanoma, prostate, and breast. Additional cancers may include cancers of the bone marrow, T or B cell malignancies, leukemias, lymphomas, blastomas, myelomas, and the like. Further examples of cancers that may be treated using the methods provided herein include, but are not limited to, carcinoma, lymphoma, blastoma, sarcoma, leukemia, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer and gastrointestinal stromal cancer), pancreatic cancer, glioblastoma, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, melanoma, superficial spreading melanoma, lentigo malignant melanoma, acral lentiginous melanomas, nodular melanomas, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's macroglobulinemia), chronic lymphocytic leukemia (CLL), acute lymphoblastic leukemia (ALL), Hairy cell leukemia, multiple myeloma, acute myeloid leukemia (AML) and chronic myeloblastic leukemia.

In some embodiments, the cancer may include, but not limited to neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the disease or disorder may be a non-venular disease. In some embodiments, non-venular disease may include, but is not be limited to, vessel coronary disease, thrombotic microangiopathy, microangiopathic hemolytic anemia, microvascular occlusion, cutaneous diabetic microangiopathy, Susac's syndrome, cerebral microangiopathy, early diabetic microangiopathy, diabetic microangiopathy, glomerular microangiopathy, non-neoplastic nevus, pulmonary microangiopathy, pulmonary capillaritis, coronary microvascular disease, chronic microvascular diseases, small vessel ischemia, thrombotic thrombocytopenic purpura, arteriolosclerosis, and scleroderma. In some embodiments, the non-venular disease may be selected from vessel coronary disease, thrombotic microangiopathy, microangiopathic hemolytic anemia, microvascular occlusion, and cutaneous diabetic microangiopathy. In some embodiments, the non-venular disease may be vessel coronary disease. In some embodiments, the non-venular disease may be thrombotic microangiopathy. In some embodiments, the non-venular disease may be microangiopathic hemolytic anemia. In some embodiments, the non-venular disease may be microvascular occlusion. In some embodiments, the non-venular disease may be cutaneous diabetic microangiopathy, endometriosis, hemangioma, diseases associated with tissue fibrosis, Raynaud syndrome, Sjogren's syndrome, scleroderma, or fibrosis of liver, lung, heart, kidney, skin, pancreas, or intestine. In some embodiments, the disease or disorder is endometriosis, hemangioma, diseases associated with tissue fibrosis, Raynaud syndrome, Sjogren's syndrome, scleroderma, or fibrosis of liver, lung, heart, kidney, skin, pancreas, or intestine.

In some embodiments, upon administration of the composition to the subject, more than about 5% of the composition that is retained in the subject within about 2 hours following administration is localized in a tumor microenvironment within the subject. In some embodiments, upon administration of the composition to the subject, more than about 5% of the composition that is retained in the subject within about 12 hours following administration is localized in a tumor microenvironment within the subject. In some embodiments, upon administration of the composition to a subject, more than about 5% of the composition is excreted from the subject within about 12 hours following administration.

In some embodiments, upon administration of the composition to the subject, more than about 10% of the composition that is retained in the subject within about 2 hours following administration is localized in a tumor microenvironment within the subject. In some embodiments, upon administration of the composition to the subject, more than about 10% of the composition that is retained in the subject within about 12 hours following administration is localized in a tumor microenvironment within the subject. In some embodiments, upon administration of the composition to a subject, more than about 10% of the composition is excreted from the subject within about 12 hours following administration.

In some embodiments, upon administration of the composition to the subject, more than about 15% of the composition that is retained in the subject within about 2 hours following administration is localized in a tumor microenvironment within the subject. In some embodiments, upon administration of the composition to the subject, more than about 15% of the composition that is retained in the subject within about 12 hours following administration is localized in a tumor microenvironment within the subject. In some embodiments, upon administration of the composition to a subject, more than about 15% of the composition is excreted from the subject within about 12 hours following administration.

In some embodiments, upon administration of a composition provided herein to the subject, at least 0.1% (e.g., at least 1%, at least 5%, at least 10%, at least 15%, at least 20%, at least 30%, at least 40%, or at least 50%) of the composition that is retained in the subject within 2 hours following administration is localized in a target area within the subject. In some embodiments, the target area is a tumor microenvironment.

In some embodiments, upon administration of a composition provided herein to the subject, at most 50% (e.g., at most 40%, at most 30%, at most 20%, at most 15%, at most 10%, at most 5%, at most 2.5%, at most 1%) of the composition that is retained in the subject within 2 hours following administration is localized in a target area within the subject. In some embodiments, the target area is a tumor microenvironment.

In some embodiments, upon administration of a composition provided herein to the subject, at least 0.1% (e.g., at least 1%, at least 5%, at least 10%, at least 15%, at least 20%, at least 30%, at least 40%, or at least 50%) of the composition that is retained in the subject within 2 (e.g., within 4, within 6, or within 12) hours following administration is localized in a tumor microenvironment within the subject.

In some embodiments, upon administration of a composition provided herein to the subject, at most 50% (e.g., at most 40%, at most 30%, at most 20%, at most 15%, at most 10%, at most 5%, at most 2.5%, at most 1%) of the composition that is retained in the subject within 2 (e.g., within 4, within 6, or within 12) hours following administration is localized in a tumor microenvironment within the subject.

In some embodiments, upon administration of a composition provided herein to the subject, at least 0.1% (e.g., at least 1%, at least 5%, at least 10%, at least 15%, at least 20%, at least 30%, at least 40%, or at least 50%) of the composition that is retained in the subject within 12 (e.g., within 16, within 20, or within 24) hours following administration is localized in a target area within the subject. In some embodiments, the target area is a tumor microenvironment.

In some embodiments, upon administration of a composition provided herein to the subject, at least 0.1% (e.g., at least 1%, at least 5%, at least 10%, at least 15%, at least 20%, at least 30%, at least 40%, or at least 50%) of the composition that is retained in the subject within 12 (e.g., within 16, within 20, or within 24) hours following administration is localized in a target area within the subject. In some embodiments, the target area is a tumor microenvironment.

In some embodiments, upon administration of a composition provided herein to the subject, at most 50% (e.g., at most 40%, at most 30%, at most 20%, at most 15%, at most 10%, at most 5%, at most 2.5%, at most 1%) of the composition that is retained in the subject within 12 (e.g., within 16, within 20, or within 24) hours following administration is localized in a target area within the subject. In some embodiments, the target area is a tumor microenvironment.

In some embodiments, upon administration of a composition provided herein to the subject, more than 5% of the composition that is retained in the subject within 12 hours following administration is localized in a tumor microenvironment within the subject. In some embodiments, upon administration of a composition provided herein to the subject, more than 50% of the composition that is retained in the subject within 12 hours following administration is localized in a tumor microenvironment within the subject.

In some embodiments, the tumor microenvironment comprises tumor-associated endothelial cells. In some embodiments, the composition has a greater binding affinity for the tumor-associated endothelial cells than a binding affinity for normal endothelial cells.

In some embodiments, the tumor microenvironment provided herein comprises tumor-associated endothelial cells. In some instances, the compositions provided herein preferentially bind to the tumor-associated endothelial cells (e.g., which express (e.g., target) proteins) via the binder. In some embodiments, the compositions provided herein have greater binding affinity for the tumor-associated endothelial cells than a binding affinity for normal endothelial cells.

An effective amount or therapeutically effective amount as provided herein may comprise any effective or therapeutically effective amount as determined by one skilled in the art. In some embodiments, the effective amount of a composition provided herein is at least 10 MBq (e.g., at least 20 MBq, at least 50 MBq, at least 100 MBq, or at least 250 MBq). In some embodiments, the effective amount of a composition provided herein is at most 1000 MBq (e.g., at most 900 MBq, at most 800 MBq, at most 700 MBq, at most 600 MBq, or at most 500 MBq). In some embodiments, the effective amount of a composition provided herein is about 20 MBq to about 1000 MBq. In some embodiments, the effective amount of a composition provided herein is about 100 MBq to about 500 MBq.

An effective or therapeutically effective amount as provided herein may comprise at least 0.1 mg (e.g., at least 0.5, at least 1 mg, at least 10 mg, at least 25 mg, at least 50 mg, or at least 100 mg). In some embodiments, an effective or therapeutically effective amount comprises at most 1000 mg (e.g., at most 500 mg, at most 250 mg, or at most 100 mg). In some embodiments, an effective or therapeutically effective amount comprises about 0.1 mg to about 1000 mg. In some embodiments, an effective or therapeutically effective amount comprises about 0.5 mg to about 50 mg. In some embodiments, an effective or therapeutically effective amount comprises about 0.5 mg to about 10 mg.

An effective or therapeutically effective amount as provided herein may comprise at least 0.01 mg/kg (e.g., at least 0.05 mg/kg, at least 0.1 mg/kg, or at least 0.5 mg/kg). In some embodiments, an effective or therapeutically effective amount comprises at most 5 mg/kg (e.g., at most 3 mg/kg, at most 2 mg/kg, at most 1 mg/kg, at most 0.75 mg/kg, or at most 0.5 mg/kg). In some embodiments, an effective or therapeutically effective amount comprises about 0.01 mg/kg to about 5 mg/kg. In some embodiments, an effective or therapeutically effective amount comprises about 0.01 mg/kg to about 0.5 mg/kg.

In some embodiments, upon administration of a composition provided herein, the renal toxicity metrics in the subject within 24 hours following administration remains within 20% of the levels of the renal toxicity metrics prior to administration. In some embodiments, upon administration of the composition to a subject, the renal toxicity metrics in the subject within about 24 hours following administration remains within about 5% of the levels of the renal toxicity metrics prior to administration.

In some embodiments, a renal toxicity metric is (e.g., serum) creatinine, glomerular filtrate rate (GFR), proteinuria, erythropoietin production, dysregulation of the renin-angiotensin system, or blood urea nitrogen (BUN).

In some embodiments, upon administration of a composition provided herein, the renal toxicity metrics in the subject following administration do not (e.g., substantially) change, such as 24 hours after administration. Use of radioisotopes, and specifically heavy metal isotopes may result in renal toxicity, and as such, compositions that provide therapeutic and or imaging capabilities that provide minimal decrease in renal function are imperative.

In some embodiments, upon administration of a composition or pharmaceutical composition provided herein, the renin-angiotensin system will not be dysregulated in the subject (e.g., 12 hours, 24 hours, 48 hours, or 72 hours) following administration.

In some embodiments, upon administration of a composition or pharmaceutical composition provided herein, the (e.g., serum) creatinine in the subject (e.g., 12 hours, 24 hours, 48 hours, or 72 hours) following administration does not (e.g., substantially) change from the (e.g., serum) creatinine levels before administration. In some embodiments, upon administration of a composition or pharmaceutical composition provided herein, the (e.g., serum) creatinine in the subject (e.g., 12 hours, 24 hours, 48 hours, or 72 hours) following administration remains within 1% (e.g., within 5%, within 10%, within 15%, within 20%, or within 25%) of the levels of (e.g., serum) creatinine before administration.

In some embodiments, upon administration of a composition or pharmaceutical composition provided herein, the proteinuria in the subject (e.g., 12 hours, 24 hours, 48 hours, or 72 hours) following administration does not (e.g., substantially) change from the proteinuria levels before administration. In some embodiments, upon administration of a composition or pharmaceutical composition provided herein, the proteinuria in the subject (e.g., 12 hours, 24 hours, 48 hours, or 72 hours) following administration remains within 1% (e.g., within 5%, within 10%, within 15%, within 20%, or within 25%) of the levels of proteinuria before administration.

In some embodiments, upon administration of a composition or pharmaceutical composition provided herein, the erythropoietin in the subject (e.g., 12 hours, 24 hours, 48 hours, or 72 hours) following administration does not (e.g., substantially) change from the erythropoietin levels before administration. In some embodiments, upon administration of a composition or pharmaceutical composition provided herein, the erythropoietin in the subject (e.g., 12 hours, 24 hours, 48 hours, or 72 hours) following administration remains within 1% (e.g., within 5%, within 10%, within 15%, within 20%, or within 25%) of the levels of erythropoietin before administration.

In some embodiments, upon administration of a composition or pharmaceutical composition provided herein, the glomerular filtration rate (GFR) in the subject (e.g., 12 hours, 24 hours, 48 hours, 72 hours) following administration does not (e.g., substantially) change from the glomerular filtration rate (GFR) levels before administration. In some embodiments, upon administration of a composition or pharmaceutical composition provided herein, the glomerular filtration rate (GFR) in the subject (e.g., 12 hours, 24 hours, 48 hours, or 72 hours) following administration remains within 1% (e.g., within 5%, within 10%, within 15%, or within 20%, within 25%) of the levels of glomerular filtration rate (GFR) before administration.

In some embodiments, upon administration of a composition or pharmaceutical composition provided herein, the blood urea nitrogen (BUN) in the subject (e.g., 12 hours, 24 hours, 48 hours, or 72 hours) following administration does not (e.g., substantially) change from the blood urea nitrogen (BUN) levels before administration. In some embodiments, upon administration of a composition or pharmaceutical composition provided herein, the blood urea nitrogen (BUN) in the subject (e.g., 12 hours, 24 hours, 48 hours, or 72 hours) following administration remains within 1% (e.g., within 5%, within 10%, within 15%, within 20%, or within 25%) of the levels of blood urea nitrogen (BUN) before administration.

### Kits

Provided herein in some embodiments, is a kit comprising a composition provided herein or a pharmaceutical composition provided herein and instructions for use. In some embodiments, the kit comprises a composition or pharmaceutical composition and instructions for use for any method provided herein. In some embodiments, the kit comprises a composition or pharmaceutical composition and instructions for use for treating a disease or disorder (e.g., such as a disease or disorder provided elsewhere herein). In some embodiments, the kit comprises a composition or pharmaceutical composition (or radiopharmaceutical composition) and instructions for use for selectively delivering a (e.g., radiopharmaceutical or a cell, e.g., a CAR T cell) composition to a tumor endothelial cell compared to a non-tumor endothelial cell in a subject.

Such kits may include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers may be formed from a variety of materials such as glass or plastic.

In some embodiments, the container(s) may include the composition described herein, or the pharmaceutical composition described herein. The container(s) optionally have a sterile access port. Such kits may optionally comprise compositions with an identifying descriptions or labels or instructions relating to their use in the methods described herein.

In some embodiments, the kit may include one or more additional containers, each with one or more of various materials (such as reagents, optionally in concentrated form, and/or devices) desirable from a commercial and user standpoint for use of the compositions described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are included for illustrative purposes only and are not intended to limit the scope of the invention.

### Example 1: Determining Expression of Genes in Tumor Vascular Endothelial Cells

A hallmark of solid tumors is the formation of new vasculature (angiogenesis). This process is required to support tumor growths beyond a few millimeters in size due to the limit of oxygen and nutrient diffusion within neoplastic tissues (Folkman, J. Tumor angiogenesis: therapeutic implications. N Engl J Med, 285, 1182-1186, (1971)). Tumor neovasculature is often poorly adhesive for blood-borne T cells, which is thought to present a major impediment to T cell dependent immunotherapy (Peske J D, Woods A B, Engelhard V H. Control of CD8 T-Cell Infiltration into Tumors by Vasculature and Microenvironment. Adv Cancer Res., 128, 263-307, (2015)). In order to gain a better understanding of this issue, normal microvasculature most be analyzed, which consists of a network of functionally specialized vessels, including arteries, arterioles, venules and veins, which are all connected by a common capillary network.

Arteries and arterioles regulate blood flow, while gas and nutrient exchange takes place at the capillary level (Potente, M., Mäkinen, T. Vascular heterogeneity and specialization in development and disease. Nat Rev Mol Cell Biol 18, 477-494 (2017)). Using intravital microscopy, it has been shown that the recruitment of blood-borne leukocytes is invariably restricted to postcapillary and collecting venules, whereas capillaries and arterioles do not support leukocyte adhesion (Halin, C., J. Rodrigo Mora, C. Sumen, and U. H. von Andrian. In vivo imaging of lymphocyte trafficking. Annu Rev Cell Dev Biol, 21, 581-603, (2005)). There is strong evidence suggesting that this functional distinction among microvessels is due to segmental specialization of endothelial cells (ECs), not hemodynamic differences (Ley, K., and P. Gaehtgens. Endothelial, not hemodynamic, differences are responsible for preferential leukocyte rolling in rat mesenteric venules. Circ Res, 69, 1034-1041, (1991)). Indeed, microvascular specialization is already apparent during embryogenesis before the initiation of blood flow (Lawson, N. D., and B. M. Weinstein. Arteries and veins: making a difference with zebrafish. Nat Rev Genet, 3, 674-682, (2002)).

Regardless of whether uninflamed microvessels that constitutively recruit leukocytes or acutely or chronically inflamed peripheral tissues are assessed, venules are the exclusive port of exit for blood-borne leukocytes that access the extravascular compartment. Accordingly, multiple studies have shown that most leukocyte adhesion receptors are restricted to venular ECs (VECs), although the expression of these molecules is not uniform in different vascular beds (von Andrian, U. H., and C. R. Mackay. 2000. T-cell function and migration. Two sides of the same coin. N. Engl. J. Med., 343, 1020-1034, (2000)). Several molecules have been identified that specify the differentiation of blood ECs and lymphatic ECs (LECs) and contribute to EC proliferation in tumors, (Rocha, S. F., and R. H. Adams. Molecular differentiation and specialization of vascular beds. Angiogenesis, 12, 139-147, (2009); Oliver, G., and R. S. Srinivasan. Endothelial cell plasticity: how to become and remain a lymphatic endothelial cell. Development, 137, 363-372, (2010)), but the mechanism(s) that render(s) VECs uniquely capable of supporting leukocyte trafficking remain(s) a mystery. A monoclonal antibody (mAb) against DARC (ACKR1) was developed, which selectively recognizes VECs in normal murine tissues (Thiriot, A. et al. Differential DARC/ACKR1 expression distinguishes venular from non-venular endothelial cells in murine tissues, both tumor and non-tumor tissues. BMC Biology, 15, 45, (2017)). In preliminary experiments, using this mAb, as well as a commercial mAb against human DARC, primary VECs, non-venular ECs (NVECs) and LECs were isolated from a variety of murine and human non-malignant tissues to compare EC subsets at the transcriptome and proteome level.

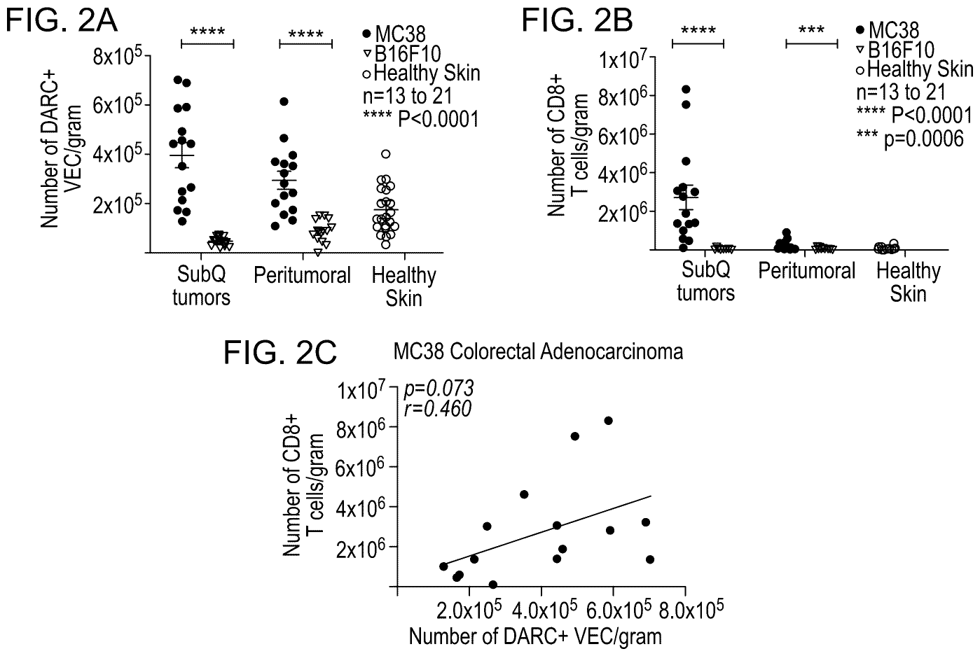

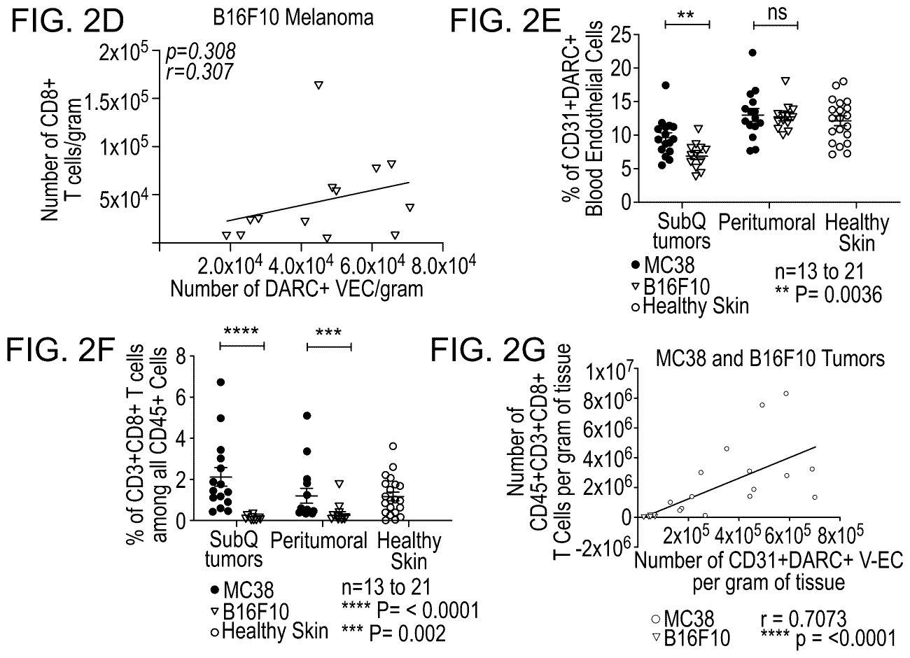

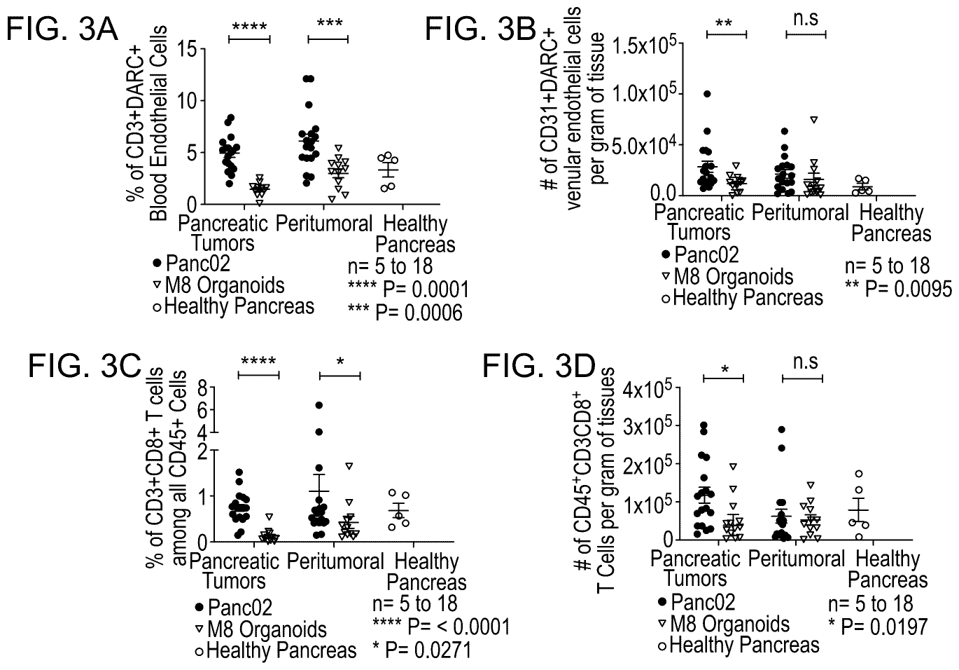

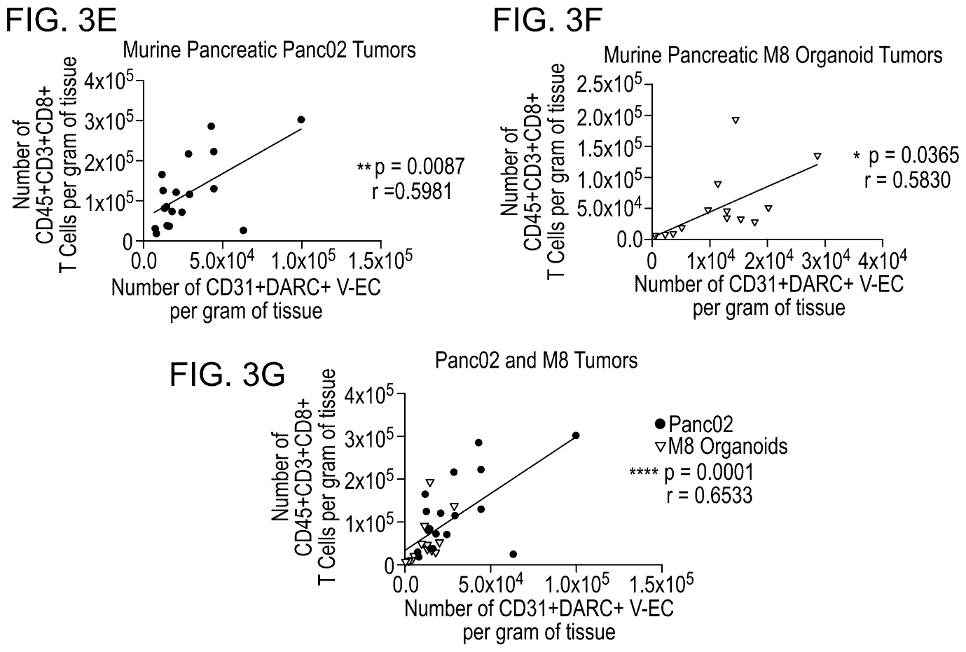

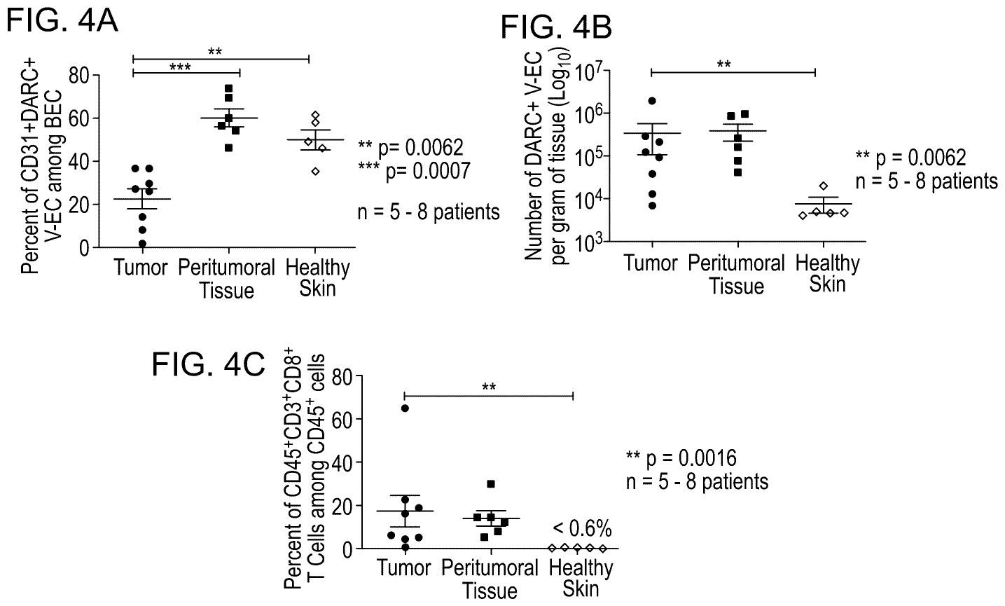

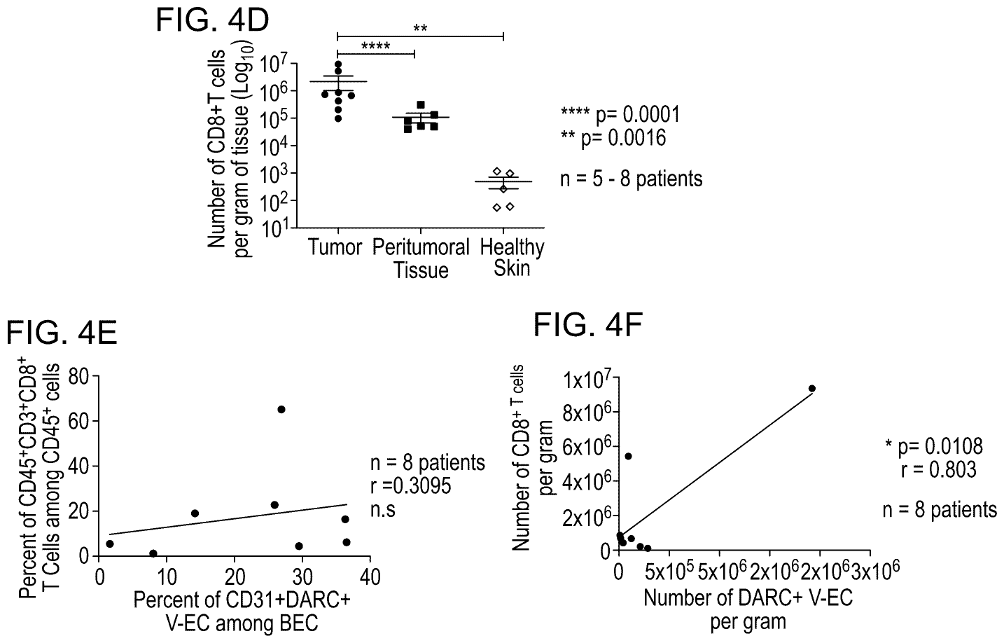

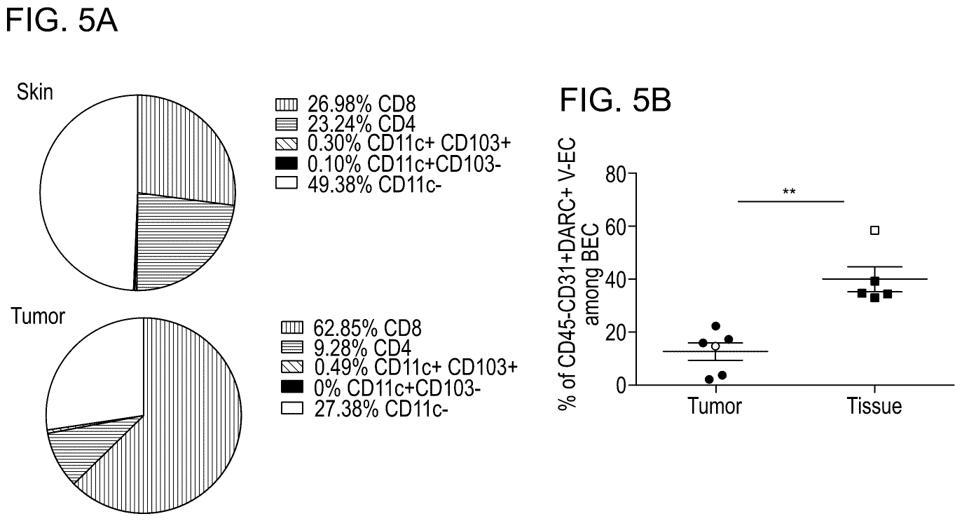

Their analysis revealed that DARC is exquisitely restricted to post-capillary and small collecting venules and completely absent from arteries, arterioles, capillaries, veins, and most lymphatic ECs in every tissue analyzed. Accordingly, intravital microscopy showed that adhesive leukocyte-endothelial interactions were restricted to DARC+ venules. DARC was detectable over the entire circumference of VECs but was more concentrated at cell-cell junctions (Thiriot, A. et al. BMC Biology, 15, 45, (2017) FIG. 2). Analysis of single-cell suspensions suggested that the frequency of VECs among the total microvascular EC pool varies considerably between different tissues.

However, preliminary findings showed that some solid tumors may contain up to 70% fewer venules than healthy tissue, which could explain the paucity of T cells in many cancers. The VECs in those tumors lacked many venular markers, suggesting that newly formed tumor microvessels may not initiate venular differentiation programs. Since VEC are the principal gatekeepers for leukocyte emigration, drugs that promote VEC differentiation could potentially boost tumor infiltration by T cells and thus enhance immunotherapy. Therefore, the hypothesis is that the neovasculature of solid tumors may be inherently suboptimal at recruiting T cells because of inadequate endothelial differentiation into functional venular type microvessels. The transcriptome and proteome of VECs in healthy and in immunogenic vs non immunogenic tumor samples were analyzed to gain a better understanding of the VEC differentiation program.

### Results

Microvascular ECs likely differ between tumors that are susceptible to immunotherapy compared to those that are resistant to treatment; thus the microvascular composition of immunogenic and non-immunogenic tumor models in mouse was characterized. For this purpose, B16 melanoma cells (B16) and MC38 colorectal adenocarcinoma cells (MC38) were used. MC38 has been reported to be susceptible to immunotherapy while B16 melanoma is non-responsive to checkpoint inhibitors. To characterize VEC and T cell content in those tumors, histology and flow cytometry were used to investigate whether tumoral VEC frequency correlates with the number of tumor-infiltrating T cells.

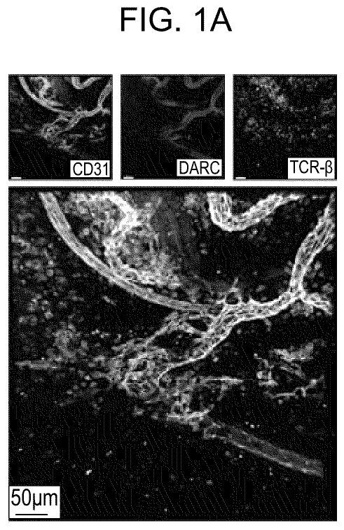

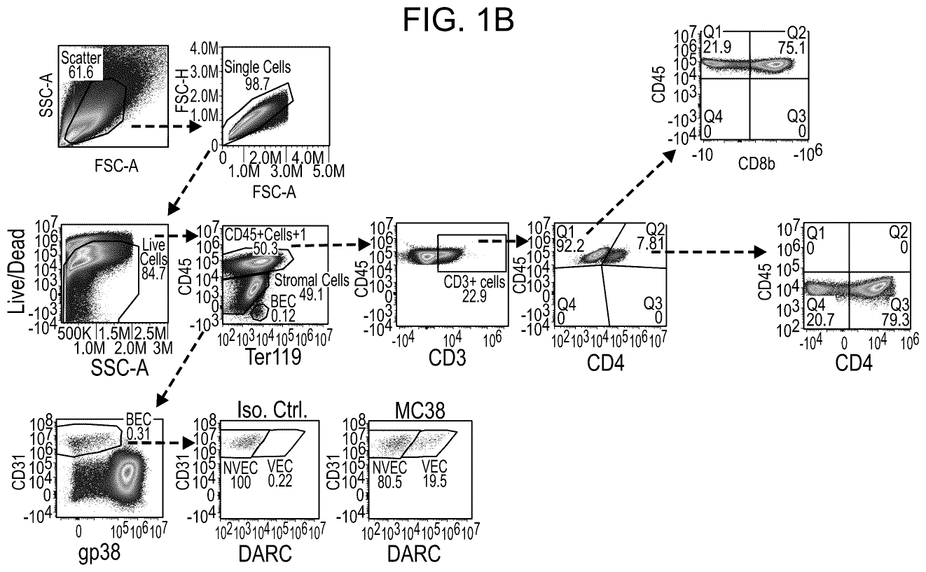

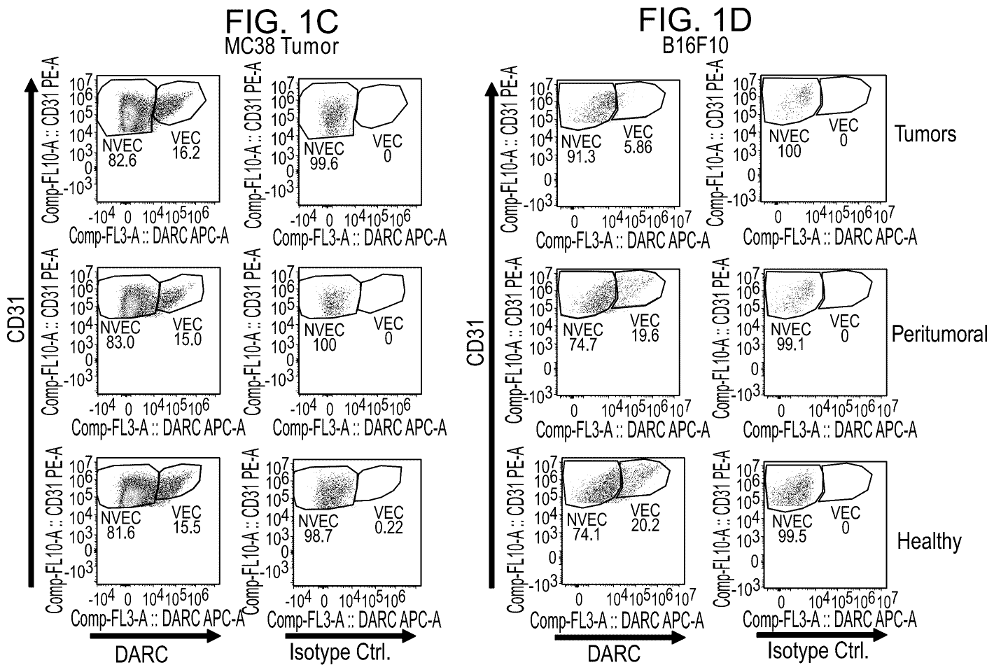

Using antibodies against CD31 (a pan-endothelial marker) and against DARC venules in subcutaneous MC38 implanted in mice were imaged (FIG. 1A). By combining this with T cell imaging through a TCR-3 antibody, the proximity between VEC and T cells as well as the extent of the T cell infiltration in this immunogenic tumor model were revealed. This was confirmed by quantification of the T cell infiltrate in both B16 and MC38.

FACS was used to analyze both CD8+ T cells and VEC from B16 and MC38 subcutaneous tumors (gating strategies in FIG. 2) and to quantify how many CD8+ T cells and VEC were observed per gram of tissue in each case. This was repeated for peritumoral tissue and healthy mouse skin (control). A very clear difference in the amount of both VEC and CD8+ T cells in the immunogenic (MC38) vs non-immunogenic model (B16) was observed. The amount of VEC in MC38 was quadrupled compared to what was observed in B16 (FIG. 2A). More strikingly B16 contains very few T cells whereas MC38 contains 3×10⁶ T cells per gram of tissue (FIG. 2B). Interestingly MC38 also showed an increase in VEC and T cell numbers when compared to healthy tissue which confirmed its increased immunogenicity and capacity to attract T cells. Both MC38 and B16 peritumoral tissue showed numbers reflecting an intermediate state between tumor and healthy skin, as expected.

Next, the direct correlation between VEC and CD8+ T cell numbers in individual animals transplanted with either tumor model was analyzed (FIGS. 2C and 2D). Both models showed a strong correlation between VEC and CD8+ T cells numbers in the tumor confirming that the presence of VEC is likely to increase T cell infiltration. However, the absolute number of CD8+ T cells and VECs is lower in B16 reflecting the increased immunogenicity of MC38.

In order to confirm that the data observed in mouse tumor models held up in human tumor, resections of tumor samples from patients were acquired. Samples of melanoma and pancreatic tumor and healthy control samples (e.g., healthy skin and non-malignant pancreas, respectively) were used.

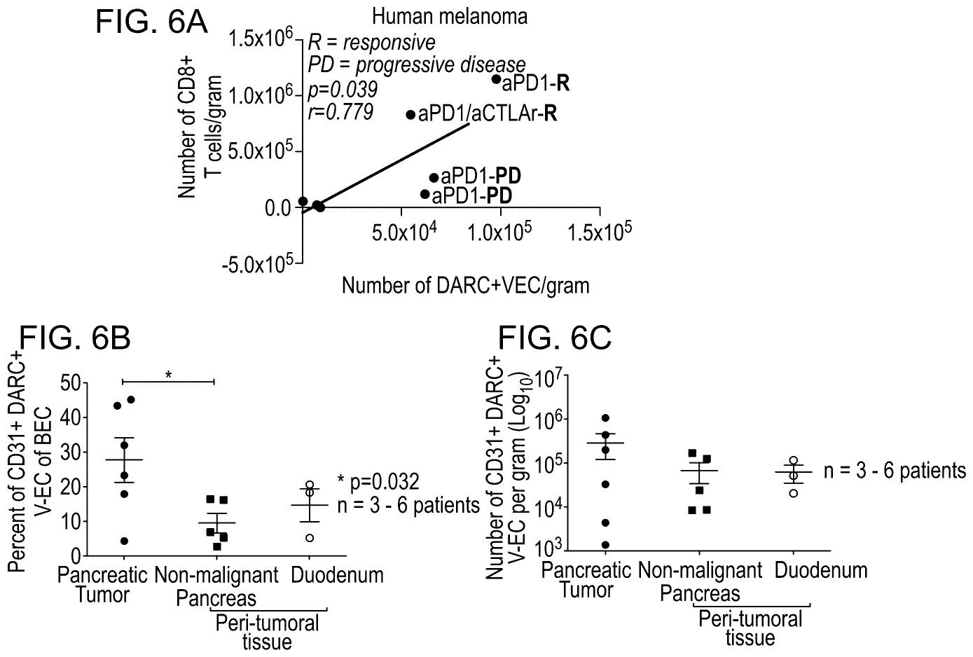

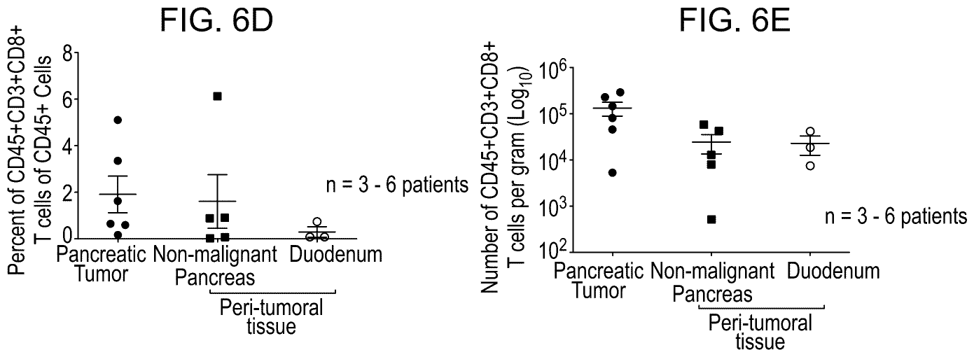

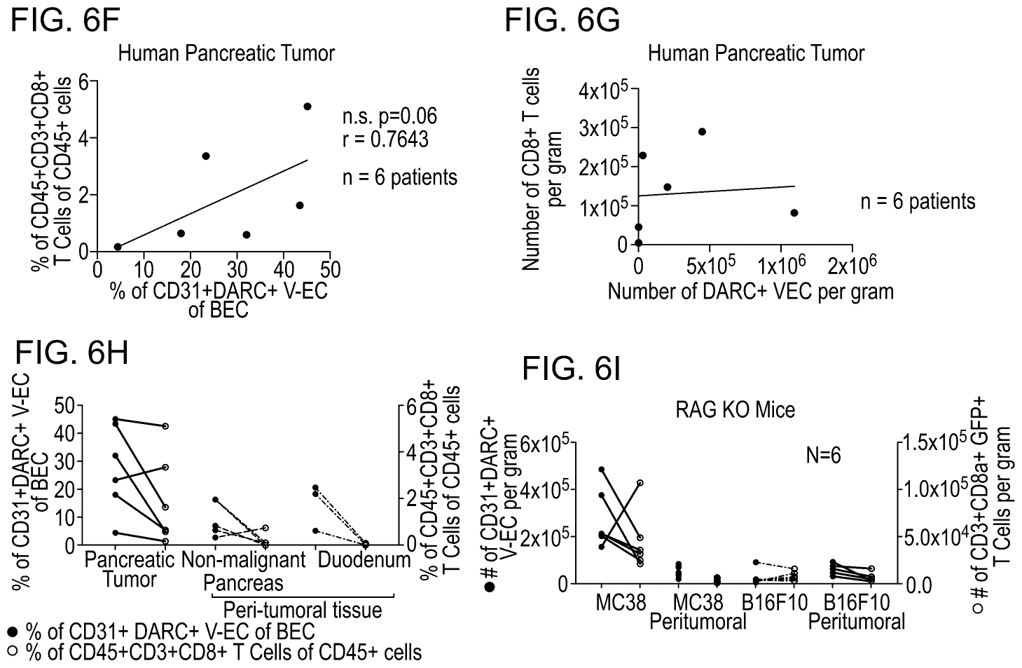

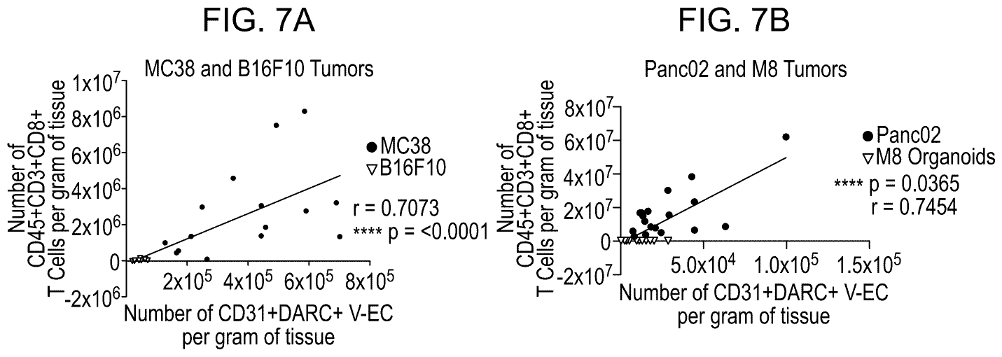

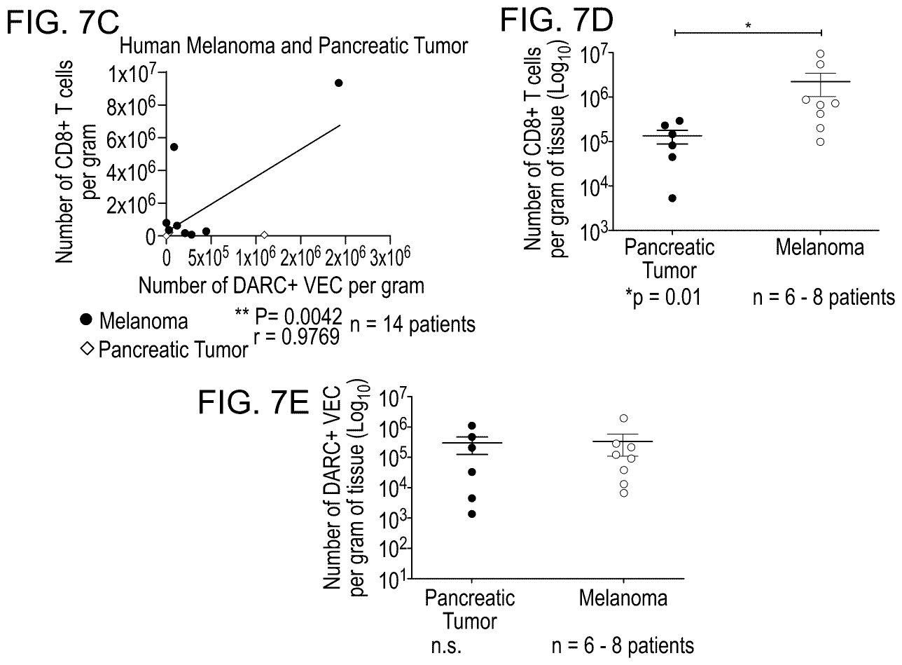

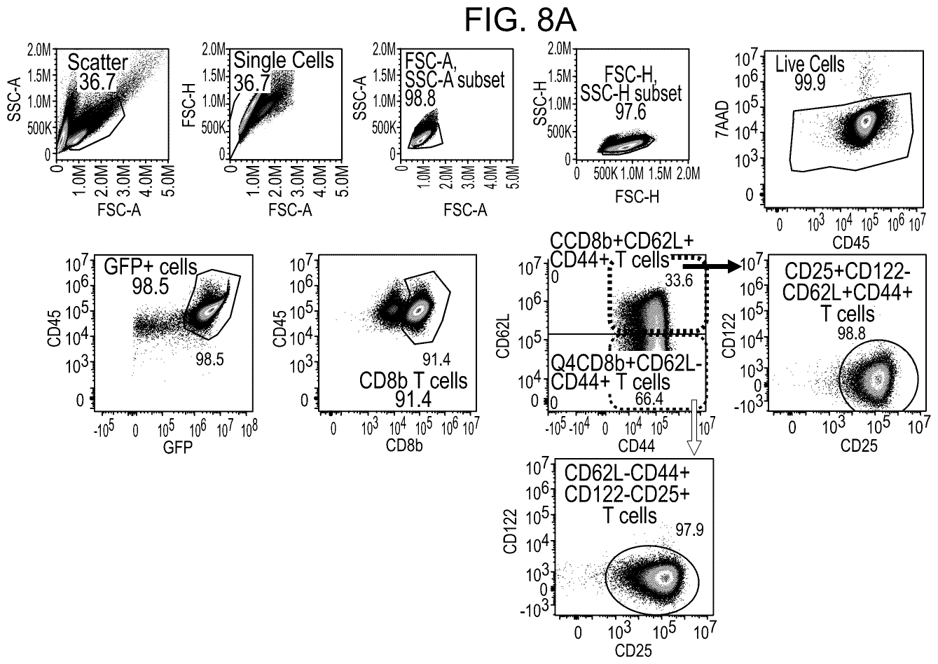

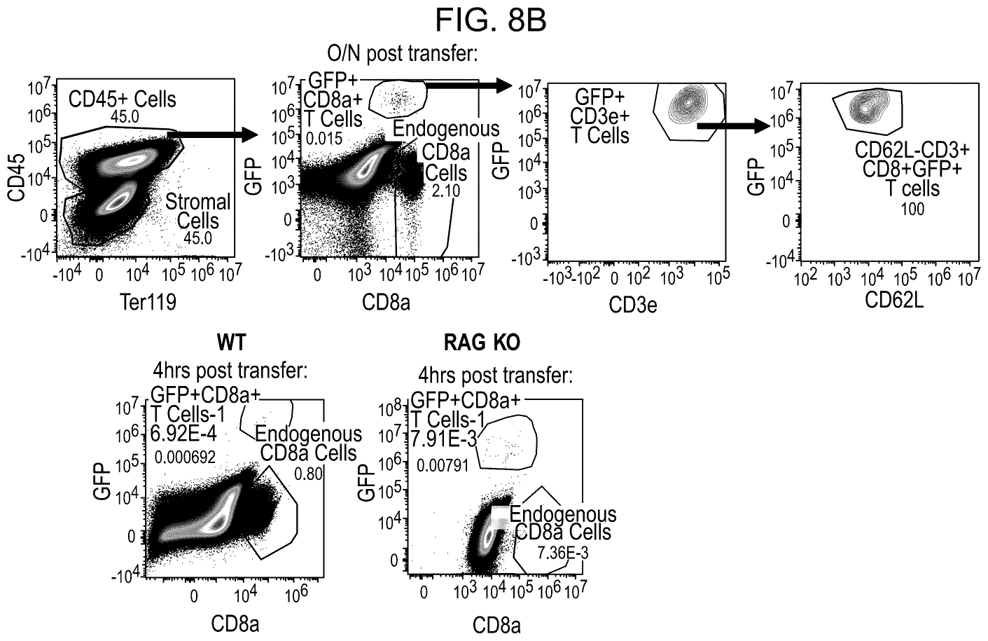

For the melanoma samples, resections from patients who were either responsive or unresponsive (progressive disease) to immunotherapy were used to directly compare the numbers of VECs and T cells in those two cases (FIG. 6A). Interestingly, a correlation was observed between the number of VECs and T cell whether or not the patients were responding to immunotherapy, but samples from responsive patients had much higher number of T cells in the tumor and the correlation with the number of VECs was stronger. This seems to indicate that in unresponsive patients the VECs might be dysfunctional and unable to facilitate the infiltration of T cells in the tumor.

Similarly, a strong correlation was observed between VEC numbers and T cell numbers in human pancreatic tumors (FIG. 6F). Pancreatic tumors were also of particular interest because non-malignant pancreas samples as well as peritumoral duodenum samples were available (FIG. 6D, FIG. 6E). Although the non-malignant (NM) pancreas samples were not from the same patients as the pancreatic tumor samples, they were obtained from “healthy” areas of diseased pancreas, which allowed for these samples to be used as a healthier control to the pancreas tumor samples. Duodenum samples were obtained from patients undergoing a Whipple procedure where part of the duodenum is removed along with the pancreatic tumor so they can be considered peritumoral samples. VEC and T cell contents were measured in all samples and matched the number for each individual sample (FIG. 6H). Again, a strong correlation was observed between VEC and T cell numbers, and similarly to what was observed in the mouse MC38 model the peritumoral and the non-malignant tissues had much lower numbers of T cell and VEC suggesting that VEC are a key component of T cell infiltration in tumor.

Generally, the melanoma samples had higher T cell infiltration than the pancreas samples, in part because they came from patients who were responsive to immunotherapy. Thus, melanoma has been used as an example of immunogenic tumor in human while the pancreas tumor samples has been used as an example of non-immunogenic tumor in human.

To focus selectively on T cell recruitment, homing experiments with adoptively transferred tumor-specific effector and memory T cell subsets were performed (Weninger, W., M. A. Crowley, N. Manjunath, and U. H. von Andrian. Migratory properties of naive, effector, and memory CD8(+) T cells. J. Exp. Med. 194, 953-966, (2001); Gerlach, C. et. al. The Chemokine Receptor CX3CR1 Defines Three Antigen-Experienced CD8 T Cell Subsets with Distinct Roles in Immune Surveillance and Homeostasis. Immunity, 45, 1270-1284, (2016)). To assess T cell interactions with tumor microvessels, RAG knockout (KO) mice were used in which fluorescent activated T cells were transferred after MC38 and B16 tumor had been allowed to grow in these mice. The absence of mature B and T cells in RAG KO mice means that all T cell infiltration in the tumor happened after the transfer of exogenous T cells and that the T cells could be tracked to compare their numbers in immunogenic versus non-immunogenic tumors. 24 hours after the transfer a sharp difference between MC38 and B16 T cell infiltration was observed (FIG. 6I). The number of VECs and T cells in B16 tissues were on par with peritumoral and healthy tissue numbers while MC38 had at least twice the same amount of both VECs and T cells. This confirmed that the presence of intratumoral T cells is mainly due to infiltration and not to intratumoral T cell proliferation.

Isolation and Characterization of Venular Endothelial Cells by scRNA-Seq

So far, the finding that an increase in T cell infiltration correlates with an increase in the number of VECs in the tumor has been established. This increase in intratumoral T cells seems to be due to an increase in T cell interactions with tumor microvessels. However, it is still unclear how these interactions take place and what makes VECs in immunogenic tumor more efficient at recruiting T cells. To address these questions, scRNA-Seq was used to compare the EC transcriptomes of the two subcutaneous murine tumors (MC38 and B16F10) described above and fresh patient-derived human melanoma and pancreatic cancer ECs. For each tumor, VECs and NVECs from tumoral and non-malignant tissues were analyzed. This strategy can be used to identify endothelial genes, including genes encoding cell surface and secreted molecules, that are uniquely upregulated in the tumor microvasculature and possibly specific to immunogenic tumor VECs.

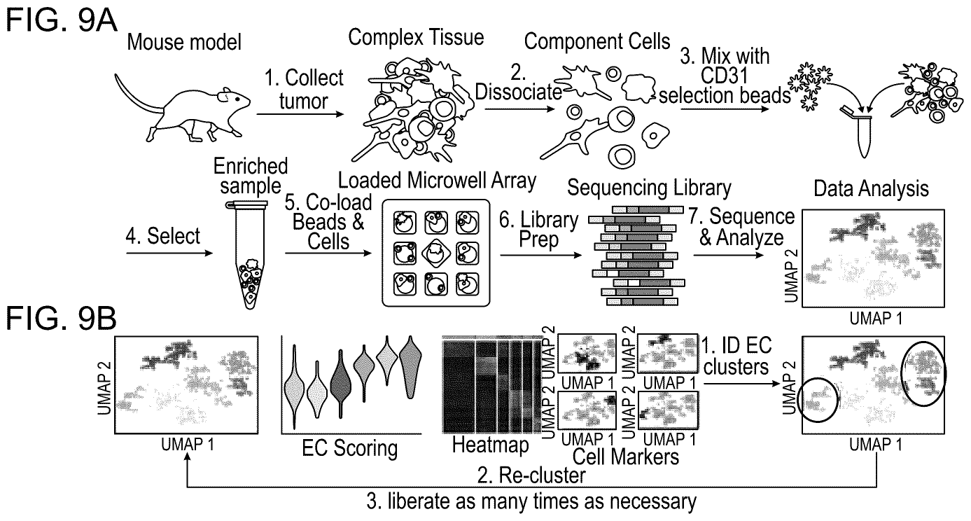

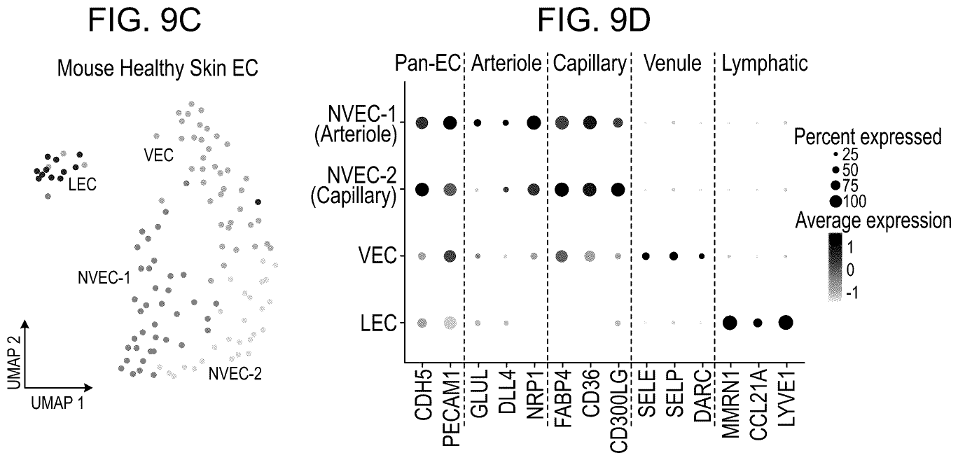

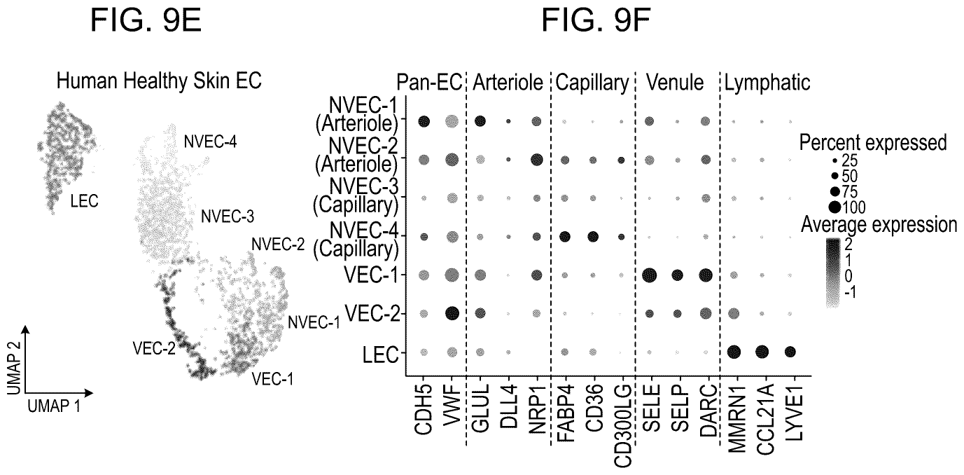

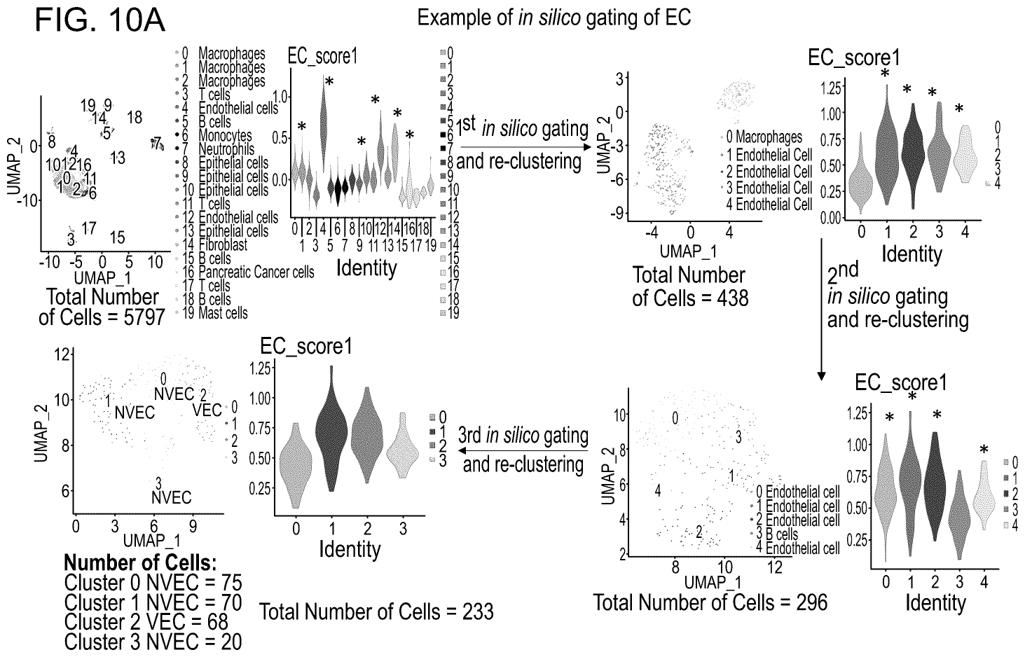

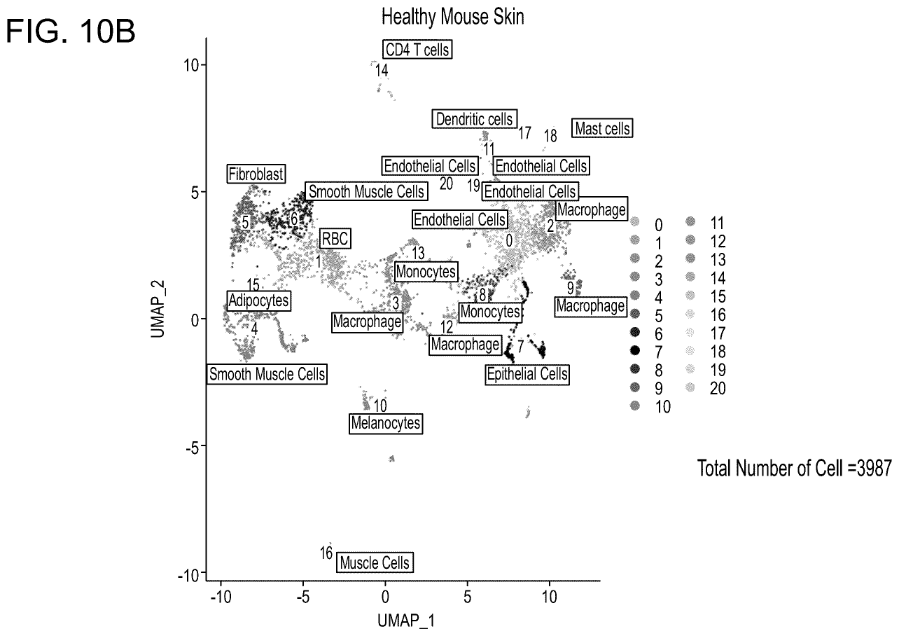

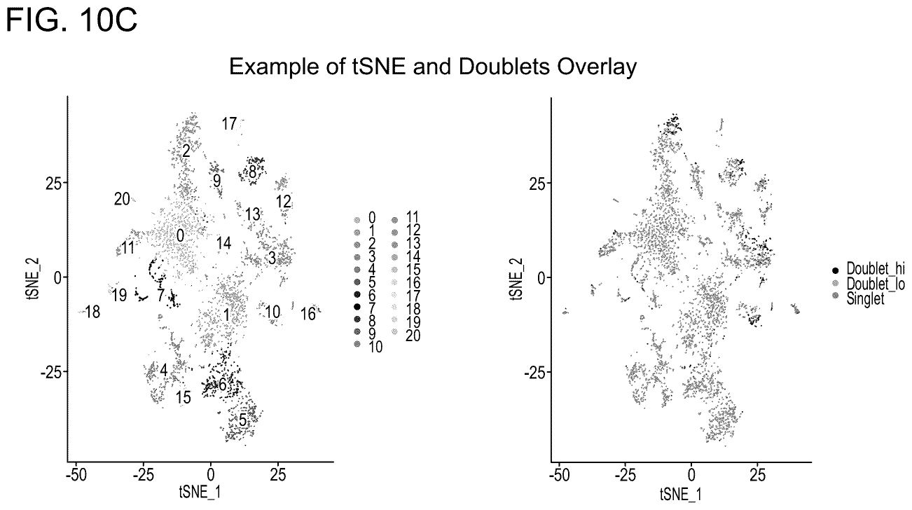

All mouse and human samples were processed using Seq-Well (FIG. 9A). Prior to Seq-Well, single-cell suspensions were prepared and were either used as is, or after CD45+ depletion or after CD31+ enrichment or a combination of both (see Table 8). Samples were then loaded on microwell arrays preloaded with barcoded beads. Libraries were prepared following the SeqWell protocol, and sequenced on an Illumina instrument. Segregation by enrichment method or by patient was never observed, so samples of the same tumor type were pooled after sequencing regardless of the preparation strategy for single cell suspension.

TABLE 8
Samples collected. M: mouse; H: Human; -ve fraction:
CD45+ fraction collected after CD45 depletion.

| Name | Sample type | Species | Enrichment |
|---|---|---|---|
| 180118 WT murine | dorsal skin | M | None |
| skin | | | |
| 180126 MC38 sort seq | Tumor MC38 | M | None |
| well | | | |
| | Healthy Skin | M | None |
| 180130 B16F10 Seq | Tumor B16F10 | M | None |
| Well | | | |
| | Healthy Skin | M | None |
| 180209 Seq Well | Tumor MC38 | M | None |
| | Tumor B16F10 | M | None |
| 180212 Seq Well | Tumor MC38 | M | None |
| | Tumor B16F10 | M | None |
| 180222 hSample PanT | Pancreatic Tumor | H | None |
| and Duo | | | |
| 180301 hSkin BP | Skin - array 1 | H | None |
| | Skin - array 2 | H | None |
| 180321 MC38 Seq | Tumor MC38 | M | CD31+ enrichment + spike with some -ve |
| Well | | | fraction |
| | Healthy | M | CD31+ enrichment + spike with some -ve |
| | | | fraction |
| 180322 hPT and Duo | Pancreatic Tumor | H | Depleted CD235a/b and CD45+ cells |
| 180327 B16F10 for | Tumor B16F10 | M | CD31+ enrichment + spike with some -ve |
| Sequencing | | | fraction |
| | Healthy | M | CD31+ enrichment + spike with some -ve |
| | | | fraction |
| 180328 B16F10 for | Tumor B16F10 | M | CD31+ enrichment + spike with some -ve |
| sequencing | | | fraction |
| 180403 MC38 Seq | Tumor MC38 | M | CD31+ enrichment + spike with some -ve |
| Well Rep2 | | | fraction |
| 180403 Healthy Pan | NM pancreas | H | CD31+ enrichment + spike with some -ve |
| | | | fraction |
| 180423 MC38 Seq | Tumor MC38 | M | CD31+ enrichment no other cells added |
| Well CD31 Pure#1 | | | |
| | Healthy | M | CD31+ enrichment no other cells added |
| 180425 B16 Seq Well | Tumor B16F10 | M | CD31+ enrichment no other cells added |
| CD31 Pure#1 | | | |
| | Healthy | M | CD31+ enrichment no other cells added |
| Healthy Panc | Healthy | H | CD31+ enrichment no other cells added |
| Human Pan Tumor | Pancreatic tumor | H | CD31+ enrichment no other cells added |
| and Healthy pancreas | | | |
| | Healthy Pancreas | H | CD31+ enrichment no other cells added |
| 180821 healthy human | Healthy Pancreas | H | CD31+ enrichment no other cells added |
| Pancreas | | | |
| 180910 Healthy | Healthy Pancreas | H | CD31+ enrichment no other cells added |
| Human Pancreas | | | |
| 181009 Human Pan | Healthy Pancreas | H | CD31+ enrichment no other cells added |
| Tumor and Healthy | | | |
| Pancreas | | | |
| | Pancreatic tumor | H | CD31+ enrichment no other cells added |
| 181009 Human Skin | Human Skin | H | CD31+ enrichment no other cells added |
| 181010 B16F10 and | B16F10 | M | CD31+ enrichment no other cells added |
| Peritumoral | | | |
| 181012 MC38 and | MC38 | M | CD31+ enrichment no other cells added |
| Peritumoral | | | |
| 181017 Human Skin | Human Skin | H | CD31+ enrichment no other cells added |
| 181018 Human Skin | Human Skin | H | CD31+ enrichment no other cells added |
| 181102 Human | Pancreatic Tumor | H | CD31+ enrichment no other cells added |
| Pancreas tumor | | | |
| 181105 Human | Melanoma | H | CD31+ enrichment no other cells added |
| Melanoma | | | |
| 181116 Human | Pancreatic Tumor | H | CD31+ enrichment no other cells added |
| Pancreatic Tumor and | | | |
| healthy pancreas | | | |
| 181116 Human | Healthy Pancreas | H | CD31+ enrichment no other cells added |
| Pancreatic Tumor and | | | |
| healthy pancreas | | | |
| 181120 Mouse | Healthy Mouse | M | CD31+ enrichment no other cells added |
| Healthy Skin | Skin | | |
| 181128 human Pan | Pancreatic Tumor | H | CD31+ enrichment no other cells added |
| Tumor and Pan | | | |
| Healthy | | | |
| 181128 human Pan | Healthy Pancreas | H | CD31+ enrichment no other cells added |
| Tumor and Pan | | | |
| Healthy | | | |
| 190130 hMelanoma | Melanoma | H | CD31+ enrichment no other cells added |
| 32719 MC38 | MC38 | M | CD31+ enrichment no other cells added |

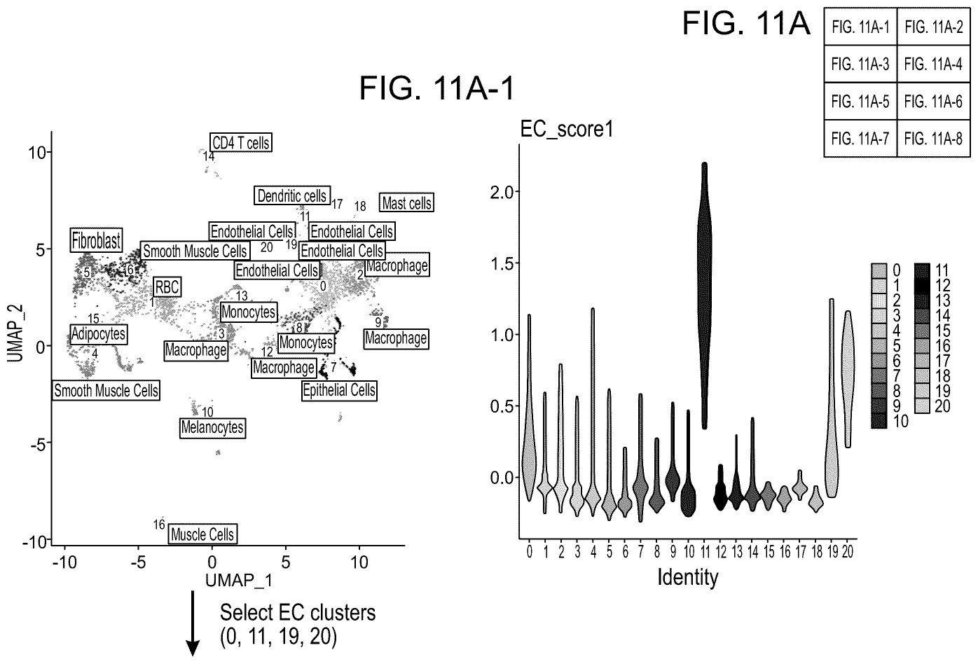

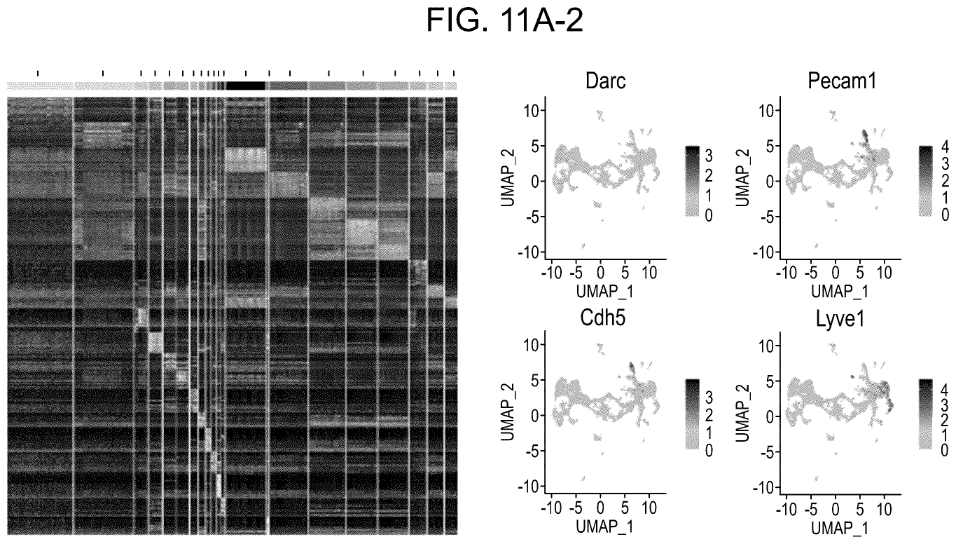

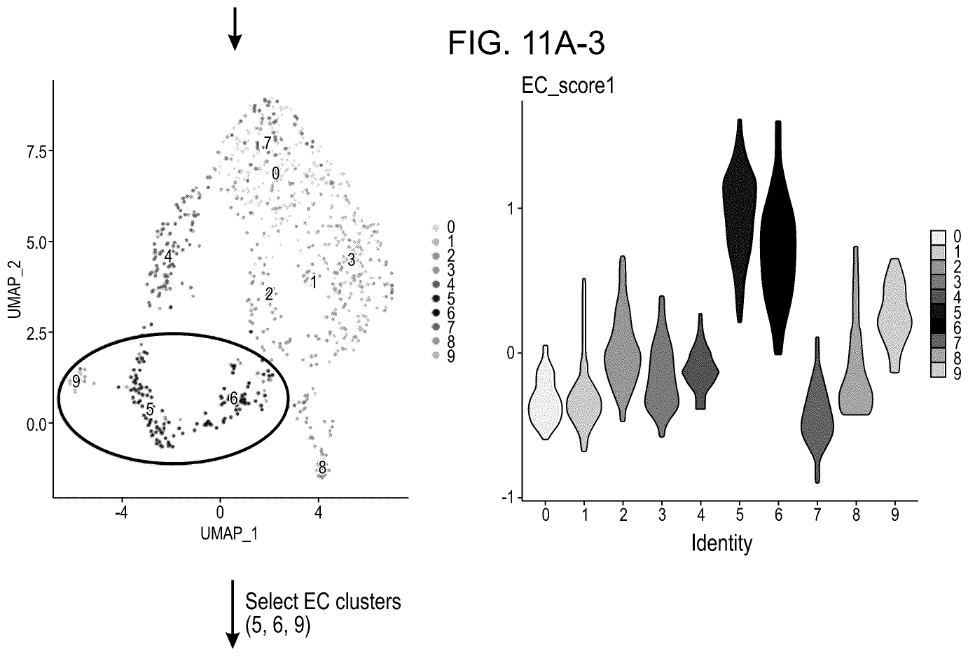

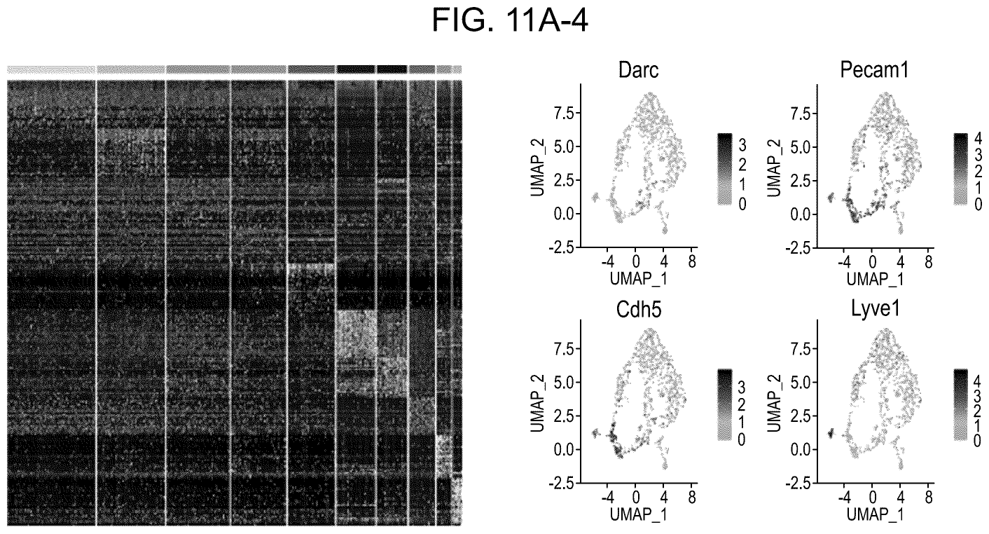

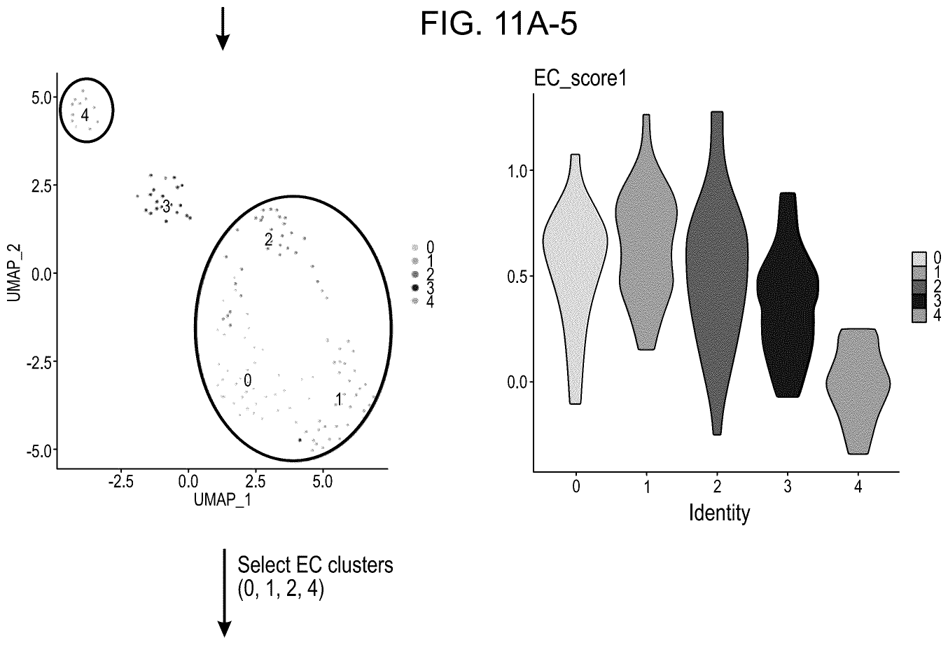

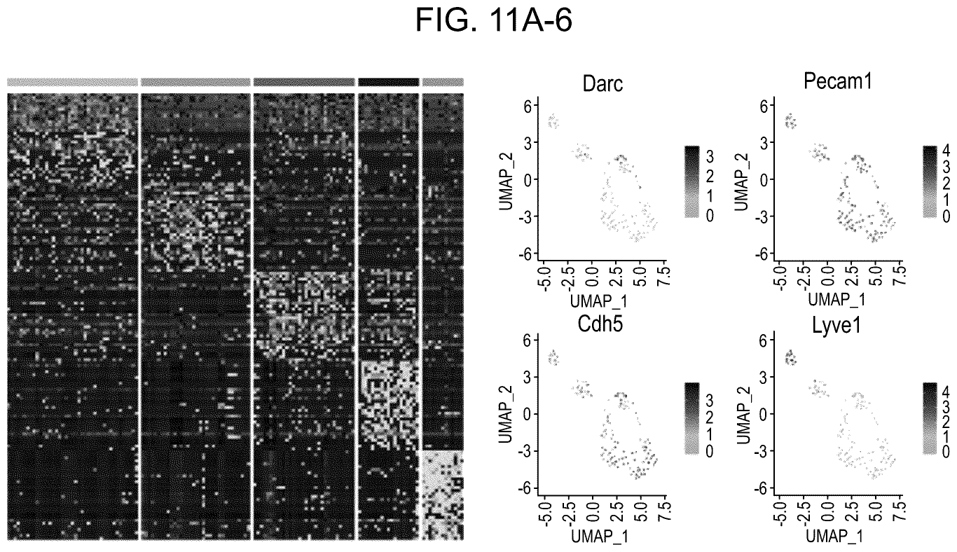

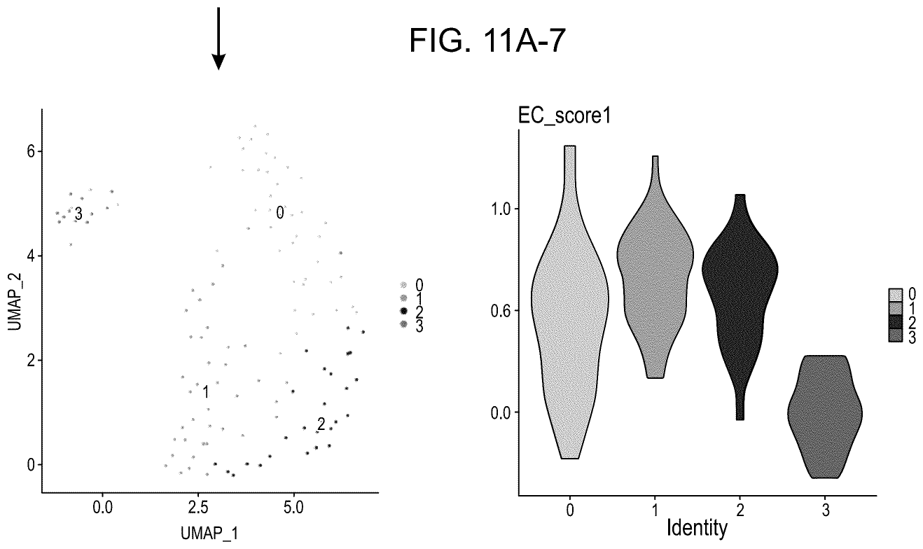

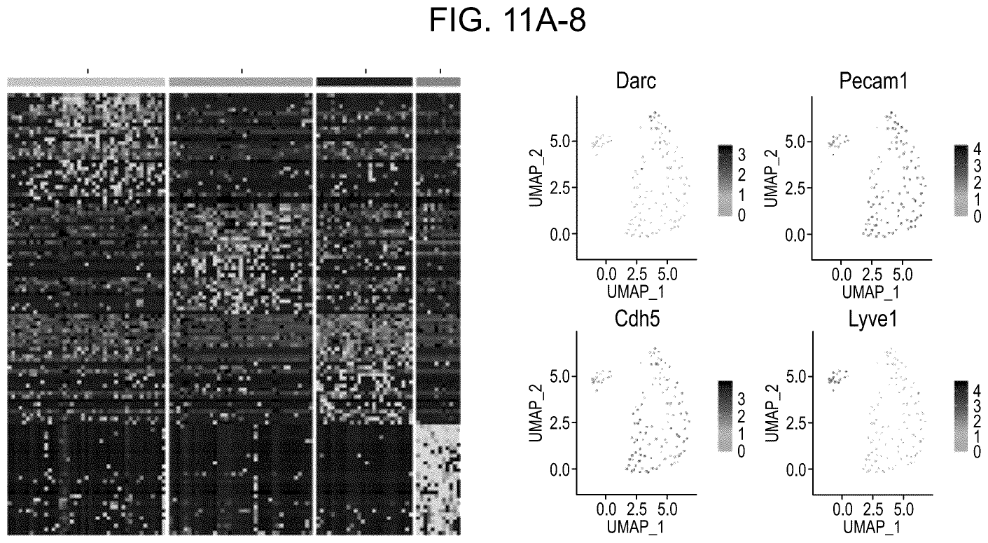

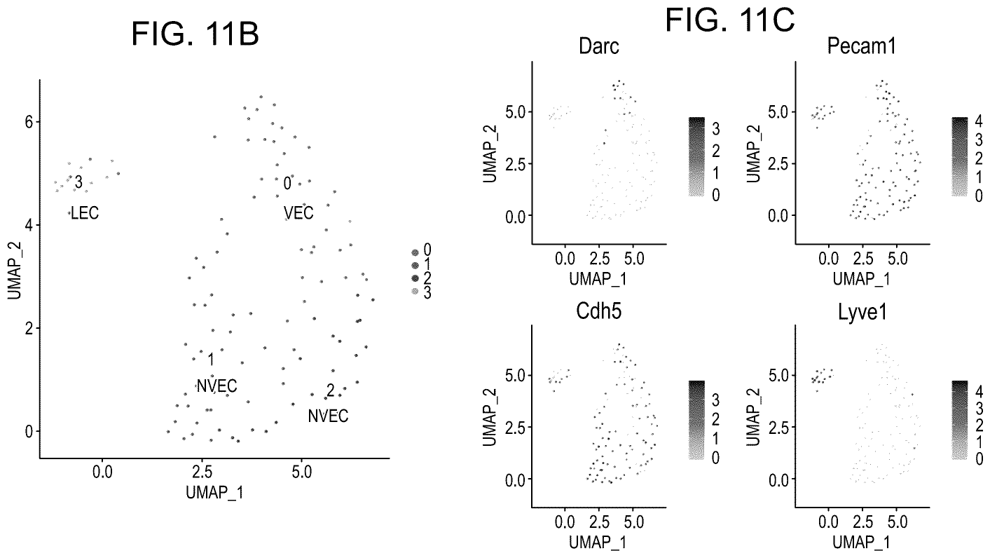

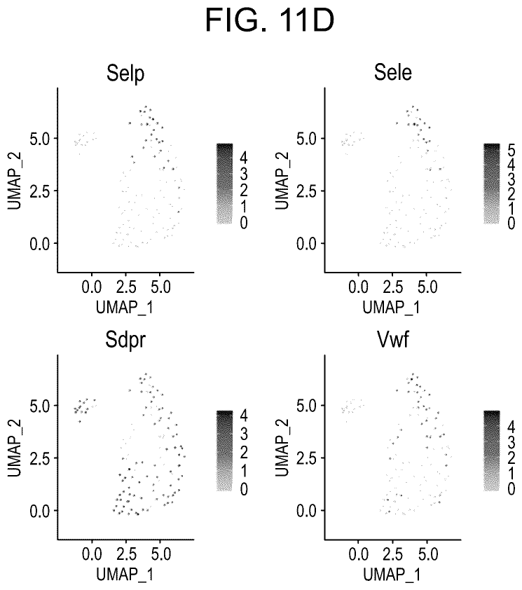

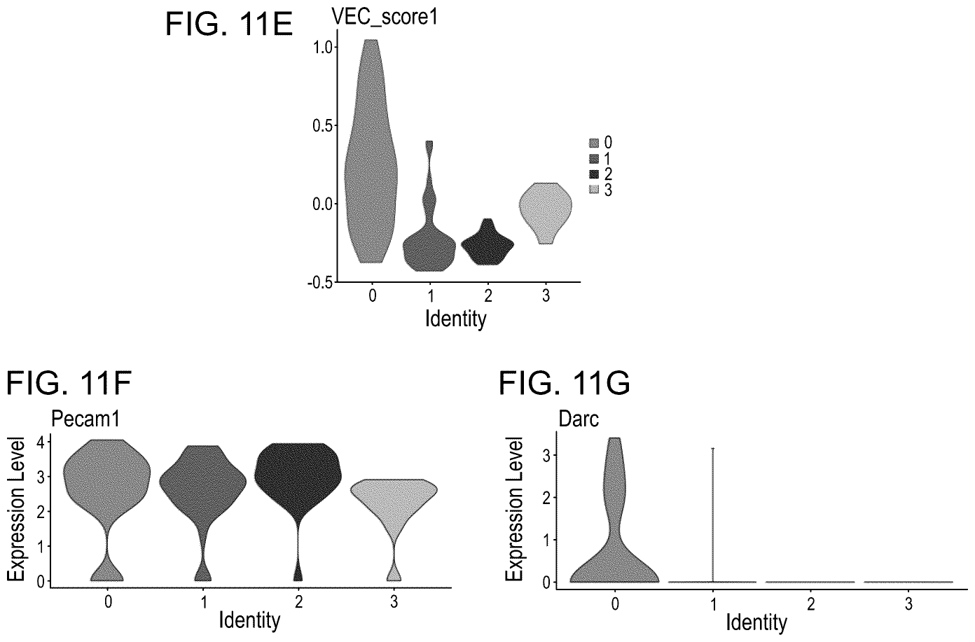

The first step was to develop an efficient methodology for isolating ECs. An iterative process that relied on several pieces of information to identify and isolate ECs was used (FIG. 9B). After clustering the cells using UMAP, the following was performed(1) differential expression to identify specific cell markers for each cluster to assess cell identity (2) heatmaps using those markers were used to help identify cluster(s) with similar gene expression patterns (3) EC scoring based on a list of previously validated markers (Table 9) to assess the EC-ness of a cluster. Based on those different criteria, clusters who were most likely to be containing ECs were isolated and re-clustered. The process was repeated until fully isolated ECs were acquired for each sample type—i.e. healthy skin, MC38 tumor, melanoma, etc. (FIG. 11A).

TABLE 9
Gene signatures used in EC and VEC module scoring.

| | |
|---|---|
| Mouse | Human |

| EC | VEC | EC | VEC |
|---|---|---|---|
| Tie1 | Selp | IFI27 | DARC |
| Mmrn2 | Sele | MGP | SELE |
| Eltd1 | Plvap | SDPR | PLVAP |
| Podxl | Vwf | RAMP2 | CLU |
| Flt1 | Icam1 | IGFBP6 | SELP |
| Selp | Lrg1 | SPARCL1 | ICAM1 |
| Pecam1 | Rasa4 | TIMP3 | LIFR |
| Gpr116 | Il6st | RGS5 | IL1R1 |
| Apold1 | Ctnnal1 | CAV1 | DUSP23 |
| Sele | Darc | VWF | LHX6 |
| Esam | Tmem252 | PTRF | UPP1 |
| Egfl7 | Upp1 | TM4SF1 | PDIA5 |
| Lyve1 | Nr2f2 | IGFBP7 | PRCP |
| Cyyr1 | Ehd4 | CLDN5 | VWF |
| Rasip1 | Syt15 | CPE | MCTP1 |
| Plvap | Sncg | SPARC | OLFM1 |
| Ptprb | Myc | IGFBP5 | LRG1 |
| Cdh5 | Tacr1 | DARC | IGFBP4 |
| Robo4 | Itgb4 | RAMP3 | MYRIP |
| Epas1 | Pdia5 | EMCN | VCAN |
| Vwf | Mctp1 | FCN3 | SYT15 |
| Gpihbp1 | Plekha7 | C7 | MYOF |
| Itga6 | Nuak1 | PTPRB | CSRP2 |
| Fgd5 | Zfp423 | ELTD1 | TACR1 |
| | Arrb1 | EGFL7 | FSTL1 |
| | Lepr | COL4A1 | IL13RA1 |
| | Pgm5 | ENG | CTNNAL1 |
| | Kank1 | AC011526.1 | NDRG1 |
| | Ptgs1 | CD34 | IL6ST |
| | Marveld1 | CDH5 | |
| | Spint2 | ADAMTS1 | |
| | Ret | TMEM100 | |
| | Lhx6 | LDB2 | |
| | Chp2 | LIFR | |
| | Igfbp4 | FBLN2 | |
| | Procr | SELE | |
| | Setbp1 | JAM2 | |
| | Bace2 | CYYR1 | |
| | Hoxd10 | IL33 | |
| | Tll1 | CTNNAL1 | |
| | Golm1 | PRSS23 | |
| | Olfml2a | PODXL | |
| | Vim | EPAS1 | |
| | Pam | PLVAP | |
| | Asap3 | PLCB4 | |
| | Slco2a1 | ENPP2 | |
| | Nmt2 | CRIP2 | |
| | Ldlrap1 | CLU | |
| | | GNG11 | |
| | | CALD1 | |
| | | ESAM | |
| | | APOLD1 | |
| | | ARHGAP29 | |
| | | FLT1 | |
| | | SOCS3 | |
| | | CNN3 | |
| | | CD36 | |
| | | CFH | |
| | | RNASE1 | |
| | | IGFBP3 | |
| | | CRIM1 | |
| | | MT1X | |
| | | RGS16 | |
| | | RND1 | |
| | | ADIRF | |
| | | CXCL2 | |
| | | YBX3 | |
| | | EMP1 | |
| | | SERPING1 | |
| | | CALCRL | |
| | | TNFSF10 | |
| | | C8orf4 | |
| | | EGR1 | |
| | | IL6 | |
| | | SPTBN1 | |
| | | IGFBP4 | |
| | | H19 | |
| | | MTUS1 | |
| | | IL1R1 | |
| | | FABP4 | |
| | | IFITM3 | |
| | | MKL2 | |
| | | CD320 | |
| | | CLIC4 | |
| | | TSC22D1 | |
| | | MAFF | |
| | | ICAM1 | |
| | | RDX | |
| | | ATF3 | |
| | | APOD | |
| | | CCL2 | |
| | | CXCL12 | |
| | | IL6ST | |
| | | GSN | |
| | | TIMP1 | |
| | | ZFP36 | |
| | | ITM2B | |
| | | NCOA7 | |
| | | GADD45B | |
| | | MT2A | |

Second, healthy skin controls for mouse and human were analyzed to ensure that known EC biology was observed and recapitulated. In the mouse healthy skin EC, 4 clusters were observed (FIG. 9C). When looking at the genes expressed by these clusters, the expression of pan-EC genes (CDH5 and PECAM1) in all cells was observed, confirming that ECs were successfully isolated. Genes known to be selectively expressed by EC subsets were used to further identify the clusters. One cluster of LECs, one cluster of VECs, one cluster of capillary ECs and finally one cluster of arteriole ECs were observed. Capillaries and arterioles presented more as a spectrum while VECs and LECs were clearly defined subsets (FIG. 9D). Similarly, out of the seven clusters observed in the human healthy skin 2 clear clusters of VEC, one cluster of LEC and 4 clusters of NVEC were observed. The 4 clusters of NVEC seemed to present a spectrum going from arterioles in NVEC-1 to capillaries in NVEC-4 (FIGS. 9E and 9F).

Immunogenic Tumor VEC More Closely Resemble Non-Tumor VEC and their Profile is Conducive to Recruit Immune Cells

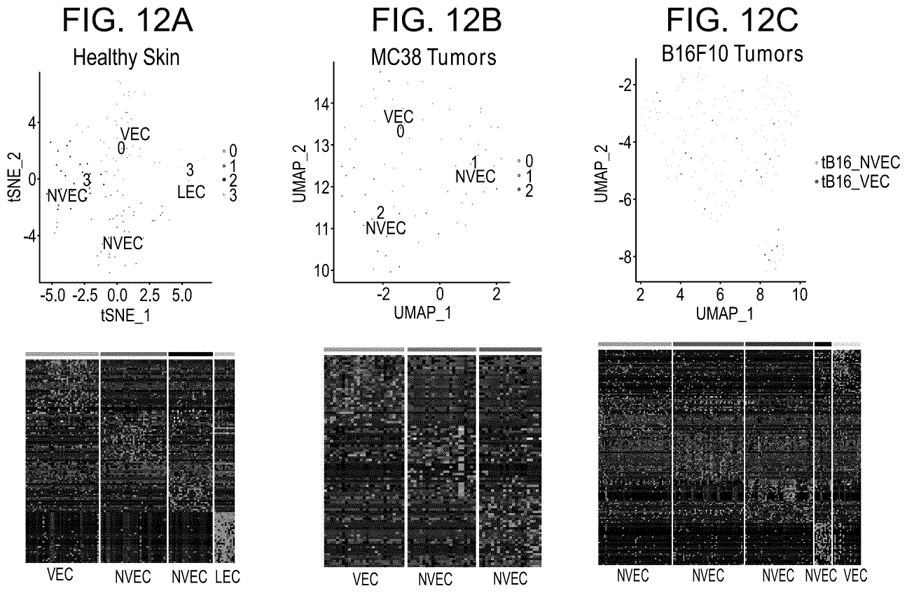

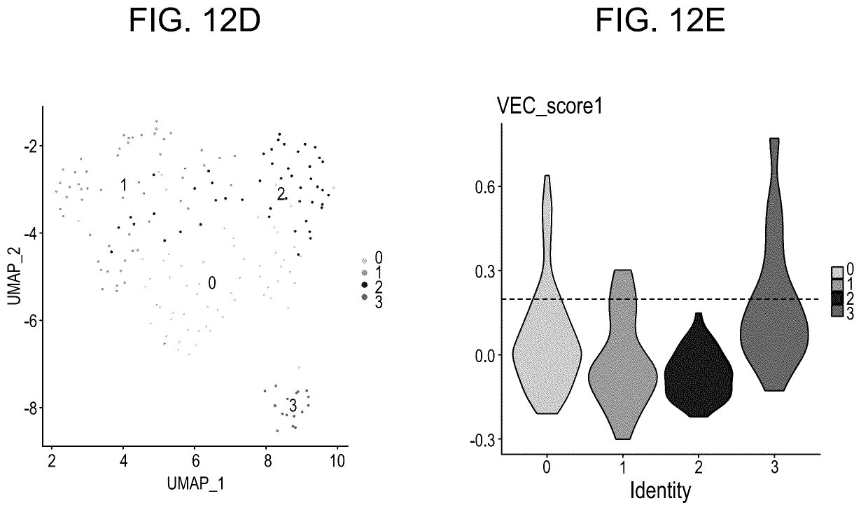

Next, upon establishing that endothelial cells were successfully isolated and analyzed, NVEC and VEC signatures in the immunogenic and non-immunogenic mouse models (MC38 and B16 respectively) were analyzed. To do so MC38 and B16 samples were processed according to the protocol described in FIGS. 9A and 9B. After final clustering, an individual VEC cluster in MC38 was identified (Similar to what was observed in the healthy skin, FIGS. 12A-12C). However, for B16 the VECs were not distinct enough from NVECs to separate into their own cluster. As a result, module scoring based on a gene list curated from genes upregulated in healthy skin VEC was used to identify VECs. Using this strategy, VECs that were spread between all the different clusters were identified (FIGS. 12A-12C).

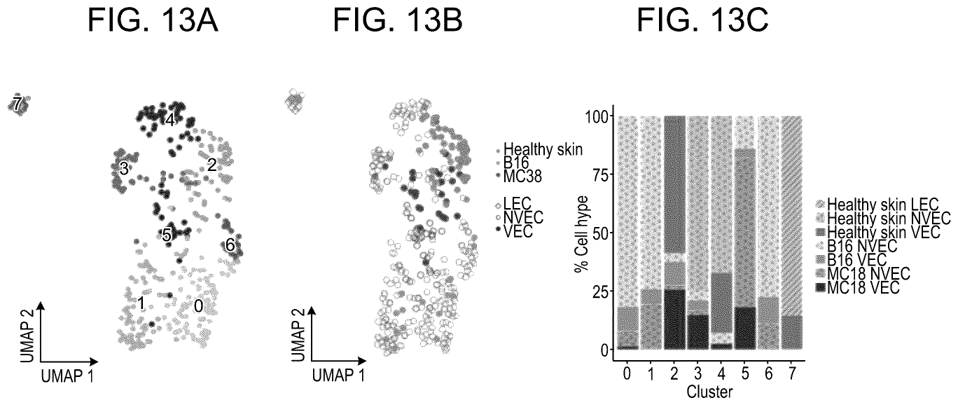

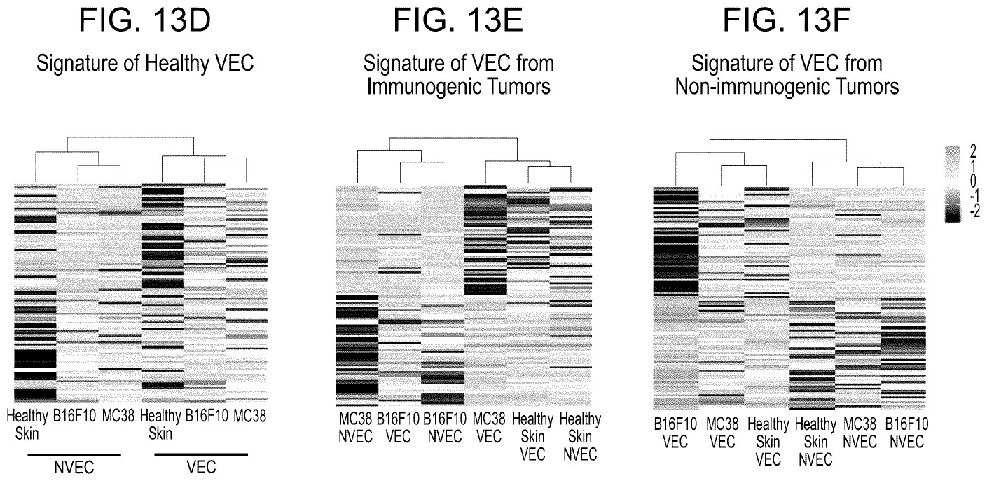

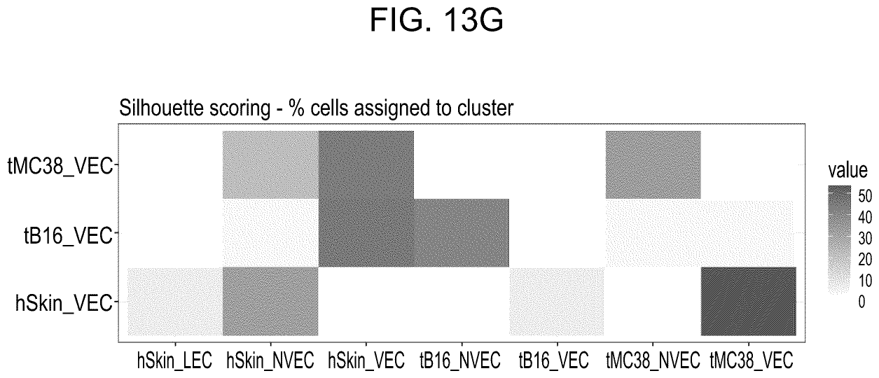

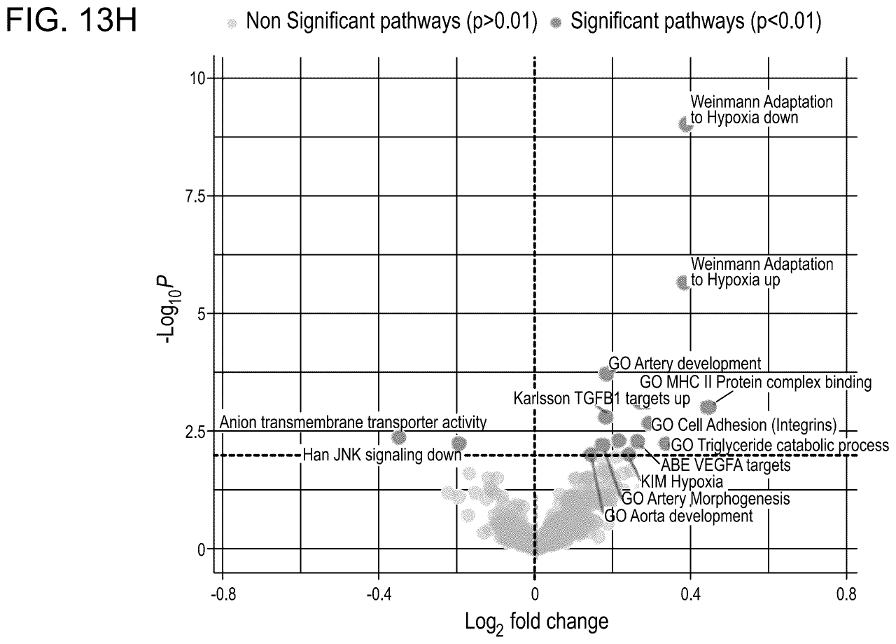

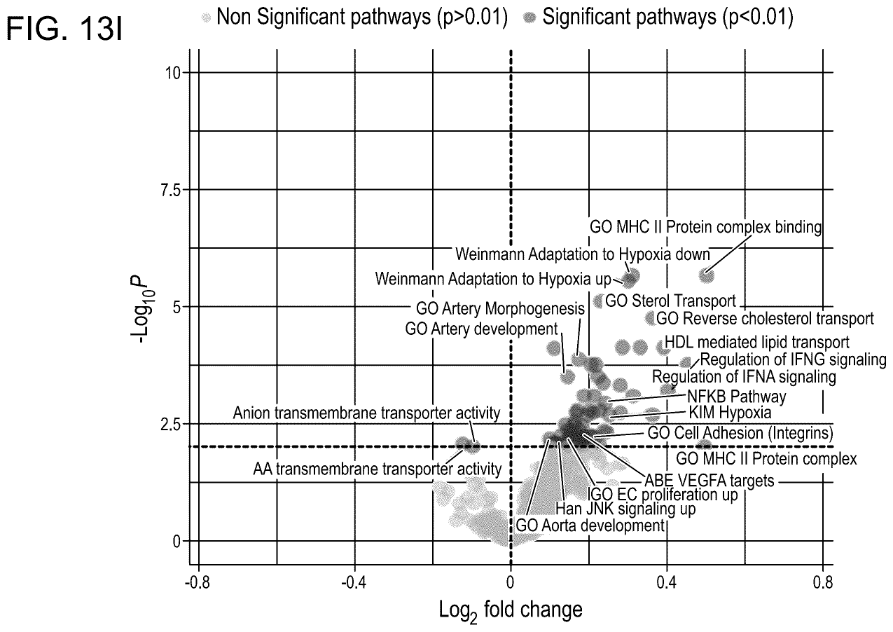

To determine the similarities between healthy, MC38 and B16 VECs and NVECs, all ECs from these samples were pooled together and an unsupervised UMAP clustering was run (FIGS. 13A and 13B). Seven distinct clusters were observed, and cells from different origins were compared to determine how they were spread between clusters. VECs from healthy skin and MC38 seemed to cluster in close proximity while VECs from B16 were spread all over the map. Checking for the exact division between clusters (FIG. 13C), cluster 2 contained mostly VECs from the healthy and MC38 samples while the VECs from B16 seemed to be spread fairly equally between clusters 0-3 and 5 and 6. This suggests that MC38 VECs are more similar to healthy VECs than B16 VECs.

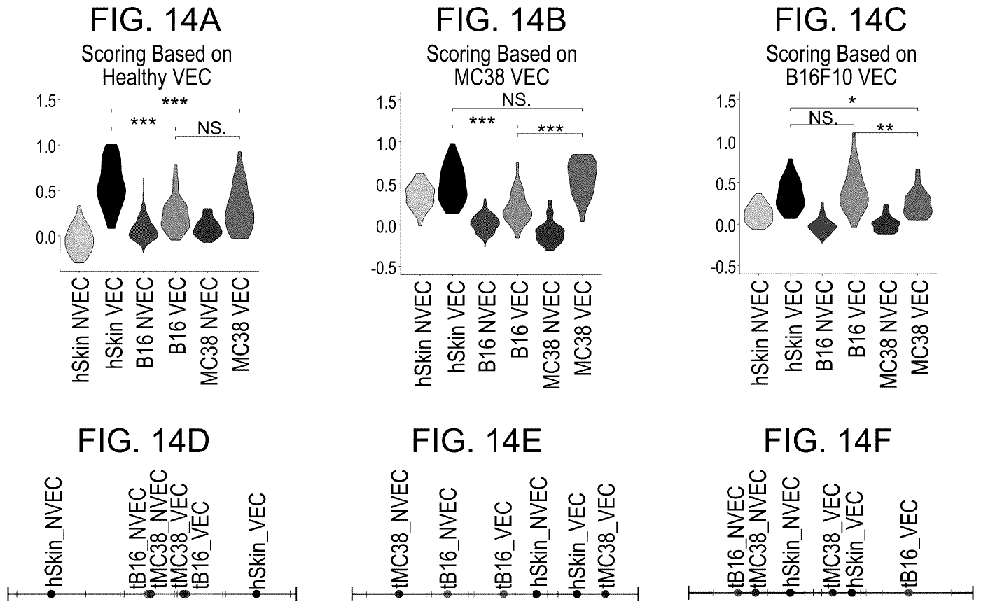

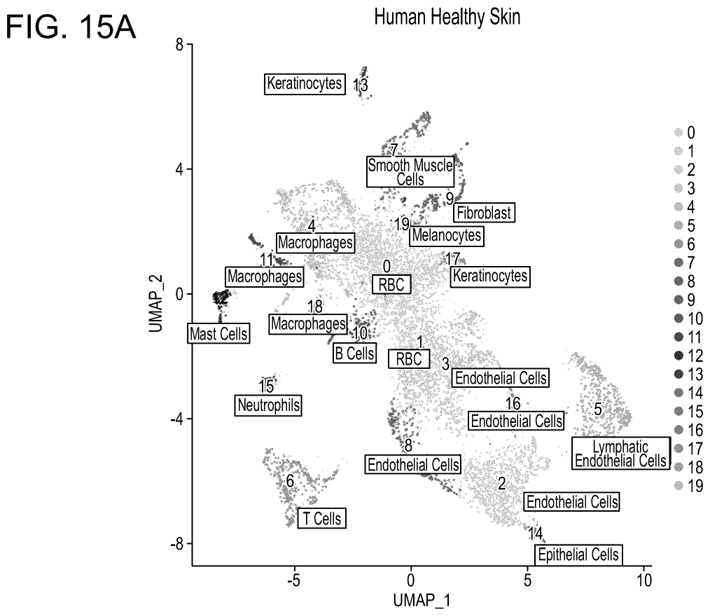

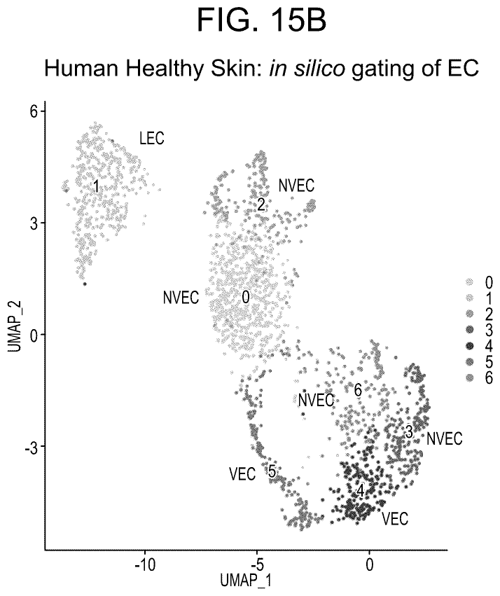

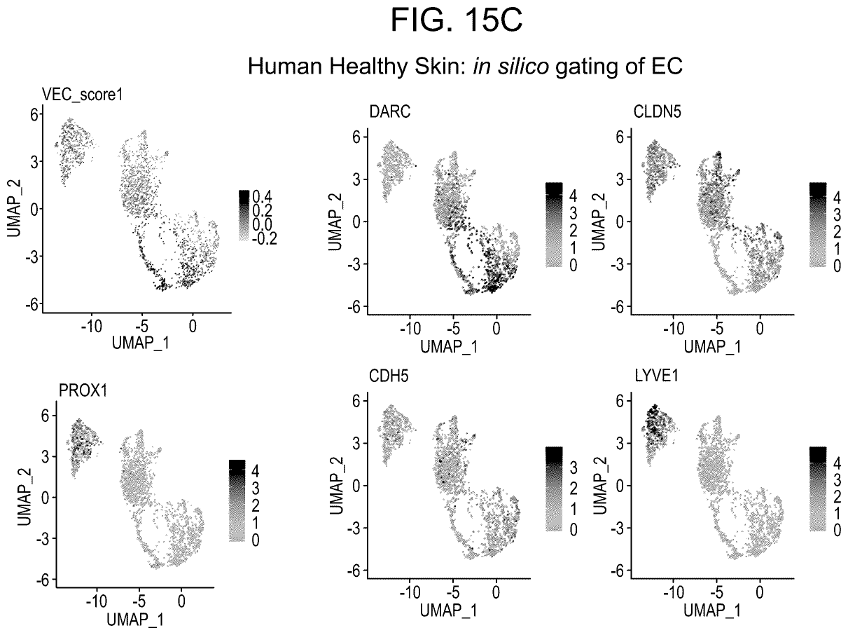

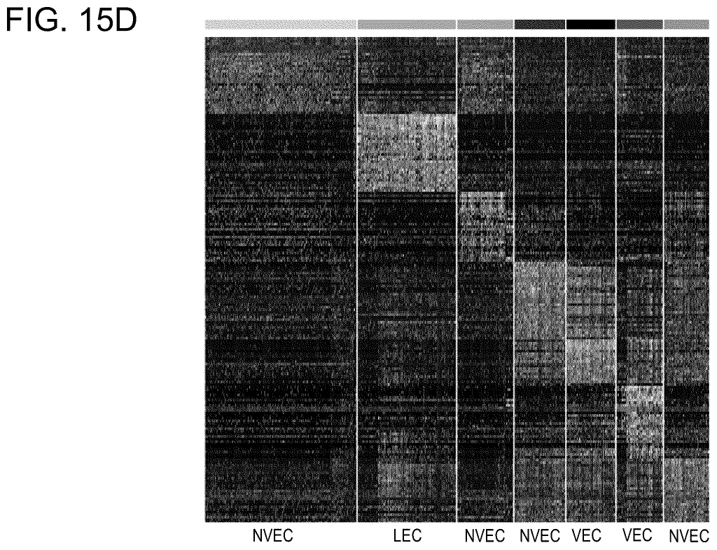

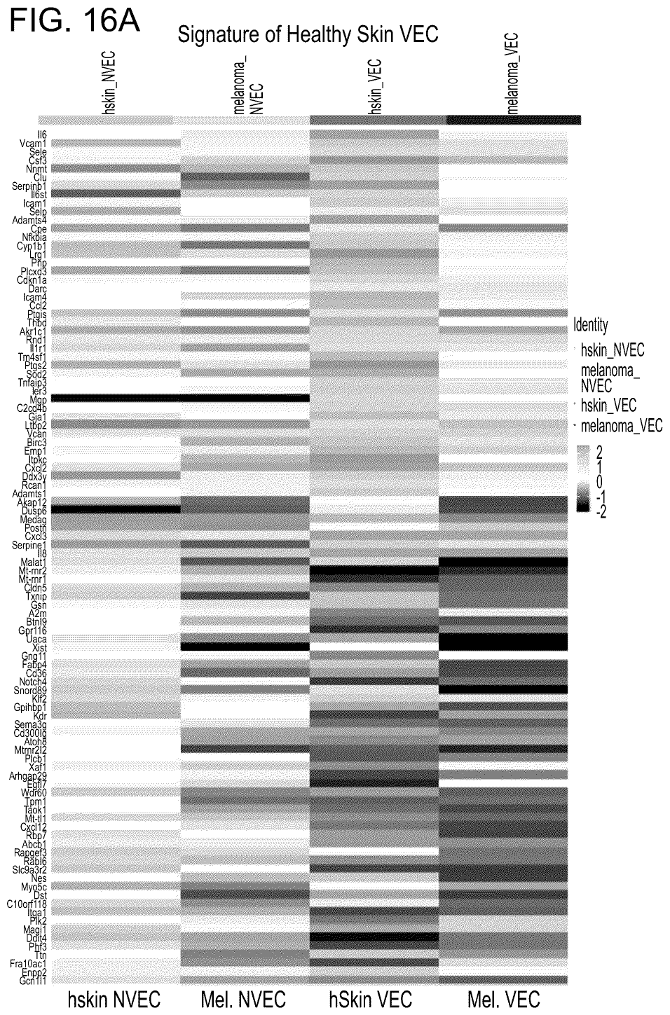

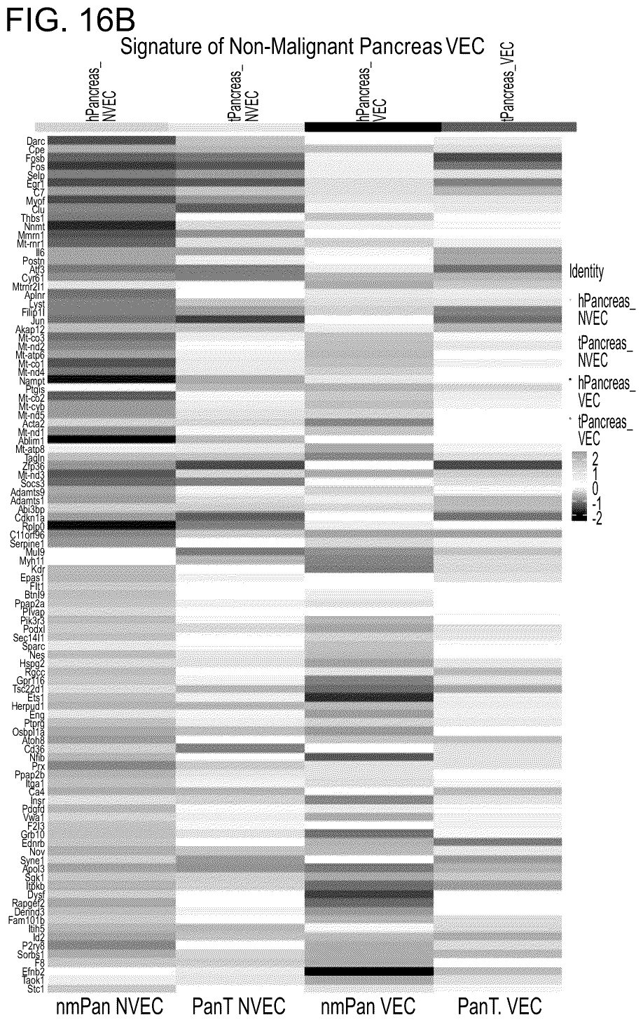

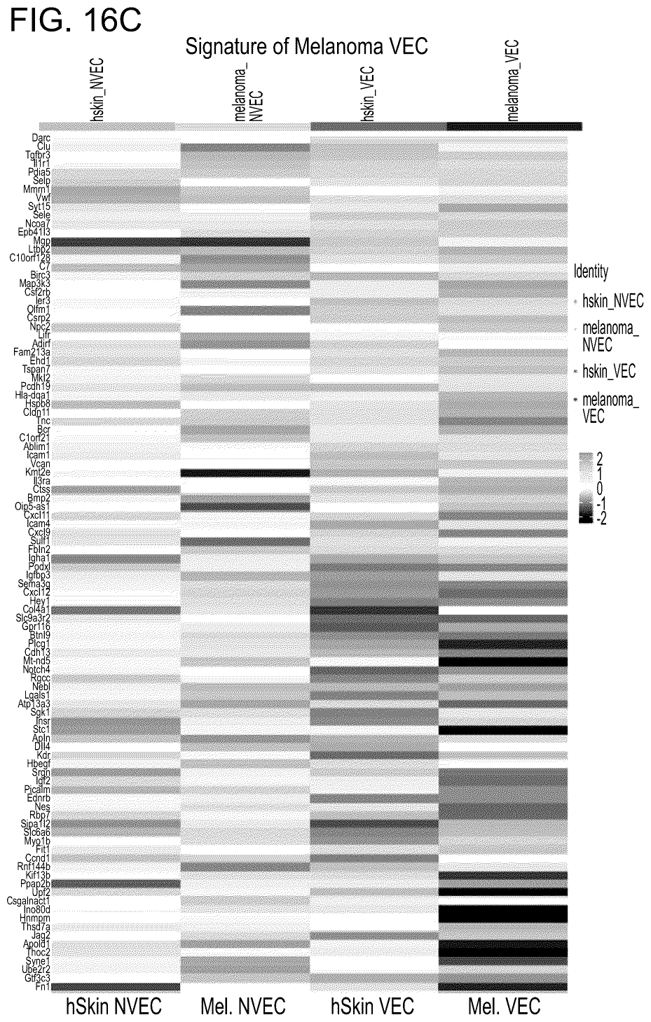

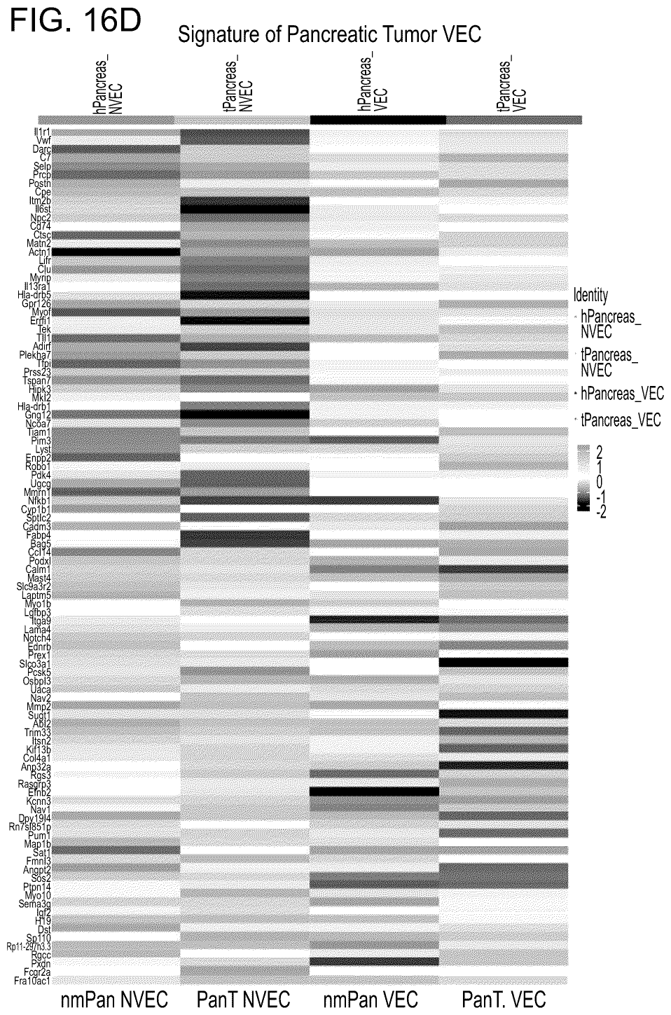

Silhouette algorithm was used to assess the similarities between different subsets. Briefly, the silhouette algorithm evaluated which group of cells a cell is more closely related to, i.e. if the cell had not been assigned to its original group of cells silhouette will determine in which group it would have been placed. Silhouette was used on VECs from healthy skin, MC38 and B16 to see where those cells would fall (FIG. 13G). Most of the healthy skin VECs ended up being placed with the MC38 VECs showing that the healthy VECs resemble MC38 VECs more closely than B16 VECs. MC38 VECs and B16 VECs were split pretty equally between healthy VECs and their matched NVECs. This suggested the presence of a core VEC signature that is maintained in health and disease states. Other strategies based on module scoring were used to assess similarities between the different subsets providing similar results (FIGS. 14A-14F).

Thus, gene signatures from each VEC subset were analyzed and compared to each other. The differentially expressed genes between VECs and NVECs in each sample were looked at and the top and bottom 50 genes were picked. Those were plotted as a heatmaps against each other. Looking at the healthy signature, most of the upregulated genes are typical VEC genes (SELP, SELE, DARC, IL6ST, VWF) that appear to also be upregulated in MC38 and B16 VECs. A few other upregulated genes that are common between samples are new genes that had not been identified as VEC specific previously (CADM3, LRG1) (FIG. 13D). CADM3 (Cell Adhesion Molecule 3) is part of the Ca2+-independent immunoglobulin (Ig) superfamily that participate in the organization of epithelial and endothelial junctions, and LRG1 (Leucine Rich Alpha-2-Glycoprotein 1) has been shown to be involved in promoting neovascularization through causing a switch in transforming growth factor beta (TGFβ) signaling in endothelial cells. Both of those genes present new interesting targets for selective identification and isolation of VEC and would require to be further validated. Overall, the genes differentially expressed in healthy skin VECs mostly revealed a core VEC signature that can be used to identify and select VECs regardless of health or disease.

The MC38 and B16 VEC signature on the other hand revealed more specific programs at play in the immunogenic and non-immunogenic context. In MC38, upregulation of genes involved in DNA damage protection (TMEM109), cell adhesion and migration (LAMB2), and proliferation and angiogenesis (TGFB, FOS) was observed (FIG. 13E). Meanwhile, in B16, upregulation of anti-inflammatory genes (NFKBIA, NFKBIZ, NKRF), as well as regulator of cell growth and proliferation (NDRG1, FOSB) was observed (FIG. 13F). This suggests that while VEC will grow and participate in angiogenesis in both MC38 and B16, they will serve different purpose in each tumor. In MC38 they seem to contribute to T cell recruitment through the expression of cell adhesion proteins, while the expression of anti-inflammatory proteins in B16 will drive down T cell infiltration.

Those preliminary observations were confirmed upon performing gene set variation analysis (GSVA) using a previously curated list of 615 vascular related gene sets that were selected from the Molecular Signatures Database (MSigDB). As expected, a set of common pathways between MC38 and B16 was observed. Those are mostly related to hypoxia, angiogenesis and EC proliferation. All of these are expected in a solid tumor environment as hypoxia is one of the main features of solid tumors and is known to promote angiogenesis and thus EC proliferation. Pathways that are specific to MC38 or B16 were also observed. In MC38, upregulation was observed of inflammation, with several interferon and virus response pathways, as well as an increase in sterol and lipid transport and production, which are known for their protective effects on EC (antioxidant, anti-protease, anti-thrombotic, anti-apoptotic, etc.). Active sterol and lipid transport are also a hallmark of healthy EC operation, highlighting once again the similarities between MC38 VECs and healthy VECs. In B16, upregulation was observed of TGFβ which is involved in cell proliferation and a downregulation of the JNK pathway which has been linked to a reduction in inflammation and T cell recruitment in ECs.

Human and Mouse Immunogenic VECs have a Common Transcriptional Profile

Human samples (healthy skin, melanoma, NM pancreas, pancreatic tumor) were used to see if a pattern similar to what was observed in mouse immunogenic and non-immunogenic tumors emerged. The comparison between healthy skin and melanoma samples was used to look for hallmarks of immunogenic tumors and the comparison between NM pancreas and pancreatic tumor was used to look for hallmarks of non-immunogenic tumors.

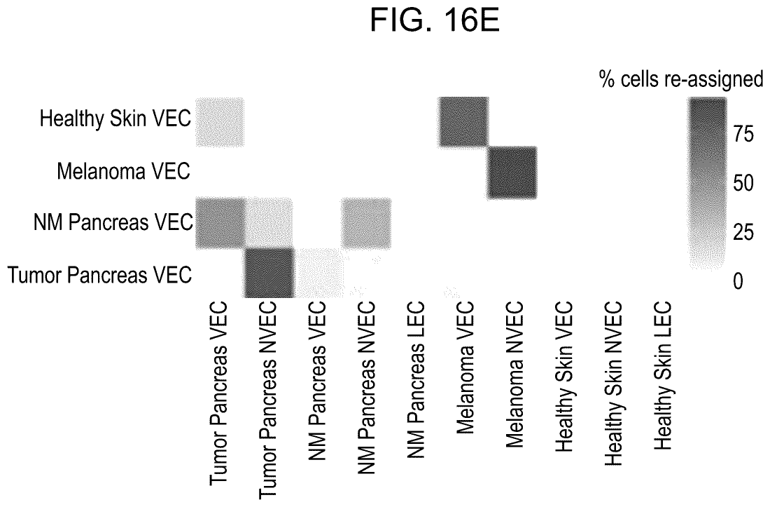

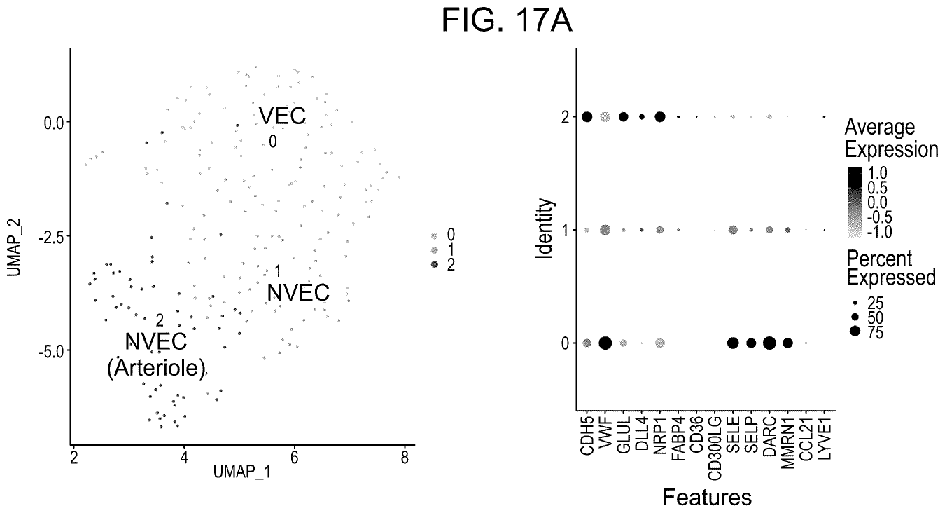

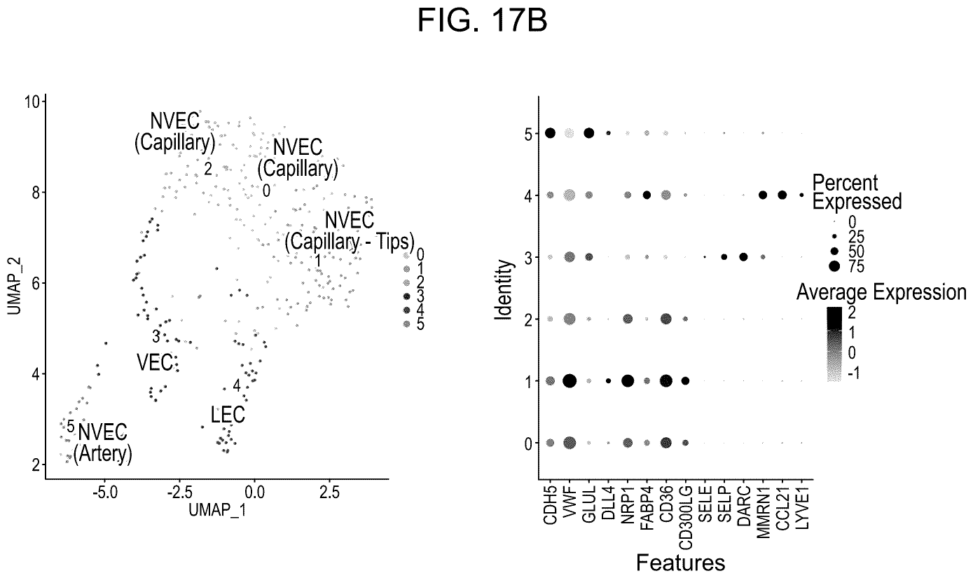

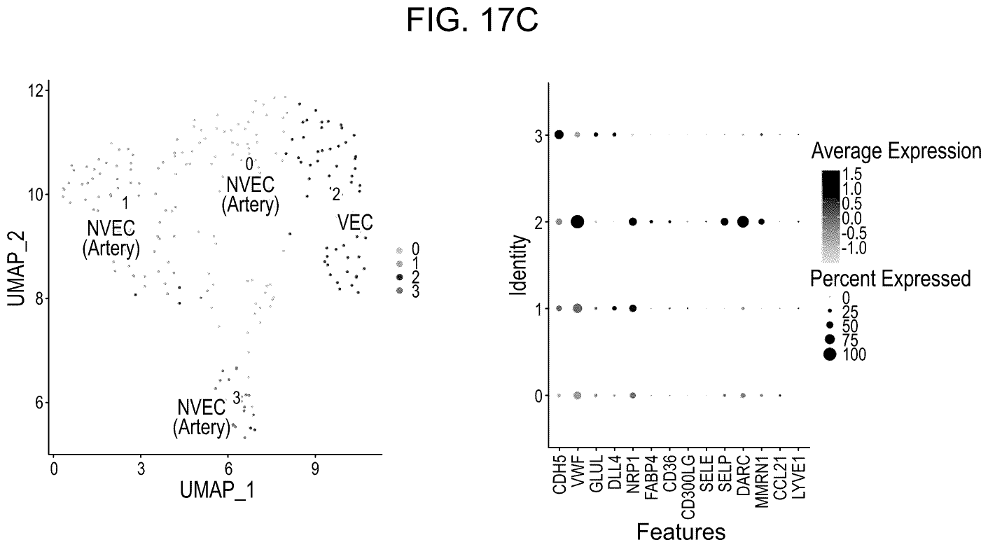

ECs were isolated from each sample, and processed by unsupervised clustering and cluster identification. In all cases, a clear separation between VEC and NVEC was observed (FIGS. 17A-17C).

Human skin and melanoma ECs were pooled together and the cells clustered. The same process was repeated for NM pancreas and pancreatic tumor ECs. The four samples were not pooled together as they're coming from very different tissues and would then separate according to tissue type rather than EC subsets.

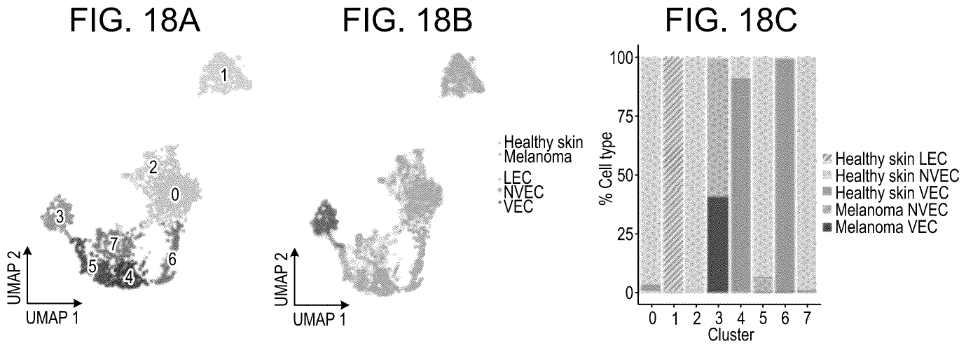

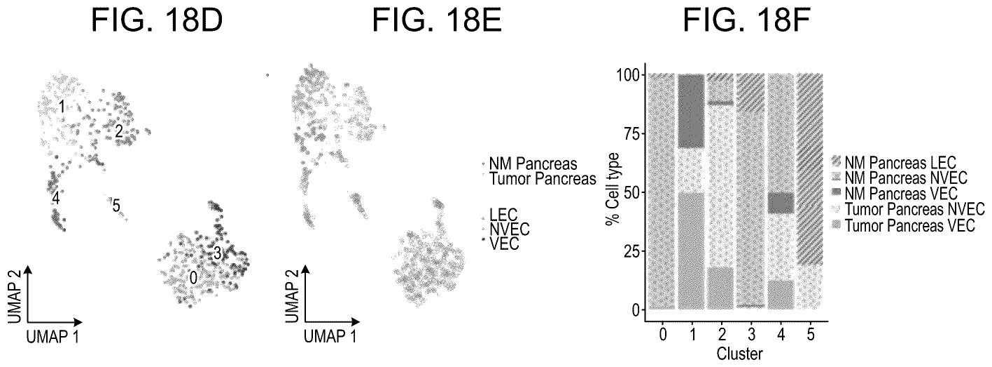

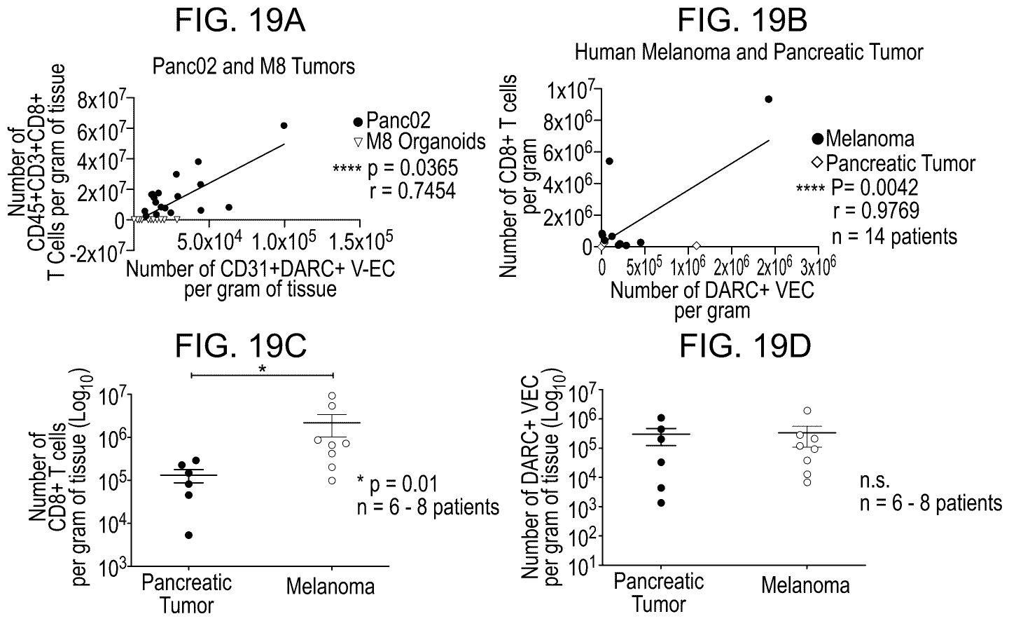

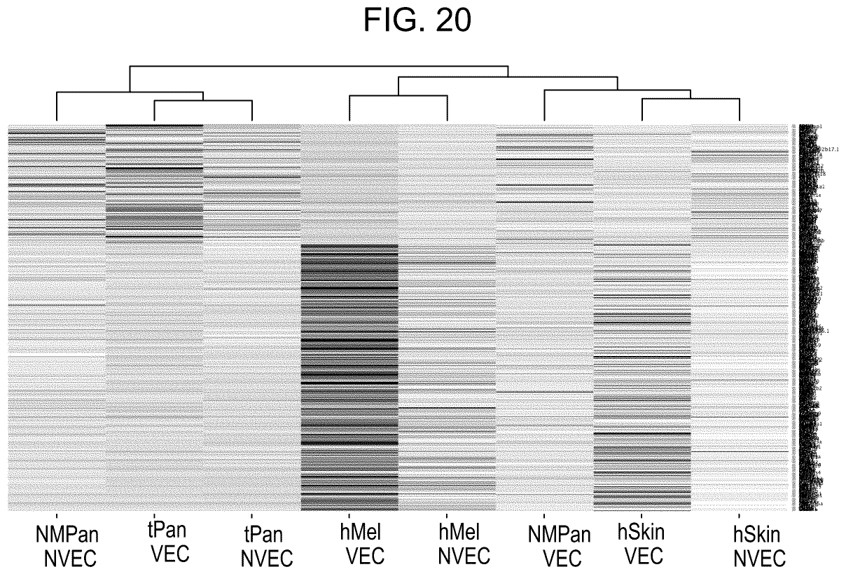

When pooling melanoma and healthy skin, a segregation by sample type was observed (FIGS. 18A-18C). The genes driving that separation are mostly heat shock protein and immune response genes that are both known to be upregulated in melanoma compared to healthy skin. The pooling of pancreas samples did not lead to a stark segregation between non-malignant and malignant samples, clustering based mostly on cell type was observed (FIGS. 18D-18F). Unlike the healthy skin control the non-malignant pancreas control was taken from a disease-free area of the pancreas of a non-healthy subject which partially explains the increased similarities between NM pancreas and pancreatic tumor when compared to healthy skin and melanoma.

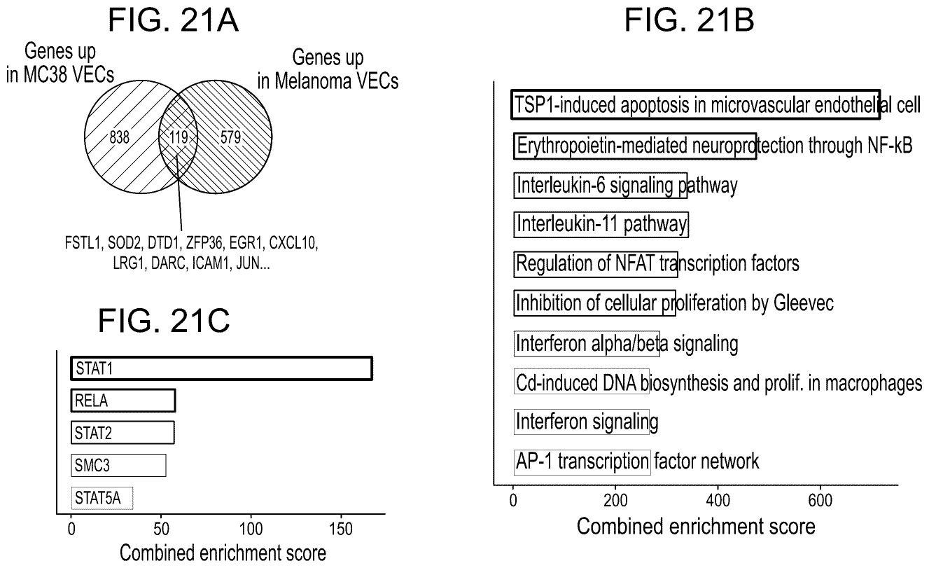

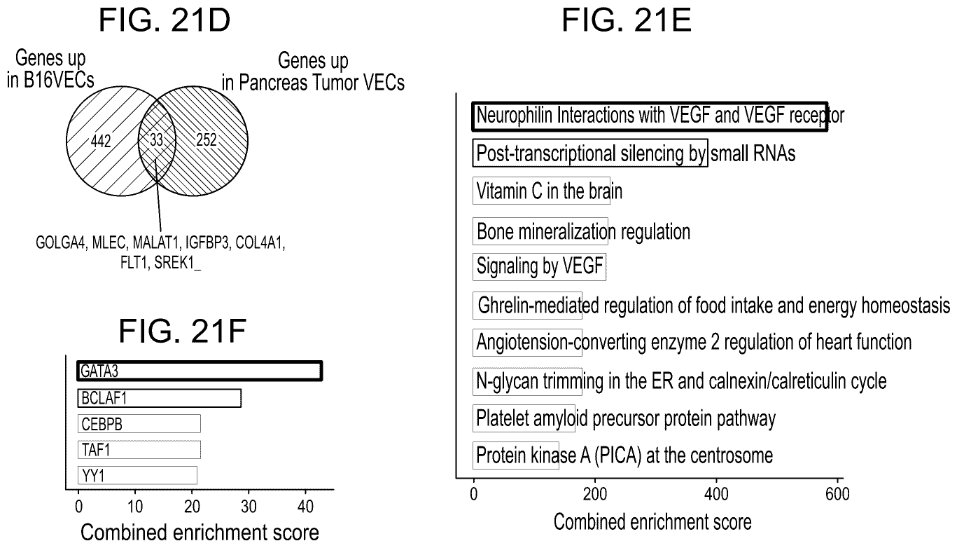

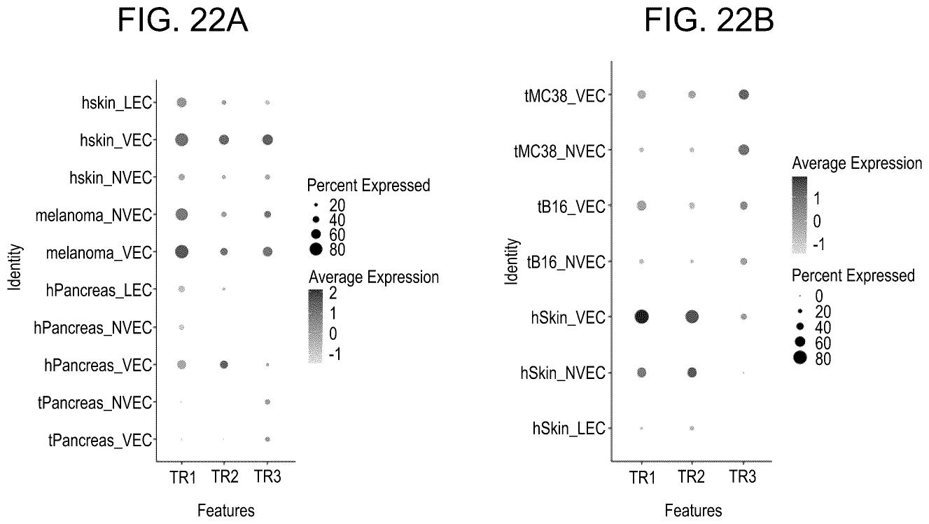

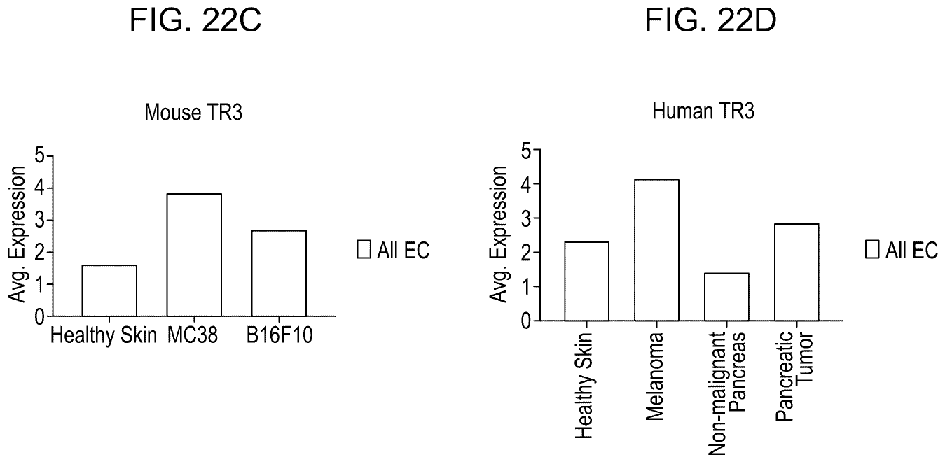

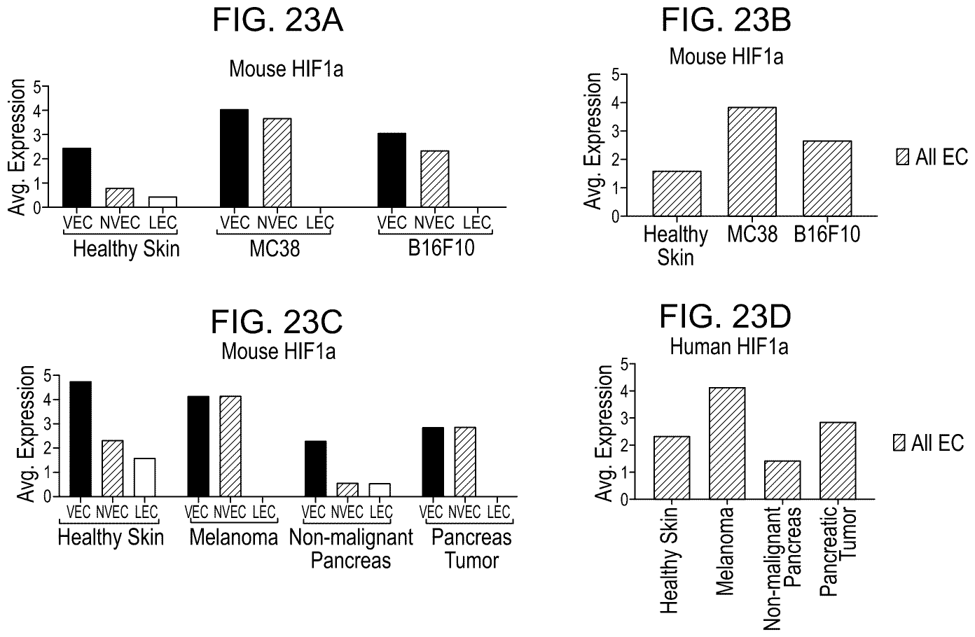

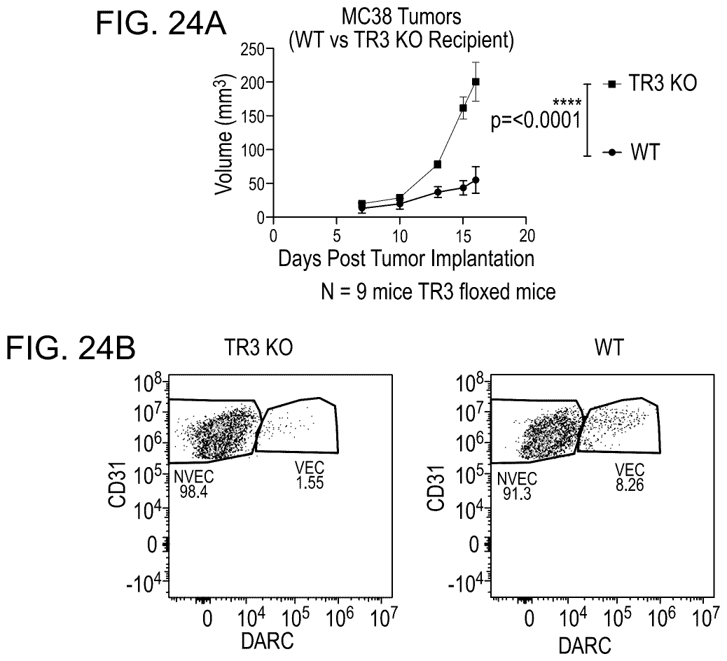

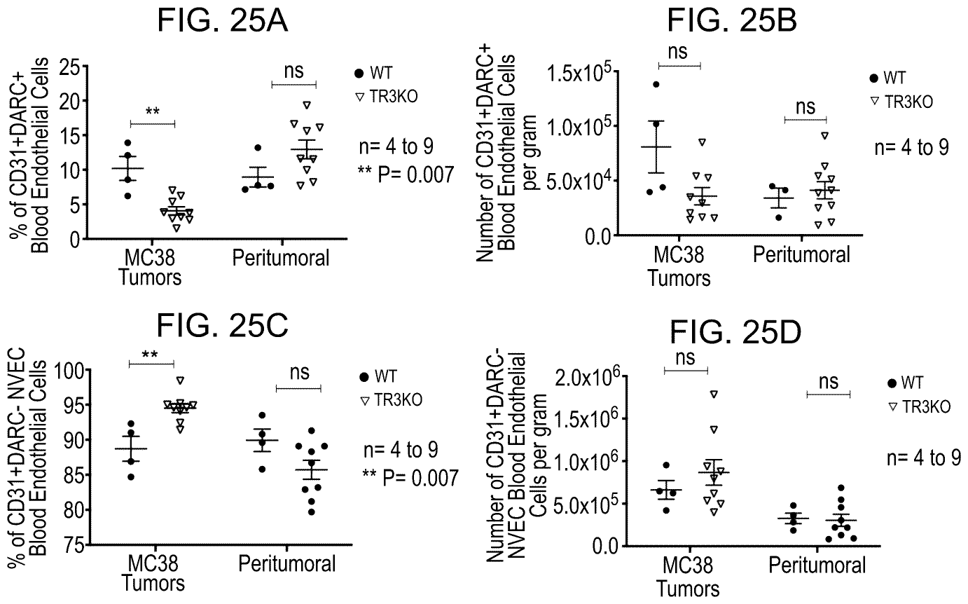

In order to look for differences between immunogenic and non-immunogenic VECs in humans, differential expression between melanoma (immunogenic) VECs and pancreatic tumor (non-immunogenic) VECs was looked at. This gene list was compared to the list of differentially expressed genes in MC38 VECs versus B16 VECs to search for common genes and pathways. 119 genes were found to be upregulated in both mouse and human immunogenic tumors (FIG. 21A) while 33 genes were found to be upregulated in both non-immunogenic tumors (FIG. 21D) (see full lists Table 3). Interestingly in the immunogenic tumor, upregulation was observed of core VEC genes such as DARC, ICAM1 and LRG1. This once again confirm that immunogenicity correlates with a healthier VEC profile. The expression of pro-inflammatory genes and chemokines (JUN, CXCL10), which are known to promote T cell adhesion and recruitment in EC, was observed.

TABLE 10
Shared genes between VECs in human and mouse tumors

| Immunogenic | Non-immunogenic |
|---|---|
| tumors | tumors |
| Vcan | Col4a1 |
| Tgfbr3 | Golga4 |
| Fstl1 | Mlec |
| Ncoa7 | Rbm39 |
| Dtd1 | Nes |
| Rps20 | Hecw2 |
| Mfhas1 | Stc1 |
| Ctss | Ankrd11 |
| Tspan4 | Srsf11 |
| Srp9 | Inpp5a |
| Psmb5 | Cdc42bpb |
| Nup50 | Atad2b |
| Kif26a | Srek1 |
| Ube216 | Evl |
| Psmd1 | Dmtf1 |
| Fos | Malat1 |
| Cst3 | Uaca |
| Fgl2 | Arglu1 |
| Ephb4 | Igfbp3 |
| Ppp1r10 | Apold1 |
| Psen1 | Flt1 |
| Vamp5 | Rbp7 |
| Lrrc16a | Itsn2 |
| B3gnt3 | Pcdh17 |
| Arl4c | Rbm25 |
| Gbp3 | Mlh3 |
| Wars | Tnrc6a |
| Irf1 | Pcnt |
| Txnl1 | Safb |
| Egr1 | Vps53 |
| Sox17 | Akap12 |
| Tuba1b | Eif5b |
| Ugcg | Ppap2a |
| Cd74 | |
| Sod2 | |
| Fcgrt | |
| Clec14a | |
| Srsf4 | |
| Pogk | |
| Syngr2 | |
| Ccni | |
| Zfp36l2 | |
| Rgs2 | |
| Samhd1 | |
| Glul | |
| Myc | |
| Rnf19b | |
| Mat2a | |
| Tnfrsf1b | |
| Tsc22d1 | |
| Pcdh19 | |
| Mob4 | |
| Rplp0 | |
| Lamb2 | |
| Kctd10 | |
| Ier2 | |
| Ddx3x | |
| Ppapdc1b | |
| Rpl10a | |
| Utp20 | |
| Cd47 | |
| Anxa5 | |
| Cd9 | |
| Vps29 | |
| Hnrnpa0 | |
| Ifnar1 | |
| Ifitm3 | |
| Socs3 | |
| Pmp22 | |
| Paf1 | |
| Arl6ip1 | |
| Sertad1 | |
| Scarb1 | |
| Csf2rb | |
| Ifi27 | |
| Ppp1r15a | |
| Sav1 | |
| Dpy19l4 | |
| Pttg1ip | |
| Fbln2 | |
| Cdc42 | |
| Lrrc8c | |
| Actr2 | |
| Ebf3 | |
| Klf2 | |
| Helz2 | |
| Meox2 | |
| Wdr82 | |
| Cxcl10 | |
| Chka | |
| Thbs1 | |
| Ppm1f | |
| Ctsl | |
| Ablim1 | |
| Lmo4 | |
| Darc | |
| Cdkn1a | |
| Ptgs2 | |
| Ly6e | |
| Jun | |
| Psmb8 | |
| Eef1a1 | |
| Nfkb2 | |
| Dusp1 | |
| Klf4 | |
| Nudt3 | |
| Lrp5 | |
| Fosb | |
| Ctgf | |
| Jak2 | |
| Crim1 | |
| Clu | |
| Imp3 | |
| C2cd2 | |
| Thbd | |
| Junb | |
| Lrg1 | |
| Pds5a | |
| Icam1 | |

In non-immunogenic tumors, expression of IGFBP3 and MALATI can be observed, both are known to be upregulated in cancer and are associated with poor prognosis which is consistent with the fact that non-immunogenic tumors are often harder to treat. They're both associated with vascular growth which suggest that the lower T-cell infiltration in non-immunogenic tumor is not due to a lack of neovascularization but to the fact that those VECs are less efficient at capturing T cells and at facilitating their transfer into the tumor microenvironment.

Next, the pathways and transcription factors that might be upregulated in immunogenic tumors were explored. Immunogenicity in a tumor may be improved by turning on relevant pathways. R implementation of EnrichR, a gene enrichment tool which currently contains a large collection of diverse gene set libraries available for analysis and download, was used. In total, EnmichR currently contains 180,184 annotated gene sets from 102 gene set libraries. The BioPlanet database, which integrates pathway annotations from publicly available, manually curated sources that have been subjected to thorough redundancy and consistency cross-evaluation via extensive manual curation, was used to analyze pathways. For transcription factor (TF) analysis the data presented here was generated using the most recent Chip-Seq ENCODE database but other TF databases gave similar results. (enrichment tables in Tables 11-14).

TABLE 11
GSVA pathway enrichment in MC38.

| Name | logFC | AveExpr | P. Value | adj.P.Val |
|---|---|---|---|---|
| GO_TRIGLYCERIDE_CATABOLIC_PROCESS | 0.50 | 0.00 | 0.00 | 0.00 |
| GO_MHC_CLASS_II_PROTEIN_COMPLEX | 0.50 | −0.19 | 0.00 | 0.01 |
| GO_MHC_CLASS_II_PROTEIN_COMPLEX_BINDING | 0.46 | −0.12 | 0.00 | 0.00 |
| REACTOME_REGULATION_OF_IFNA_SIGNALING | 0.40 | −0.23 | 0.00 | 0.00 |
| REACTOME_HDL_MEDIATED_LIPID_TRANSPORT | 0.39 | −0.03 | 0.00 | 0.00 |
| GO_REVERSE_CHOLESTEROL_TRANSPORT | 0.36 | 0.05 | 0.00 | 0.00 |
| RAFFEL_VEGFA_TARGETS_UP | 0.36 | 0.00 | 0.00 | 0.00 |
| GO_ACYLGLYCEROL_CATABOLIC_PROCESS | 0.33 | 0.03 | 0.00 | 0.00 |
| REACTOME_REGULATION_OF_IFNG_SIGNALING | 0.31 | −0.17 | 0.00 | 0.00 |
| WEINMANN_ADAPTATION_TO_HYPOXIA_DN | 0.31 | −0.08 | 0.00 | 0.00 |
| REACTOME_LIPID_DIGESTION_MOBILIZA- | 0.30 | −0.02 | 0.00 | 0.00 |
| TION_AND_TRANSPORT | | | | |
| REACTOME_LIPOPROTEIN_METABOLISM | 0.29 | −0.04 | 0.00 | 0.00 |
| GO_RENAL_SYSTEM_VASCULATURE_DEVELOPMENT | 0.28 | −0.08 | 0.00 | 0.00 |
| WEINMANN_ADAPTATION_TO_HYPOXIA_UP | 0.28 | −0.10 | 0.00 | 0.00 |
| KIM_HYPOXIA | 0.25 | −0.12 | 0.00 | 0.00 |
| GO_CELL_ADHESION_MEDIATED_BY_INTEGRIN | 0.25 | −0.06 | 0.00 | 0.00 |
| BIOCARTA_NFKB_PATHWAY | 0.24 | −0.08 | 0.00 | 0.00 |
| ABE_VEGFA_TARGETS_30 MIN | 0.24 | −0.14 | 0.00 | 0.00 |
| GO_LONG_CHAIN_FATTY_ACID_TRANSPORT | 0.24 | 0.00 | 0.00 | 0.00 |
| GO_STEROL_TRANSPORT | 0.23 | 0.00 | 0.00 | 0.00 |
| HALLMARK_ANGIOGENESIS | 0.23 | −0.09 | 0.00 | 0.00 |
| HAN_JNK_SINGALING_UP | 0.23 | −0.17 | 0.00 | 0.01 |
| GO_POSITIVE_REGULATION_OF_OXIDORE- | 0.22 | −0.04 | 0.00 | 0.00 |
| DUCTASE_ACTIVITY | | | | |
| GO_REGULATION_OF_NITRIC_OXIDE_SYN- | 0.22 | −0.09 | 0.00 | 0.00 |
| THASE_ACTIVITY | | | | |
| FRIDMAN_SENESCENCE_DN | 0.22 | −0.06 | 0.00 | 0.01 |
| GO_ENDOTHELIAL_CELL_PROLIFERATION | 0.21 | −0.10 | 0.00 | 0.00 |
| FRIDMAN_SENESCENCE_UP | 0.21 | −0.13 | 0.00 | 0.00 |
| GO_NEGATIVE_REGULATION_OF_ENDOTHE- | 0.21 | −0.10 | 0.00 | 0.01 |
| LIAL_CELL_PROLIFERATION | | | | |
| GO_RESPIRATORY_GASEOUS_EXCHANGE | 0.20 | −0.10 | 0.00 | 0.00 |
| MENSSEN_MYC_TARGETS | 0.20 | −0.12 | 0.00 | 0.01 |
| DAUER_STAT3_TARGETS_DN | 0.20 | −0.13 | 0.00 | 0.00 |
| GO_RESPONSE_TO_INTERFERON_BETA | 0.20 | −0.04 | 0.00 | 0.00 |
| GO_ARTERY_MORPHOGENESIS | 0.19 | −0.10 | 0.00 | 0.00 |
| ZHANG_PROLIFERATING_VS_QUIESCENT | 0.19 | −0.14 | 0.00 | 0.01 |
| GO_ANTIGEN_PROCESSING_AND_PRESENTA- | 0.19 | −0.12 | 0.00 | 0.01 |
| TION_OF_EXOGENOUS_PEPTID_E_ANTI- | | | | |
| GEN_VIA_MHC_CLASS_I | | | | |
| DAUER_STAT3_TARGETS_UP | 0.19 | −0.08 | 0.00 | 0.01 |
| GO_POSITIVE_REGULATION_OF_LIPID_BIO- | 0.18 | −0.06 | 0.00 | 0.01 |
| SYNTHETIC_PROCESS | | | | |
| GO_FATTY_ACID_TRANSPORT | 0.18 | 0.01 | 0.00 | 0.01 |
| GO_POSITIVE_REGULATION_OF_COAGULATION | 0.18 | −0.03 | 0.00 | 0.00 |
| GO_POSITIVE_REGULATION_OF_ATPASE_ACTIVITY | 0.18 | −0.10 | 0.00 | 0.01 |
| GO_ARTERY_DEVELOPMENT | 0.17 | −0.08 | 0.00 | 0.00 |
| GO_POSITIVE_REGULATION_OF_STEROID_META- | 0.17 | −0.02 | 0.00 | 0.00 |
| BOLIC_PROCESS | | | | |
| GO_REGULATION_OF_ENDOTHELIAL_CELL_PRO- | 0.17 | −0.08 | 0.00 | 0.00 |
| LIFERATION | | | | |
| SANA_TNF_SIGNALING_UP | 0.17 | −0.06 | 0.00 | 0.01 |
| DER_IFN_BETA_RESPONSE_UP | 0.17 | −0.13 | 0.00 | 0.00 |
| GO_ANTIGEN_BINDING | 0.16 | −0.05 | 0.00 | 0.00 |
| PETROVA_PROX1_TARGETS_DN | 0.16 | −0.09 | 0.00 | 0.00 |
| GO_POSITIVE_REGULATION_OF_ENDOTHE- | 0.16 | −0.08 | 0.00 | 0.01 |
| LIAL_CELL_PROLIFERATION | | | | |
| DER_IFN_ALPHA_RESPONSE_UP | 0.15 | −0.11 | 0.00 | 0.01 |
| GO_POSITIVE_REGULATION_OF_REACTIVE_OXY- | 0.15 | −0.07 | 0.00 | 0.00 |
| GEN_SPECIES_BIOSYNTHETIC_PROCESS | | | | |
| HARRIS_HYPOXIA | 0.15 | −0.06 | 0.00 | 0.01 |
| GO_REGULATION_OF_NITRIC_OXIDE_BIO- | 0.15 | −0.06 | 0.00 | 0.01 |
| SYNTHETIC_PROCESS | | | | |
| GO_REGULATION_OF_CGMP_METABOLIC_PROCESS | 0.15 | 0.06 | 0.00 | 0.01 |
| GO_RESPONSE_TO_VIRUS | 0.15 | −0.10 | 0.00 | 0.00 |
| GO_DEFENSE_RESPONSE_TO_VIRUS | 0.14 | −0.09 | 0.00 | 0.00 |
| GO_REGULATION_OF_OXIDOREDUCTASE_ACTIVITY | 0.14 | −0.06 | 0.00 | 0.00 |
| GO_ANTIGEN_PROCESSING_AND_PRESENTA- | 0.13 | −0.12 | 0.00 | 0.01 |
| TION_OF_PEPTIDE_ANTIGEN | | | | |
| GO_AORTA_DEVELOPMENT | 0.13 | −0.07 | 0.00 | 0.01 |
| GO_POSITIVE_REGULATION_OF_REAC- | 0.12 | −0.07 | 0.00 | 0.01 |
| TIVE_OXYGEN_SPECIES_METABOLIC_PROCESS | | | | |
| ZHENG_IL22_SIGNALING_UP | 0.11 | 0.03 | 0.00 | 0.01 |
| WINTER_HYPOXIA_METAGENE | 0.11 | −0.09 | 0.00 | 0.01 |
| GO_ORGANIC_HYDROXY_COMPOUND_TRANSPORT | 0.11 | 0.02 | 0.00 | 0.00 |
| GO_REGULATION_OF_REACTIVE_OXYGEN_SPE- | 0.11 | −0.06 | 0.00 | 0.01 |
| CIES_METABOLIC_PROCESS | | | | |
| GO_REGULATION_OF_LIPID_BIOSYNTHETIC_PROCESS | 0.10 | −0.02 | 0.00 | 0.01 |
| GO_MONOCARBOXYLIC_ACID_TRANSPORT | 0.10 | 0.03 | 0.00 | 0.01 |
| GO_ORGANIC_ANION_TRANSMEMBRANE_TRANS- | −0.10 | 0.11 | 0.00 | 0.01 |
| PORTER_ACTIVITY | | | | |
| GO_AMINO_ACID_TRANSMEMBRANE_TRANS- | −0.12 | 0.10 | 0.00 | 0.01 |
| PORTER_ACTIVITY | | | | |

Adj.P.Val: adjusted p value calculated using the Benjamini-Hochberg procedure.

TABLE 12
GSVA pathway enrichment in B16.

| Name | logFC | AveExpr | P.Value | adj.P.Val |
|---|---|---|---|---|
| GO_MHC_CLASS_II_PROTEIN_COMPLEX_BINDING | 0.44 | −0.12 | 0.00 | 0.00 |
| WEINMANN_ADAPTATION_TO_HYPOXIA_DN | 0.39 | −0.08 | 0.00 | 0.00 |
| WEINMANN_ADAPTATION_TO_HYPOXIA_UP | 0.38 | −0.10 | 0.00 | 0.00 |
| GO_TRIGLYCERIDE_CATABOLIC_PROCESS | 0.34 | 0.00 | 0.00 | 0.01 |
| GO_CELL_ADHESION_MEDIATED_BY_INTEGRIN | 0.29 | −0.06 | 0.00 | 0.00 |
| FRIDMAN_SENESCENCE_DN | 0.29 | −0.06 | 0.00 | 0.00 |
| MENSSEN_MYC_TARGETS | 0.27 | −0.12 | 0.00 | 0.00 |
| ABE_VEGFA_TARGETS_30 MIN | 0.26 | −0.14 | 0.00 | 0.01 |
| KIM_HYPOXIA | 0.24 | −0.12 | 0.00 | 0.01 |
| FRIDMAN_SENESCENCE_UP | 0.21 | −0.13 | 0.00 | 0.00 |
| GO_ARTERY_DEVELOPMENT | 0.18 | −0.08 | 0.00 | 0.00 |
| KARLSSON_TGFB1_TARGETS_UP | 0.18 | −0.12 | 0.00 | 0.00 |
| GO_ARTERY_MORPHOGENESIS | 0.17 | −0.10 | 0.00 | 0.01 |
| GO_AORTA_DEVELOPMENT | 0.14 | −0.07 | 0.00 | 0.01 |
| HAN_JNK_SINGALING_DN | −0.19 | −0.10 | 0.00 | 0.01 |
| GO_SODIUM_INDEPENDENT_ORGANIC_AN- | −0.35 | −0.11 | 0.00 | 0.00 |
| ION_TRANSMEMBRANE_TRANSPORTER_ACTIVITY | | | | |

Adj.P.Val: adjusted p value calculated using the Benjamini-Hochberg procedure.

TABLE 13
EnrichR pathway enrichment in Immunogenic and non-immunogenic mouse and human tumors.

| | | | | | |
|---|---|---|---|---|---|
| Term | Overlap | P.value | Adj.P.Val | CS | Genes |

IMMUNOGENIC

| | | | | | |
|---|---|---|---|---|---|
| TSP1-induced apoptosis in | 3/8 | 1.13E−05 | 0.000894 | 718 | JUN; FOS; THBS1 |
| microvascular endothelial cell | | | | | |
| Erythropoietin-mediated | 3/11 | 3.27E−05 | 0.001765 | 473 | CDKN1A; JAK2; SOD2 |
| neuroprotection through NF- | | | | | |
| kB | | | | | |
| Interleukin-6 signaling | 8/71 | 9.64E−09 | 2.08E−06 | 350 | SOCS3; JUN; LMO4; |
| pathway | | | | | MYC; IRF1; FOS; JAK2; |
| | | | | | JUNB |
| Interleukin-11 pathway | 4/23 | 9.67E−06 | 0.000859 | 338 | SOCS3; JUN; FOS; ICAM1 |
| Regulation of NFAT | 6/47 | 3.44E−07 | 3.71E−05 | 319 | EGR1; JUN; FOS; PTGS2; |
| transcription factors | | | | | JUNB; GBP3 |
| Inhibition of cellular | 4/24 | 1.15E−05 | 0.000872 | 318 | JUN; MYC; FOS; JAK2 |
| proliferation by Gleevec | | | | | |
| Interferon alpha/beta signaling | 7/64 | 1.04E−07 | 1.74E−05 | 296 | IFITM3; SOCS3; EGR1; |
| | | | | | IFI27; IRF1; PSMB8; |
| | | | | | IFNAR1 |
| Cadmium-induced DNA | 3/17 | 0.00013 | 0.005222 | 265 | JUN; MYC; FOS |
| biosynthesis and proliferation | | | | | |
| in macrophages | | | | | |
| Interferon signaling | 12/168 | 3.62E−10 | 1.82E−07 | 261 | IFITM3; SOCS3; EGR1; |
| | | | | | IFI27; NUP50; IRF1; |
| | | | | | UBE2L6; JAK2; PSMB8; |
| | | | | | ICAM1; IFNAR1; GBP3 |
| AP-1 transcription factor | 7/70 | 1.94E−07 | 2.44E−05 | 260 | EGR1; JUN; DUSP1; |
| network | | | | | MYC; FOSB; FOS; JUNB |
| T cell receptor calcium | 4/29 | 2.52E−05 | 0.001523 | 245 | JUN; FOS; PTGS2; JUNB |
| pathway | | | | | |
| BDNF signaling pathway | 15/261 | 4.48E−11 | 3.38E−08 | 230 | EGR1; JUN; CDKN1A; |
| | | | | | LMO4; DUSP1; FOS; |
| | | | | | PTGS2; CLU; RGS2; |
| | | | | | VCAN; ABLIM1; |
| | | | | | MYC; FOSB; JUNB; IER2 |
| TSH regulation of gene | 8/97 | 1.15E−07 | 1.74E−05 | 22 | PPP1R15A; EGR1; RGS2; |
| expression | | | | | JUN; MYC; FOS; PTGS2; |
| | | | | | ICAM1 |
| Activation of the AP-1 family | 2/10 | 0.00153 | 0.027202 | 218 | JUN; FOS |
| of transcription factors | | | | | |
| ID regulation of gene | 3/20 | 0.00022 | 0.006841 | 213 | CDKN1A; THBS1; ICAM1 |
| expression | | | | | |
| T cell receptor regulation of | 24/603 | 1.21E−13 | 1.83E−10 | 199 | IFITM3; EGR1; JUN; |
| apoptosis | | | | | CDKN1A; DUSP1; POGK; |
| | | | | | CSF2RB; FOS; RPL10A; |
| | | | | | TNFRSF1B; SOD2; CLU; |
| | | | | | PSMB8; CDC42; CXCL10; |
| | | | | | FCGRT; MYC; IRF1; FOSB; |
| | | | | | RPS20; JUNB; IER2; |
| | | | | | IFNAR1; GBP3 |
| Interleukin-5 signaling | 5/49 | 1.06E−05 | 0.000889 | 196 | JUN; MYC; CSF2RB; |
| pathway | | | | | FOS; JAK2 |
| Type II interferon signaling | 5/50 | 1.17E−05 | 0.000843 | 191 | SOCS3; CXCL10; IRF1; |
| (interferon-gamma) | | | | | JAK2; ICAM1 |
| Interleukin-4 regulation of | 14/267 | 6.89E−10 | 2.60E−07 | 186 | JUN; CDKN1A; FGL2; |
| apoptosis | | | | | RNF19B; UBE2L6; |
| | | | | | CSF2RB; FOS; PTGS2; |
| | | | | | ARL4C; RGS2; VCAN; |
| | | | | | CTSL; MYC; CD9 |
| Signaling events mediated by PRL | 3/23 | 0.00033 | 0.009683 | 176 | EGR1; CDKN1A; TUBA1B |

NON-IMMUNOGENIC

| | | | | | |
|---|---|---|---|---|---|
| Neurophilin interactions with | 1/5 | 0.00822 | 1 | 582 | FLT1 |
| VEGF and VEGF receptor | | | | | |
| Post-transcriptional silencing | 1/7 | 0.01149 | 1 | 387 | TNRC6A |
| by small RNAs | | | | | |
| Vitamin C in the brain | 1/11 | 0.01801 | 1 | 221 | COL4A1 |
| Bone mineralization regulation | 1/11 | 0.01801 | 1 | 221 | COL4A1 |
| Signaling by VEGF | 1/11 | 0.01801 | 1 | 221 | FLT1 |
| Ghrelin-mediated regulation of | 1/13 | 0.02125 | 1 | 180 | IGFBP3 |
| food intake and energy | | | | | |
| homeostasis | | | | | |
| Angiotensin-converting | 1/13 | 0.02125 | 1 | 180 | COL4A1 |
| enzyme 2 regulation of heart | | | | | |
| function | | | | | |
| N-glycan trimming in the ER | 1/13 | 0.02125 | 1 | 180 | MLEC |
| and calnexin/calreticulin cycle | | | | | |
| Platelet amyloid precursor | 1/14 | 0.02286 | 1 | 164 | COL4A1 |
| protein pathway | | | | | |
| Protein kinase A (PKA) at the | 1/16 | 0.02609 | 1 | 138 | PCNT |
| centrosome | | | | | |
| Insulin-like growth factor | 1/17 | 0.02769 | 1 | 128 | IGFBP3 |
| (IGF) activity regulation by | | | | | |
| insulin-like growth factor | | | | | |
| binding proteins (IGFBPs) | | | | | |
| Eukaryotic protein translation | 1/17 | 0.02769 | 1 | 128 | EIF5B |
| Acute myocardial infarction | 1/20 | 0.0325 | 1 | 104 | COL4A1 |
| Mismatch repair | 1/23 | 0.03729 | 1 | 87 | MLH3 |
| Angiogenesis | 1/23 | 0.03729 | 1 | 87 | FLT1 |
| Hypoxia and p53 in the | 1/23 | 0.03729 | 1 | 87 | IGFBP3 |
| cardiovascular system | | | | | |
| Triacylglyceride biosynthesis | 1/24 | 0.03888 | 1 | 82 | PPAP2A |
| Regulatory RNA pathways | 1/25 | 0.04047 | 1 | 78 | TNRC6A |
| VEGFR1 pathway | 1/27 | 0.04363 | 1 | 70 | FLT1 |
| S1P/S1P3 pathway | 1/29 | 0.04679 | 1 | 64 | FLT1 |

Overlap: overlap between the input set of genes and the annotated gene sets.
Adj.P.Val: adjusted p value calculated using the Benjamini-Hochberg procedure.
CS: combined score. Combination of the p-value and z-score calculated by multiplying the two scores as follows: c = ln(p) * z. Where c is the combined score, p is the p-value computed using Fisher's exact test, and z is the z-score computed to assess the deviation from the expected rank.

TABLE 14
EnrichR TF enrichment in Immunogenic and non-immunogenic mouse and human tumors.

| | | | | | |
|---|---|---|---|---|---|
| Term | Overlap | P.value | Adj.P.Val | CS | Genes |

IMMUNOGENIC

| | | | | | |
|---|---|---|---|---|---|
| STAT1 | 30/945 | 2.46E−14 | 2.01E−11 | 167 | IFITM3; CDKN1A; LRP5; UBE2L6; RPL10A; |
| HeLa-S3 | | | | | PTGS2; NUDT3; CTSS; ICAM1; SOCS3; |
| hg19 | | | | | ABLIM1; SYNGR2; TSPAN4; JAK2; JUNB; |
| | | | | | EGR1; JUN; WARS; FOS; TNFRSF1B; SOD2; |
| | | | | | PSMB8; NFKB2; EEF1A1; LRG1; IFI27; |
| | | | | | IRF1; NCOA7; LY6E; IFNAR1 |
| RELA | 26/1302 | 3.72E−08 | 6.08E−06 | 57 | CDKN1A; DDX3X; CCNI; ARL6IP1; RPL10A; |
| GM12892 | | | | | ZFP36L2; ICAM1; SYNGR2; MAT2A; JAK2; |
| hg19 | | | | | GLUL; JUNB; IER2; HNRNPA0; CD74; |
| | | | | | JUN; ANXA5; TNFRSF1B; PSMB8; NFKB2; |
| | | | | | EEF1A1; MOB4; LRG1; DPY19L4; IRF1; |
| | | | | | IFNAR1 |
| STAT2 | 9/271 | 3.56E−05 | 0.001383 | 57 | IFITM3; DDX3X; WARS; IFI27; IRF1; UBE2L6; |
| K562 hg19 | | | | | NCOA7; SAMHD1; ICAM1 |
| SMC3 | 34/2000 | 1.03E−08 | 2.80E−06 | 53 | CDKN1A; DDX3X; C2CD2; RPLP0; FGL2; |
| CH12.LX | | | | | RPL10A; CLU; PPM1F; SAV1; CST3; SYNGR2; |
| mm9 | | | | | JAK2; GLUL; PPAPDC1B; DTD1; CHKA; |
| | | | | | LMO4; DUSP1; ANXA5; TNFRSF1B; |
| | | | | | SRP9; PSMB8; NFKB2; EEF1A1; TGFBR3; |
| | | | | | ARL4C; MFHAS1; DPY19L4; IRF1; PAF1; |
| | | | | | CD9; RPS20; CD47; IFNAR1 |
| UBTF | 30/2000 | 1.43E−06 | 0.00013 | 34 | VPS29; CCNI; FGL2; PSEN1; PDS5A; ZFP36L2; |
| CH12.LX | | | | | SAV1; SOCS3; JAK2; PPAPDC1B; DTD1; |
| mm9 | | | | | EPHB4; HNRNPA0; ACTR2; DUSP1; KLF4; |
| | | | | | PSMB8; NFKB2; MOB4; ARL4C; KCTD10; |
| | | | | | DPY19L4; NUP50; IRF1; PAF1; RPS20; |
| | | | | | NCOA7; CD47; VAMP5; IFNAR1 |
| CHD1 | 14/691 | 6.29E−05 | 0.001975 | 33 | DDX3X; PPP1R10; FOS; RPL10A; SOD2; |
| MEL cell | | | | | ZFP36L2; EEF1A1; MAT2A; MYC; TSPAN4; |
| line mm9 | | | | | CD47; JUNB; VAMP5; HNRNPA0 |
| STAT5A | 6/217 | 0.00191 | 0.019008 | 29 | PPP1R15A; SOCS3; WARS; LRG1; IMP3; |
| K562 hg19 | | | | | SRSF4 |
| STAT3 | 28/1974 | 1.03E−05 | 0.000599 | 27 | IFITM3; CCNI; ARL6IP1; UBE2L6; PTGS2; |
| HeLa-S3 | | | | | FSTL1; CTGF; ICAM1; UGCG; SOCS3; |
| hg19 | | | | | ABLIM1; MYC; JUNB; GBP3; EGR1; JUN; |
| | | | | | WARS; CHKA; IMP3; DUSP1; CRIM1; FOS; |
| | | | | | SOD2; PSMB8; IRF1; NCOA7; VAMP5; |
| | | | | | IFNAR1 |
| CEBPB | 23/1513 | 2.56E−05 | 0.001099 | 27 | IFITM3; CHKA; LMO4; PPP1R10; PSEN1; |
| C2C12 | | | | | RPL10A; MEOX2; TNFRSF1B; PTGS2; |
| mm9 | | | | | SAMHD1; FSTL1; PPM1F; MOB4; SYNGR2; |
| | | | | | WDR82; CTSL; DPY19L4; MYC; CD9; |
| | | | | | RPS20; CD47; JUNB; IER2 |
| ATF3 | 28/1991 | 1.21E−05 | 0.000656 | 27 | VPS29; CCNI; ARL6IP1; RPLP0; RPL10A; |
| A549 hg19 | | | | | PTGS2; SAMHD1; CLU; SAV1; SOCS3; THBD; |
| | | | | | SERTAD1; MYC; TSPAN4; PSMD1; JUNB; |
| | | | | | HNRNPA0; ACTR2; JUN; IMP3; SRP9; |
| | | | | | EEF1A1; MOB4; KCTD10; DPY19L4; PAF1; |
| | | | | | FOSB; RPS20 |

NON-IMMUNOGENIC

| | | | | | |
|---|---|---|---|---|---|
| GATA3 | 9/1185 | 9.22E−05 | 0.037614 | 43 | RBM39; IGFBP3; HECW2; STC1; MALAT1; |
| SK-N-SH | | | | | UACA; APOLD1; PCDH17; MLH3 |
| hg19 | | | | | |
| BCLAF1 | 7/1007 | 0.00108 | 0.126471 | 29 | EIF5B; RBM25; DMTF1; MLEC; MALAT1; |
| K562 hg19 | | | | | SREK1; ATAD2B |
| CEBPB | 7/1173 | 0.00261 | 0.177375 | 22 | RBM39; EIF5B; ANKRD11; STC1; MALAT1; |
| MCF-7 | | | | | SREK1; APOLD1 |
| hg19 | | | | | |
| TAF1 | 9/1695 | 0.00131 | 0.118625 | 21 | RBM39; RBM25; GOLGA4; DMTF1; STC1; |
| MCF-7 | | | | | SREK1; PCNT; UACA; ATAD2B |
| hg19 | | | | | |
| YY1 | 10/2000 | 0.00105 | 0.17073 | 21 | RBM39; EIF5B; RBM25; INPP5A; VPS53; |
| GM12878 | | | | | EVL; MALAT1; CDC42BPB; SREK1; SRSF11 |
| hg19 | | | | | |
| SP2 | 8/1507 | 0.00258 | 0.191335 | 19 | RBM39; EIF5B; RBM25; ANKRD11; VPS53; |
| HepG2 | | | | | PCNT; UACA; ATAD2B |
| hg19 | | | | | |
| EP300 | 6/1155 | 0.01059 | 0.375767 | 14 | RBM39; RBM25; DMTF1; MALAT1; TNRC6A; |
| T47D hg19 | | | | | ATAD2B |
| SP1 K562 | 6/1249 | 0.01518 | 0.364235 | 12 | RBM39; ANKRD11; MLEC; MALAT1; PCNT; |
| hg19 | | | | | APOLD1 |
| MEF2A | 5/970 | 0.02038 | 0.426409 | 12 | DMTF1; MLEC; MALAT1; APOLD1; ATAD2B |
| GM12878 | | | | | |
| hg19 | | | | | |
| EGR1 | 4/741 | 0.03264 | 0.532647 | 11 | RBM39; RBM25; MALAT1; UACA |
| MCF-7 | | | | | |
| hg19 | | | | | |

Overlap: overlap between the input set of genes and the annotated gene sets.
Adj.P.Val: adjusted p value calculated using the Benjamini-Hochberg procedure.
CS: combined score. Combination of the p-value and z-score calculated by multiplying the two scores as follows: c = In(p) * z. Where c is the combined score, p is the p-value computed using Fisher's exact test, and z is the z-score computed to assess the deviation from the expected rank.

Similarly, to what was observed in mouse data alone, the combined human and mice immunogenic data showed and enrichment for inflammatory pathways (interferon, NF-κb, interleukin, RELA) (FIG. 21C). Down regulation of EC proliferation pathways was observed, which, once again, highlight the importance of proper EC function versus out of control neovascularization. Interestingly many TF involved in immune surveillance and T cell recruitment are upregulated (FIG. 21E) such as the STAT TFs which are intracellular TF that mediate many aspects of cellular immunity, proliferation, apoptosis and differentiation through interferon signaling. Defects in STAT signaling can lead to increased susceptibility to infections while overexpression has been linked to autoimmune diseases. This highlights their key role in immune recruitment.

Meanwhile, in non-immunogenic tumors an enrichment was observed for endothelial proliferation pathways (VEGF, Angiotensin) (FIG. 21D). Similarly, expression of BCLAF1 and CEBPB have been shown to promote angiogenesis by controlling the expression of the hypoxia inducible factor-1α (HIF-1α) (FIG. 21F). The presence of GATA3 is also of particular interest as GATA3 has been linked to the inhibition of Ang-1-Tie2 signaling, thus contributing to endothelial cell dysfunction. The upregulation of post-transcriptional silencing by small RNAs also suggest a disruption of proper VEC function.

Finally, it is of interest to note that EGR1 and ZFP36 are both upregulated in immunogenic tumors VECs even though they have diametrically opposed effects. EGR1 has been well documented as a key mediator to induce the expression of cytokines and growth factors. EGR1 targets genes related to inflammation in vasculature, more specifically TECK and IP-30. TECK is a CC chemokine that functions as a chemoattractant for lymphocytes and IP-30 was originally cloned as an interferon-regulated protein and suggested as playing an important role in IFN-induced inflammation. Meanwhile ZFP36 has been shown to control inflammation in EC by inhibiting the expression of pro-inflammatory mRNA transcripts. Those opposing effects could be a key factor is explaining why immunogenic VECs are so efficient at T cell recruitment since ZFP36 could help keep inflammation under control while EGR1 enhances T cell recruitment. ZFP36 and EGR1 could therefore present great targets for induction in non-immunogenic VECs to increase T cell recruitment. Monoclonal antibodies that specifically recognize DARC (Duffy Antigen Receptor for Chemokines, a.k.a. ACRK1 or CD234) were used to study the distribution of VEC and T cells in healthy and diseased tissues. (See Thiriot et al.) More specifically, the DARC antibodies were used to elucidate the differences between VECs in immunogenic and non-immunogenic tumors environment as VECs regulate T cell infiltration in tissue.

First, a significant correlation between the number VEC and the number of CD8+ T cell in both immunogenic and non-immunogenic tumors in two mouse models, MC38 (immunogenic) and B16 (non-immunogenic) was established. This observation was further confirmed in human melanoma and pancreatic tumors. Furthermore, it became obvious that the number of VEC, and consequently T cells, was much lower in non-immunogenic tumors. Thus, the transcriptional differences between VECs in several tumor microenvironments were determined.

Using SeqWell, a microwell based scRNA-Seq technology, isolated ECs from healthy and tumor samples in both mouse and human were sequenced. After validating the protocol in healthy cells by controlling that usual EC expression patterns could be observed, this platform was used to compare MC38 and B16 VECs. This comparison revealed that (1) regardless of the microenvironment tumor, VECs upregulated hypoxia, angiogenesis and proliferation pathways, (2) immunogenic VECs closely resembled healthy VEC and (3) inflammation pathways (interferon and virus response) were upregulated in immunogenic VECs, thereby promoting T cell recruitment.

Finally, human melanoma (immunogenic) and pancreatic tumor (non-immunogenic) samples in combination with the mouse models were used to identify a common transcriptional signature for immunogenic VECs. They appear to be characterized by the upregulation of STAT and EGR1 transcription factor, which both promote T cell recruitment through the expression of specialized chemokines. Immunogenic VECs also express high levels of ZFP36, which might help keep inflammation under control while maintaining active T cell recruitment. This data suggests that these transcription factors could be targeted in non-immunogenic VECs to improve T cell infiltration in those tumors.

These data provide a snapshot of the anti-tumoral immune response, which is also determined by other factors such as intratumoral T cell proliferation, survival and egress into draining lymphatics.

### Methods

### Tumor Implantation and Harvest

Tumor cells were cultured in DMEM supplemented with 10% FBS, 1% of glutamine, penicillin/streptomycin, HEPES (1M stock), sodium pyruvate and non-essential amino acids unless cells were about 75% to 80% confluent. Cell suspension for tumor implantation were prepared at al density of 1×10\hat{ }6 cells per 50 ul for MC38 and B16F10 and 1×10\hat{ }5 cells and 1×10\hat{ }5 cells per 30 ul for murine pancreatic tumor models. MC38 and B16F10 tumors were implanted subcutaneously in the dorsal area of the mouse. KPC and Panc02 tumors were implanted orthotopically in the pancreatic tail by making a small incision and injecting 30 ul in the extravasated pancreas. The pancreas is returned into place and the skin sutured together. All tumors were implanted in C57B16J mice and in RAG KO for functional studies, these mice were purchased from Jackson Laboratories. All tumors harvested were about 100 to 200 mg. The peritumoral tissue were careful removed, tumors, peritumoral tissue and healthy tissue from non-tumor bearing mouse were analyzed separately.

### Flow Cytometry and Cell Preparation for Single Cell RNA Sequencing

### Digestion:

Harvested tissue were minced and incubated in digestion media containing a combination of collagenase, DNase, dispase and hyaluronidase with 2% of fetal bovine serum. Sample were incubated on an orbital shaker at 37 C for 10 to 30 mins depending on the density of the supernatant which was transferred to a secondary tube on ice containing enriched media. Undigested tissues bits were put through another digestion cycle for 10 to 20 mins and this process was repeated until all tissues were digested.

### Single Cell RNA Seq Samples for Seq Well:

Non-enriched samples were transferred directly onto arrays for sequencing. For cell enrichment, CD45 positive cells were depleted by staining the samples with biotinylated anti-CD45 antibody followed by incubation with Dynabeads Biotin Binder (Cat #11047) as per manufacturers protocol. The supernatant was stained with anti-CD31 followed by incubation with Dynabeads, the supernatants were discarded and beads-bounded cells were washed and loaded onto seq well arrays for sequencing.

### Flow Cytometry Analysis

Cells were treated with Fc block followed by staining with either mouse or human antibodies for immune cell and endothelial cell profiling. Anti-Ter 19 (for mouse) or anti-CD235a/b (for human) antibodies were used to gate out red blood cells. Samples were also stained with antibodies against CD45, CD11b, CD11c, CD31, gp38, DARC, CD3, CD8b and CD4. For endothelial cell subsets, CD45+ter119+(or CD235a/b) cells were gated out and CD31+gp38− cells were selected for blood endothelial cells (BEC) followed by gating on CD31+ and DARC+ for the venular endothelial cell (VEC) subset and CD31+DARC− non-venular endothelial cell subsets (NVEC) as described in Thiriot et al. BMC biology (10). For CD8 T cell gating, CD45+CD3+CD4−CD8b+ T cells were selected.

### Functional Adoptive Cell Transfer Experiment

Tumors were implanted in RAG KO mice as described above. Splenocytes from b-actin GFP mice were harvested and differentiated into effector CD8 T cells as described in (Weninger et al. JEM 2001). 5×10\hat{ }6 GFP+CD621−CD8b+CD44+ cells were transferred via retro-orbital route in RAG KO mice bearing tumors. Four hours after transfer, tumors were harvested as described above, digested and prepared for flow cytometric analysis as describe above and by gating on CD45+CD3+GFP+CD8b+ T cells, T cell homing in tumors was assessed.

### Single-Cell Transcriptional Profiling

High-throughput single-cell mRNA sequencing by Seq-Well was performed on the single-cell suspensions as described above. Approximately 20,000 viable cells per sample were applied directly to the surface of a Seq-Well device. Depending on sample sizes 1, 2, 3 or 4 arrays were run for each sample.

### Sequencing and Alignment

Sequencing for all samples was performed either on an Illumina NovaSeq or an Illumina NextSeq. No batch effect related to the type of sequencer used was observed. Reads were aligned to the Mus musculus genome (mm10) or the Homo sapiens (hg19) genome using STAR, and the aligned reads were then collapsed by cell barcode and unique molecular identifier (UMI) sequences using DropSeq Tools v.1 to generate digital gene expression (DGE) matrices, as previously described. To account for potential index swapping, all cell barcodes from the same sequencing run that were within a hamming distance of 1 were merged.

### Analysis of Single-Cell Sequencing Data

For each array, the quality of constructed libraries was assessed by examining the distribution of reads, genes and transcripts per cell. For each sample, dimensionality reduction (PCA) and clustering was performed as previously described. Results were visualized in a two-dimensional space using UMAP, and annotated each cluster based on the identity of highly expressed genes. Prior to any further analysis doublets were removed from each sample using DoubletFinder as previously described. Normalization, RNA counts regression, clustering and further gene analysis such as violin plots, heatmaps, module scoring, differential expression . . . etc. were performed using Seurat tools unless specified otherwise. In some cases cluster identity was further assessed using module scoring. The gene lists used to define the modules are in Table 9. After EC isolation, to further characterize substructure within EC, dimensionality reduction (PCA) and clustering over those cells alone were performed as previously described. Results in two-dimensional space using UMAP were visualized. Clusters were further annotated (that is, as endothelial cells subsets, such as venular cells, capillary cells . . . etc) by cross-referencing cluster-defining genes with curated gene lists or by using module scoring.

### B16 VEC Isolation Strategy

For most samples, a VEC cluster was identified. However, this wasn't the case for B16 as B16 VECs were too similar to B16 NVECs. To identify VECs, VEC scoring based on the gene list in Table 9 with a 0.2 cutoff value was used. To define a score cutoff the B16 data matrix was shuffled and randomized first, then a sample of 100 cells was taken, their VEC score was calculated and the score value for the 95 quantiles was recorded. This process was repeated 10 times over 50 different permutations of the data matrix. The 95 quantiles value was averaged over all these iterations and used as the cutoff.

### Silhouette Analysis

Silhouette can be used to define the proximity between clusters by scoring the similarity between cells of those clusters. Here for each cell in a given cluster the silhouette algorithm was used to define a closest neighbor cluster (i.e. the cluster the cell should belong to if it didn't belong to the cluster it is currently in). Similarities between clusters were assessed by looking at the percentage of cells from one cluster reassigned to other clusters.

### GSVA Analysis

Gene Set Variation Analysis (GSVA) was used by using a collection of expert annotated vascular-related gene sets from the Molecular Signatures Database (MSigDB version 5.2) to identify pathways and cellular processes enriched in different samples. GSVA was performed as implemented in the GSVA R-package (default parameters), where the gene-by-cell matrix is converted into a gene-set-by-cell matrix. The difference between the GSVA enrichment scores from each sample was analyzed by using a simple linear model and moderated t-statistics computed by the limma package using an empirical Bayes shrinkage method. Using a p=0.01 cutoff, the differentially activated pathways between samples were examined.

### EnrichR Analysis

Genes differentially expressed between MC38 VECs and B16 VECs or melanoma VECs and pancreatic tumor VECs were determined using Seurat, as described above. These lists were compared to look for common elements. The resulting list of common elements was used for further gene set analysis with EnrichR. The EnrichR R package provides an interface to the EnrichR database. Two databases were selected: “BioPlanet_2019” for pathways and “ENCODE_TF_ChIP-seq_2015” for transcription factors and looked for enrichment. Full enrichment tables in Tables 13 and 14. The transcription factor analysis was repeated using “ChEA_2016”, “ARCHS4_TFs_Coexp”, “ENCODE_and_ChEA_Consensus_TFs_from_ChIP-X”, “Enrichr_Submissions_TF-Gene_Coocurrence”, and “TRRUST_Transcription_Factors_2019” databases, similar enrichment were observed.

### Example 2: Characterize Expression of Transmembrane Proteins on Microvascular ECs of Solid Tumors

A hallmark of solid tumors is the formation of new vasculature (angiogenesis). This process is required to support tumor growths beyond a few millimeters in size due to the limit of oxygen and nutrient diffusion within neoplastic tissues (Folkman, J. 1971. N Engl J Med 285: 1182-1186). Tumor neovasculature is often poorly adhesive for blood-borne T cells, which is thought to present a major impediment to T cell dependent immunotherapy (Peske J D, Woods A B, Engelhard V H. Adv Cancer Res. 2015; 128:263-307). Normal microvasculature, which consists of a network of functionally specialized vessels, including arteries, arterioles, venules and veins, which are all connected by a common capillary network was observed. Arteries and arterioles regulate blood flow, while gas and nutrient exchange takes place at the capillary level. Using intravital microscopy, it has been previously shown that the recruitment of blood-borne leukocytes is invariably restricted to postcapillary and collecting venules, whereas capillaries and arterioles do not support leukocyte adhesion (Halin, C., J. Rodrigo Mora, C. Sumen, and U. H. von Andrian. 2005. Annu Rev Cell Dev Biol 21: 581-603). There is strong evidence suggesting that this functional distinction among microvessels is due to segmental specialization of endothelial cells (ECs), not hemodynamic differences (Ley, K., and P. Gaehtgens. 1991. Circ Res 69: 1034-1041). Indeed, microvascular specialization is already apparent during embryogenesis before the initiation of blood flow (Lawson, N. D., and B. M. Weinstein. 2002. Nat Rev Genet 3: 674-682). Both in uninflamed microvessels that constitutively recruit leukocytes, and in acutely or chronically inflamed peripheral tissues, venules are the exclusive port of exit for blood-borne leukocytes that access the extravascular compartment. Accordingly, multiple studies have shown that most leukocyte adhesion receptors are restricted to venular ECs (VECs), although the expression of these molecules is not uniform in different vascular beds (von Andrian, U. H., and C. R. Mackay. 2000. N. Engl. J. Med. 343: 1020-1034). Several molecules have been identified that specify the differentiation of blood ECs and lymphatic ECs (LECs) and contribute to EC proliferation in tumors (Rocha, S. F., and R. H. Adams. 2009. Angiogenesis 12: 139-147; Oliver, G., and R. S. Srinivasan. 2010. Development 137: 363-372), but the mechanism(s) that render(s) VECs uniquely capable of supporting leukocyte trafficking remain(s) a mystery.

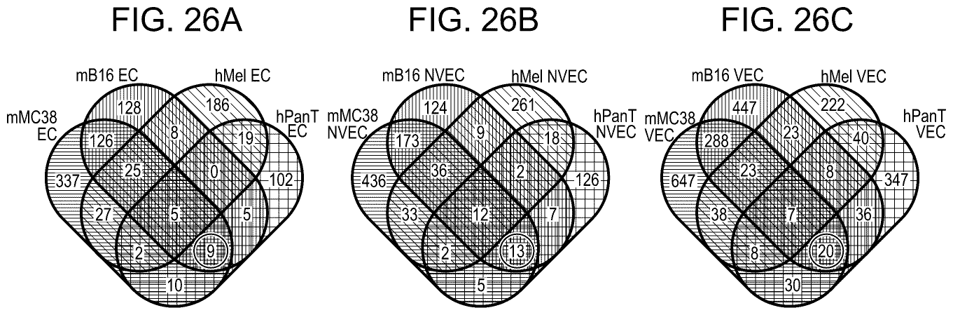

To address this issue, a monoclonal antibody (mAb) against DARC (ACKR1), which selectively recognizes VECs in normal murine tissues was used (Thiriot, A. et al. BMC Biology, 2017 May 19; 15(1):45). In recent experiments, using this mAb as well as a commercial mAb against human DARC, primary VECs, non-venular ECs (NVECs) and L-ECs were isolated from a variety of murine and human non-malignant tissues to compare EC subsets at the transcriptome and proteome level. In addition, single-cell RNAseq was used to compare EC transcriptomes of two subcutaneous murine tumors (MC38 colorectal adenocarcinoma and B16F10 melanoma) and fresh patient-derived human melanoma and pancreatic cancer. For each tumor, VECs and NVECs from peri-tumoral non-malignant tissue were analyzed. MC38 was used because it is an immunogenic tumor (T-cell rich and respond to checkpoint blockade) and B16F10 melanoma because it is a non-immunogenic (T-cell poor and do not respond to checkpoint blockade). Several endothelial genes, including genes encoding cell surface molecules that are uniquely upregulated in the tumor microvasculature were identified (FIGS. 26A-26C).

The mechanisms that enable venular ECs to recruit leukocytes but prohibit capillary and arteriolar endothelium to do so are entirely unknown. The differences between the venular phenotype of an immunogenic tumor and a non-immunogenic tumor is also not known. Identifying gene products that specify endothelial “venuleness” represent a novel class of attractive targets for tumor-specific EC targets and venular inducers for onco-immunotherapy. The only current treatments targeting tumor vasculature aim to inhibit angiogenesis by targeting VEGF, but this approach does not promote venular differentiation. There are no FDA-approved anti-tumoral drugs that can selectively boost endothelial cell dependent immune cell recruitment. This invention emerges from a proprietary discovery platform to generate novel anti-tumoral therapy that aims to differentiate non-adhesive endothelium within tumors into venular endothelium and identify endothelial cells (EC) specific surface markers for targeted treatments of solid tumors with minimum off target effects. Since VECs are the principal gatekeepers for leukocyte emigration, drugs that are able to target the intra-tumoral venular segment can be used to promote VEC differentiation could potentially boost tumor infiltration by T cells and thus enhance onco-immunotherapy. Therefore, the neovasculature of solid tumors may be inherently suboptimal at recruiting T cells because of inadequate endothelial differentiation into functional venular type microvessels. The present invention disclosure provides a proprietary discovery platform that will lead to a new generation of drugs that specifically target clinically relevant plasma membrane molecules from venular and non-venular endothelium, in both murine and human solid tumors for the targeted delivery of therapeutics with minimum off target effects. Additionally, it reveals the transcription programming of venular endothelial cells from immunogenic tumors (which are poised for immune cells recruitment), the molecules identified are not just restricted to plasma membrane but also includes novel transcription factors, miRNA and long non-coding RNA and list of genes that confer the programming needed to allow immune cells to extravasate into tumors.

The disclosed invention comprises lists of clinically relevant plasma membrane molecules that are overrepresented in all endothelial cells (Table 1) and specific segment of the vasculature such as venular endothelial cells (Table 2) and non-venular endothelial cells (Table 3) from murine and human tumors compared to their respective non-malignant tissues.

A more comprehensive list of genes including but not limited to transcription factors (TF), extracellular molecules, cytoplasmic molecules including miRNA. The tumor specific endothelial plasma membrane molecules will be use as targets for 1.) intra-tumoral specific gene delivery to venular endothelial cells to induce a venular programming (TF identified from the scRNA sequencing) that will increase intra-tumoral CD8 T cells and 2.) targeted delivery of therapeutics (CAR-T cells, TIL therapy, therapy comprising a cell expressing an antigen recognizing a tumor antigen, checkpoint blockade therapy and tumor specific chemotherapy delivery) with minimum off target effects. Molecules that are unique to human melanoma and/or human pancreatic tumors were identified. Many of these molecules are also upregulated in murine melanoma and/or colorectal adenocarcinoma tumor models, these molecules could be used for in vivo studies to characterize and demonstrate its tumor specificity with minimum off target effects and its ability to change the microvascular phenotype by increasing the “venuleness” of the venular segment of the vasculature. This will allow for increase immune cell recruitment in solid tumors. Molecules that are only over-represented in human tumors and not murine tumors will also be evaluated in other murine tumor models and other fresh human neoplastic tissues. Molecules that are in both human tumors could be also over-represented in other human solid tumors. The tables above list novel and unique plasma membrane molecules identified in human tumors. Other molecules identified are not just restricted to plasma membrane but also includes novel transcription factors, miRNA and long noncoding RNA and list of genes that confer the programming needed to allow immune cells to extravasate into tumors.

### Example 3: Characterize PMEPA-1 Expression on Microvascular ECs of Murine and Human Solid Tumors

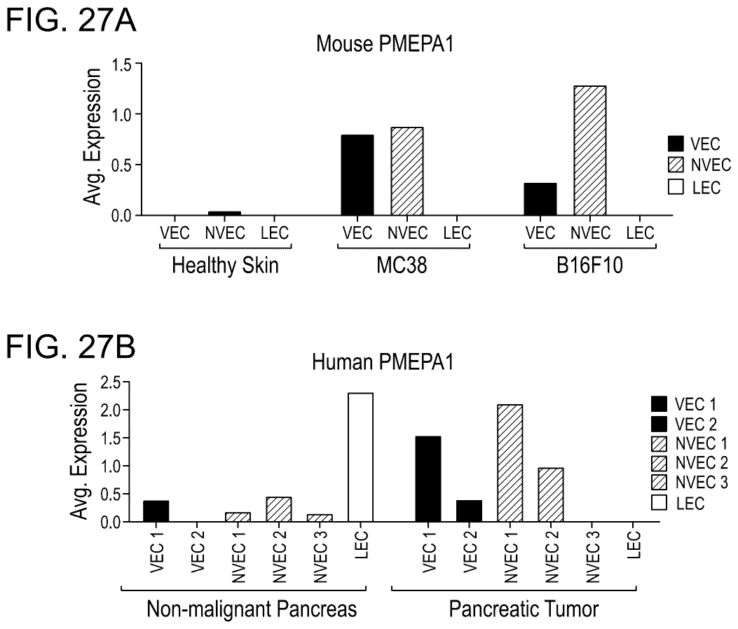

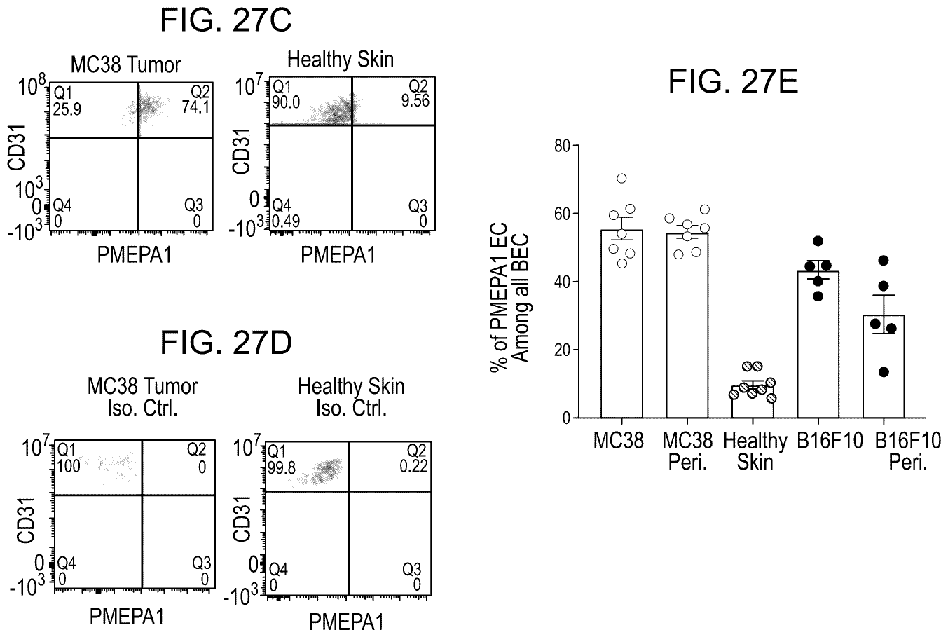

To determine PMEPA-1 mRNA and protein expression and localization in primary human and murine tumors qPCR, FISH, flow cytometry and histology will be used. For murine tumor models, syngeneic colorectal adenocarcinoma (MC38) and melanoma (B16F10) tumor models were used, whereby tumor cells are implanted subcutaneously in the dorsal skin. MC38 is an immunogenic tumor with high T cell infiltrates (TILs) and responds to checkpoint blockade while B16F10 has the opposite characteristics. In FACS analysis of ECs from subcutaneously implanted syngeneic murine MC38 and B16F10 tumors, 74.1% of intratumoral ECs were PMEPA-1+, whereas only 9.6% of ECs in normal skin expressed PMEPA-1, and only at a very low level (FIGS. 27A-27E). For FACS analysis, the gating strategy for PMEPA-1+ events was based on a polyclonal control antibody (Iso). The apparent basal level of PMEPA-1 in healthy murine skin could reflect high background binding that is typical for many polyclonal Abs rather than true protein expression (FIG. 27A-27E). Indeed, healthy skin does not appear to express PMEPA-1 at the mRNA level (FIG. 27A). PMEPA-1 expression in fresh samples of human melanoma and peri-tumoral non-malignant skin, as well as, human pancreatic cancer and non-malignant pancreas at the protein level by flow cytometry and IHC were accessed.

### Example 4: Yeast Display sdAb Library to Generate and Validate sdAbs Against the PMEPA-1 Ectodomain

Among these tumor EC restricted genes is Prostate Transmembrane Protein, Androgen Induced 1 (PMEPA-1), a regulator of tissue responses to cytokines. PMEPA-1 mRNA was significantly upregulated in ECs from MC38, B16F10 and human pancreatic cancer samples compared to ECs in nonmalignant peri-tumoral tissue (FIGS. 26A-26B and 27A-27E). PMEPA-1 is also upregulated in human melanoma compared to healthy skin, however, it did not reach statistical significance in the analysis (red circles in FIG. 26A-26B). Additionally, PMEPA-1 is present in 17 types of solid human tumors according to The Cancer Genome Atlas (TCGA) database. In addition to TCGA data, computational analysis of publicly available data set (S. Can et al, 2023, bioRxiv; Y. Xie et al, 2021, JCI Insight) revealed high PMEPA-1 expression on microvessels in human glioblastoma. Although EC expression of PMEPA-1 in these human tumors remains to be validated, it appears to be an attractive candidate to target ECs in a wide variety of tumors.

PMEPA-1 mRNA and protein expression levels in normal and malignant human and murine tissues was validated. Mouse and human PMEPA-1 have a single transmembrane domain and an ectodomain with 79% aa homology. While there are no reagents that specifically recognize this ectodomain, a commercially available polyclonal antibody to the cytoplasmic tail was used to validate at the protein level that PMEPA-1 is preferentially expressed on tumor ECs (FIGS. 27A-27E).

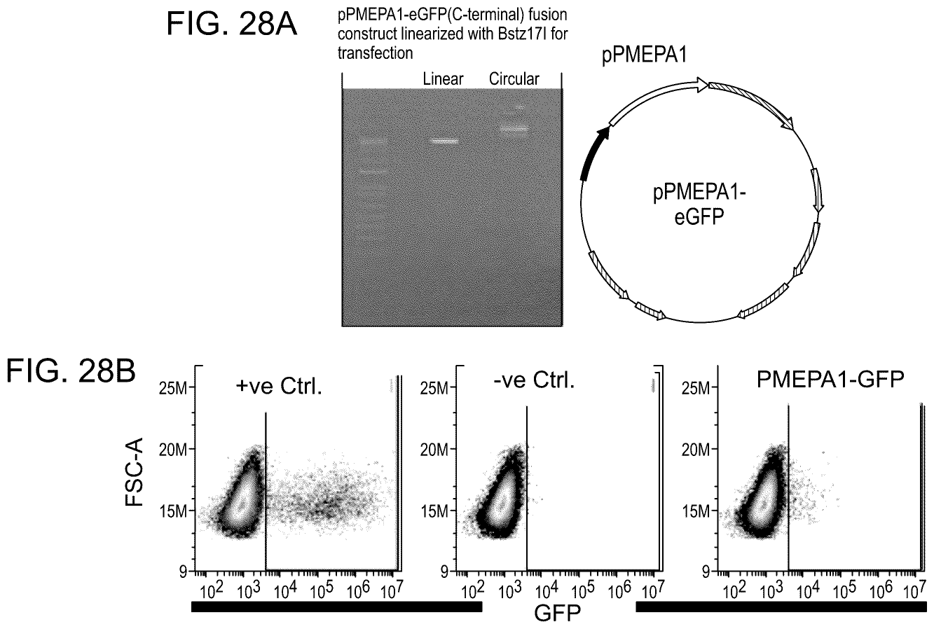

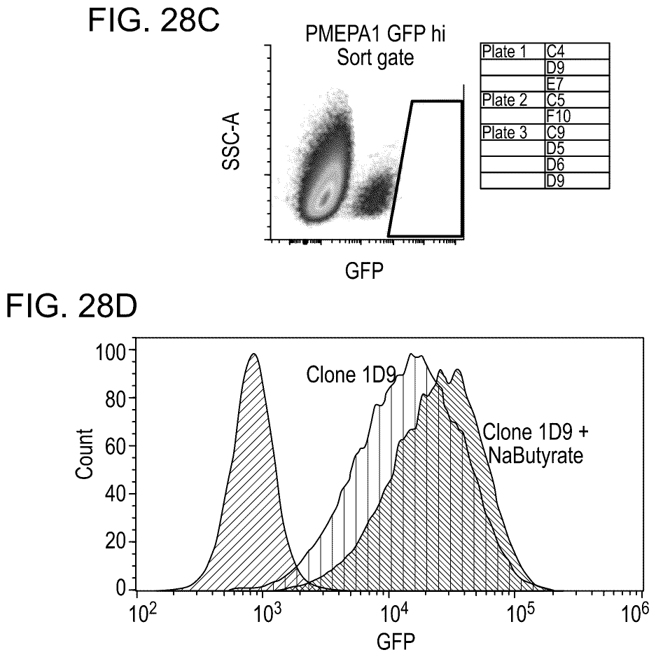

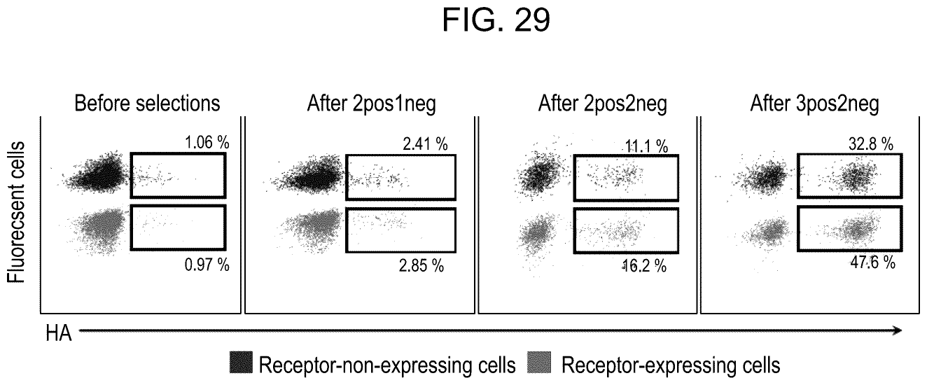

A yeast display library was used to raise sdAbs against human and murine PMEPA-1 transfectants (FIGS. 28A-28D). Stably transfected L1.2 cells that express PMEPA-1 fused with intracellular GFP were used. As negative and positive controls, L1.2 cells were transfected with empty vector or GFP alone (FIGS. 28A-28B). PMEPA-1-GFP high cells were FACS sorted and subcloned by limiting dilution in 96-well plates and expanded in selection medium containing G418 (FIG. 28C). Clones which displayed consistently the highest mean fluorescence intensity were further expanded. Clone 1D9 that demonstrated the highest PMEPA-1-GFP expression, which could be further enhanced by addition of sodium butyrate, an HDAC inhibitor, to the culture medium was selected (FIG. 28D). This clone is ready for use as ‘bait’ for the yeast display library to identify sdAb with reactivity against the ectodomain of PMEPA-1. FIG. 29 shows an example where the yeast library was subjected alternatingly to three and two cycles of positive and negative selection, respectively. sdAb mediated yeast binding to target cells is readily detectable by FACS because sdAb expressing yeast cells coexpress a surface epitope from hemagglutinin (HA).

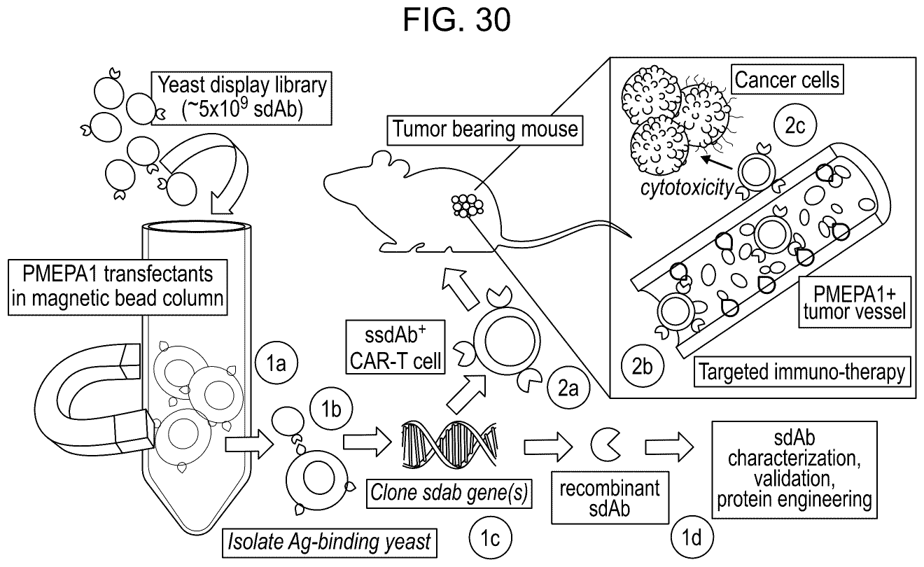

The yeast display library approach is based on performing a series of alternating magnetic-activated cell sorting (MACS)-based positive and negative selection steps followed by fluorescence-activating cell sorting (FACS)-based sorts (FIG. 30, Step 1a). For positive selections, PMEPA-1-GFP expressing L1.2 cells will be labeled with anti-CD45 magnetic beads and loaded on magnetic columns. Next, sdAb-expressing yeast (which contains ˜5×109 distinct sdAb clones) will be loaded on the same columns, and columns will be washed extensively. The contents of the columns will be retrieved, and yeast bound to L1.2 cells (determined as HA+ cells) will be sorted. Thus, the clones expressing relevant sdAb will be enriched. Each positive selection is followed by a negative selection cycle, whereby PMEPA-1-negative L1.2 cells will be loaded on magnetic columns, followed by loading of sdAb-expressing yeast. The columns will be washed, and the unbound fraction will be collected. and HA+ yeast cells will be sorted. Yeast clones that bind to irrelevant surface antigens on L1.2 cells remain in the column and are eliminated (FIG. 30, Step 1b). Repeated cycles of positive and negative selection will result in decreased sdAb library diversity and increased efficiency of formation of cell-yeast conjugates, with enrichment for yeast clones that preferentially bind PMEPA-1-expressing L1.2 cells. Upon reaching a high frequency of cell-yeast conjugates, the yeast sdAb library will be subcloned (FIG. 30, Step 1c), and the clones with maximum binding to PMEPA-1-expressing L1.2 cells and no binding to control L1.2 cells will be identified. The sdAb encoding cDNAs will be subcloned into an expression vector and modified to append an N-terminal FLAG tag for protein purification and/or a C-terminal LPETG motif to allow for sortase A-mediated “click chemistry” linkage to acceptor moieties of interest. Recombinant sdAb will be expressed in E. coli and extensively characterized for reactivity with PMEPA-1 in vitro (FACS, Western blot) and in situ using IHC and/or intravital microscopy of microvessels in tumors and non-malignant tissues at various anatomical location (FIG. 30, Step 1d). In addition, sdAbs will be engineered to allow surface expression/immobilization on CAR-T cells to test their ability to selectively target tumor ECs (FIG. 30, Steps 2a-2c).

### Example 5: Demonstration of Tumor Vessel Imaging In Vivo Using Nanobodies Against PMEPA-1

A composition comprising an anti-PMEPA-1 binder for targeting PMEPA-1 on endothelial cells, a hydrazone linker capable of hydrolyzing in tumor microenvironments, a Cobalt-57 radioactive isotope, is intravenously injected into tumor bearing mice to assess targeting effectiveness and imaging efficacy by PET. 60 minutes after IV administration, the tumors are harvested and subjected to immunohistochemistry and ICP-MS analysis to determine metal content.

In addition, a composition comprising an anti-PMEPA-1 binder for targeting PMEPA-1 on endothelial cells, a hydrazone linker capable of hydrolyzing in tumor microenvironments, a Cobalt-57 radioactive isotope, is intravenously injected into tumor bearing mice to assess targeting effectiveness and imaging efficacy by PET. 60 minutes after IV administration, they are imaged by SPECT and PET scan to assess distribution and targeting efficacy of the composition comprising the binder.

This example will demonstrate that the radiopharmaceutical composition described herein selectively binds to endothelial cells in tumor microenvironment and can serve as an imaging agent for tumor detection.

### Example 6: Demonstration of Tumor Vessel Imaging and Therapeutic Agent Delivery In Vivo Using Nanobodies Against PMEPA-1

A composition comprising an anti-PMEPA-1 binder for targeting PMEPA-1 on endothelial cells, a hydrazone linker capable of hydrolyzing in tumor microenvironments, a Cobalt-57 radioactive isotope, is formulated in an aqueous solution with a chemotherapeutic agent, methotrexate. The formulation is intravenously injected into tumor bearing mice to assess targeting effectiveness and imaging efficacy by PET. The targeting efficacy and imaging efficacy additionally should reflect the delivery of the methotrexate to the tumors of the mice. 60 minutes after IV administration, the tumors are harvested and subjected to immunohistochemistry and ICP-MS analysis to determine metal content.

In addition, a composition comprising an anti-PMEPA-1 binder for targeting PMEPA-1 on endothelial cells, a hydrazone linker capable of hydrolyzing in tumor microenvironments, a Cobalt-57 radioactive isotope, is formulated in an aqueous solution with a chemotherapeutic agent, methotrexate. The formulation is intravenously injected into tumor bearing mice to assess targeting effectiveness and imaging efficacy by PET. The targeting efficacy and imaging efficacy additionally should reflect the delivery of the methotrexate to the tumors of the mice. 60 minutes after IV administration, mice are imaged via PET and SPECT scanning to assess distribution and targeting efficacy of the formulation comprising the composition to the tumors of the mice.

This example will demonstrate that the radiopharmaceutical composition described herein selectively binds to endothelial cells in tumor microenvironment and can be used for cancer treatment.

### Example 7: Target CAR-T Cells with PMEPA1 sdAb to Tumor Microvessels and Assess Anti-Tumor Efficacy

Immobilized PMEPA1 sdAb on the surface of CART cells will be used to determine if these CAR T cells can be used as an effective targeted cell therapy. After IV infusion, the sdAb will enable CAR T cells to adhere selectively to tumor ECs that are normally non-adhesive for circulating T cells. Because of the immobilized PMEPA1 sdAb on the CAR T cells, it is expected that the CAR T cells will accumulate in solid tumors that are currently resistant to CAR T cell therapy. In particular, the lack of tumor targeting specificity of traditional CAR T cell therapies increases the risk for off-target effects and exhaustion. It is expected that sdAb-mediated targeting of such second-generation CAR T cells to tumors will further boost therapeutic efficacy.

The surface of the T cells that express a CAR specific for a tumor antigen will be decorated with PMEPA1 sdAb at a high density. In this setting, the sdAb will confer mechanical stability to CAR T cell binding to PMEPA1+ tumor microvessels, without transmitting an activating signal. As described above, at the transcriptional level, among all intravascular cells in mice only ECs lining the vasculature within tumors express robust levels of PMEPA1. Indeed, according to RNASeq data published by Immgen, among all murine cell types tested, the only healthy cells that express PMEPA1 are brain microglia (and, to a lesser degree, alveolar macrophages) (www.rstats.immgen.org/Skyline/skyline.html). After adoptive transfer, CAR T cell are unlikely to access the CNS because normal brain ECs do not express PMEPA1 and do not support substantial T cell trafficking. Therefore, sdAb decoration should focus CAR T cells onto tumor ECs without redirecting them to other cell types or anatomic sites. In humans, according to the human cell atlas website (www.humancellatlas.org), only a subset of PBMCs (Siglec6-CD123+CD11c-PBMC) from healthy patients had mRNA levels slightly above baseline. Detection of PMEPA1 has been reported only at the mRNA level in these databases, which does not always correlate with the presence of protein, especially if the RNA level is low. For the most rigorous analysis, a reliable antibody detecting the ectodomain of this transmembrane molecule is needed to allow for sensitive assessment by flow cytometry and immuno-histochemistry. Due to the absence of such a reagent, very few studies have been performed on this molecule to date.

Although it is expected that the sdAb-targeted CAR T cells will be highly selective for tumor ECs, there may be other PMEPA1+ target cells. However, even if this were the case, expression of PMEPA1 sdAb without a signaling domain should not cause increased toxicity as long as such hypothetical PMEPA1+ cells do not also express the CAR antigen or other means to activate CAR T cells.

A schematic of the proposed protocol is shown in FIG. 30. Briefly, CAR-T cells express chimeric Ag receptors (CARs) that link an extracellular Ag recognition component to an intracellular signaling domain resulting in T cell activation when a tumor Ag is encountered. CAR T cells will be generated against human CD20. These CAR T cells will be modified to display one or more PMEPA1 sdAbs on their surface by transfecting CAR T cells with chimeric sdAbs containing either a cytoplasmic and transmembrane domain and a linker region or a Gpi anchor (FIG. 30, Step 2a). The PMEPA1 sdAb constructs will not include an activating signaling domain. The sdAb may function as an anchor to immobilize CAR T cells within tumor microvessels upon adoptive transfer into tumor bearing mice. Thus, adoptive transfer experiments and in situ imaging in tumor bearing mice will be performed to determine whether surface displayed sdAbs enhance CAR T cell accumulation in tumors (FIG. 30, Step 2b). MC38 or B16F10 solid tumors will be transduced to express human CD20 with anti-human CD20 CAR T cells that will be surface modified either with anti-PMEPA1 sdAbs or a non-binding control sdAb. Assessment whether CAR T cell decoration with anti-PMEPA1 sdAb confers selective targeting of CAR T cells to tumors and enhancement of anti-tumor immunity will be performed (i.e. suppression of tumor growth, survival etc.) (FIG. 30, Step 2c).

The anti-PMEPA1 sdAb serving as the Ag binding domain of a CAR will also be tested to determine if T cell activation after recognition of PMEPA1 by the sdAb-CAR results in tumor EC killing. In this setting, the CAR T cells would exert cytotoxic activity towards tumor ECs, destroying the tumor by going after these vital stromal cells rather than the tumor cells themselves. While this approach would likely result in rapid killing of host tumors since CAR T cells will initially be present at a high density in the blood stream, there may be a greater risk for on-target off-tumor side effects due to recognition of PMEPA1 on cells other than intra-tumoral ECs. Thus, recipient animals' health and potential organ damage will be monitored. If off-target toxicity is unacceptable, it could offer a powerful new treatment modality for solid tumors because the CAR T cells could function within the tumor vessel lumen, without the need to extravasate.

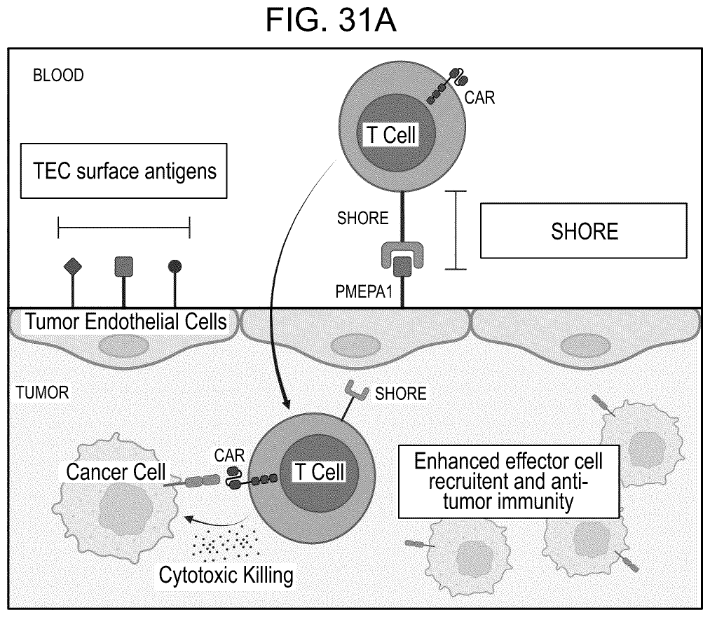

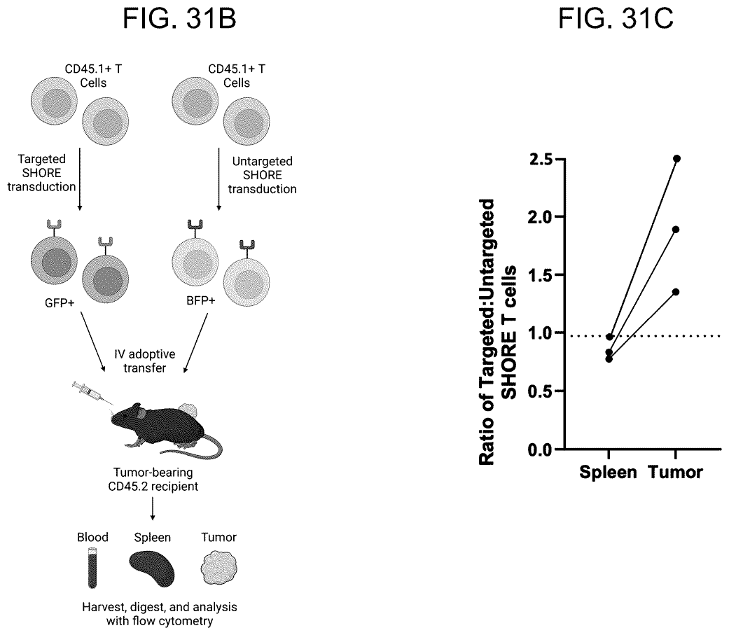

To assess the capacity of SHORE T cells to selectively drive trafficking into tumor parenchyma, the relative accumulation in tumors of PMEPA1-targeted SHORE T cells was compared to untargeted, nonspecific SHORE T cells was compared through a competitive adoptive transfer experiment. Primary T cells were isolated from CD45.1 splenocytes and transduced with either PMEPA1-targeted SHOREs and GFP or untargeted SHOREs and BFP and expanded in the presence of cytokines IL-15 and IL-7. The SHORE T cells were then injected intravenously into CD45.2 recipient mice bearing subcutaneous MC38 tumors 14 days after tumor implantation. Twenty hours after adoptive transfer, the tumor, spleen, and blood were taken for digestion and analysis via flow cytometry (FIG. 31B). Comparative analysis revealed a relative enrichment of targeted SHORE T cells to untargeted SHORE T cells in the tumor relative to the ratio detected in the spleen, suggesting that the PMEPA 1-targeted SHOREs selectively increased tumor infiltration (FIG. 31C).

### Example 8: Demonstration of Therapeutic Agent Delivery In Vivo Using Bispecific Antibodies

A composition comprising an anti-PMEPA-1 binder, anti-CEA binder, and a hydrazone linker capable of hydrolyzing in tumor microenvironments is formulated in an aqueous solution with a chemotherapeutic agent, methotrexate. The formulation is intravenously injected into tumor bearing mice to assess targeting effectiveness. The targeting efficacy should reflect the delivery of the methotrexate to the tumors of the mice. 60 minutes after IV administration, the tumors are harvested and subjected to immunohistochemistry and ICP-MS analysis to determine metal content.

This example will demonstrate that the composition described herein both binds to PMEPA-1 and CEA and can be used as an effective therapeutic agent for cancer treatment.

### Example 9: Demonstration of Tumor Vessel Targeting In Vivo Using Antibody Drug Conjugates

Antibody drug conjugates comprising an anti-PMEPA-1 binder linked to MMEA is intravenously injected into tumor bearing mice (MB49 model) on Day 12. Five minutes after IV administration, the tumors are harvested and subjected to immunohistochemistry. As expected, the antibody drug conjugates described herein is accumulated in the tumor vessels in the mouse, but not accumulate in the vessels in the periphery. Thus, the antibody drug conjugates described herein specifically targets the tumor vasculature in the living mouse.

These data not only demonstrate the utility of the above gene lists in the identification of targetable tumor-specific genes, but also the ability to generate highly specific immunoreagents targeting these identified genes, such as PMEPA-1.

### Example 10: Alternative Cell-Free Approach on Generation of sdABs Against Human and Murine PMEPA-1 from Yeast Display Library and Validation of their PMEPA-1 Ectodomain Specificity

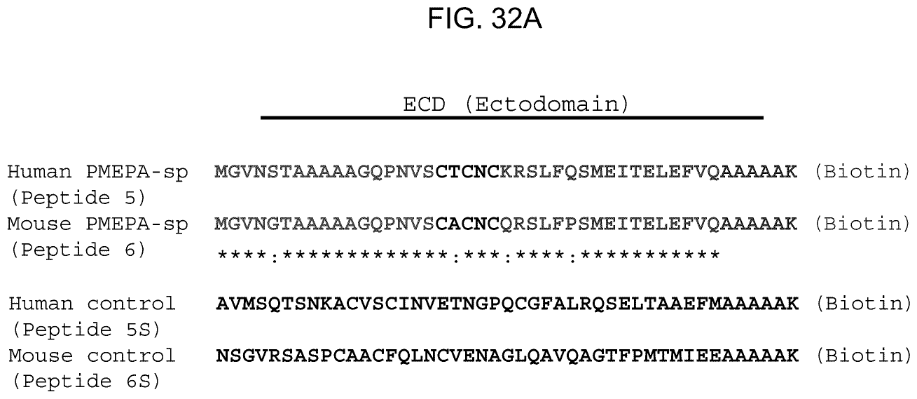

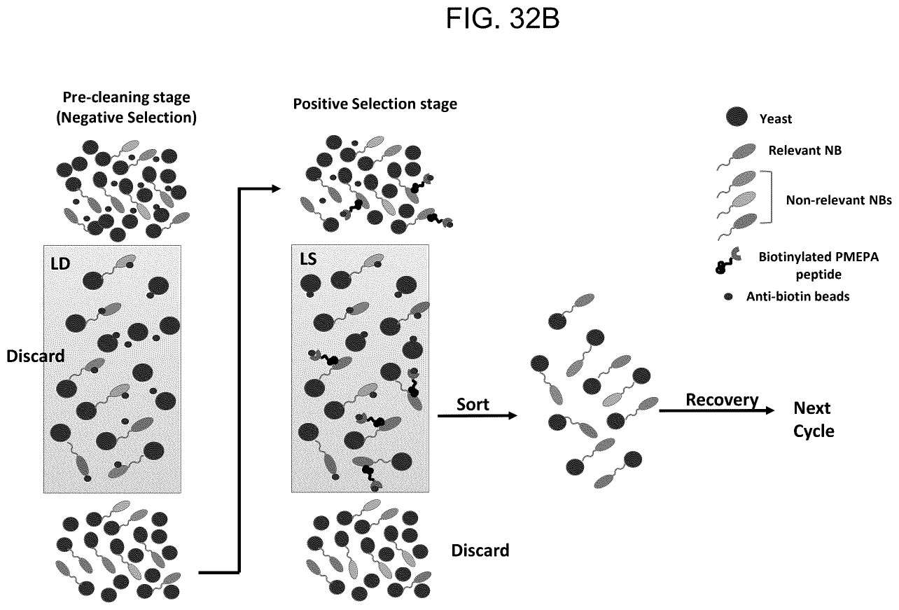

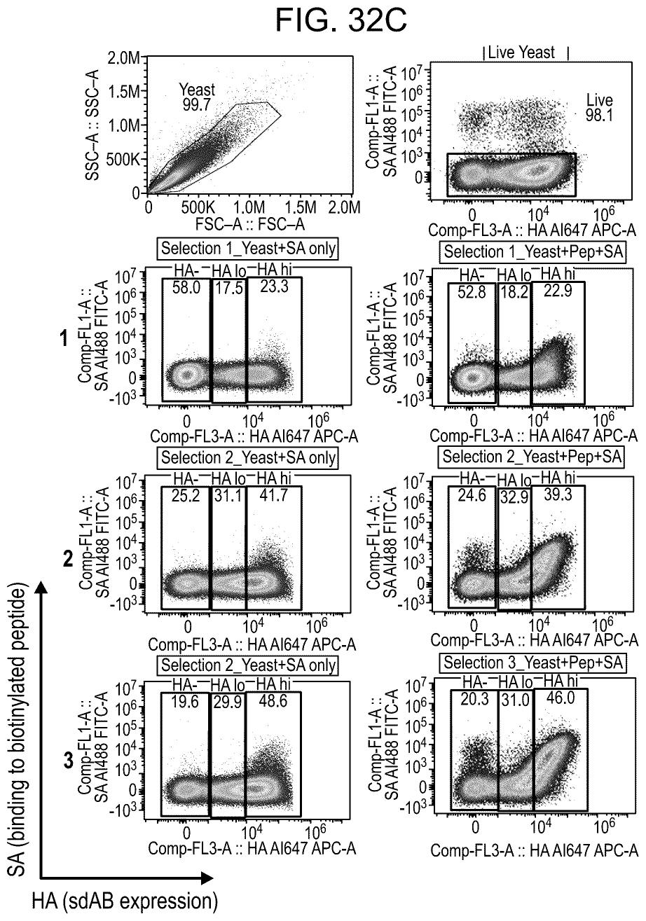

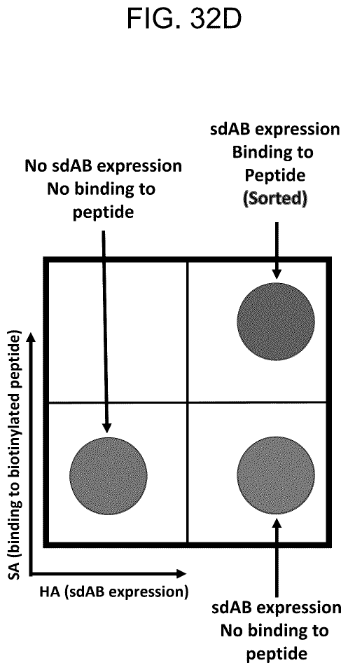

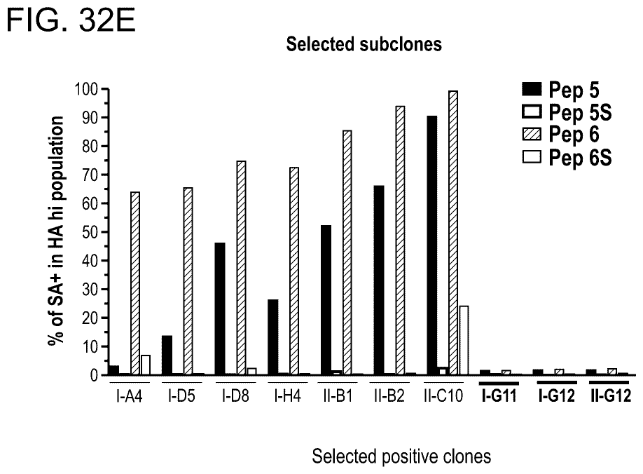

Synthetized peptides (each with 5× Alanine linker at C terminus, followed by biotinylated Lysine) corresponding to human and mouse extracellular domains of PMEPA-1 (90% identity), as well as control (scrambled) biotinylated human and mouse peptides were used (FIG. 32A). Initial selection rounds and FACS sort were performed based on binding of mouse PMEPA-1 specific peptide (Peptide 6) to sdAB-expressing yeast. Down the road, human PMEPA-1 specific peptide (Peptide 5) and control human and mouse peptides (Peptides 5S and 6S) were used for screening of subclones. Induction of sdAB expression in naive yeast display library was assessed by expression of sdAB epitope tag hemagglutinin (HA). Series of negative (using anti-biotin beads only) and positive (using a specific peptide and then anti-biotin beads) MACS purifications to enrich the sdAB yeast library for PMEPA-1 specific clones and decrease library diversity were performed (FIG. 32B). Binding efficiency to PMEPA-1 specific mouse peptide was assessed by FACS after each round of selection (FIG. 32C). After four rounds of selection, enriched sdAB library was incubated with biotinylated peptide, and HA+(sdAB-expressing) Streptavidin (SA)+(PMEPA-1-specific peptide bound) double positive clones were single cell sorted in 96 well plates by FACS sort (schematic of sort shown in FIG. 32D). All subclones were analyzed by FACS for binding to PMEPA-1 specific mouse (Peptide 6), PMEPA-1 specific human (Peptide 5) and control (scrambled) mouse (Peptide 6S) and human (Peptide 5S) peptides. Subclones with the highest binding to peptides 6 and 5 and no binding to peptides 6S and 5S were identified as “positive” subclones (IA4, ID5, ID8, IH4, IIB1, IIB2, IIC10). Subclones with the lowest binding to peptides 6 and 5 were identified as “negative” or “control” subclones (IG11, IG12, IIG12) (FIG. 32E).

After “positive” and “negative” subclones were identified and expanded, sdAB-encoding yeast plasmid was extracted and migrated into bacterial expression vector pET-26b(+). E. Coli was transformed with the construct, expanded, lysed and sdABs were purified by size exclusion FPLC chromatography.

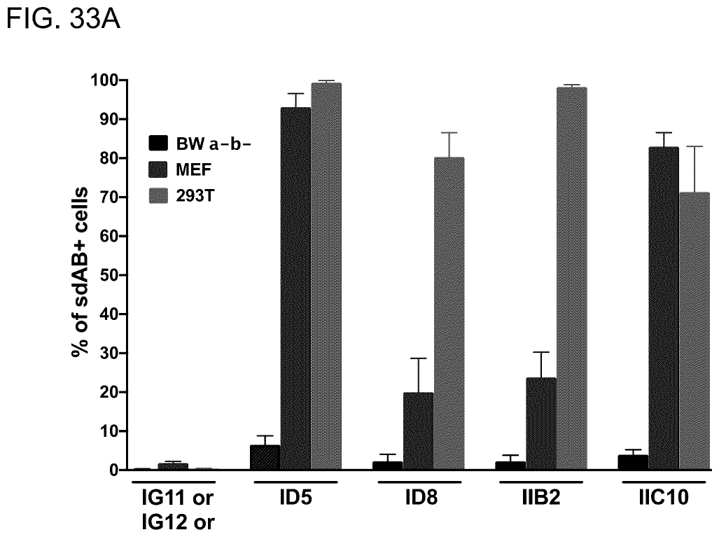

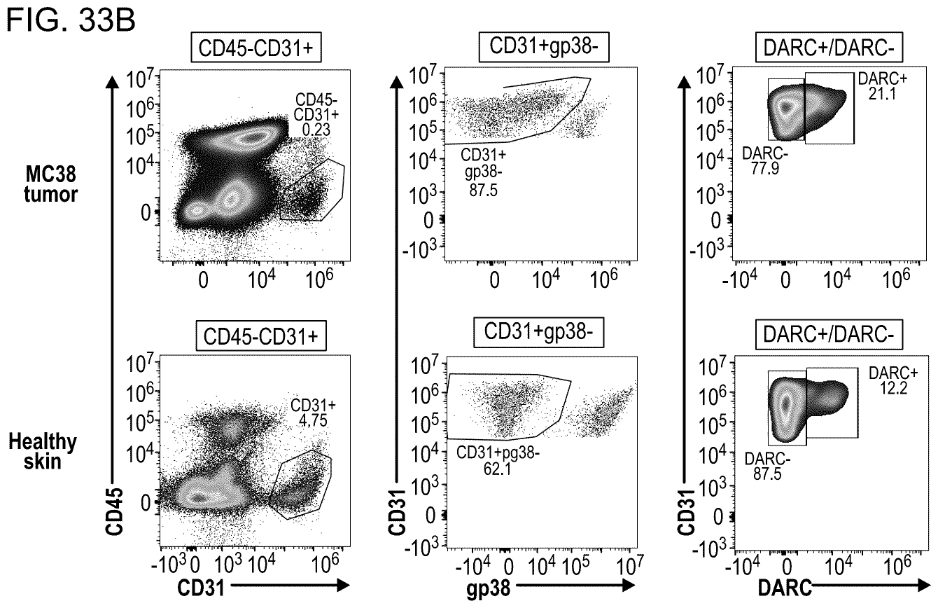

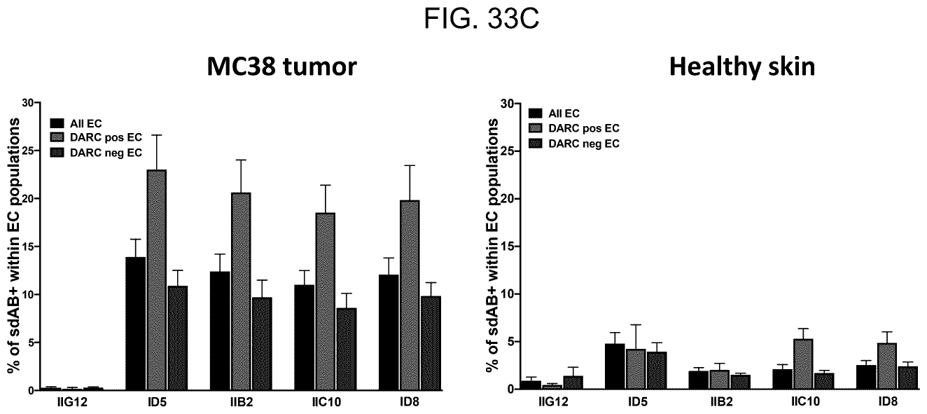

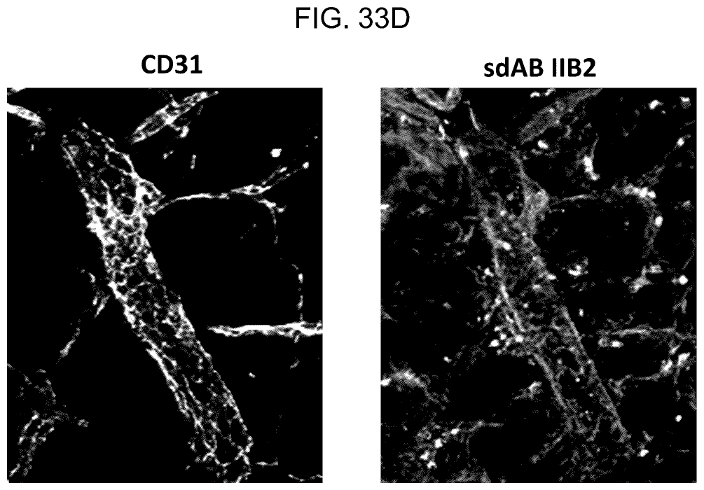

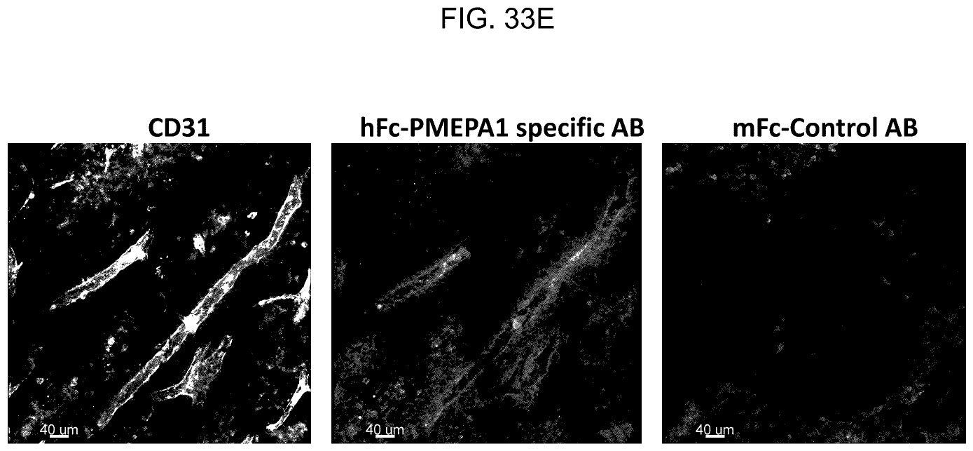

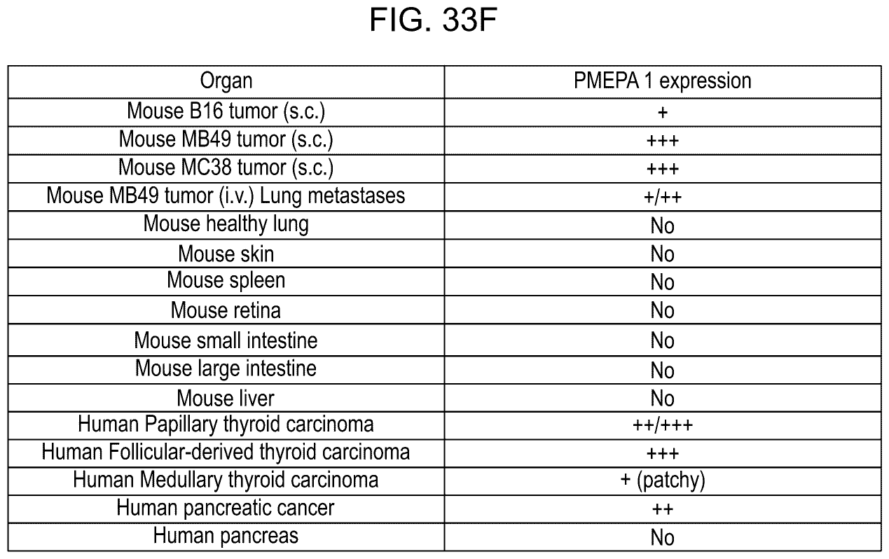

Generated sdABs were tested by FACS for binding to mouse (MEF) and human (293T) PMEPA-1 expressing (the expression was confirmed by RT-PCR (not shown)) and BW a-b-PMEPA-1 negative cells lines (FIG. 33A). While all “negative” subclones did not show any binding to all three cell lines tested, “positive” sdABs displayed high binding to human 293T cells and various degree of binding to mouse MEFs. Next, MC38 tumor and healthy skin BEC were stained with sdABs (FIGS. 33B and 33C). BEC were defined as live CD45-CD31+gp38−population, which was further subdivided on DARC+ VEC and DARC− NVEC (FIG. 33B). In MC38 tumor, all “positive” sdAB subclones showed higher binding to VEC compared to NVEC. Some insufficient staining of skin BEC was observed. However, binding efficiency to skin BEC was much lower than to their MC38 tumor counterparts (FIG. 33C). To corroborate FACS data, we performed IHC staining of MC38 tumor microvessels (FIG. 33D). Tumor frozen sections were co-stained with anti-CD31 and sdAB 1B2. Images reveal co-localization of two ABs, confirming binding of PMEPA-1 specific sdAB to tumor EC.

Additionally, sdABs were fused to mouse or human IgG1, and mFc-sdAB and hFc-sdAB fusions were generated. One of the advantages of the fusion proteins over sdABs is their bigger size (75 kD versus 10 kD of sdAB), which allows them to stay longer in circulation after intravenous (IV.) injection, rather than being rapidly cleared from the blood. Mouse Fc-IIG12 (control AB) and human Fc-1B2 (PMEPA-1 specific AB) were co-injected in MC38 tumor-bearing mice. Twenty minutes later, mice were sacrificed and perfused with 50 ml of PBS to wash off all unbound AB. Tumors were dissected, frozen and sectioned. Sections were co-stained with anti-mouse IgG1 and anti-human IgG1 conjugated with different colors fluorochromes. This approach allows to determine binding of control and PMEPA-1 specific ABs in the same field of view (FIG. 33E). While hFc-PMEPA-1 specific AB bound to most MC38 tumor microvessels (depicted by CD31 staining), no binding of mFc-Control AB to EC was observed.

Using the established panel of sdAB and Fc fusion with sdABs, IHC staining of microvessels was performed in various mouse and human tumors, as well as in healthy tissues. While different levels of PMEPA-1 expression were observed in all mouse and human tumors checked, no PMEPA-1 expression was detected in any healthy tissues analyzed (FIG. 33F).

Amino Acid and Polynucleotide Sequences

| SEQ ID NO | Amino Acid or Polynucleotide Sequence |
|---|---|
| SEQ ID NO: 1 | QAGGSLRLSCAASGYIFSDTYMGWYRQAPGKEREFVAGINGGGTTNYADSVKG |
| | RFTISRDNAKNTVYLQMNSLKPEDTAVYYCAVYYGSFSWSLL |
| SEQ ID NO: 2 | AGGSLRLSCAASGNISYSYGMGWYRQAPGKEREFVAGITFGGSTYYADSVKGR |
| | FTISRDNAKNTVYLQMNSLKPEDTAVYYCAVYTRHVDTTFARHWYWGQGTQVT |
| | VSSLEHHH |
| SEQ ID NO: 3 | AGGSLRLSCAASGNIFYGQPMGWYRQAPGKEREFVAGIGRGGSTYYADSVKGR |
| | FTISRDNAKNTVYLQMNSLKPEDTAVYYCAVLSQYPYRHTYWGQGTQVTVSSL |
| | EHH |
| SEQ ID NO: 4 | QAGGSLRLSCAASGTISTYGMGWYRQAPGKEREFVAGIATGGTTYYADSVKGR |
| | FTISRDNAKNTVYLQMNSLKPEDTAVYYCAALDKYARHYVYWGQGTQVTVSS |
| | LEHHHHHH |
| SEQ ID NO: 5 | QAGGSLRLSCAASGTIFYRYSMGWYRQAPGKEREFVAGITEGSNTYYADSVKGR |
| | FTISRDNAKNTVYLQMNSLKPEDTAVYYCAVVQRVDLTYWGQGTQVTVSSLEH |
| | HHHHH |
| SEQ ID NO: 6 | QAGGSLRLSCAASGNIFRVIGMGWYRQAPGKEREFVAGIGSGSSTYYADSVKGR |
| | FTISRDNAKNTVYLQMNSLKPEDTAVYYCAAFTYPYYDQTKLLPYWGQGTQVT |
| | VSSLEHHHHHH |
| SEQ ID NO: 7 | QAGGSLRLSCAASGTIFPRANMGWYRQAPGKEREFVAGITLGGTTYYADSVKG |
| | RFTISRDNAKNTVYLQMNSLKPEDTAVYYCAVVYKTYRYQEILYYYWGQGTQV |
| | TVSSLEHHHHHH |
| SEQ ID NO: 8 | NISYSYG |
| SEQ ID NO: 9 | GITFGGS |
| SEQ ID NO: 10 | VYTRHVDTTFARHW |
| SEQ ID NO: 11 | NIFYGQP |
| SEQ ID NO: 12 | GIGRGGS |
| SEQ ID NO: 13 | VLSQYPYRHT |
| SEQ ID NO: 14 | TISTYG |
| SEQ ID NO: 15 | GIATGGT |
| SEQ ID NO: 16 | ALDKYARHYV |
| SEQ ID NO: 17 | TIFYRYS |
| SEQ ID NO: 18 | GITEGSN |
| SEQ ID NO: 19 | VVQRVDLT |
| SEQ ID NO: 20 | NIFRVIG |
| SEQ ID NO: 21 | GIGSGSS |
| SEQ ID NO: 22 | AFTYPYYDQTKLLP |
| SEQ ID NO: 23 | TIFPRAN |
| SEQ ID NO: 24 | GITLGGT |
| SEQ ID NO: 25 | VVYKTYRYQEILYY |
| SEQ ID NO: 26 | GCGGGCGGCAGCCTGCGCCTGAGCTGCGCGGCGAGCGGCAATATTTCTTACT |
| | CTTACGGTATGGGCTGGTATCGCCAGGCGCCGGGCAAAGAACGCGAATTTGT |
| | TGCCGGTATTACTTTCGGTGGTAGTACCTATTATGCGGATAGCGTGAAAGGC |
| | CGCTTTACCATTAGCCGCGATAACGCGAAAAACACCGTGTATCTGCAGATGA |
| | ACAGCCTGAAACCGGAAGATACCGCGGTGTATTATTGCGCGGTTTACACTCG |
| | TCACGTTGACACTACTTTCGCTCGTCATTGGTATTGGGGCCAGGGCACCCAG |
| | GTGACCGTGAGCAGCCTCGAGCACCACCACCACCACCAC |
| SEQ ID NO: 27 | GCGGGCGGCAGCCTGCGCCTGAGCTGCGCGGCGAGCGGCAATATTTTTTACG |
| | GTCAGCCGATGGGCTGGTATCGCCAGGCGCCGGGCAAAGAACGCGAATTTGT |
| | TGCCGGTATTGGTCGTGGTGGTAGTACCTATTATGCGGATAGCGTGAAAGGC |
| | CGCTTTACCATTAGCCGCGATAACGCGAAAAACACCGTGTATCTGCAGATGA |
| | ACAGCCTGAAACCGGAAGATACCGCGGTGTATTATTGCGCGGTTCTGTCTCA |
| | GTACCCGTACCGTCATACTTATTGGGGCCAGGGCACCCAGGTGACCGTGAGC |
| | AGCCTCGAGCACCACCACCACCACCAC |
| SEQ ID NO: 28 | GGCAGCCTGCGCCTGAGCTGCGCGGCGAGCGGCACTATTTCTACTTACGGTA |
| | TGGGCTGGTATCGCCAGGCGCCGGGCAAAGAACGCGAATTTGTTGCCGGTAT |
| | TGCTACTGGTGGTACTACCTATTATGCGGATAGCGTGAAAGGCCGCTTTACC |
| | ATTAGCCGCGATAACGCGAAAAACACCGTGTATCTGCAGATGAACAGCCTGA |
| | AACCGGAAGATACCGCGGTGTATTATTGCGCGGCTCTGGACAAATACGCTCG |
| | TCATTATGTTTATTGGGGCCAGGGCACCCAGGTGACCGTGAGCAGCCTCGAG |
| | CACCACCACCACCACCAC |
| SEQ ID NO: 29 | GCGGGCGGCAGCCTGCGCCTGAGCTGCGCGGCGAGCGGCACTATTTTTTACC |
| | GTTACTCTATGGGCTGGTATCGCCAGGCGCCGGGCAAAGAACGCGAATTTGT |
| | TGCCGGTATTACTGAAGGTAGTAATACCTATTATGCGGATAGCGTGAAAGGC |
| | CGCTTTACCATTAGCCGCGATAACGCGAAAAACACCGTGTATCTGCAGATGA |
| | ACAGCCTGAAACCGGAAGATACCGCGGTGTATTATTGCGCGGTTGTTCAGCG |
| | TGTTGACCTTACTTATTGGGGCCAGGGCACCCAGGTGACCGTGAGCAGCCTC |
| | GAGCACCACCACCACCACCAC |
| SEQ ID NO: 30 | GCGGGCGGCAGCCTGCGCCTGAGCTGCGCGGCGAGCGGCAATATTTTTCGTG |
| | TTATCGGTATGGGCTGGTATCGCCAGGCGCCGGGCAAAGAACGCGAATTTGT |
| | TGCCGGTATTGGTTCTGGTAGTAGTACCTATTATGCGGATAGCGTGAAAGGC |
| | CGCTTTACCATTAGCCGCGATAACGCGAAAAACACCGTGTATCTGCAGATGA |
| | ACAGCCTGAAACCGGAAGATACCGCGGTGTATTATTGCGCGGCTTTCACTTA |
| | CCCGTACTACGACCAGACTAAACTGCTTCCGTATTGGGGCCAGGGCACCCAG |
| | GTGACCGTGAGCAGCCTCGAGCACCACCACCACCACCAC |
| SEQ ID NO: 31 | GCGGGCGGCAGCCTGCGCCTGAGCTGCGCGGCGAGCGGCACTATTTTTCCGC |
| | GTGCTAACATGGGCTGGTATCGCCAGGCGCCGGGCAAAGAACGCGAATTTGT |
| | TGCCGGTATTACTCTGGGTGGTACTACCTATTATGCGGATAGCGTGAAAGGC |
| | CGCTTTACCATTAGCCGCGATAACGCGAAAAACACCGTGTATCTGCAGATGA |
| | ACAGCCTGAAACCGGAAGATACCGCGGTGTATTATTGCGCGGTTGTTTACAA |
| | AACTTACCGTTACCAGGAAATCCTGTATTACTATTGGGGCCAGGGCACCCAG |
| | GTGACCGTGAGCAGCCTCGAGCACCACCACCACCACCAC |
| SEQ ID NO: 32 | (GGGGS)ₙ (n = 1, 2, 3, or 4) |
| SEQ ID NO: 33 | (Gly)₈ |
| SEQ ID NO: 34 | (Gly)₆ |
| SEQ ID NO: 35 | EAAAKEAAAKEAAAK |
| SEQ ID NO: 36 | (EAAAK)ₙ (n = 1-3) |
| SEQ ID NO: 37 | A(EAAAK)4ALEA(EAAAK)4A |
| SEQ ID NO: 38 | PAPAP |
| SEQ ID NO: 39 | AEAAAKEAAAKA |
| SEQ ID NO: 40 | (Ala-Pro)n (10-34 aa) |
| SEQ ID NO: 41 | GGGGSGGGGS |
| SEQ ID NO: 42 | GGGGS |

What is claimed is:

1. A composition comprising a binder connected to a radioactive isotope via a first linker, wherein the binder specifically binds to a protein expressed on an endothelial cell.

2. A composition comprising a first binder connected to a second binder via a first linker, wherein the first binder specifically binds to a protein expressed on an endothelial cell.

3. A composition comprising a binder connected to a payload via a first linker, wherein the binder specifically binds to a protein expressed on an endothelial cell.

4. The composition of any one of claims 1-3, wherein the protein is expressed on a venule endothelial cell (VEC) or a non-venule endothelial cell (NVEC).

5. The composition of any one of claims 1-3, wherein the protein is expressed on a microvascular endothelial cell.

6. The composition of any one of claims 1-3, wherein the protein expression is upregulated in VEC compared to NVEC.

7. The composition of any one of claims 1-3, wherein the protein is overexpressed in VEC by at least about 50% compared to NVEC.

8. The composition of any one of claims 1-3, wherein the protein expression is upregulated in NVEC compared to VEC.

9. The composition of any one of claims 1-3, wherein the protein is overexpressed in NVEC by at least about 50% compared to VEC.

10. The composition of any one of the preceding claims, wherein the protein is an organ-restricted endothelial cell protein or tissue-specific endothelial cell protein.

11. The composition of claim 10, wherein the organ-restricted endothelial cell protein or tissue-specific endothelial cell protein is in the liver, kidney, brain, retina, lymph node, bone marrow, small intestine, colon, adipose tissue, skin, lung, heart, any other organ, or a combination thereof.

12. The composition of any one of the preceding claims, wherein the protein has low expression levels on non-tumor endothelial cells.

13. The composition of any one of the preceding claims, wherein the protein is not expressed in circulating blood cells.

14. The composition of any one of the preceding claims, wherein the protein has greater expression levels on tumor endothelial cells than on non-tumor endothelial cells.

15. The composition of any one of the preceding claims, wherein the protein is encoded by a gene selected from the group consisting of genes set forth in Tables 1-3.

16. The composition of any one of the preceding claims, wherein the protein is encoded by a gene selected from VMP1, LAPTM5, EVL, PCDH17, ARRDC3, PMEPA-1, MYOF, MMP14, or PLEKHO1.

17. The composition of any one of the preceding claims, wherein the protein is PMEPA-1.

18. The composition of any one of the preceding claims, wherein the binder, first binder, and/or the second binder are an antibody or an antigen-binding fragment thereof.

19. The composition of any one of the preceding claims, wherein the binder, first binder, and/or the second binder
(a) is a monoclonal antibody, a polyclonal antibody, a bispecific antibody, a trispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a nanobody, or an antigen-binding fragment thereof; or
(b) an aptamer, a dendrimer, a peptide, RNAi, siRNA, shRNA, miRNA.

20. The composition of any one of the preceding claims, wherein the binder, first binder, and/or the second binder are a nanobody, a single chain variable fragment (scFv), a single-chain antibody, a single-domain antibody, a diabody, a Fab fragment, or a combination thereof.

21. The composition of any one of the preceding claims, wherein the binder, first binder, and/or the second binder are a nanobody.

22. The composition of any one of the preceding claims, wherein the binder, first binder, and/or the second binder are is an endothelial cell specific antibody or an antigen-binding fragment thereof.

23. The composition of any one of the preceding claims, wherein the binder, first binder, and/or the second binder are is an anti-PMEPA-1 antibody or an antigen-binding fragment thereof.

24. The composition of claim 23, wherein the anti-PMEPA-1 antibody or the antigen-binding fragment thereof comprises (a) a CDR1 having at least 80% sequence identity to the amino acid sequence set out in any one of SEQ ID NOs: 8, 11, 14, 17, 20, or 23, (b) a CDR2 having at least 80% sequence identity to the amino acid sequence set out in any one of SEQ ID NOs: 9, 12, 15, 18, 21, or 24, and (c) a CDR3 having at least 80% sequence identity to the amino acid sequence set out in any one of SEQ ID NOs: 10, 13, 16, 19, 22, or 25.

25. The composition of claim 23 or 24, wherein the anti-PMEPA-1 antibody or the antigen-binding fragment thereof comprises (a) a CDR1 having the amino acid sequence set out in any one of SEQ ID NOs: 8, 11, 14, 17, 20, or 23, (b) a CDR2 having the amino acid sequence set out in any one of SEQ ID NOs: 9, 12, 15, 18, 21, or 24, and (c) a CDR3 having the amino acid sequence set out in any one of SEQ ID NOs: 10, 13, 16, 19, 22, or 25.

26. The composition of any one of claims 23-25, wherein the anti-PMEPA-1 antibody or the antigen-binding fragment thereof comprises having at least 80% sequence identity to an amino acid sequence set out in any one of SEQ ID NOs: 1-7.

27. The composition of any one of claims 23-26, wherein the anti-PMEPA-1 antibody or the antigen-binding fragment thereof comprises an amino acid sequence set out in any one of SEQ ID NOs: 1-7.

28. The composition of any one of claims 1-27, wherein the binder, first binder, and/or the second binder specifically bind to the protein expressed on the endothelial cell with a Kd value of 1 mM or less, as measured by surface plasmon resonance (SPR).

29. The composition of any one of claims 1-28, wherein the binder, first binder, and/or the second binder specifically bind to the protein expressed on the endothelial cell with a Ka value of 1 mM or less, as measured by surface plasmon resonance (SPR).

30. The composition of any one of claims 2 and 4-29, wherein the second binder specifically binds to an antigen on the surface of a T cell.

31. The composition of claim 30, wherein the antigen on the surface of a T cell is selected from CD8b, CD4, CD2, CD28, CD45RA, CD45RO, and CD58.

32. The composition of any one of claims 1-31, wherein the second binder specifically binds to an antigen on the surface of a CAR cell, an NK cell, a granulocyte, a macrophage or a monocyte.

33. The composition of claim 32, wherein the antigen on the surface of a CAR cell, an NK cell, a granulocyte, a macrophage or a monocyte comprises CD15, CD11b, NKG2D, CD16, NKp30, NKp44, NKp46, DNAM, PSGL-1, CD44, CD11a, or CD49a.

34. The composition of any one of claims 2 and 4-33, wherein the second binder binds to a viral epitope.

35. The composition of claim 34, wherein the viral epitope is expressed on an AAV.

36. The composition of claim 35, wherein the AAV is selected form AAV1, AAV2, AAV3, AAV4, AAV5, AAV6, AAV7, AAV8, AAV9, AAV10, AAV11.

37. The composition of any one of claims 2 and 4-36, wherein the second binder binds to a nanoparticle.

38. The composition of claim 37, wherein the nanoparticle is a lipid nanoparticle.

39. The composition of claim 37, wherein the nanoparticle is a polymer nanoparticle.

40. The composition of any one of the preceding claims, wherein
(a) the binder further comprises a masking moiety; and/or
(b) the first binder further comprises a first masking moiety; and/or
(c) the second binder further comprises a second masking moiety.

41. The composition of claim 40, wherein:
(a) the masking moiety is covalently attached to a binding domain of the binder via a second linker; or
(b) the first masking moiety is covalently attached to a binding domain of the first binder via a second linker; or
(c) the second masking moiety is covalently attached to a binding domain of the second binder via a third linker.

42. The composition of claim 40 or 41, wherein
(a) the masking moiety is covalently attached to a heavy chain variable domain and/or a light chain variable chain domain of the binder via a second linker; or
(b) the first masking moiety is covalently attached to a heavy chain variable domain and/or a light chain variable chain domain of the first binder via a second linker; or
(c) the second masking moiety is covalently attached to a heavy chain variable domain and/or a light chain variable chain domain of the second binder via a third linker.

43. The composition of claim 40 or 41, wherein the masking moiety, the first masking moiety, and/or the second masking moiety are an anti-idiotypic antibody or fragment thereof.

44. The composition of claim 40 or 41, wherein the first masking moiety, and/or the second masking moiety are is an anti-idiotypic scFv or fragment thereof.

45. The composition of any one of claims 41-44, wherein the first linker and/or the second linker is a non-cleavable linker or a cleavable linker.

46. The composition of any one of claims 41-45, wherein the first linker and/or the second linker is a non-cleavable linker.

47. The composition of any one of claims 41-46, wherein the first linker and/or the second linker is a polypeptide linker.

48. The composition of claim 47, wherein the polypeptide linker comprises about 2-20 amino acids.

49. The composition of claim 47 or 48, wherein the polypeptide linker comprises (G4S)n, (SG4)n, G4(SG4)n or G2(SG2)n, wherein n is selected from 1 to 10.

50. The composition of any one of claims 47-49, wherein the polypeptide linker comprises GGGGSGGGGS (SEQ ID NO: 41) or GGGGS (SEQ ID NO: 42).

51. The composition of claim 41-50, wherein the first linker and/or the second linker comprises at least one group selected from the group consisting of alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene is optionally substituted.

52. The composition of claim 51, wherein the first linker and/or the second linker comprises substituted or unsubstituted C₁-C₆ alkylene or substituted or unsubstituted C₁-C₆ heteroalkylene.

53. The composition of claim 51 or 52, wherein the first linker and/or the second linker comprises one or more groups selected from the group consisting of —O—, —S—, —NH—, —NH—(CH₂)_{y}—NH, —NH—(CH₂)_{y}—O, —O—(CH₂)_{y}—O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH₂CH₂)_{w}—(C═O)—, —(C═O)—(CH═CH)_{w}—(C═O), —(C═O)—(OCH₂CH₂O)_{w}—(C═O)—, —(CH₂CH₂O)_{w}—, —(C═O)—(CH2CH2O)_{w}—, and —(CH(CH3)C(═O)O)_{w}—, wherein w is 1-20 and y is 1-20.

54. The composition of any one of claims 45 or 47-53, wherein the first linker and/or the second linker is a cleavable linker.

55. The composition of any one of claims 45 or 47-53, wherein the cleavable linker is a protease-cleavable linker, a self-immolative linker, or a pH-sensitive linker.

56. The composition of claim 55, wherein the protease-cleavable linker comprises at least one protease recognition site.

57. The composition of claims 55 or 56, wherein the protease is selected from metalloproteinase (MMP) 1-28; A Disintegrin And Metalloproteinase (ADAM) 2, 7-12, 15, 17-23, 28-30 and 33; serine protease; urokinase-type plasminogen activator; Matriptase; cysteine protease; aspartic protease; and cathepsin protease.

58. The composition of claim 57, wherein the protease is MMP2 or MMP9.

59. The composition of claim 55, wherein the self-immolative linker is selected from para-amino benzoic acid (PAB), para-aminobenzyl alcohol (PABA), 3,3-dimethyl-4-hydroxybutyric acid, ethylenediamine, γ-aminobutyric acid (GABA), 2-hydroxycinnamic acid, “Trimethyl Lock”, or ethanolamine.

60. The composition of claim 55 or 59, wherein the self-immolative linker is para-amino benzoic acid (PAB).

61. The composition of claim 55, wherein the pH-sensitive linker is cleaved upon exposure to a target pH.

62. The composition of claim 61, wherein the target pH is less than about 7.

63. The composition of any one of claims 55 or 61-62, wherein the pH-sensitive linker is selected from an optionally substituted tetrahydropyranyl ether, an optionally substituted tetrahydropyranyl ester, an optionally substituted azide, an optionally substituted histidine, an optionally substituted hydrazone, or an optionally substituted D-amino ester.

64. The composition of any one of claims 55 or 61-63, wherein the pH-sensitive linker is selected from -(tetrahydropyran ether)-(azide), -(hydrazone)-, -(hydrazone)-(azide)-, -(β-amino ester)-, -(β-amino ester)-(azide)-, or -(tetrahydropyran ester)-.

65. The composition of any one of claims 1 or 4-64, wherein the radioactive isotope is an alpha emitter, beta emitter, or gamma emitter.

66. The composition of any one of claims 1 or 4-65, wherein the radioactive isotope emits a particle or ray in the range of 10-7,000 keV.

67. The composition of any one of claims 1 or 4-66, wherein the radioactive isotope emits a particle or ray in the range of 50-1,500 keV.

68. The composition of any one of claims 1 or 4-67, wherein the radioactive isotope emits a particle or ray in the range of 80-250 keV.

69. The composition of any one of claims 1 or 4-68, wherein the radioactive isotope is selected from Actinium-225, Astatine-211, Iodine-123, Iodine-125, Iodine-126, Iodine-131, Iodine-133, Bismuth-212, Bromine-77, Indium-111, Indium-113m, Gallium-67, Gallium-68, Lead-212, Ruthenium-95, Ruthenium-97, Ruthenium-103, Ruthenium-105, Mercury-107, Mercury-203, Rhenium-186, Rhenium-188, Tellurium-121m, Tellurium-122m, Tellurium-125m, Thulium-165, Thulium-167, Thulium-168, Technetium-99m, Fluorine-18, Silver-111, Platinum-197, Palladium-109, Copper-67, Phosphorus-32, Phosphorus-33, Yttrium-90, Scandium-47, Samarium-153, Lutetium-177, Rhodium-105, Praseodymium-142, Praseodymium-143, Terbium-161, Holmium-166, Gold-199, Cobalt-57, Cobalt-58, Chromium-51, Iron-59, Selenium-75, Thallium-201, Zirconium-89, and Ytterbium-169.

70. The composition of any one of claims 1 or 4-69, wherein the radioactive isotope is selected from Iodine-123, Iodine-131, Indium-111, Gallium-67, Lead-212, Ruthenium-97, Technetium-99m, Cobalt-57, Cobalt-58, Chromium-51, Iron-59, Selenium-75, Thallium-201, and Ytterbium-169.

71. The composition of any one of claims 1 or 4-70, wherein the radioactive isotope is Actinium-225, Gallium-67, Lead-21, or Lutetium-177.

72. The composition of any one of claims 1 or 4-71, wherein the radioactive isotope is Technetium-99m.

73. The composition of any one of claims 3-64, wherein the payload is a toxin.

74. The composition of any one of claims 3-64 or 73, wherein the payload is a cytokine.

75. The composition of any one of claims 3-64 or 73-74, wherein the payload is a cytotoxic payload.

76. The composition of any one of claims 3-64 or 73-75, wherein the payload is cytotoxic to a tumor cell upon internalization into the tumor cell.

77. The composition of any one of claims 3-64 or 73-76, wherein the payload comprises a antitumor antibiotic, microtubule inhibitor, cytotoxic or cytostatic, topoisomerase inhibitor, a pyrrolobenzodiazepine, a DNA-alkylating drug, a DNA-binding drug, a DNA-cleaving drug, or an RNA polymerase inhibitor.

78. The composition of any one of claims 3-64 or 73-77, wherein the payload comprises pyrrolobenzodiazepine, duocarmycin, auristatin, maytansinoid, uncialamycin, dynemicin, thailanstatin, camptothecin, exatecan, tubulysin compound, lurbinectedin, trabectedin, safracin, lenalidomide, eribulin, vincristine, vinblastine, vindesine, vinorelbine, an epothilone, a taxane (e.g., paclitaxel, docetaxel, cabazitaxel, etc.), a cryptophycin, a hemiasterlin, an anthracyclin, a bisnaphthylamide (e.g., elinafide), or a cytotoxic molecular glue/PROTAC compound.

79. The composition of any one of claims 3-64 or 73-78, wherein the payload is camptothecin, exatecan, lurbinectedin, trabectedin, belotecan, atiratecan, namitecan, 7-nbutyl-10-amino-camptothecin, or 7-n-butyl-9-amino-10,11-methylenedixoy-camptothecin.

80. The composition of any one of claims 3-64 or 73-79, wherein the payload comprises dolastatin 10, auristatin E, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), or 5-benzoylvaleric acid-AE ester (AEVB).

81. The composition of any one of claims 3-64 or 73-80, wherein DAR (drug to antibody ratio) of the composition is 2, 3, 4, 5, 6, 8, 10, or 20.

82. A composition having a structure of Formula (XII):
AB-(L)m-(P)n (XII)
wherein:
AB is an antibody or antigen-binding fragment;
L is a linker;
P is a payload;
m is 0 to 10; and
n is 1 to 20.

83. The composition of claim 82, wherein the antibody or antigen-binding fragment is
(a) a monoclonal antibody, a polyclonal antibody, a bispecific antibody, a trispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a nanobody, or an antigen-binding fragment; or
(b) an aptamer, a dendrimer, a peptide, RNAi, siRNA, shRNA, miRNA.

84. The composition of claim 82 or 83, wherein the antibody or antigen-binding fragment is a nanobody, a single chain variable fragment (scFv), a single-chain antibody, a single-domain antibody, a diabody, a Fab fragment, or a combination thereof.

85. The composition of any one of claims 82-84, wherein the antibody or antigen-binding fragment is a nanobody.

86. The composition of any one of claims 82-85, wherein the antibody or antigen-binding fragment is an endothelial cell specific antibody.

87. The composition of any one of claims 82-86, wherein the antibody or antigen-binding fragment is an anti-PMEPA-1 antibody or antigen-binding fragment.

88. The composition of claim 87, wherein the anti-PMEPA-1 antibody or antigen-binding fragment comprises (a) a CDR1 having at least about 80% sequence identity to the amino acid sequence set out in any one of SEQ ID NOs: 8, 11, 14, 17, 20, or 23, (b) a CDR2 having at least about 80% sequence identity to the amino acid sequence set out in any one of SEQ ID NOs: 9, 12, 15, 18, 21, or 24, and (c) a CDR3 having at least about 80% sequence identity to the amino acid sequence set out in any one of SEQ ID NOs: 10, 13, 16, 19, 22, or 25.

89. The composition of claim 87, wherein the anti-PMEPA-1 antibody or antigen-binding fragment comprises (a) a CDR1 having the amino acid sequence set out in any one of SEQ ID NOs: 8, 11, 14, 17, 20, or 23, (b) a CDR2 having the amino acid sequence set out in any one of SEQ ID NOs: 9, 12, 15, 18, 21, or 24, and (c) a CDR3 having the amino acid sequence set out in any one of SEQ ID NOs: 10, 13, 16, 19, 22, or 25.

90. The composition of any one of claims 82-89, wherein the anti-PMEPA-1 antibody or antigen-binding fragment comprises having at least about 80% sequence identity to an amino acid sequence set out in any one of SEQ ID NOs: 1-7.

91. The composition of any one of claims 82-89, wherein the anti-PMEPA-1 antibody or antigen-binding fragment comprises an amino acid sequence set out in any one of SEQ ID NOs: 1-7.

92. The composition of any one of claims 82-91, wherein the antibody or antigen-binding fragment specifically binds to the protein expressed on the endothelial cell with a Kd value of about 1 mM or less, as measured by surface plasmon resonance (SPR).

93. The composition of any one of claims 82-91, wherein the antibody or antigen-binding fragment specifically binds to the protein expressed on the endothelial cell with a Ka value of about 1 mM or less, as measured by surface plasmon resonance (SPR).

94. The composition of any one of claims 82-91, wherein the linker is a non-cleavable linker or a cleavable linker.

95. The composition of claim 94, wherein the linker is a non-cleavable linker.

96. The composition of any one of claims 82-95, wherein the linker is polypeptide linker.

97. The composition of claim 96, wherein the polypeptide linker comprises about 2-20 amino acids.

98. The composition of claim 96 or 97, wherein the polypeptide linker comprises (G4S)n, (SG4)n, G4(SG4)n or G2(SG2)n, wherein n is selected from 1 to 10.

99. The composition of any one of claims 96-98, wherein the polypeptide linker comprises GGGGSGGGGS (SEQ ID NO: 41) or GGGGS (SEQ ID NO: 42).

100. The composition of any one of claims 82-99, wherein the linker comprises at least one group selected from the group consisting of alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene is optionally substituted.

101. The composition of any one of claims 82-100, wherein the linker comprises substituted or unsubstituted C₁-C₆ alkylene or substituted or unsubstituted C₁-C₆ heteroalkylene.

102. The composition of any one of claims 82-101, wherein the linker comprises one or more groups selected from the group consisting of —O—, —S—, —NH—, —NH—(CH2)p-NH, —NH—(CH2)p-O, —O—(CH2)p-O, —(C═O)—, —(C═O)—O—, —O(C═O)—, —O(C═O)—O—, —OC(═O)—NH—, —C(═O)NH—, —NHC(═O)—, —NHC(═O)—O—, or —NHC(═O)—NH—, —(C═O)—(CH2CH2)q-(C═O)—, —(C═O)—(CH═CH)q-(C═O), —(C═O)—(OCH2CH2O)q-(C═O)—, —(CH2CH2O)q-, —(C═O)—(CH2CH2O)q-, and —(CH(CH3)C(═O)O)q-, wherein q is 1-20 and p is 1-20.

103. The composition of claim 94, wherein the linker is a cleavable linker.

104. The composition of claim 103, wherein the cleavable linker is a protease-cleavable linker, a self-immolative linker, or a pH-sensitive linker.

105. The composition of claim 104, wherein the protease-cleavable linker comprises at least one protease recognition site.

106. The composition of claim 105, wherein the protease is selected from metalloproteinase (MMP) 1-28; A Disintegrin And Metalloproteinase (ADAM) 2, 7-12, 15, 17-23, 28-30 and 33; serine protease; urokinase-type plasminogen activator; Matriptase; cysteine protease; aspartic protease; and cathepsin protease.

107. The composition of claim 106, wherein the protease is MMP2 or MMP9.

108. The composition of claim 104, wherein the self-immolative linker is selected from para-amino benzoic acid (PAB), para-aminobenzyl alcohol (PABA), 3,3-dimethyl-4-hydroxybutyric acid, ethylenediamine, γ-aminobutyric acid (GABA), 2-hydroxycinnamic acid, “Trimethyl Lock”, or ethanolamine.

109. The composition of claim 104 or 108, wherein the self-immolative linker is para-amino benzoic acid (PAB).

110. The composition of claim 104, wherein the pH-sensitive linker is cleaved upon exposure to a target pH.

111. The composition of claim 110, wherein the target pH is less than about 7.

112. The composition of claim 104, wherein the pH-sensitive linker is selected from an optionally substituted tetrahydropyranyl ether, an optionally substituted tetrahydropyranyl ester, an optionally substituted azide, an optionally substituted histidine, an optionally substituted hydrazone, or an optionally substituted D-amino ester.

113. The composition of claim 104, wherein the pH-sensitive linker is selected from -(tetrahydropyran ether)-(azide), -(hydrazone)-, -(hydrazone)-(azide)-, -(β-amino ester)-, -(β-amino ester)-(azide)-, or -(tetrahydropyran ester)-.

114. The composition of any one of claims 82-113, wherein the payload is a toxin.

115. The composition of any one of claims 82-114, wherein the payload is a cytokine.

116. The composition of any one of claims 82-115, wherein the payload is a cytotoxic payload.

117. The composition of any one of claims 82-116, wherein the payload is cytotoxic to a tumor cell upon internalization into the tumor cell.

118. The composition of any one of claims 82-117, wherein the payload comprises a antitumor antibiotic, microtubule inhibitor, cytotoxic or cytostatic, topoisomerase inhibitor, a pyrrolobenzodiazepine, a DNA-alkylating drug, a DNA-binding drug, a DNA-cleaving drug, or an RNA polymerase inhibitor.

119. The composition of any one of claims 82-118, wherein the payload comprises pyrrolobenzodiazepine, duocarmycin, auristatin, maytansinoid, uncialamycin, dynemicin, thailanstatin, camptothecin, exatecan, tubulysin compound, lurbinectedin, trabectedin, safracin, lenalidomide, eribulin, vincristine, vinblastine, vindesine, vinorelbine, an epothilone, a taxane (e.g., paclitaxel, docetaxel, cabazitaxel, etc.), a cryptophycin, a hemiasterlin, an anthracyclin, a bisnaphthylamide (e.g., elinafide), or a cytotoxic molecular glue/PROTAC compound.

120. The composition of any one of claims 82-119, wherein the payload is camptothecin, exatecan, lurbinectedin, trabectedin, belotecan, atiratecan, namitecan, 7-nbutyl-10-amino-camptothecin, or 7-n-butyl-9-amino-10,11-methylenedixoy-camptothecin.

121. The composition of any one of claims 82-120, wherein the payload comprises dolastatin 10, auristatin E, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), or 5-benzoylvaleric acid-AE ester (AEVB).

122. The composition of any one of claims 82-121, wherein DAR (drug to antibody ratio) of the composition is composition is 2, 3, 4, 5, 6, 8, 10, or 20.

123. A cell comprising a first binder that specifically binds to a protein expressed on an endothelial cell.

124. The cell of claim 123, wherein the cell is an immune cell or an engineered immune cell.

125. The cell of claim 124, wherein the immune cell is a T cell, macrophage, monocyte, granulocyte, or natural killer (NK) cell, or natural killer T (NKT) cell.

126. The cell of claim 125, wherein the T cell is a tumor-infiltrating lymphocyte (TIL) or a cytotoxic T lymphocyte (CTL).

127. The cell of claim 126, wherein the cell expresses a tumor-specific T-cell receptor.

128. The cell of claim 124, wherein the immune cell is CD4+ or CD8+ T cell.

129. The cell of claim 124, wherein the engineered immune call is a CAR-immune cell.

130. The cell of claim 129, wherein the CAR-immune cell is a CAR-T cell, CAR-macrophages, CAR-monocyte, CAR-granulocyte, CAR-NK cell, or a CAR-NKT cell.

131. The cell of any one of claims 123-130, wherein the first binder is not a chimeric antigen receptor.

132. The cell of claim any one of claims 123-131, wherein the cell further comprises a second binder that specifically binds to a tumor-associated antigen.

133. The cell of claim 132, wherein the tumor-associated antigen is selected from CD19, CD20, CD22, CD30, CD37, CD38, CEA, EpCAM, or BCMA.

134. The cell of claim 132 or 133, wherein the second binder is linked to the cell via a first linker.

135. The cell of any one of claims 123-134, wherein the protein is expressed on a venule endothelial cell (VEC) or a non-venule endothelial cell (NVEC).

136. The cell of any one of claims 123-134, wherein the protein is expressed on a microvascular endothelial cell.

137. The cell of any one of claims 123-136, wherein the protein expression is upregulated in VEC compared to NVEC.

138. The cell of any one of claims 123-137, wherein the protein is overexpressed in VEC by at least 50% compared to NVEC.

139. The cell of any one of claims 123-138, wherein the protein expression is upregulated in NVEC compared to VEC.

140. The cell of any one of claims 123-139, wherein the protein is overexpressed in NVEC by at least 50% compared to VEC.

141. The cell of any one of claims 123-140, wherein the protein is an organ-restricted or tissue-specific endothelial cell protein.

142. The cell of claim 141, wherein the organ-restricted or tissue-specific endothelial cell protein is in the brain, liver, kidney, retina, lymph node, bone marrow, small intestine, colon, adipose tissue, skin, lung, heart, or any other organ.

143. The cell of any one of claims 123-142, wherein the protein has low expression levels on non-tumor endothelial cells.

144. The cell of any one of claims 123-143, wherein the protein is not expressed in circulating blood cells.

145. The cell of any one of claims 123-144, wherein the protein has greater expression levels on tumor endothelial cells than on non-tumor endothelial cells.

146. The cell of any one of claims 123-145, wherein the protein is encoded by a gene selected from the group consisting of molecules set forth in Tables 1-3.

147. The cell of any one of claims 123-146, wherein the protein is encoded by a gene selected from VMP1, LAPTM5, EVL, PCDH17, ARRDC3, PMEPA1, MYOF, MMP14, or PLEKHO1.

148. The cell of any one of claims 123-147, wherein the protein is PMEPA-1.

149. The cell of any one of claims 123-148, wherein the first binder and/or the second binder is an antibody or antigen-binding fragment.

150. The cell of any one of claims 123-149, wherein the first binder and/or the second binder is
(a) a monoclonal antibody, a polyclonal antibody, a bispecific antibody, a trispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a nanobody, or an antigen-binding fragment thereof; or
(b) aptamer, dendrimer, a peptide, RNAi, siRNA, shRNA, or miRNA or ab antigen-binding fragments thereof.

151. The cell of any one of claims 123-150, wherein the first binder and/or the second binder is a nanobody, a single chain variable fragment (scFv), a single-chain antibody, a single-domain antibody, a diabody, a Fab fragment, or a combination thereof.

152. The cell of any one of claims 123-151, wherein the first binder and/or the second binder is a nanobody.

153. The cell of any one of claims 123-152, wherein the first binder is an endothelial cell specific antibody.

154. The cell of any one of claims 123-153, wherein the first binder is an anti-PMEPA-1 antibody or antigen-binding fragment.

155. The cell of claim 154, wherein the anti-PMEPA-1 antibody or antigen-binding fragment thereof comprises (a) a CDR1 having at least 80% sequence identity to the amino acid sequence set out in any one of SEQ ID NOs: 8, 11, 14, 17, 20, or 23, (b) a CDR2 having at least 80% sequence identity to the amino acid sequence set out in any one of SEQ ID NOs: 9, 12, 15, 18, 21, or 24, and (c) a CDR3 having at least 80% sequence identity to the amino acid sequence set out in any one of SEQ ID NOs: 9, 12, 15, 18, 21, or 24.

156. The cell of claim 154 or 155, wherein the anti-PMEPA-1 antibody or antigen-binding fragment thereof comprises (a) a CDR1 having the amino acid sequence set out in any one of SEQ ID NOs: 8, 11, 14, 17, 20, or 23, (b) a CDR2 having the amino acid sequence set out in any one of SEQ ID NOs: 9, 12, 15, 18, 21, or 24, and (c) a CDR3 having the amino acid sequence set out in any one of SEQ ID NOs: 10, 13, 16, 19, 22, or 25.

157. The cell of any one of claims 154-156, wherein the anti-PMEPA-1 antibody or antigen-binding fragment thereof comprises having at least 80% sequence identity to an amino acid sequence set out in any one of SEQ ID NOs: 1-7.

158. The cell of any one of claims 154-157, wherein the anti-PMEPA-1 antibody or antigen-binding fragment thereof comprises an amino acid sequence set out in any one of SEQ ID NOs: 1-7.

159. The cell of any one of claims 123-158, wherein the first binder specifically binds to the protein expressed on the endothelial cell with a Kd value of 1 mM or less, as measured by surface plasmon resonance (SPR).

160. The cell of any one of claims 123-159, wherein the first binder specifically binds to the protein expressed on the endothelial cell with a Ka value of 1 mM or less, as measured by surface plasmon resonance (SPR).

161. The cell of any one of claims 123-160, wherein the first binder and/or the second binder further comprises a masking moiety.

162. The cell of claim 161, wherein the masking moiety is covalently attached to a binding domain of the first binder and/or the second binder via a second linker.

163. The cell of claim 161 or 162, wherein the masking moiety is covalently attached to a heavy chain variable domain and/or a light chain variable chain domain of the first binder and/or the second binder via a second linker.

164. The cell of claim 161 or 162, wherein the masking moiety is an anti-idiotypic antibody or fragment thereof.

165. The cell of any one of claims 161-163, wherein the masking moiety is an anti-idiotypic scFv or fragment thereof.

166. The cell of any one of claims 162-165, wherein the first linker and/or the second linker is a non-cleavable linker or a cleavable linker.

167. The cell of any one of claims 162-166, wherein the first linker and/or the second linker is a non-cleavable linker.

168. The cell of any one of claims 162-167, wherein the first linker and/or the second linker is polypeptide linker.

169. The cell of claim 168, wherein the polypeptide linker comprises about 2-20 amino acids.

170. The cell of 168 or 169, wherein the polypeptide linker comprises (G4S)n, (SG4)n, G4(SG4)n or G2(SG2)n, wherein n is selected from 1 to 10.

171. The cell of any one of claims 168-170, wherein the polypeptide linker comprises GGGGSGGGGS (SEQ ID NO: 41) or GGGGS (SEQ ID NO: 42).

172. The cell of claim 161-167, wherein the first linker and/or the second linker comprises at least one group selected from the group consisting of alkylene, alkenylene, alkynylene, cycloalkylene, arylene, heteroalkylene, heterocycloalkylene and heteroarylene, wherein each of the alkylene, alkenylene, alkynylene, cycloalkylene, arylene, heteroalkylene, heterocycloalkylene or heteroarylene is optionally substituted.

173. The cell of claim 172, wherein the first linker and/or the second linker comprises substituted or unsubstituted C1-C6 alkylene or substituted or unsubstituted C1-C6 heteroalkylene.

174. The cell of any one of claims 172 or 173, wherein the first linker and/or the second linker comprises one or more groups selected from the group consisting of —O—, —S—, —NH—, —NH—(CH2)p-NH, —NH—(CH2)p-O, —O—(CH2)p-O, —(C═O)—, —(C═O)—O—, —O(C═O)—, —O(C═O)—O—, —OC(═O)—NH—, —C(═O)NH—, NHC(═O)—, —NHC(═O)—O—, or —NHC(═O)—NH—, —(C═O)—(CH2CH2)q-(C═O)—, —(C═O)—(CH═CH)q-(C═O), —(C═O)—(OCH2CH2O)q-(C═O)—, —(CH2CH2O)q-, —(C═O)—(CH2CH2O)q-, and —(CH(CH3)C(═O)O)q-, wherein q is 1-20 and p is 1-20.

175. The cell of any one of claims 161-165, wherein the first linker and/or the second linker is a cleavable linker.

176. The cell of claim 175, wherein the cleavable linker is a protease-cleavable linker, a self-immolative linker, or a pH-sensitive linker.

177. The cell of claim 176, wherein the protease-cleavable linker comprises at least one protease recognition site.

178. The cell of claim 176 or 177, wherein the protease is selected from metalloproteinase (MMP) 1-28; A Disintegrin And Metalloproteinase (ADAM) 2, 7-12, 15, 17-23, 28-30 and 33; serine protease; urokinase-type plasminogen activator; Matriptase; cysteine protease; aspartic protease; and cathepsin protease.

179. The cell of any one of claims 176-178, wherein the protease is MMP2 or MMP9.

180. The cell of claim 176, wherein the self-immolative linker is selected from para-amino benzoic acid (PAB), para-aminobenzyl alcohol (PABA), 3,3-dimethyl-4-hydroxybutyric acid, ethylenediamine, γ-aminobutyric acid (GABA), 2-hydroxycinnamic acid, “Trimethyl Lock”, or ethanolamine.

181. The cell of claim 180, wherein the self-immolative linker is para-amino benzoic acid (PAB).

182. The cell of claim 176, wherein the pH-sensitive linker is cleaved upon exposure to a target pH.

183. The cell of claim 182, wherein the target pH is less than about 7.

184. The cell of any one of claims 176, 182 or 183, wherein the pH-sensitive linker is selected from an optionally substituted tetrahydropyranyl ether, an optionally substituted tetrahydropyranyl ester, an optionally substituted azide, an optionally substituted histidine, an optionally substituted hydrazone, or an optionally substituted β-amino ester.

185. The cell of any one of claims 176 or 182-184, wherein the pH-sensitive linker is selected from -(tetrahydropyran ether)-(azide), -(hydrazone)-, -(hydrazone)-(azide)-, -(D-amino ester)-, -(D-amino ester)-(azide)-, or -(tetrahydropyran ester)-.

186. An engineered immune cell comprising:
(a) a first polynucleotide that encodes a first binder that specifically binds to a protein expressed on an endothelial cell; and
(b) a second polynucleotide that encodes a chimeric antigen receptor (CAR).

187. The engineered immune cell of claim 186, wherein the immune cell is a T cell, macrophage, monocyte, granulocyte, natural killer (NK) cell, or natural killer T (NKT) cell.

188. The engineered immune cell of claim 186 or 187, wherein the immune cell is CD4+ or CD8+ T cell.

189. The engineered immune cell of any one of claims 186-188, wherein the first binder is not a CAR.

190. The engineered immune cell of any one of claims 186-188, wherein the first binder comprises a membrane anchoring domain.

191. The engineered immune cell of claim 190, wherein the membrane anchoring domain is a transmembrane domain or a Gpi linker.

192. The engineered immune cell of claim 191, wherein the transmembrane domain comprises a sequence from L-selectin (CD62L), PSGL-1, or alpha4-integrin transmembrane domain.

193. The engineered immune cell of any one of claims 190-192, wherein the first binder further comprises an intracellular segment.

194. The engineered immune cell of claim 193, wherein the intracellular segment comprise a sequence from an intracellular region of L-selectin (CD62L), PSGL-1, or alpha4-integrin.

195. The engineered immune cell of any one of claims 186-188, wherein the CAR comprises a target domain, a hinge domain, a transmembrane domain, and an intracellular signaling domain.

196. The engineered immune cell of claim 195, wherein the target domain comprises a second binder that specifically binds to a tumor-associated antigen.

197. The engineered immune cell of claim 196, wherein the tumor-associated antigen is selected from CD19, CD20, CD22, CD30, CD37, CD38, CEA, EpCAM, or BCMA.

198. The engineered immune cell of claim 197, wherein the hinge domain is selected from IgG1, IgG2, IgG3, IgG4, IgA, IgD, IgE, IgM, CD28, or CD8.

199. The engineered immune cell of claim 198, wherein the hinge domain is CD28 or CD8.

200. The engineered immune cell of claim 195, wherein the transmembrane domain is selected from alpha, beta or zeta chain of the T-cell receptor, CD28, CD3 epsilon, CD45, CD4, CD5, CD8, CD9, CD16, CD22, CD33, CD37, CD64, CD80, CD86, CD123, CD134, CD137, or CD154.

201. The engineered immune cell of claim 195, wherein the intracellular signaling domain is selected from CD3ζ, CD3γ, CD3δ, CD3ε, CD5, CD22, CD79a, CD79b, CD66d, CD2, CD4, CD5, CD28, CD134, CD137, ICOS, CD154, 41-BB, or OX40.

202. The engineered immune cell of any one of claims 195-201, wherein the CAR further comprises a co-stimulatory domain.

203. The engineered immune cell of claim 202, wherein the co-stimulatory domain is selected from CD3ζ, CD3γ, CD3δ, CD3ε, CD5, CD22, CD79a, CD79b, CD66d, CD2, CD4, CD5, CD28, CD134, CD137, ICOS, CD154, 41-BB, or OX40.

204. The engineered immune cell of 202 or 203, wherein the co-stimulatory domain is 41-BB or OX40.

205. The engineered immune cell of any one of claims 186-203, wherein the first binder is not connected to the intracellular signaling domain.

206. A pharmaceutical composition comprising the composition of any one of claims 1-122, the cell of any one of claims 123-185, or the engineered immune cell of any one of claims 186-205, and a pharmaceutically acceptable carrier and/or excipient.

207. The pharmaceutical composition of claim 206, wherein the pharmaceutical composition further comprises at least one additional therapeutic agent.

208. A method of selectively delivering a radiopharmaceutical composition to a tumor endothelial cell compared to a non-tumor endothelial cell in a subject in need thereof, the method comprising administering to the subject an effective amount of the composition of any one of claims 1-72 or the pharmaceutical composition of claim 206 or 207 to the subject.

209. A method of selectively delivering a therapeutic composition to a tumor endothelial cell compared to a non-tumor endothelial cell in a subject in need thereof, the method comprising administering to the subject an effective amount of the composition of any one of claims 1-122, the cell of any one of claims 123-185, the engineered immune cell of any one of claims 186-205, or the pharmaceutical composition of claim 206 or 207 to the subject.

210. The method of claim 208 or 209, wherein the composition, cell, engineered immune cell, or pharmaceutical composition has a greater binding affinity for a tumor endothelial cell compared to a binding affinity for a non-tumor endothelial cell.

211. A method of treating a disease or disorder in a subject in a need thereof, the method comprising administering to the subject an effective amount of the composition of any one of claims 1-122, the cell of any one of claims 123-185, the engineered immune cell of any one of claims 186-205, or the pharmaceutical composition of claim 206-207 to the subject.

212. The method of claim 211, wherein the disease or disorder is endotoxemia, sepsis, cancer, obesity-related insulin resistance, diabetes, polycystic ovary syndrome, metabolic syndrome, hypertension, cerebrovascular accident, myocardial infarction, congestive heart failure, cholecystitis, gout, osteoarthritis, Pickwickian syndrome, sleep apnea, atherosclerosis, inflammatory bowel disease, rheumatoid arthritis, vasculitis, transplant rejection, asthma, ischemic heart disease, appendicitis, peptic, gastric and duodenal ulcers, peritonitis, pancreatitis, ulcerative, pseudomembranous, acute and ischemic colitis, diverticulitis, epiglottitis, achalasia, cholangitis, hepatitis, Crohn's disease, enteritis, Whipple's disease, allergy, anaphylactic shock, immune complex disease, organ ischemia, reperfusion injury, organ necrosis, hay fever, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, alveolitis, bronchiolitis, pharyngitis, pleurisy, sinusitis, a parasitic infection, a bacterial infection, a viral infection, an autoimmune disease, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasculitis, angiitis, endocarditis, arteritis, thrombophlebitis, pericarditis, myocarditis, myocardial ischemia, periarteritis nodosa, rheumatic fever, celiac disease, adult respiratory distress syndrome, meningitis, encephalitis, cerebral infarction, cerebral embolism, Guillain-Barre syndrome, neuritis, neuralgia, spinal cord injury, paralysis, uveitis, arthritides, arthralgias, osteomyelitis, fasciitis, Paget's disease, periodontal disease, synovitis, myasthenia gravis, thyroiditis, systemic lupus erythematosus, Goodpasture's syndrome, Behcets's syndrome, allograft rejection, graft-versus-host disease, ankylosing spondylitis, Berger's disease, Reiter's syndrome, Hodgkin's disease, endometriosis, hemangioma, diseases associated with tissue fibrosis, Raynaud syndrome, Sjogren's syndrome, scleroderma, or fibrosis of liver, lung, heart, kidney, skin, pancreas, or intestine.

213. The method of claim 211, wherein the disease or disorder is non-venular disease.

214. The method of claim 213, wherein the non-venular disease is selected from vessel coronary disease, thrombotic microangiopathy, microangiopathic hemolytic anemia, microvascular occlusion, cutaneous diabetic microangiopathy, Susac's syndrome, cerebral microangiopathy, early diabetic microangiopathy, diabetic microangiopathy, glomerular microangiopathy, non-neoplastic nevus, pulmonary microangiopathy, pulmonary capillaritis, coronary microvascular disease, chronic microvascular diseases, small vessel ischemia, thrombotic thrombocytopenic purpura, endometriosis, arteriolosclerosis, hemangioma, diseases associated with tissue fibrosis, Raynaud syndrome, Sjogren's syndrome, scleroderma, or fibrosis of liver, lung, heart, kidney, skin, pancreas, or intestine.

215. The method of claim 213 or 214, wherein the non-venular disease is hemangioma, endometriosis, diseases associated with tissue fibrosis, Raynaud syndrome, Sjogren's syndrome, scleroderma, or fibrosis of liver, lung, heart, kidney, skin, pancreas, or intestine.

216. The method of any one of claims 211-215, whereby upon administration of the composition, cell, engineered immune cell, or pharmaceutical composition in the subject, more than 50% of the composition, cell, engineered immune cell, or pharmaceutical composition that is retained in the subject within 2 hours following administration is localized in a tumor microenvironment within the subject.

217. The method of claim 216, wherein the tumor microenvironment comprises tumor-associated endothelial cells.

218. The method of any one of claims 211-217, wherein the composition, cell, engineered immune cell, or pharmaceutical composition has a greater binding affinity for the tumor-associated endothelial cells than a binding affinity for normal endothelial cells.

219. The method of any one of claims 211-218, whereby upon administration of the composition, cell, engineered immune cell, or pharmaceutical composition in the subject, more than 5% of the composition, cell, engineered immune cell, or pharmaceutical composition that is retained in the subject within 12 hours following administration is localized in a tumor microenvironment within the subject.

220. The method of any one of claims 210-218, whereby upon administration of the composition, cell, engineered immune cell, or pharmaceutical composition in a subject, more than 50% of the composition, cell, engineered immune cell, or pharmaceutical composition is excreted from the subject within 12 hours following administration.

221. The method of any one of claims 211-220, whereby upon administration of the composition, cell, engineered immune cell, or pharmaceutical composition in a subject, the renal toxicity metrics in the subject within 24 hours following administration remains within 20% of the levels of the renal toxicity metrics prior to administration.

222. The method of claim 221, wherein the renal toxicity metrics are one or more of serum creatinine, glomerular filtration rate, and blood urea nitrogen.

222. A kit comprising the composition of any one of claims 1-122, the cell of any one of claims 123-185, the engineered immune cell of any one of claims 186-205, or the pharmaceutical composition of claim 206-207, and instructions for use.
